SEQUENCE LISTING (677 sequences)

SEQ ID NO: 1 - RNA, 36 nt, synthetic construct
  source 1..36: mol_type = other RNA
  modified_base order(1..3,33,36): mod_base = OTHER; note = 2-Prime-O-methyl nucleotides
  modified_base order(1..3,34..36): mod_base = OTHER; note = Phosphorothioate linkage
  modified_base 34..35: mod_base = OTHER; note = Locked nucleic acid
gagtccgagcagaagaagaagttttagagctatgct

SEQ ID NO: 2 - DNA, 36 bp, synthetic construct
  misc_feature order(1,4,7..9,11,13..16,18,20..36): note = RNA
  source 1..36: mol_type = other DNA
  misc_feature order(2..3,5..6,10,12,17,19): note = DNA
gagtccgagcagaagaagaagttttagaggattgct

SEQ ID NO: 3 - DNA, 66 bp, synthetic construct
  misc_feature order(1..3,51..54,60..63): note = DNA
  source 1..66: mol_type = other DNA
  misc_feature order(4..50,55..59,64..66): note = RNA
agcaatccaagttaaaataaggctagtccgttatcaacttgaaaaagtggcaccgagtcggtgctt

SEQ ID NO: 4 - RNA, 37 nt, synthetic construct
  source 1..37: mol_type = other RNA
  modified_base 35..37: mod_base = OTHER; note = Phosphorothioate linkage
  modified_base 35..36: mod_base = OTHER; note = Locked nucleic acid
  modified_base 1: mod_base = OTHER; note = 2-Prime-Unlocked Nucleic Acid
  modified_base order(34,37): mod_base = OTHER; note = 2-Prime-O-methyl nucleotides
ngagtccgagcagaagaagaagttttagagctatgct

SEQ ID NO: 5 - RNA, 37 nt, synthetic construct
  source 1..37: mol_type = other RNA
  modified_base 35..37: mod_base = OTHER; note = Phosphorothioate linkage
  modified_base 35..36: mod_base = OTHER; note = Locked nucleic acid
  modified_base 2: mod_base = OTHER; note = Unlocked Nucleic Acid
  modified_base order(34,37): mod_base = OTHER; note = 2-Prime-O-methyl nucleotides
gnagtccgagcagaagaagaagttttagagctatgct

SEQ ID NO: 6 - RNA, 37 nt, synthetic construct
  source 1..37: mol_type = other RNA
  modified_base 35..37: mod_base = OTHER; note = Phosphorothioate linkage
  modified_base 35..36: mod_base = OTHER; note = Locked nucleic acid
  modified_base 3: mod_base = OTHER; note = Unlocked Nucleic Acid
  modified_base order(34,37): mod_base = OTHER; note = 2-Prime-O-methyl nucleotides
gangtccgagcagaagaagaagttttagagctatgct

SEQ ID NO: 7 - RNA, 37 nt, synthetic construct
  source 1..37: mol_type = other RNA
  modified_base 35..37: mod_base = OTHER; note = Phosphorothioate linkage
  modified_base 35..36: mod_base = OTHER; note = Locked nucleic acid
  modified_base 4: mod_base = OTHER; note = Unlocked Nucleic Acid
  modified_base order(34,37): mod_base = OTHER; note = 2-Prime-O-methyl nucleotides
gagntccgagcagaagaagaagttttagagctatgct

SEQ ID NO: 8 - RNA, 37 nt, synthetic construct
  source 1..37: mol_type = other RNA
  modified_base 35..37: mod_base = OTHER; note = Phosphorothioate linkage
  modified_base 35..36: mod_base = OTHER; note = Locked nucleic acid
  modified_base 5: mod_base = OTHER; note = Unlocked Nucleic Acid
  modified_base order(34,37): mod_base = OTHER; note = 2-Prime-O-methyl nucleotides
gagtnccgagcagaagaagaagttttagagctatgct

SEQ ID NO: 9 - RNA, 37 nt, synthetic construct
  source 1..37: mol_type = other RNA
  modified_base 35..37: mod_base = OTHER; note = Phosphorothioate linkage
  modified_base 35..36: mod_base = OTHER; note = Locked nucleic acid
  modified_base 6: mod_base = OTHER; note = Unlocked Nucleic Acid
  modified_base order(34,37): mod_base = OTHER; note = 2-Prime-O-methyl nucleotides
gagtcncgagcagaagaagaagttttagagctatgct

SEQ ID NO: 10 - RNA, 37 nt, synthetic construct
  source 1..37: mol_type = other RNA
  modified_base 35..37: mod_base = OTHER; note = Phosphorothioate linkage
  modified_base 35..36: mod_base = OTHER; note = Locked nucleic acid
  modified_base 7: mod_base = OTHER; note = Unlocked Nucleic Acid
  modified_base order(34,37): mod_base = OTHER; note = 2-Prime-O-methyl nucleotides
gagtccngagcagaagaagaagttttagagctatgct

SEQ ID NO: 11 - RNA, 37 nt, synthetic construct
  source 1..37: mol_type = other RNA
  modified_base 35..37: mod_base = OTHER; note = Phosphorothioate linkage
  modified_base 35..36: mod_base = OTHER; note = Locked nucleic acid
  modified_base 8: mod_base = OTHER; note = Unlocked Nucleic Acid
  modified_base order(34,37): mod_base = OTHER; note = 2-Prime-O-methyl nucleotides
gagtccgnagcagaagaagaagttttagagctatgct

SEQ ID NO: 12 - RNA, 37 nt, synthetic construct
  source 1..37: mol_type = other RNA
  modified_base 35..37: mod_base = OTHER; note = Phosphorothioate linkage
  modified_base 35..36: mod_base = OTHER; note = Locked nucleic acid
  modified_base 9: mod_base = OTHER; note = Unlocked Nucleic Acid
  modified_base order(34,37): mod_base = OTHER; note = 2-Prime-O-methyl nucleotides
gagtccgangcagaagaagaagttttagagctatgct

SEQ ID NO: 13 - RNA, 37 nt, synthetic construct
  source 1..37: mol_type = other RNA
  modified_base 35..37: mod_base = OTHER; note = Phosphorothioate linkage
  modified_base 35..36: mod_base = OTHER; note = Locked nucleic acid
  modified_base 10: mod_base = OTHER; note = Unlocked Nucleic Acid
  modified_base order(34,37): mod_base = OTHER; note = 2-Prime-O-methyl nucleotides
gagtccgagncagaagaagaagttttagagctatgct

SEQ ID NO: 14 - RNA, 37 nt, synthetic construct
  source 1..37: mol_type = other RNA
  modified_base 35..37: mod_base = OTHER; note = Phosphorothioate linkage
  modified_base 35..36: mod_base = OTHER; note = Locked nucleic acid
  modified_base 11: mod_base = OTHER; note = Unlocked Nucleic Acid
  modified_base order(34,37): mod_base = OTHER; note = 2-Prime-O-methyl nucleotides
gagtccgagcnagaagaagaagttttagagctatgct

SEQ ID NO: 15 - RNA, 37 nt, synthetic construct
  source 1..37: mol_type = other RNA
  modified_base 35..37: mod_base = OTHER; note = Phosphorothioate linkage
  modified_base 35..36: mod_base = OTHER; note = Locked nucleic acid
  modified_base 12: mod_base = OTHER; note = Unlocked Nucleic Acid
  modified_base order(34,37): mod_base = OTHER; note = 2-Prime-O-methyl nucleotides
gagtccgagcangaagaagaagttttagagctatgct

SEQ ID NO: 16 - RNA, 37 nt, synthetic construct
  source 1..37: mol_type = other RNA
  modified_base 35..37: mod_base = OTHER; note = Phosphorothioate linkage
  modified_base 35..36: mod_base = OTHER; note = Locked nucleic acid
  modified_base 13: mod_base = OTHER; note = Unlocked Nucleic Acid
  modified_base order(34,37): mod_base = OTHER; note = 2-Prime-O-methyl nucleotides
gagtccgagcagnaagaagaagttttagagctatgct

SEQ ID NO: 17 - RNA, 37 nt, synthetic construct
  source 1..37: mol_type = other RNA
  modified_base 35..37: mod_base = OTHER; note = Phosphorothioate linkage
  modified_base 35..36: mod_base = OTHER; note = Locked nucleic acid
  modified_base 14: mod_base = OTHER; note = Unlocked Nucleic Acid
  modified_base order(34,37): mod_base = OTHER; note = 2-Prime-O-methyl nucleotides
gagtccgagcaganagaagaagttttagagctatgct

SEQ ID NO: 18 - RNA, 37 nt, synthetic construct
  source 1..37: mol_type = other RNA
  modified_base 35..37: mod_base = OTHER; note = Phosphorothioate linkage
  modified_base 35..36: mod_base = OTHER; note = Locked nucleic acid
  modified_base 15: mod_base = OTHER; note = Unlocked Nucleic Acid
  modified_base order(34,37): mod_base = OTHER; note = 2-Prime-O-methyl nucleotides
gagtccgagcagaangaagaagttttagagctatgct

SEQ ID NO: 19 - RNA, 37 nt, synthetic construct
  source 1..37: mol_type = other RNA
  modified_base 35..37: mod_base = OTHER; note = Phosphorothioate linkage
  modified_base 35..36: mod_base = OTHER; note = Locked nucleic acid
  modified_base 16: mod_base = OTHER; note = Unlocked Nucleic Acid
  modified_base order(34,37): mod_base = OTHER; note = 2-Prime-O-methyl nucleotides
gagtccgagcagaagnaagaagttttagagctatgct

SEQ ID NO: 20 - RNA, 37 nt, synthetic construct
  source 1..37: mol_type = other RNA
  modified_base 35..37: mod_base = OTHER; note = Phosphorothioate linkage
  modified_base 35..36: mod_base = OTHER; note = Locked nucleic acid
  modified_base 17: mod_base = OTHER; note = Unlocked Nucleic Acid
  modified_base order(34,37): mod_base = OTHER; note = 2-Prime-O-methyl nucleotides
gagtccgagcagaaganagaagttttagagctatgct

SEQ ID NO: 21 - RNA, 37 nt, synthetic construct
  source 1..37: mol_type = other RNA
  modified_base 35..37: mod_base = OTHER; note = Phosphorothioate linkage
  modified_base 35..36: mod_base = OTHER; note = Locked nucleic acid
  modified_base 18: mod_base = OTHER; note = Unlocked Nucleic Acid
  modified_base order(34,37): mod_base = OTHER; note = 2-Prime-O-methyl nucleotides
gagtccgagcagaagaangaagttttagagctatgct

SEQ ID NO: 22 - RNA, 37 nt, synthetic construct
  source 1..37: mol_type = other RNA
  modified_base 35..37: mod_base = OTHER; note = Phosphorothioate linkage
  modified_base 35..36: mod_base = OTHER; note = Locked nucleic acid
  modified_base 19: mod_base = OTHER; note = Unlocked Nucleic Acid
  modified_base order(34,37): mod_base = OTHER; note = 2-Prime-O-methyl nucleotides
gagtccgagcagaagaagnaagttttagagctatgct

SEQ ID NO: 23 - RNA, 37 nt, synthetic construct
  source 1..37: mol_type = other RNA
  modified_base 35..37: mod_base = OTHER; note = Phosphorothioate linkage
  modified_base 35..36: mod_base = OTHER; note = Locked nucleic acid
  modified_base 20: mod_base = OTHER; note = Unlocked Nucleic Acid
  modified_base order(34,37): mod_base = OTHER; note = 2-Prime-O-methyl nucleotides
gagtccgagcagaagaaganagttttagagctatgct

SEQ ID NO: 24 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base order(1..3,33,36): mod_base = OTHER; note = 2-Prime-O-methyl nucleotides
gttggagcatctgagtccaggttttagagctatgct

SEQ ID NO: 25 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base order(1,33,36,2..3): mod_base = OTHER; note = 2-Prime-O-methyl nucleotides
gaaggctgagatcctggagggttttagagctatgct

SEQ ID NO: 26 - RNA, 37 nt, synthetic construct
  source 1..37: mol_type = other RNA
  modified_base order(2..4, 35..37): mod_base = OTHER; note = Phosphorothioate linkage
  modified_base 35..36: mod_base = OTHER; note = Locked nucleic acid
  modified_base 1: mod_base = OTHER; note = 2-Prime-Unlocked Nucleic Acid
  modified_base order(3..4,34,37): mod_base = OTHER; note = 2-Prime-O-methyl nucleotides
ngttggagcatctgagtccaggttttagagctatgct

SEQ ID NO: 27 - RNA, 37 nt, synthetic construct
  source 1..37: mol_type = other RNA
  modified_base order(1^2,3^4, 35..37): mod_base = OTHER; note = Phosphorothioate linkage
  modified_base 35..36: mod_base = OTHER; note = Locked nucleic acid
  modified_base 2: mod_base = OTHER; note = Unlocked Nucleic Acid
  modified_base order(1,4,34,37): mod_base = OTHER; note = 2-Prime-O-methyl nucleotides
gnttggagcatctgagtccaggttttagagctatgct

SEQ ID NO: 28 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 3: mod_base = OTHER; note = Unlocked Nucleic Acid
  modified_base order(1..2,34,37): mod_base = OTHER; note = 2-Prime-O-methyl nucleotides
gtntggagcatctgagtccaggttttagagctatgct

SEQ ID NO: 29 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 4: mod_base = OTHER; note = Unlocked Nucleic Acid
  modified_base order(1..3,34,37): mod_base = OTHER; note = 2-Prime-O-methyl nucleotides
gttnggagcatctgagtccaggttttagagctatgct

SEQ ID NO: 30 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 5: mod_base = OTHER; note = Unlocked Nucleic Acid
  modified_base order(1..3,34,37): mod_base = OTHER; note = 2-Prime-O-methyl nucleotides
gttgngagcatctgagtccaggttttagagctatgct

SEQ ID NO: 31 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 6: mod_base = OTHER; note = Unlocked Nucleic Acid
  modified_base order(1..3,34,37): mod_base = OTHER; note = 2-Prime-O-methyl nucleotides
gttggnagcatctgagtccaggttttagagctatgct

SEQ ID NO: 32 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 7: mod_base = OTHER; note = Unlocked Nucleic Acid
  modified_base order(1..3,34,37): mod_base = OTHER; note = 2-Prime-O-methyl nucleotides
gttggangcatctgagtccaggttttagagctatgct

SEQ ID NO: 33 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 8: mod_base = OTHER; note = Unlocked Nucleic Acid
  modified_base order(1..3,34,37): mod_base = OTHER; note = 2-Prime-O-methyl nucleotides
gttggagncatctgagtccaggttttagagctatgct

SEQ ID NO: 34 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 9: mod_base = OTHER; note = Unlocked Nucleic Acid
  modified_base order(1..3,34,37): mod_base = OTHER; note = 2-Prime-O-methyl nucleotides
gttggagcnatctgagtccaggttttagagctatgct

SEQ ID NO: 35 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 10: mod_base = OTHER; note = Unlocked Nucleic Acid
  modified_base order(1..3,34,37): mod_base = OTHER; note = 2-Prime-O-methyl nucleotides
gttggagcantctgagtccaggttttagagctatgct

SEQ ID NO: 36 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 11: mod_base = OTHER; note = Unlocked Nucleic Acid
  modified_base order(1..3,34,37): mod_base = OTHER; note = 2-Prime-O-methyl nucleotides
gttggagcatnctgagtccaggttttagagctatgct

SEQ ID NO: 37 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 12: mod_base = OTHER; note = Unlocked Nucleic Acid
  modified_base order(1..3,34,37): mod_base = OTHER; note = 2-Prime-O-methyl nucleotides
gttggagcatcntgagtccaggttttagagctatgct

SEQ ID NO: 38 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 13: mod_base = OTHER; note = Unlocked Nucleic Acid
  modified_base order(1..3,34,37): mod_base = OTHER; note = 2-Prime-O-methyl nucleotides
gttggagcatctngagtccaggttttagagctatgct

SEQ ID NO: 39 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 14: mod_base = OTHER; note = Unlocked Nucleic Acid
  modified_base order(1..3,34,37): mod_base = OTHER; note = 2-Prime-O-methyl nucleotides
gttggagcatctgnagtccaggttttagagctatgct

SEQ ID NO: 40 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 15: mod_base = OTHER; note = Unlocked Nucleic Acid
  modified_base order(1..3,34,37): mod_base = OTHER; note = 2-Prime-O-methyl nucleotides
gttggagcatctgangtccaggttttagagctatgct

SEQ ID NO: 41 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 16: mod_base = OTHER; note = Unlocked Nucleic Acid
  modified_base order(1..3,34,37): mod_base = OTHER; note = 2-Prime-O-methyl nucleotides
gttggagcatctgagntccaggttttagagctatgct

SEQ ID NO: 42 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 17: mod_base = OTHER; note = Unlocked Nucleic Acid
  modified_base order(1..3,34,37): mod_base = OTHER; note = 2-Prime-O-methyl nucleotides
gttggagcatctgagtnccaggttttagagctatgct

SEQ ID NO: 43 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 18: mod_base = OTHER; note = Unlocked Nucleic Acid
  modified_base order(1..3,34,37): mod_base = OTHER; note = 2-Prime-O-methyl nucleotides
gttggagcatctgagtcncaggttttagagctatgct

SEQ ID NO: 44 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 19: mod_base = OTHER; note = Unlocked Nucleic Acid
  modified_base order(1..3,34,37): mod_base = OTHER; note = 2-Prime-O-methyl nucleotides
gttggagcatctgagtccnaggttttagagctatgct

SEQ ID NO: 45 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 20: mod_base = OTHER; note = Unlocked Nucleic Acid
  modified_base order(1..3,34,37): mod_base = OTHER; note = 2-Prime-O-methyl nucleotides
gttggagcatctgagtccanggttttagagctatgct

SEQ ID NO: 46 - RNA, 37 nt, synthetic construct
  source 1..37: mol_type = other RNA
  modified_base order(2..4, 35..37): mod_base = OTHER; note = Phosphorothioate linkage
  modified_base 35..36: mod_base = OTHER; note = Locked nucleic acid
  modified_base 1: mod_base = OTHER; note = 2-Prime-Unlocked Nucleic Acid
  modified_base order(34,37,3..4): mod_base = OTHER; note = 2-Prime-O-methyl nucleotides
ngaaggctgagatcctggagggttttagagctatgct

SEQ ID NO: 47 - RNA, 37 nt, synthetic construct
  source 1..37: mol_type = other RNA
  modified_base order(1^2,3^4, 35..37): mod_base = OTHER; note = Phosphorothioate linkage
  modified_base 35..36: mod_base = OTHER; note = Locked nucleic acid
  modified_base 2: mod_base = OTHER; note = Unlocked Nucleic Acid
  modified_base order(1,5,34,37,4): mod_base = OTHER; note = 2-Prime-O-methyl nucleotides
gnaaggctgagatcctggagggttttagagctatgct

SEQ ID NO: 48 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 3: mod_base = OTHER; note = Unlocked Nucleic Acid
  modified_base order(1,34,37,2): mod_base = OTHER; note = 2-Prime-O-methyl nucleotides
ganaggctgagatcctggagggttttagagctatgct

SEQ ID NO: 49 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 4: mod_base = OTHER; note = Unlocked Nucleic Acid
  modified_base order(1,34,37,2..3): mod_base = OTHER; note = 2-Prime-O-methyl nucleotides
gaanggctgagatcctggagggttttagagctatgct

SEQ ID NO: 50 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 5: mod_base = OTHER; note = Unlocked Nucleic Acid
  modified_base order(1,34,37,2..3): mod_base = OTHER; note = 2-Prime-O-methyl nucleotides
gaagngctgagatcctggagggttttagagctatgct

SEQ ID NO: 51 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 6: mod_base = OTHER; note = Unlocked Nucleic Acid
  modified_base order(1,34,37,2..3): mod_base = OTHER; note = 2-Prime-O-methyl nucleotides
gaaggnctgagatcctggagggttttagagctatgct

SEQ ID NO: 52 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 7: mod_base = OTHER; note = Unlocked Nucleic Acid
  modified_base order(1,34,37,2..3): mod_base = OTHER; note = 2-Prime-O-methyl nucleotides
gaaggcntgagatcctggagggttttagagctatgct

SEQ ID NO: 53 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 8: mod_base = OTHER; note = Unlocked Nucleic Acid
  modified_base order(1,34,37,2..3): mod_base = OTHER; note = 2-Prime-O-methyl nucleotides
gaaggctngagatcctggagggttttagagctatgct

SEQ ID NO: 54 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 9: mod_base = OTHER; note = Unlocked Nucleic Acid
  modified_base order(1,34,37,2..3): mod_base = OTHER; note = 2-Prime-O-methyl nucleotides
gaaggctgnagatcctggagggttttagagctatgct

SEQ ID NO: 55 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 10: mod_base = OTHER; note = Unlocked Nucleic Acid
  modified_base order(1,34,37,2..3): mod_base = OTHER; note = 2-Prime-O-methyl nucleotides
gaaggctgangatcctggagggttttagagctatgct

SEQ ID NO: 56 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 11: mod_base = OTHER; note = Unlocked Nucleic Acid
  modified_base order(1,34,37,2..3): mod_base = OTHER; note = 2-Prime-O-methyl nucleotides
gaaggctgagnatcctggagggttttagagctatgct

SEQ ID NO: 57 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 12: mod_base = OTHER; note = Unlocked Nucleic Acid
  modified_base order(1,34,37,2..3): mod_base = OTHER; note = 2-Prime-O-methyl nucleotides
gaaggctgagantcctggagggttttagagctatgct

SEQ ID NO: 58 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 13: mod_base = OTHER; note = Unlocked Nucleic Acid
  modified_base order(1,34,37,2..3): mod_base = OTHER; note = 2-Prime-O-methyl nucleotides
gaaggctgagatncctggagggttttagagctatgct

SEQ ID NO: 59 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 14: mod_base = OTHER; note = Unlocked Nucleic Acid
  modified_base order(1,34,37,2..3): mod_base = OTHER; note = 2-Prime-O-methyl nucleotides
gaaggctgagatcnctggagggttttagagctatgct

SEQ ID NO: 60 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 15: mod_base = OTHER; note = Unlocked Nucleic Acid
  modified_base order(1,34,37,2..3): mod_base = OTHER; note = 2-Prime-O-methyl nucleotides
gaaggctgagatccntggagggttttagagctatgct

SEQ ID NO: 61 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 16: mod_base = OTHER; note = Unlocked Nucleic Acid
  modified_base order(1,34,37,2..3): mod_base = OTHER; note = 2-Prime-O-methyl nucleotides
gaaggctgagatcctnggagggttttagagctatgct

SEQ ID NO: 62 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 17: mod_base = OTHER; note = Unlocked Nucleic Acid
  modified_base order(1,34,37,2..3): mod_base = OTHER; note = 2-Prime-O-methyl nucleotides
gaaggctgagatcctgngagggttttagagctatgct

SEQ ID NO: 63 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 18: mod_base = OTHER; note = Unlocked Nucleic Acid
  modified_base order(1,34,37,2..3): mod_base = OTHER; note = 2-Prime-O-methyl nucleotides
gaaggctgagatcctggnagggttttagagctatgct

SEQ ID NO: 64 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 19: mod_base = OTHER; note = Unlocked Nucleic Acid
  modified_base order(1,34,37,2..3): mod_base = OTHER; note = 2-Prime-O-methyl nucleotides
gaaggctgagatcctggangggttttagagctatgct

SEQ ID NO: 65 - RNA, 37 nt, synthetic construct
  source 1..37: mol_type = other RNA
  modified_base order(1..3, 35..37): mod_base = OTHER; note = Phosphorothioate linkage
  modified_base 35..36: mod_base = OTHER; note = Locked nucleic acid
  modified_base 20: mod_base = OTHER; note = Unlocked Nucleic Acid
  modified_base order(1,34,37,2..3): mod_base = OTHER; note = 2-Prime-O-methyl nucleotides
gaaggctgagatcctggagnggttttagagctatgct

SEQ ID NO: 66 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base order(1,34..35): mod_base = OTHER; note = Locked nucleic acid
  modified_base order(33,36): mod_base = OTHER; note = 2-Prime-O-methyl nucleotides

SEQ ID NO: 67 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base order(2,34..35): mod_base = OTHER; note = Locked nucleic acid
  modified_base order(33,36): mod_base = OTHER; note = 2-Prime-O-methyl nucleotides

SEQ ID NO: 68 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base order(3,34..35): mod_base = OTHER; note = Locked nucleic acid
  modified_base order(33,36): mod_base = OTHER; note = 2-Prime-O-methyl nucleotides

SEQ ID NO: 69 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base order(4,34..35): mod_base = OTHER; note = Locked nucleic acid
  modified_base order(33,36): mod_base = OTHER; note = 2-Prime-O-methyl nucleotides

SEQ ID NO: 70 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base order(5,34..35): mod_base = OTHER; note = Locked nucleic acid
  modified_base order(33,36): mod_base = OTHER; note = 2-Prime-O-methyl nucleotides

SEQ ID NO: 71 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base order(6,34..35): mod_base = OTHER; note = Locked nucleic acid
  modified_base order(33,36): mod_base = OTHER; note = 2-Prime-O-methyl nucleotides

SEQ ID NO: 72 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base order(7,34..35): mod_base = OTHER; note = Locked nucleic acid
  modified_base order(33,36): mod_base = OTHER; note = 2-Prime-O-methyl nucleotides

SEQ ID NO: 73 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base order(8,34..35): mod_base = OTHER; note = Locked nucleic acid
  modified_base order(33,36): mod_base = OTHER; note = 2-Prime-O-methyl nucleotides

SEQ ID NO: 74 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base order(9,34..35): mod_base = OTHER; note = Locked nucleic acid
  modified_base order(33,36): mod_base = OTHER; note = 2-Prime-O-methyl nucleotides

SEQ ID NO: 75 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base order(10,34..35): mod_base = OTHER; note = Locked nucleic acid
  modified_base order(33,36): mod_base = OTHER; note = 2-Prime-O-methyl nucleotides

SEQ ID NO: 76 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base order(11,34..35): mod_base = OTHER; note = Locked nucleic acid
  modified_base order(33,36): mod_base = OTHER; note = 2-Prime-O-methyl nucleotides

SEQ ID NO: 77 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base order(12,34..35): mod_base = OTHER; note = Locked nucleic acid
  modified_base order(33,36): mod_base = OTHER; note = 2-Prime-O-methyl nucleotides

SEQ ID NO: 78 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base order(13,34..35): mod_base = OTHER; note = Locked nucleic acid
  modified_base order(33,36): mod_base = OTHER; note = 2-Prime-O-methyl nucleotides

SEQ ID NO: 79 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base order(14,34..35): mod_base = OTHER; note = Locked nucleic acid
  modified_base order(33,36): mod_base = OTHER; note = 2-Prime-O-methyl nucleotides

SEQ ID NO: 80 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base order(15,34..35): mod_base = OTHER; note = Locked nucleic acid
  modified_base order(33,36): mod_base = OTHER; note = 2-Prime-O-methyl nucleotides

SEQ ID NO: 81 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base order(16,34..35): mod_base = OTHER; note = Locked nucleic acid
  modified_base order(33,36): mod_base = OTHER; note = 2-Prime-O-methyl nucleotides

SEQ ID NO: 82 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base order(17,34..35): mod_base = OTHER; note = Locked nucleic acid
  modified_base order(33,36): mod_base = OTHER; note = 2-Prime-O-methyl nucleotides

SEQ ID NO: 83 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base order(18,34..35): mod_base = OTHER; note = Locked nucleic acid
  modified_base order(33,36): mod_base = OTHER; note = 2-Prime-O-methyl nucleotides

SEQ ID NO: 84 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base order(19,34..35): mod_base = OTHER; note = Locked nucleic acid
  modified_base order(33,36): mod_base = OTHER; note = 2-Prime-O-methyl nucleotides

SEQ ID NO: 85 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base order(20,34..35): mod_base = OTHER; note = Locked nucleic acid
  modified_base order(33,36): mod_base = OTHER; note = 2-Prime-O-methyl nucleotides

SEQ ID NO: 86 - RNA, 36 nt, synthetic construct; identical to SEQ ID NO: 1
  source 1..36: mol_type = other RNA
  modified_base 36: mod_base = OTHER; note = Phosphorothioate linkage
  modified_base 34..35: mod_base = OTHER; note = Locked nucleic acid
  modified_base order(34,36): mod_base = OTHER; note = 2-Prime-O-methyl nucleotides

SEQ ID NO: 87 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 2: mod_base = OTHER; note = 2-Prime-fluoro nucleotides

SEQ ID NO: 88 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 3: mod_base = OTHER; note = 2-Prime-fluoro nucleotides

SEQ ID NO: 89 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 4: mod_base = OTHER; note = 2-Prime-fluoro nucleotides

SEQ ID NO: 90 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 5: mod_base = OTHER; note = 2-Prime-fluoro nucleotides

SEQ ID NO: 91 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 6: mod_base = OTHER; note = 2-Prime-fluoro nucleotides

SEQ ID NO: 92 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 7: mod_base = OTHER; note = 2-Prime-fluoro nucleotides

SEQ ID NO: 93 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 8: mod_base = OTHER; note = 2-Prime-fluoro nucleotides

SEQ ID NO: 94 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 9: mod_base = OTHER; note = 2-Prime-fluoro nucleotides

SEQ ID NO: 95 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 10: mod_base = OTHER; note = 2-Prime-fluoro nucleotides

SEQ ID NO: 96 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 11: mod_base = OTHER; note = 2-Prime-fluoro nucleotides

SEQ ID NO: 97 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 12: mod_base = OTHER; note = 2-Prime-fluoro nucleotides

SEQ ID NO: 98 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 13: mod_base = OTHER; note = 2-Prime-fluoro nucleotides

SEQ ID NO: 99 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 14: mod_base = OTHER; note = 2-Prime-fluoro nucleotides

SEQ ID NO: 100 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 15: mod_base = OTHER; note = 2-Prime-fluoro nucleotides

SEQ ID NO: 101 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 16: mod_base = OTHER; note = 2-Prime-fluoro nucleotides

SEQ ID NO: 102 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 17: mod_base = OTHER; note = 2-Prime-fluoro nucleotides

SEQ ID NO: 103 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 18: mod_base = OTHER; note = 2-Prime-fluoro nucleotides

SEQ ID NO: 104 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 19: mod_base = OTHER; note = 2-Prime-fluoro nucleotides

SEQ ID NO: 105 - RNA, 36 nt, synthetic construct; identical to SEQ ID NO: 1
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 20: mod_base = OTHER; note = 2-Prime-fluoro nucleotides

SEQ ID NO: 106 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base order(1,34..35): mod_base = OTHER; note = Locked nucleic acid
  modified_base order(33,36): mod_base = OTHER; note = 2-Prime-O-methyl nucleotides

SEQ ID NO: 107 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base order(2,34..35): mod_base = OTHER; note = Locked nucleic acid
  modified_base order(33,36): mod_base = OTHER; note = 2-Prime-O-methyl nucleotides

SEQ ID NO: 108 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base order(3,34..35): mod_base = OTHER; note = Locked nucleic acid
  modified_base order(33,36): mod_base = OTHER; note = 2-Prime-O-methyl nucleotides

SEQ ID NO: 109 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base order(4,34..35): mod_base = OTHER; note = Locked nucleic acid
  modified_base order(33,36): mod_base = OTHER; note = 2-Prime-O-methyl nucleotides

SEQ ID NO: 110 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base order(5,34..35): mod_base = OTHER; note = Locked nucleic acid
  modified_base order(33,36): mod_base = OTHER; note = 2-Prime-O-methyl nucleotides

SEQ ID NO: 111 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base order(6,34..35): mod_base = OTHER; note = Locked nucleic acid
  modified_base order(33,36): mod_base = OTHER; note = 2-Prime-O-methyl nucleotides

SEQ ID NO: 112 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base order(7,34..35): mod_base = OTHER; note = Locked nucleic acid
  modified_base order(33,36): mod_base = OTHER; note = 2-Prime-O-methyl nucleotides

SEQ ID NO: 113 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base order(8,34..35): mod_base = OTHER; note = Locked nucleic acid
  modified_base order(33,36): mod_base = OTHER; note = 2-Prime-O-methyl nucleotides

SEQ ID NO: 114 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base order(9,34..35): mod_base = OTHER; note = Locked nucleic acid
  modified_base order(33,36): mod_base = OTHER; note = 2-Prime-O-methyl nucleotides

SEQ ID NO: 115 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base order(10,34..35): mod_base = OTHER; note = Locked nucleic acid
  modified_base order(33,36): mod_base = OTHER; note = 2-Prime-O-methyl nucleotides

SEQ ID NO: 116 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base order(11,34..35): mod_base = OTHER; note = Locked nucleic acid
  modified_base order(33,36): mod_base = OTHER; note = 2-Prime-O-methyl nucleotides

SEQ ID NO: 117 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base order(12,34..35): mod_base = OTHER; note = Locked nucleic acid
  modified_base order(33,36): mod_base = OTHER; note = 2-Prime-O-methyl nucleotides

SEQ ID NO: 118 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base order(13,34..35): mod_base = OTHER; note = Locked nucleic acid
  modified_base order(33,36): mod_base = OTHER; note = 2-Prime-O-methyl nucleotides

SEQ ID NO: 119 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base order(14,34..35): mod_base = OTHER; note = Locked nucleic acid
  modified_base order(33,36): mod_base = OTHER; note = 2-Prime-O-methyl nucleotides

SEQ ID NO: 120 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base order(15,34..35): mod_base = OTHER; note = Locked nucleic acid
  modified_base order(33,36): mod_base = OTHER; note = 2-Prime-O-methyl nucleotides

SEQ ID NO: 121 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base order(16,34..35): mod_base = OTHER; note = Locked nucleic acid
  modified_base order(33,36): mod_base = OTHER; note = 2-Prime-O-methyl nucleotides

SEQ ID NO: 122 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base order(17,34..35): mod_base = OTHER; note = Locked nucleic acid
  modified_base order(33,36): mod_base = OTHER; note = 2-Prime-O-methyl nucleotides

SEQ ID NO: 123 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base order(18,34..35): mod_base = OTHER; note = Locked nucleic acid
  modified_base order(33,36): mod_base = OTHER; note = 2-Prime-O-methyl nucleotides

SEQ ID NO: 124 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base order(19,34..35): mod_base = OTHER; note = Locked nucleic acid
  modified_base order(33,36): mod_base = OTHER; note = 2-Prime-O-methyl nucleotides

SEQ ID NO: 125 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base order(20,34..35): mod_base = OTHER; note = Locked nucleic acid
  modified_base order(33,36): mod_base = OTHER; note = 2-Prime-O-methyl nucleotides

SEQ ID NO: 126 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base order(1,34..35): mod_base = OTHER; note = Locked nucleic acid
  modified_base order(33,36): mod_base = OTHER; note = 2-Prime-O-methyl nucleotides

SEQ ID NO: 127 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base order(2,34..35): mod_base = OTHER; note = Locked nucleic acid
  modified_base order(33,36): mod_base = OTHER; note = 2-Prime-O-methyl nucleotides

SEQ ID NO: 128 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base order(3,34..35): mod_base = OTHER; note = Locked nucleic acid
  modified_base order(33,36): mod_base = OTHER; note = 2-Prime-O-methyl nucleotides

SEQ ID NO: 129 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base order(4,34..35): mod_base = OTHER; note = Locked nucleic acid
  modified_base order(33,36): mod_base = OTHER; note = 2-Prime-O-methyl nucleotides

SEQ ID NO: 130 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base order(5,34..35): mod_base = OTHER; note = Locked nucleic acid
  modified_base order(33,36): mod_base = OTHER; note = 2-Prime-O-methyl nucleotides

SEQ ID NO: 131 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base order(6,34..35): mod_base = OTHER; note = Locked nucleic acid
  modified_base order(33,36): mod_base = OTHER; note = 2-Prime-O-methyl nucleotides

SEQ ID NO: 132 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base order(7,34..35): mod_base = OTHER; note = Locked nucleic acid
  modified_base order(33,36): mod_base = OTHER; note = 2-Prime-O-methyl nucleotides

SEQ ID NO: 133 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base order(8,34..35): mod_base = OTHER; note = Locked nucleic acid
  modified_base order(33,36): mod_base = OTHER; note = 2-Prime-O-methyl nucleotides

SEQ ID NO: 134 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base order(9,34..35): mod_base = OTHER; note = Locked nucleic acid
  modified_base order(33,36): mod_base = OTHER; note = 2-Prime-O-methyl nucleotides

SEQ ID NO: 135 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base order(10,34..35): mod_base = OTHER; note = Locked nucleic acid
  modified_base order(33,36): mod_base = OTHER; note = 2-Prime-O-methyl nucleotides

SEQ ID NO: 136 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base order(11,34..35): mod_base = OTHER; note = Locked nucleic acid
  modified_base order(33,36): mod_base = OTHER; note = 2-Prime-O-methyl nucleotides

SEQ ID NO: 137 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base order(12,34..35): mod_base = OTHER; note = Locked nucleic acid
  modified_base order(33,36): mod_base = OTHER; note = 2-Prime-O-methyl nucleotides

SEQ ID NO: 138 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base order(13,34..35): mod_base = OTHER; note = Locked nucleic acid
  modified_base order(33,36): mod_base = OTHER; note = 2-Prime-O-methyl nucleotides

SEQ ID NO: 139 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base order(14,34..35): mod_base = OTHER; note = Locked nucleic acid
  modified_base order(33,36): mod_base = OTHER; note = 2-Prime-O-methyl nucleotides

SEQ ID NO: 140 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base order(15,34..35): mod_base = OTHER; note = Locked nucleic acid
  modified_base order(33,36): mod_base = OTHER; note = 2-Prime-O-methyl nucleotides

SEQ ID NO: 141 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base order(16,34..35): mod_base = OTHER; note = Locked nucleic acid
  modified_base order(33,36): mod_base = OTHER; note = 2-Prime-O-methyl nucleotides

SEQ ID NO: 142 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base order(17,34..35): mod_base = OTHER; note = Locked nucleic acid
  modified_base order(33,36): mod_base = OTHER; note = 2-Prime-O-methyl nucleotides

SEQ ID NO: 143 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base order(18,34..35): mod_base = OTHER; note = Locked nucleic acid
  modified_base order(33,36): mod_base = OTHER; note = 2-Prime-O-methyl nucleotides

SEQ ID NO: 144 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base order(19,34..35): mod_base = OTHER; note = Locked nucleic acid
  modified_base order(33,36): mod_base = OTHER; note = 2-Prime-O-methyl nucleotides

SEQ ID NO: 145 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base order(20,34..35): mod_base = OTHER; note = Locked nucleic acid
  modified_base order(33,36): mod_base = OTHER; note = 2-Prime-O-methyl nucleotides

SEQ ID NO: 146 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(34,36): mod_base = OTHER; note = 2-Prime-O-methyl nucleotides

SEQ ID NO: 147 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 2: mod_base = OTHER; note = 2-Prime-fluoro nucleotides

SEQ ID NO: 148 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 3: mod_base = OTHER; note = 2-Prime-fluoro nucleotides

SEQ ID NO: 149 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 4: mod_base = OTHER; note = 2-Prime-fluoro nucleotides

SEQ ID NO: 150 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 5: mod_base = OTHER; note = 2-Prime-fluoro nucleotides

SEQ ID NO: 151 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 6: mod_base = OTHER; note = 2-Prime-fluoro nucleotides

SEQ ID NO: 152 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 7: mod_base = OTHER; note = 2-Prime-fluoro nucleotides

SEQ ID NO: 153 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 8: mod_base = OTHER; note = 2-Prime-fluoro nucleotides

SEQ ID NO: 154 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 9: mod_base = OTHER; note = 2-Prime-fluoro nucleotides

SEQ ID NO: 155 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 10: mod_base = OTHER; note = 2-Prime-fluoro nucleotides

SEQ ID NO: 156 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 11: mod_base = OTHER; note = 2-Prime-fluoro nucleotides

SEQ ID NO: 157 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 12: mod_base = OTHER; note = 2-Prime-fluoro nucleotides

SEQ ID NO: 158 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 13: mod_base = OTHER; note = 2-Prime-fluoro nucleotides

SEQ ID NO: 159 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 14: mod_base = OTHER; note = 2-Prime-fluoro nucleotides

SEQ ID NO: 160 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 15: mod_base = OTHER; note = 2-Prime-fluoro nucleotides

SEQ ID NO: 161 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 16: mod_base = OTHER; note = 2-Prime-fluoro nucleotides

SEQ ID NO: 162 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 17: mod_base = OTHER; note = 2-Prime-fluoro nucleotides

SEQ ID NO: 163 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 18: mod_base = OTHER; note = 2-Prime-fluoro nucleotides

SEQ ID NO: 164 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 19: mod_base = OTHER; note = 2-Prime-fluoro nucleotides

SEQ ID NO: 165 - RNA, 36 nt, synthetic construct; identical to SEQ ID NO: 24
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 20: mod_base = OTHER; note = 2-Prime-fluoro nucleotides

SEQ ID NO: 166 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(34,36): mod_base = OTHER; note = 2-Prime-O-methyl nucleotides

SEQ ID NO: 167 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 2: mod_base = OTHER; note = 2-Prime-fluoro nucleotides

SEQ ID NO: 168 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 3: mod_base = OTHER; note = 2-Prime-fluoro nucleotides

SEQ ID NO: 169 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 4: mod_base = OTHER; note = 2-Prime-fluoro nucleotides

SEQ ID NO: 170 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 5: mod_base = OTHER; note = 2-Prime-fluoro nucleotides

SEQ ID NO: 171 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 6: mod_base = OTHER; note = 2-Prime-fluoro nucleotides

SEQ ID NO: 172 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 7: mod_base = OTHER; note = 2-Prime-fluoro nucleotides

SEQ ID NO: 173 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 8: mod_base = OTHER; note = 2-Prime-fluoro nucleotides

SEQ ID NO: 174 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 9: mod_base = OTHER; note = 2-Prime-fluoro nucleotides

SEQ ID NO: 175 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 10: mod_base = OTHER; note = 2-Prime-fluoro nucleotides

SEQ ID NO: 176 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 11: mod_base = OTHER; note = 2-Prime-fluoro nucleotides

SEQ ID NO: 177 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 12: mod_base = OTHER; note = 2-Prime-fluoro nucleotides

SEQ ID NO: 178 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 13: mod_base = OTHER; note = 2-Prime-fluoro nucleotides

SEQ ID NO: 179 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 14: mod_base = OTHER; note = 2-Prime-fluoro nucleotides

SEQ ID NO: 180 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 15: mod_base = OTHER; note = 2-Prime-fluoro nucleotides

SEQ ID NO: 181 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 16: mod_base = OTHER; note = 2-Prime-fluoro nucleotides

SEQ ID NO: 182 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 17: mod_base = OTHER; note = 2-Prime-fluoro nucleotides

SEQ ID NO: 183 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 18: mod_base = OTHER; note = 2-Prime-fluoro nucleotides

SEQ ID NO: 184 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 19: mod_base = OTHER; note = 2-Prime-fluoro nucleotides

SEQ ID NO: 185 - RNA, 36 nt, synthetic construct; identical to SEQ ID NO: 25
  source 1..36: mol_type = other RNA
  modified_base 34..36: mod_base = OTHER; note = Phosphorothioate linkage
  modified_base 34..35: mod_base = OTHER; note = Locked nucleic acid
  modified_base order(33,36): mod_base = OTHER; note = 2-Prime-O-methyl nucleotides
  modified_base 20: mod_base = OTHER; note = 2-Prime-fluoro nucleotides

SEQ ID NO: 186 - RNA, 35 nt, synthetic construct
  source 1..35: mol_type = other RNA
  modified_base order(1^2, 33..35): mod_base = OTHER; note = Phosphorothioate linkage
  modified_base 33..34: mod_base = OTHER; note = Locked nucleic acid
  modified_base order(1..2,32,35): mod_base = OTHER; note = 2-Prime-O-methyl nucleotides
  misc_feature 1: note = U at location 1 is joined to another nucleic acid via a C3 spacer
ttggagcatctgagtccaggttttagagctatgct

SEQ ID NO: 187 - RNA, 34 nt, synthetic construct
  source 1..34: mol_type = other RNA
  modified_base 32..34: mod_base = OTHER; note = Phosphorothioate linkage
  modified_base 32..33: mod_base = OTHER; note = Locked nucleic acid
  modified_base order(1,31,34): mod_base = OTHER; note = 2-Prime-O-methyl nucleotides
  misc_feature 1: note = U at location 1 is joined to another nucleic acid G via a C3 spacer
tggagcatctgagtccaggttttagagctatgct

SEQ ID NO: 188 - RNA, 33 nt, synthetic construct
  source 1..33: mol_type = other RNA
  modified_base 31..33: mod_base = OTHER; note = Phosphorothioate linkage
  modified_base 31..32: mod_base = OTHER; note = Locked nucleic acid
  modified_base order(30,33): mod_base = OTHER; note = 2-Prime-O-methyl nucleotides
  misc_feature 1: note = G at location 1 is joined to another nucleic acid U via a C3 spacer
ggagcatctgagtccaggttttagagctatgct

SEQ ID NO: 189 - RNA, 32 nt, synthetic construct
  source 1..32: mol_type = other RNA
  modified_base 30..32: mod_base = OTHER; note = Phosphorothioate linkage
  modified_base 30..31: mod_base = OTHER; note = Locked nucleic acid
  modified_base order(29,32): mod_base = OTHER; note = 2-Prime-O-methyl nucleotides
  misc_feature 1: note = G at location 1 is joined to another nucleic acid U via a C3 spacerG
gagcatctgagtccaggttttagagctatgct

SEQ ID NO: 190 - RNA, 31 nt, synthetic construct
  source 1..31: mol_type = other RNA
  modified_base 29..31: mod_base = OTHER; note = Phosphorothioate linkage
  modified_base 29..30: mod_base = OTHER; note = Locked nucleic acid
  modified_base order(28,31): mod_base = OTHER; note = 2-Prime-O-methyl nucleotides
  misc_feature 1: note = A at location 1 is joined to another nucleic acid G via a C3 spacer
agcatctgagtccaggttttagagctatgct

SEQ ID NO: 191 - RNA, 30 nt, synthetic construct
  source 1..30: mol_type = other RNA
  modified_base 28..30: mod_base = OTHER; note = Phosphorothioate linkage
  modified_base 28..29: mod_base = OTHER; note = Locked nucleic acid
  modified_base order(27,30): mod_base = OTHER; note = 2-Prime-O-methyl nucleotides
  misc_feature 1: note = G at location 1 is joined to another nucleic acid G via a C3 spacer
gcatctgagtccaggttttagagctatgct

SEQ ID NO: 192 - RNA, 29 nt, synthetic construct
  source 1..29: mol_type = other RNA
  modified_base 27..29: mod_base = OTHER; note = Phosphorothioate linkage
  modified_base 27..28: mod_base = OTHER; note = Locked nucleic acid
  modified_base order(26,29): mod_base = OTHER; note = 2-Prime-O-methyl nucleotides
  misc_feature 1: note = C at location 1 is joined to another nucleic acid A via a C3 spacer
catctgagtccaggttttagagctatgct

SEQ ID NO: 193 - RNA, 28 nt, synthetic construct
  source 1..28: mol_type = other RNA
  modified_base 26..28: mod_base = OTHER; note = Phosphorothioate linkage
  modified_base 26..27: mod_base = OTHER; note = Locked nucleic acid
  modified_base order(25,28): mod_base = OTHER; note = 2-Prime-O-methyl nucleotides
  misc_feature 1: note = A at location 1 is joined to another nucleic acid G via a C3 spacer
atctgagtccaggttttagagctatgct

SEQ ID NO: 194 - RNA, 27 nt, synthetic construct
  source 1..27: mol_type = other RNA
  modified_base 25..27: mod_base = OTHER; note = Phosphorothioate linkage
  modified_base 25..26: mod_base = OTHER; note = Locked nucleic acid
  modified_base order(24,27): mod_base = OTHER; note = 2-Prime-O-methyl nucleotides
  misc_feature 1: note = U at location 1 is joined to another nucleic acid C via a C3 spacer
tctgagtccaggttttagagctatgct

SEQ ID NO: 195 - RNA, 26 nt, synthetic construct
  source 1..26: mol_type = other RNA
  modified_base 24..26: mod_base = OTHER; note = Phosphorothioate linkage
  modified_base 24..25: mod_base = OTHER; note = Locked nucleic acid
  modified_base order(23,26): mod_base = OTHER; note = 2-Prime-O-methyl nucleotides
  misc_feature 1: note = C at location 1 is joined to another nucleic acid A via a C3 spacer
ctgagtccaggttttagagctatgct

SEQ ID NO: 196 - RNA, 10 nt, synthetic construct
  source 1..10: mol_type = other RNA
  modified_base 1..3: mod_base = OTHER; note = Phosphorothioate linkage
  modified_base 1..3: mod_base = OTHER; note = 2-Prime-O-methyl nucleotides
  misc_feature 10: note = U at location 10 is joined to another nucleic acid U via a C3 spacer
gttggagcat

SEQ ID NO: 197 - RNA, 11 nt, synthetic construct
  source 1..11: mol_type = other RNA
  modified_base 1..3: mod_base = OTHER; note = Phosphorothioate linkage
  modified_base 1..3: mod_base = OTHER; note = 2-Prime-O-methyl nucleotides
  misc_feature 11: note = C at location 11 is joined to another nucleic acid G via a C3 spacer
gttggagcatc

SEQ ID NO: 198 - RNA, 12 nt, synthetic construct
  source 1..12: mol_type = other RNA
  modified_base 1..3: mod_base = OTHER; note = Phosphorothioate linkage
  modified_base 1..3: mod_base = OTHER; note = 2-Prime-O-methyl nucleotides
  misc_feature 12: note = U at location 11 is joined to another nucleic acid A via a C3 spacer
gttggagcatct

SEQ ID NO: 199 - RNA, 13 nt, synthetic construct
  source 1..13: mol_type = other RNA
  modified_base 1..3: mod_base = OTHER; note = Phosphorothioate linkage
  modified_base 1..3: mod_base = OTHER; note = 2-Prime-O-methyl nucleotides
  misc_feature 13: note = G at location 13 is joined to another nucleic acid G via a C3 spacer
gttggagcatctg

SEQ ID NO: 200 - RNA, 14 nt, synthetic construct
  source 1..14: mol_type = other RNA
  modified_base 1..3: mod_base = OTHER; note = Phosphorothioate linkage
  modified_base 1..3: mod_base = OTHER; note = 2-Prime-O-methyl nucleotides
  misc_feature 14: note = A at location 14 is joined to another nucleic acid U via a C3 spacer
gttggagcatctga

SEQ ID NO: 201 - RNA, 15 nt, synthetic construct
  source 1..15: mol_type = other RNA
  modified_base 1..3: mod_base = OTHER; note = Phosphorothioate linkage
  modified_base 1..3: mod_base = OTHER; note = 2-Prime-O-methyl nucleotides
  misc_feature 15: note = G at location 15 is joined to another nucleic acid C via a C3 spacer
gttggagcatctgag

SEQ ID NO: 202 - RNA, 16 nt, synthetic construct
  source 1..16: mol_type = other RNA
  modified_base 1..3: mod_base = OTHER; note = Phosphorothioate linkage
  modified_base 1..3: mod_base = OTHER; note = 2-Prime-O-methyl nucleotides
  misc_feature 16: note = U at location 16 is joined to another nucleic acid C via a C3 spacer
gttggagcatctgagt

SEQ ID NO: 203 - RNA, 17 nt, synthetic construct
  source 1..17: mol_type = other RNA
  modified_base 1..3: mod_base = OTHER; note = Phosphorothioate linkage
  modified_base 1..3: mod_base = OTHER; note = 2-Prime-O-methyl nucleotides
  misc_feature 17: note = C at location 17 is joined to another nucleic acid A via a C3 spacer
gttggagcatctgagtc

SEQ ID NO: 204 - RNA, 18 nt, synthetic construct
  source 1..18: mol_type = other RNA
  modified_base 1..3: mod_base = OTHER; note = Phosphorothioate linkage
  modified_base 1..3: mod_base = OTHER; note = 2-Prime-O-methyl nucleotides
  misc_feature 18: note = C at location 18 is joined to another nucleic acid G via a C3 spacer
gttggagcatctgagtcc

SEQ ID NO: 205 - RNA, 19 nt, synthetic construct
  source 1..19: mol_type = other RNA
  modified_base 1..3: mod_base = OTHER; note = Phosphorothioate linkage
  modified_base 1..3: mod_base = OTHER; note = 2-Prime-O-methyl nucleotides
  misc_feature 19: note = A at location 19 is joined to another nucleic acid G via a C3 spacer
gttggagcatctgagtcca

SEQ ID NO: 206 - RNA, 35 nt, synthetic construct
  source 1..35: mol_type = other RNA
  modified_base order(1^2, 33..35): mod_base = OTHER; note = Phosphorothioate linkage
  modified_base 33..34: mod_base = OTHER; note = Locked nucleic acid
  modified_base order(32,35,1..2): mod_base = OTHER; note = 2-Prime-O-methyl nucleotides
  misc_feature 1: note = A at location 1 is joined to another nucleic acid via a C3 spacer
aaggctgagatcctggagggttttagagctatgct

SEQ ID NO: 207 - RNA, 34 nt, synthetic construct
  source 1..34: mol_type = other RNA
  modified_base 32..34: mod_base = OTHER; note = Phosphorothioate linkage
  modified_base 32..33: mod_base = OTHER; note = Locked nucleic acid
  modified_base order(2,31,34,1): mod_base = OTHER; note = 2-Prime-O-methyl nucleotides
  misc_feature 1: note = A at location 1 is joined to another nucleic acid G via a C3 spacer
aggctgagatcctggagggttttagagctatgct

SEQ ID NO: 208 - RNA, 33 nt, synthetic construct
  source 1..33: mol_type = other RNA
  modified_base 31..33: mod_base = OTHER; note = Phosphorothioate linkage
  modified_base 31..32: mod_base = OTHER; note = Locked nucleic acid
  modified_base order(30,33): mod_base = OTHER; note = 2-Prime-O-methyl nucleotides
  misc_feature 1: note = G at location 1 is joined to another nucleic acid A via a C3 spacer
ggctgagatcctggagggttttagagctatgct

SEQ ID NO: 209 - RNA, 32 nt, synthetic construct
  source 1..32: mol_type = other RNA
  modified_base 30..32: mod_base = OTHER; note = Phosphorothioate linkage
  modified_base 30..31: mod_base = OTHER; note = Locked nucleic acid
  modified_base order(29,32): mod_base = OTHER; note = 2-Prime-O-methyl nucleotides
  misc_feature 1: note = G at location 1 is joined to another nucleic acid A via a C3 spacer
gctgagatcctggagggttttagagctatgct

SEQ ID NO: 210 - RNA, 31 nt, synthetic construct
  source 1..31: mol_type = other RNA
  modified_base 29..31: mod_base = OTHER; note = Phosphorothioate linkage
  modified_base 29..30: mod_base = OTHER; note = Locked nucleic acid
  modified_base order(28,31): mod_base = OTHER; note = 2-Prime-O-methyl nucleotides
  misc_feature 1: note = C at location 1 is joined to another nucleic acid G via a C3 spacer
ctgagatcctggagggttttagagctatgct

SEQ ID NO: 211 - RNA, 30 nt, synthetic construct
  source 1..30: mol_type = other RNA
  modified_base 28..30: mod_base = OTHER; note = Phosphorothioate linkage
  modified_base 28..29: mod_base = OTHER; note = Locked nucleic acid
  modified_base order(27,30): mod_base = OTHER; note = 2-Prime-O-methyl nucleotides
  misc_feature 1: note = U at location 1 is joined to another nucleic acid G via a C3 spacer
tgagatcctggagggttttagagctatgct

SEQ ID NO: 212 - RNA, 29 nt, synthetic construct
  source 1..29: mol_type = other RNA
  modified_base 27..29: mod_base = OTHER; note = Phosphorothioate linkage
  modified_base 27..28: mod_base = OTHER; note = Locked nucleic acid
  modified_base order(26,29): mod_base = OTHER; note = 2-Prime-O-methyl nucleotides
  misc_feature 1: note = G at location 1 is joined to another nucleic acid C via a C3 spacer
gagatcctggagggttttagagctatgct

SEQ ID NO: 213 - RNA, 28 nt, synthetic construct
  source 1..28: mol_type = other RNA
  modified_base 26..28: mod_base = OTHER; note = Phosphorothioate linkage
  modified_base 26..27: mod_base = OTHER; note = Locked nucleic acid
  modified_base order(25,28): mod_base = OTHER; note = 2-Prime-O-methyl nucleotides
  misc_feature 1: note = A at location 1 is joined to another nucleic acid U via a C3 spacer
agatcctggagggttttagagctatgct

SEQ ID NO: 214 - RNA, 27 nt, synthetic construct
  source 1..27: mol_type = other RNA
  modified_base 25..27: mod_base = OTHER; note = Phosphorothioate linkage
  modified_base 25..26: mod_base = OTHER; note = Locked nucleic acid
  modified_base order(24,27): mod_base = OTHER; note = 2-Prime-O-methyl nucleotides
  misc_feature 1: note = G at location 1 is joined to another nucleic acid G via a C3 spacer
gatcctggagggttttagagctatgct

SEQ ID NO: 215 - RNA, 26 nt, synthetic construct
  source 1..26: mol_type = other RNA
  modified_base 24..26: mod_base = OTHER; note = Phosphorothioate linkage
  modified_base 24..25: mod_base = OTHER; note = Locked nucleic acid
  modified_base order(23,26): mod_base = OTHER; note = 2-Prime-O-methyl nucleotides
  misc_feature 1: note = A at location 1 is joined to another nucleic acid A via a C3 spacer
atcctggagggttttagagctatgct

SEQ ID NO: 216 - RNA, 10 nt, synthetic construct
  source 1..10: mol_type = other RNA
  modified_base 1..3: mod_base = OTHER; note = Phosphorothioate linkage
  modified_base 1..3: mod_base = OTHER; note = 2-Prime-O-methyl nucleotides
  misc_feature 10: note = G at location 10 is joined to another nucleic acid U via a C3 spacer
gaaggctgag

SEQ ID NO: 217 - RNA, 11 nt, synthetic construct
  source 1..11: mol_type = other RNA
  modified_base 1..3: mod_base = OTHER; note = Phosphorothioate linkage
  modified_base 1..3: mod_base = OTHER; note = 2-Prime-O-methyl nucleotides
  misc_feature 11: note = A at location 11 is joined to another nucleic acid C via a C3 spacer
gaaggctgaga

SEQ ID NO: 218 - RNA, 12 nt, synthetic construct
  source 1..12: mol_type = other RNA
  modified_base 1..3: mod_base = OTHER; note = Phosphorothioate linkage
  modified_base 1..3: mod_base = OTHER; note = 2-Prime-O-methyl nucleotides
  misc_feature 12: note = U at location 12 is joined to another nucleic acid C via a C3 spacer
gaaggctgagat

SEQ ID NO: 219 - RNA, 13 nt, synthetic construct
  source 1..13: mol_type = other RNA
  modified_base 1..3: mod_base = OTHER; note = Phosphorothioate linkage
  modified_base 1..3: mod_base = OTHER; note = 2-Prime-O-methyl nucleotides
  misc_feature 13: note = C at location 13 is joined to another nucleic acid U via a C3 spacer
gaaggctgagatc

SEQ ID NO: 220 - RNA, 14 nt, synthetic construct
  source 1..14: mol_type = other RNA
  modified_base 1..3: mod_base = OTHER; note = Phosphorothioate linkage
  modified_base 1..3: mod_base = OTHER; note = 2-Prime-O-methyl nucleotides
  misc_feature 14: note = C at location 14 is joined to another nucleic acid G via a C3 spacer
gaaggctgagatcc

SEQ ID NO: 221 - RNA, 15 nt, synthetic construct
  source 1..15: mol_type = other RNA
  modified_base 1..3: mod_base = OTHER; note = Phosphorothioate linkage
  modified_base 1..3: mod_base = OTHER; note = 2-Prime-O-methyl nucleotides
  misc_feature 15: note = U at location 15 is joined to another nucleic acid G via a C3 spacer
gaaggctgagatcct

SEQ ID NO: 222 - RNA, 16 nt, synthetic construct
  source 1..16: mol_type = other RNA
  modified_base 1..3: mod_base = OTHER; note = Phosphorothioate linkage
  modified_base 1..3: mod_base = OTHER; note = 2-Prime-O-methyl nucleotides
  misc_feature 16: note = G at location 16 is joined to another nucleic acid A via a C3 spacer
gaaggctgagatcctg

SEQ ID NO: 223 - RNA, 17 nt, synthetic construct
  source 1..17: mol_type = other RNA
  modified_base 1..3: mod_base = OTHER; note = Phosphorothioate linkage
  modified_base 1..3: mod_base = OTHER; note = 2-Prime-O-methyl nucleotides
  misc_feature 17: note = G at location 17 is joined to another nucleic acid G via a C3 spacer
gaaggctgagatcctgg

SEQ ID NO: 224 - RNA, 18 nt, synthetic construct
  source 1..18: mol_type = other RNA
  modified_base 1..3: mod_base = OTHER; note = Phosphorothioate linkage
  modified_base 1..3: mod_base = OTHER; note = 2-Prime-O-methyl nucleotides
  misc_feature 18: note = A at location 18 is joined to another nucleic acid G via a C3 spacer
gaaggctgagatcctgga

SEQ ID NO: 225 - RNA, 19 nt, synthetic construct
  source 1..19: mol_type = other RNA
  modified_base 1..3: mod_base = OTHER; note = Phosphorothioate linkage
  modified_base 1..3: mod_base = OTHER; note = 2-Prime-O-methyl nucleotides
  misc_feature 19: note = G at location 19 is joined to another nucleic acid G via a C3 spacer
gaaggctgagatcctggag

SEQ ID NO: 226 - RNA, 35 nt, synthetic construct
  source 1..35: mol_type = other RNA
  modified_base order(1^2, 33..35): mod_base = OTHER; note = Phosphorothioate linkage
  modified_base 33..34: mod_base = OTHER; note = Locked nucleic acid
  modified_base order(2,32,35,1): mod_base = OTHER; note = 2-Prime-O-methyl nucleotides
  misc_feature 1: note = A at location 1 is joined to another nucleic acid via a C3 spacer
agtccgagcagaagaagaagttttagagctatgct

SEQ ID NO: 227 - RNA, 34 nt, synthetic construct
  source 1..34: mol_type = other RNA
  modified_base 32..34: mod_base = OTHER; note = Phosphorothioate linkage
  modified_base 32..33: mod_base = OTHER; note = Locked nucleic acid
  modified_base order(1,31,34): mod_base = OTHER; note = 2-Prime-O-methyl nucleotides
  misc_feature 1: note = G at location 1 is joined to another nucleic acid G via a C3 spacer
gtccgagcagaagaagaagttttagagctatgct

SEQ ID NO: 228 - RNA, 33 nt, synthetic construct
  source 1..33: mol_type = other RNA
  modified_base 31..33: mod_base = OTHER; note = Phosphorothioate linkage
  modified_base 31..32: mod_base = OTHER; note = Locked nucleic acid
  modified_base order(30,33): mod_base = OTHER; note = 2-Prime-O-methyl nucleotides
  misc_feature 1: note = U at location 1 is joined to another nucleic acid A via a C3 spacer
tccgagcagaagaagaagttttagagctatgct

SEQ ID NO: 229 - RNA, 32 nt, synthetic construct
  source 1..32: mol_type = other RNA
  modified_base 30..32: mod_base = OTHER; note = Phosphorothioate linkage
  modified_base 30..31: mod_base = OTHER; note = Locked nucleic acid
  modified_base order(29,32): mod_base = OTHER; note = 2-Prime-O-methyl nucleotides
  misc_feature 1: note = C at location 1 is joined to another nucleic acid G via a C3 spacer
ccgagcagaagaagaagttttagagctatgct

SEQ ID NO: 230 - RNA, 31 nt, synthetic construct
  source 1..31: mol_type = other RNA
  modified_base 29..31: mod_base = OTHER; note = Phosphorothioate linkage
  modified_base 29..30: mod_base = OTHER; note = Locked nucleic acid
  modified_base order(28,31): mod_base = OTHER; note = 2-Prime-O-methyl nucleotides
  misc_feature 1: note = C at location 1 is joined to another nucleic acid U via a C3 spacer
cgagcagaagaagaagttttagagctatgct

SEQ ID NO: 231 - RNA, 30 nt, synthetic construct
  source 1..30: mol_type = other RNA
  modified_base 28..30: mod_base = OTHER; note = Phosphorothioate linkage
  modified_base 28..29: mod_base = OTHER; note = Locked nucleic acid
  modified_base order(27,30): mod_base = OTHER; note = 2-Prime-O-methyl nucleotides
  misc_feature 1: note = G at location 1 is joined to another nucleic acid C via a C3 spacer
gagcagaagaagaagttttagagctatgct

SEQ ID NO: 232 - RNA, 29 nt, synthetic construct
  source 1..29: mol_type = other RNA
  modified_base 27..29: mod_base = OTHER; note = Phosphorothioate linkage
  modified_base 27..28: mod_base = OTHER; note = Locked nucleic acid
  modified_base order(26,29): mod_base = OTHER; note = 2-Prime-O-methyl nucleotides
  misc_feature 1: note = A at location 1 is joined to another nucleic acid C via a C3 spacer
agcagaagaagaagttttagagctatgct

SEQ ID NO: 233 - RNA, 28 nt, synthetic construct
  source 1..28: mol_type = other RNA
  modified_base 26..28: mod_base = OTHER; note = Phosphorothioate linkage
  modified_base 26..27: mod_base = OTHER; note = Locked nucleic acid
  modified_base order(25,28): mod_base = OTHER; note = 2-Prime-O-methyl nucleotides
  misc_feature 1: note = G at location 1 is joined to another nucleic acid G via a C3 spacer
gcagaagaagaagttttagagctatgct

SEQ ID NO: 234 - RNA, 27 nt, synthetic construct
  source 1..27: mol_type = other RNA
  modified_base 25..27: mod_base = OTHER; note = Phosphorothioate linkage
  modified_base 25..26: mod_base = OTHER; note = Locked nucleic acid
  modified_base order(24,27): mod_base = OTHER; note = 2-Prime-O-methyl nucleotides
  misc_feature 1: note = C at location 1 is joined to another nucleic acid A via a C3 spacer
cagaagaagaagttttagagctatgct

SEQ ID NO: 235 - RNA, 26 nt, synthetic construct
  source 1..26: mol_type = other RNA
  modified_base 24..26: mod_base = OTHER; note = Phosphorothioate linkage
  modified_base 24..25: mod_base = OTHER; note = Locked nucleic acid
  modified_base order(23,26): mod_base = OTHER; note = 2-Prime-O-methyl nucleotides
  misc_feature 1: note = A at location 1 is joined to another nucleic acid G via a C3 spacer
agaagaagaagttttagagctatgct

SEQ ID NO: 236 - RNA, 10 nt, synthetic construct
  source 1..10: mol_type = other RNA
  modified_base 1..3: mod_base = OTHER; note = Phosphorothioate linkage
  modified_base order(1,3,2): mod_base = OTHER; note = 2-Prime-O-methyl nucleotides
  misc_feature 10: note = C at location 10 is joined to another nucleic acid G via a C3 spacer
gagtccgagc

SEQ ID NO: 237 - RNA, 11 nt, synthetic construct
  source 1..11: mol_type = other RNA
  modified_base 1..3: mod_base = OTHER; note = Phosphorothioate linkage
  modified_base order(1,3,2): mod_base = OTHER; note = 2-Prime-O-methyl nucleotides
  misc_feature 11: note = A at location 11 is joined to another nucleic acid A via a C3 spacer
gagtccgagca

SEQ ID NO: 238 - RNA, 12 nt, synthetic construct
  source 1..12: mol_type = other RNA
  modified_base 1..3: mod_base = OTHER; note = Phosphorothioate linkage
  modified_base order(1,3,2): mod_base = OTHER; note = 2-Prime-O-methyl nucleotides
  misc_feature 12: note = G at location 12 is joined to another nucleic acid A via a C3 spacer
gagtccgagcag

SEQ ID NO: 239 - RNA, 13 nt, synthetic construct
  source 1..13: mol_type = other RNA
  modified_base 1..3: mod_base = OTHER; note = Phosphorothioate linkage
  modified_base order(1,3,2): mod_base = OTHER; note = 2-Prime-O-methyl nucleotides
  misc_feature 13: note = A at location 13 is joined to another nucleic acid G via a C3 spacer
gagtccgagcaga

SEQ ID NO: 240 - RNA, 14 nt, synthetic construct
  source 1..14: mol_type = other RNA
  modified_base 1..3: mod_base = OTHER; note = Phosphorothioate linkage
  modified_base order(1,3,2): mod_base = OTHER; note = 2-Prime-O-methyl nucleotides
  misc_feature 14: note = A at location 14 is joined to another nucleic acid A via a C3 spacer
gagtccgagcagaa

SEQ ID NO: 241 - RNA, 15 nt, synthetic construct
  source 1..15: mol_type = other RNA
  modified_base 1..3: mod_base = OTHER; note = Phosphorothioate linkage
  modified_base order(1,3,2): mod_base = OTHER; note = 2-Prime-O-methyl nucleotides
  misc_feature 15: note = G at location 15 is joined to another nucleic acid A via a C3 spacer
gagtccgagcagaag

SEQ ID NO: 242 - RNA, 16 nt, synthetic construct
  source 1..16: mol_type = other RNA
  modified_base 1..3: mod_base = OTHER; note = Phosphorothioate linkage
  modified_base order(1,3,2): mod_base = OTHER; note = 2-Prime-O-methyl nucleotides
  misc_feature 16: note = A at location 16 is joined to another nucleic acid G via a C3 spacer
gagtccgagcagaaga

SEQ ID NO: 243 - RNA, 17 nt, synthetic construct
  source 1..17: mol_type = other RNA
  modified_base 1..3: mod_base = OTHER; note = Phosphorothioate linkage
  modified_base order(1,3,2): mod_base = OTHER; note = 2-Prime-O-methyl nucleotides
  misc_feature 17: note = A at location 17 is joined to another nucleic acid A via a C3 spacer
gagtccgagcagaagaa

SEQ ID NO: 244 - RNA, 18 nt, synthetic construct
  source 1..18: mol_type = other RNA
  modified_base 1..3: mod_base = OTHER; note = Phosphorothioate linkage
  modified_base order(1,3,2): mod_base = OTHER; note = 2-Prime-O-methyl nucleotides
  misc_feature 18: note = G at location 18 is joined to another nucleic acid A via a C3 spacer
gagtccgagcagaagaag

SEQ ID NO: 245 - RNA, 19 nt, synthetic construct
  source 1..19: mol_type = other RNA
  modified_base 1..3: mod_base = OTHER; note = Phosphorothioate linkage
  modified_base order(1,3,2): mod_base = OTHER; note = 2-Prime-O-methyl nucleotides
  misc_feature 19: note = A at location 19 is joined to another nucleic acid G via a C3 spacer
gagtccgagcagaagaaga

SEQ ID NO: 246 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 1: mod_base = OTHER; note = dSpacer (abasic site)
  modified_base order(2..3,33,36): mod_base = OTHER; note = 2-Prime-O-methyl nucleotides
nttggagcatctgagtccaggttttagagctatgct

SEQ ID NO: 247 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 2: mod_base = OTHER; note = dSpacer (abasic site)
  modified_base order(1,3,33,36): mod_base = OTHER; note = 2-Prime-O-methyl nucleotides
gntggagcatctgagtccaggttttagagctatgct

SEQ ID NO: 248 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 3: mod_base = OTHER; note = dSpacer (abasic site)
  modified_base order(1..2,33,36): mod_base = OTHER; note = 2-Prime-O-methyl nucleotides
gtnggagcatctgagtccaggttttagagctatgct

SEQ ID NO: 249 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 4: mod_base = OTHER; note = dSpacer (abasic site)
  modified_base order(1..3,33,36): mod_base = OTHER; note = 2-Prime-O-methyl nucleotides
gttngagcatctgagtccaggttttagagctatgct

SEQ ID NO: 250 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 5: mod_base = OTHER; note = dSpacer (abasic site)
  modified_base order(1..3,33,36): mod_base = OTHER; note = 2-Prime-O-methyl nucleotides
gttgnagcatctgagtccaggttttagagctatgct

SEQ ID NO: 251 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 6: mod_base = OTHER; note = dSpacer (abasic site)
  modified_base order(1..3,33,36): mod_base = OTHER; note = 2-Prime-O-methyl nucleotides
gttggngcatctgagtccaggttttagagctatgct

SEQ ID NO: 252 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 7: mod_base = OTHER; note = dSpacer (abasic site)
  modified_base order(1..3,33,36): mod_base = OTHER; note = 2-Prime-O-methyl nucleotides
gttggancatctgagtccaggttttagagctatgct

SEQ ID NO: 253 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 8: mod_base = OTHER; note = dSpacer (abasic site)
  modified_base order(1..3,33,36): mod_base = OTHER; note = 2-Prime-O-methyl nucleotides
gttggagnatctgagtccaggttttagagctatgct

SEQ ID NO: 254 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 9: mod_base = OTHER; note = dSpacer (abasic site)
  modified_base order(1..3,33,36): mod_base = OTHER; note = 2-Prime-O-methyl nucleotides
gttggagcntctgagtccaggttttagagctatgct

SEQ ID NO: 255 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 10: mod_base = OTHER; note = dSpacer (abasic site)
  modified_base order(1..3,33,36): mod_base = OTHER; note = 2-Prime-O-methyl nucleotides
gttggagcanctgagtccaggttttagagctatgct

SEQ ID NO: 256 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 11: mod_base = OTHER; note = dSpacer (abasic site)
  modified_base order(1..3,33,36): mod_base = OTHER; note = 2-Prime-O-methyl nucleotides
gttggagcatntgagtccaggttttagagctatgct

SEQ ID NO: 257 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 12: mod_base = OTHER; note = dSpacer (abasic site)
  modified_base order(1..3,33,36): mod_base = OTHER; note = 2-Prime-O-methyl nucleotides
gttggagcatcngagtccaggttttagagctatgct

SEQ ID NO: 258 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 13: mod_base = OTHER; note = dSpacer (abasic site)
  modified_base order(1..3,33,36): mod_base = OTHER; note = 2-Prime-O-methyl nucleotides
gttggagcatctnagtccaggttttagagctatgct

SEQ ID NO: 259 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 14: mod_base = OTHER; note = dSpacer (abasic site)
  modified_base order(1..3,33,36): mod_base = OTHER; note = 2-Prime-O-methyl nucleotides
gttggagcatctgngtccaggttttagagctatgct

SEQ ID NO: 260 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 15: mod_base = OTHER; note = dSpacer (abasic site)
  modified_base order(1..3,33,36): mod_base = OTHER; note = 2-Prime-O-methyl nucleotides
gttggagcatctgantccaggttttagagctatgct

SEQ ID NO: 261 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 16: mod_base = OTHER; note = dSpacer (abasic site)
  modified_base order(1..3,33,36): mod_base = OTHER; note = 2-Prime-O-methyl nucleotides
gttggagcatctgagnccaggttttagagctatgct

SEQ ID NO: 262 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 17: mod_base = OTHER; note = dSpacer (abasic site)
  modified_base order(1..3,33,36): mod_base = OTHER; note = 2-Prime-O-methyl nucleotides
gttggagcatctgagtncaggttttagagctatgct

SEQ ID NO: 263 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 18: mod_base = OTHER; note = dSpacer (abasic site)
  modified_base order(1..3,33,36): mod_base = OTHER; note = 2-Prime-O-methyl nucleotides
gttggagcatctgagtcnaggttttagagctatgct

SEQ ID NO: 264 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 19: mod_base = OTHER; note = dSpacer (abasic site)
  modified_base order(1..3,33,36): mod_base = OTHER; note = 2-Prime-O-methyl nucleotides
gttggagcatctgagtccnggttttagagctatgct

SEQ ID NO: 265 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 20: mod_base = OTHER; note = dSpacer (abasic site)
  modified_base order(1..3,33,36): mod_base = OTHER; note = 2-Prime-O-methyl nucleotides
gttggagcatctgagtccangttttagagctatgct

SEQ ID NO: 266 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 1: mod_base = OTHER; note = dSpacer (abasic site)
  modified_base order(33,36,2..3): mod_base = OTHER; note = 2-Prime-O-methyl nucleotides
naaggctgagatcctggagggttttagagctatgct

SEQ ID NO: 267 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 2: mod_base = OTHER; note = dSpacer (abasic site)
  modified_base order(1,4,33,36,3): mod_base = OTHER; note = 2-Prime-O-methyl nucleotides
gnaggctgagatcctggagggttttagagctatgct

SEQ ID NO: 268 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 3: mod_base = OTHER; note = dSpacer (abasic site)
  modified_base order(1,33,36,2): mod_base = OTHER; note = 2-Prime-O-methyl nucleotides
ganggctgagatcctggagggttttagagctatgct

SEQ ID NO: 269 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 4: mod_base = OTHER; note = dSpacer (abasic site)
  modified_base order(1,33,36,2..3): mod_base = OTHER; note = 2-Prime-O-methyl nucleotides
gaangctgagatcctggagggttttagagctatgct

SEQ ID NO: 270 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 5: mod_base = OTHER; note = dSpacer (abasic site)
  modified_base order(1,33,36,2..3): mod_base = OTHER; note = 2-Prime-O-methyl nucleotides
gaagnctgagatcctggagggttttagagctatgct

SEQ ID NO: 271 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 6: mod_base = OTHER; note = dSpacer (abasic site)
  modified_base order(1,33,36,2..3): mod_base = OTHER; note = 2-Prime-O-methyl nucleotides
gaaggntgagatcctggagggttttagagctatgct

SEQ ID NO: 272 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 7: mod_base = OTHER; note = dSpacer (abasic site)
  modified_base order(1,33,36,2..3): mod_base = OTHER; note = 2-Prime-O-methyl nucleotides
gaaggcngagatcctggagggttttagagctatgct

SEQ ID NO: 273 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 8: mod_base = OTHER; note = dSpacer (abasic site)
  modified_base order(1,33,36,2..3): mod_base = OTHER; note = 2-Prime-O-methyl nucleotides
gaaggctnagatcctggagggttttagagctatgct

SEQ ID NO: 274 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 9: mod_base = OTHER; note = dSpacer (abasic site)
  modified_base order(1,33,36,2..3): mod_base = OTHER; note = 2-Prime-O-methyl nucleotides
gaaggctgngatcctggagggttttagagctatgct

SEQ ID NO: 275 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 10: mod_base = OTHER; note = dSpacer (abasic site)
  modified_base order(1,33,36,2..3): mod_base = OTHER; note = 2-Prime-O-methyl nucleotides
gaaggctganatcctggagggttttagagctatgct

SEQ ID NO: 276 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 11: mod_base = OTHER; note = dSpacer (abasic site)
  modified_base order(1,33,36,2..3): mod_base = OTHER; note = 2-Prime-O-methyl nucleotides
gaaggctgagntcctggagggttttagagctatgct

SEQ ID NO: 277 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 12: mod_base = OTHER; note = dSpacer (abasic site)
  modified_base order(1,33,36,2..3): mod_base = OTHER; note = 2-Prime-O-methyl nucleotides
gaaggctgagancctggagggttttagagctatgct

SEQ ID NO: 278 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 13: mod_base = OTHER; note = dSpacer (abasic site)
  modified_base order(1,33,36,2..3): mod_base = OTHER; note = 2-Prime-O-methyl nucleotides
gaaggctgagatnctggagggttttagagctatgct

SEQ ID NO: 279 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 14: mod_base = OTHER; note = dSpacer (abasic site)
  modified_base order(1,33,36,2..3): mod_base = OTHER; note = 2-Prime-O-methyl nucleotides
gaaggctgagatcntggagggttttagagctatgct

SEQ ID NO: 280 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 15: mod_base = OTHER; note = dSpacer (abasic site)
  modified_base order(1,33,36,2..3): mod_base = OTHER; note = 2-Prime-O-methyl nucleotides
gaaggctgagatccnggagggttttagagctatgct

SEQ ID NO: 281 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 16: mod_base = OTHER; note = dSpacer (abasic site)
  modified_base order(1,33,36,2..3): mod_base = OTHER; note = 2-Prime-O-methyl nucleotides
gaaggctgagatcctngagggttttagagctatgct

SEQ ID NO: 282 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 17: mod_base = OTHER; note = dSpacer (abasic site)
  modified_base order(1,33,36,2..3): mod_base = OTHER; note = 2-Prime-O-methyl nucleotides
gaaggctgagatcctgnagggttttagagctatgct

SEQ ID NO: 283 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 18: mod_base = OTHER; note = dSpacer (abasic site)
  modified_base order(1,33,36,2..3): mod_base = OTHER; note = 2-Prime-O-methyl nucleotides
gaaggctgagatcctggngggttttagagctatgct

SEQ ID NO: 284 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 19: mod_base = OTHER; note = dSpacer (abasic site)
  modified_base order(1,33,36,2..3): mod_base = OTHER; note = 2-Prime-O-methyl nucleotides
gaaggctgagatcctgganggttttagagctatgct

SEQ ID NO: 285 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 20: mod_base = OTHER; note = dSpacer (abasic site)
  modified_base order(1,33,36,2..3): mod_base = OTHER; note = 2-Prime-O-methyl nucleotides
gaaggctgagatcctggagngttttagagctatgct

SEQ ID NO: 286 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 1: mod_base = OTHER; note = dSpacer (abasic site)
  modified_base order(3,33,36,2): mod_base = OTHER; note = 2-Prime-O-methyl nucleotides
nagtccgagcagaagaagaagttttagagctatgct

SEQ ID NO: 287 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 2: mod_base = OTHER; note = dSpacer (abasic site)
  modified_base order(1,3,33,36): mod_base = OTHER; note = 2-Prime-O-methyl nucleotides
gngtccgagcagaagaagaagttttagagctatgct

SEQ ID NO: 288 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 3: mod_base = OTHER; note = dSpacer (abasic site)
  modified_base order(1,33,36,2): mod_base = OTHER; note = 2-Prime-O-methyl nucleotides
gantccgagcagaagaagaagttttagagctatgct

SEQ ID NO: 289 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 4: mod_base = OTHER; note = dSpacer (abasic site)
  modified_base order(1,3,33,36,2): mod_base = OTHER; note = 2-Prime-O-methyl nucleotides
gagnccgagcagaagaagaagttttagagctatgct

SEQ ID NO: 290 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 5: mod_base = OTHER; note = dSpacer (abasic site)
  modified_base order(1,3,33,36,2): mod_base = OTHER; note = 2-Prime-O-methyl nucleotides
gagtncgagcagaagaagaagttttagagctatgct

SEQ ID NO: 291 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 6: mod_base = OTHER; note = dSpacer (abasic site)
  modified_base order(1,3,33,36,2): mod_base = OTHER; note = 2-Prime-O-methyl nucleotides
gagtcngagcagaagaagaagttttagagctatgct

SEQ ID NO: 292 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 7: mod_base = OTHER; note = dSpacer (abasic site)
  modified_base order(1,3,33,36,2): mod_base = OTHER; note = 2-Prime-O-methyl nucleotides
gagtccnagcagaagaagaagttttagagctatgct

SEQ ID NO: 293 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 8: mod_base = OTHER; note = dSpacer (abasic site)
  modified_base order(1,3,33,36,2): mod_base = OTHER; note = 2-Prime-O-methyl nucleotides
gagtccgngcagaagaagaagttttagagctatgct

SEQ ID NO: 294 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 9: mod_base = OTHER; note = dSpacer (abasic site)
  modified_base order(1,3,33,36,2): mod_base = OTHER; note = 2-Prime-O-methyl nucleotides
gagtccgancagaagaagaagttttagagctatgct

SEQ ID NO: 295 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 10: mod_base = OTHER; note = dSpacer (abasic site)
  modified_base order(1,3,33,36,2): mod_base = OTHER; note = 2-Prime-O-methyl nucleotides
gagtccgagnagaagaagaagttttagagctatgct

SEQ ID NO: 296 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 11: mod_base = OTHER; note = dSpacer (abasic site)
  modified_base order(1,3,33,36,2): mod_base = OTHER; note = 2-Prime-O-methyl nucleotides
gagtccgagcngaagaagaagttttagagctatgct

SEQ ID NO: 297 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 12: mod_base = OTHER; note = dSpacer (abasic site)
  modified_base order(1,3,33,36,2): mod_base = OTHER; note = 2-Prime-O-methyl nucleotides
gagtccgagcanaagaagaagttttagagctatgct

SEQ ID NO: 298 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 13: mod_base = OTHER; note = dSpacer (abasic site)
  modified_base order(1,3,33,36,2): mod_base = OTHER; note = 2-Prime-O-methyl nucleotides
gagtccgagcagnagaagaagttttagagctatgct

SEQ ID NO: 299 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 14: mod_base = OTHER; note = dSpacer (abasic site)
  modified_base order(1,3,33,36,2): mod_base = OTHER; note = 2-Prime-O-methyl nucleotides
gagtccgagcagangaagaagttttagagctatgct

SEQ ID NO: 300 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 15: mod_base = OTHER; note = dSpacer (abasic site)
  modified_base order(1,3,33,36,2): mod_base = OTHER; note = 2-Prime-O-methyl nucleotides
gagtccgagcagaanaagaagttttagagctatgct

SEQ ID NO: 301 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 16: mod_base = OTHER; note = dSpacer (abasic site)
  modified_base order(1,3,33,36,2): mod_base = OTHER; note = 2-Prime-O-methyl nucleotides
gagtccgagcagaagnagaagttttagagctatgct

SEQ ID NO: 302 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 17: mod_base = OTHER; note = dSpacer (abasic site)
  modified_base order(1,3,33,36,2): mod_base = OTHER; note = 2-Prime-O-methyl nucleotides
gagtccgagcagaagangaagttttagagctatgct

SEQ ID NO: 303 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 18: mod_base = OTHER; note = dSpacer (abasic site)
  modified_base order(1,3,33,36,2): mod_base = OTHER; note = 2-Prime-O-methyl nucleotides
gagtccgagcagaagaanaagttttagagctatgct

SEQ ID NO: 304 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 19: mod_base = OTHER; note = dSpacer (abasic site)
  modified_base order(1,3,33,36,2): mod_base = OTHER; note = 2-Prime-O-methyl nucleotides
gagtccgagcagaagaagnagttttagagctatgct

SEQ ID NO: 305 - RNA, 36 nt, synthetic construct
  source 1..36: mol_type = other RNA
  modified_base order(1..3, 34..36): mod_base = OTHER; note = Phosphorothioate linkage
  modified_base 34..35: mod_base = OTHER; note = Locked nucleic acid
  modified_base 20: mod_base = OTHER; note = dSpacer (abasic site)
  modified_base order(1,3,33,36,2): mod_base = OTHER; note = 2-Prime-O-methyl nucleotides
gagtccgagcagaagaagangttttagagctatgct

SEQ ID NO: 306 - RNA, 36 nt, synthetic construct; identical to SEQ ID NO: 1
  source 1..36: mol_type = other RNA

SEQ ID NO: 307 - RNA, 36 nt, synthetic construct
  source 1..36: mol_type = other RNA
ggggccactagggacaggatgttttagagctatgct

SEQ ID NO: 308 - RNA, 37 nt, synthetic construct
  source 1..37: mol_type = other RNA
  modified_base 3: mod_base = OTHER; note = Unlocked nucleic acid
ggnggccactagggacaggatgttttagagctatgct

SEQ ID NO: 309-334 - skipped (26 entries)

SEQ ID NO: 335 - DNA, 20 bp, synthetic construct
  source 1..20: mol_type = other DNA
gagtccgagcagaagaagaa

SEQ ID NO: 336 - DNA, 20 bp, synthetic construct
  source 1..20: mol_type = other DNA
gagtctaagcagaagaagaa

SEQ ID NO: 337 - DNA, 20 bp, synthetic construct
  source 1..20: mol_type = other DNA
gagttagagcagaagaagaa

SEQ ID NO: 338 - DNA, 20 bp, synthetic construct
  source 1..20: mol_type = other DNA
aagtctgagcacaagaagaa

SEQ ID NO: 339 - DNA, 20 bp, synthetic construct
  source 1..20: mol_type = other DNA
gttggagcatctgagtccag

SEQ ID NO: 340 - DNA, 20 bp, synthetic construct
  source 1..20: mol_type = other DNA
aatggggcatctgagtccat

SEQ ID NO: 341 - DNA, 20 bp, synthetic construct
  source 1..20: mol_type = other DNA
gttggagaaactgagtccag

SEQ ID NO: 342 - DNA, 20 bp, synthetic construct
  source 1..20: mol_type = other DNA
gatggagcaaccgagtccag

SEQ ID NO: 343 - DNA, 20 bp, synthetic construct
  source 1..20: mol_type = other DNA
gaaggctgagatcctggagg

SEQ ID NO: 344 - DNA, 20 bp, synthetic construct
  source 1..20: mol_type = other DNA
taaggcagagaccctagagg

SEQ ID NO: 345 - DNA, 20 bp, synthetic construct
  source 1..20: mol_type = other DNA
aagggctgagttcctggagg

SEQ ID NO: 346 - DNA, 20 bp, synthetic construct
  source 1..20: mol_type = other DNA
agaggctgagaacctggaga

SEQ ID NO: 347 - DNA, 20 bp, synthetic construct
  source 1..20: mol_type = other DNA
ggggccactagggacaggat

SEQ ID NO: 348 - DNA, 20 bp, synthetic construct
  source 1..20: mol_type = other DNA
gggaccatcagggacaggat

SEQ ID NO: 349 - DNA, 20 bp, synthetic construct
  source 1..20: mol_type = other DNA
ggggccaattaggacaggat

SEQ ID NO: 350-351 - skipped (2 entries)

SEQ ID NO: 352 - RNA, 20 nt, synthetic construct
  source 1..20: mol_type = other RNA
gagcagggctggggagaagg

SEQ ID NO: 353 - RNA, 20 nt, synthetic construct
  source 1..20: mol_type = other RNA
gaaggctgagatcctggagg

SEQ ID NO: 354 - RNA, 20 nt, synthetic construct
  source 1..20: mol_type = other RNA
gctgcagaagggattccatg

SEQ ID NO: 355 - RNA, 20 nt, synthetic construct
  source 1..20: mol_type = other RNA
tgtgctagacatgaggtcta

SEQ ID NO: 356 - RNA, 20 nt, synthetic construct
  source 1..20: mol_type = other RNA
gagtccgagcagaagaagaa

SEQ ID NO: 357 - RNA, 20 nt, synthetic construct
  source 1..20: mol_type = other RNA
cttgccccacagggcagtaa

SEQ ID NO: 358 - RNA, 20 nt, synthetic construct
  source 1..20: mol_type = other RNA
gttggagcatctgagtccag

SEQ ID NO: 359 - RNA, 20 nt, synthetic construct
  source 1..20: mol_type = other RNA
ggcccagactgagcacgtga

SEQ ID NO: 360 - RNA, 20 nt, synthetic construct
  source 1..20: mol_type = other RNA
aattatggggattactagga

SEQ ID NO: 361 - RNA, 20 nt, synthetic construct
  source 1..20: mol_type = other RNA
ggcgccctggccagtcgtct

SEQ ID NO: 362 - RNA, 20 nt, synthetic construct
  source 1..20: mol_type = other RNA
cttaccccacttaactatct

SEQ ID NO: 363 - RNA, 20 nt, synthetic construct
  source 1..20: mol_type = other RNA
cccgcaccttggcgcagcgg

SEQ ID NO: 364 - RNA, 20 nt, synthetic construct
  source 1..20: mol_type = other RNA
cggtcaagatggaccagcac

SEQ ID NO: 365 - RNA, 20 nt, synthetic construct
  source 1..20: mol_type = other RNA
ttgggacaacgttgtccagc

SEQ ID NO: 366 - RNA, 20 nt, synthetic construct
  source 1..20: mol_type = other RNA
ctacgaggagcatttgcact

SEQ ID NO: 367 - RNA, 20 nt, synthetic construct
  source 1..20: mol_type = other RNA
gcggaattgacaagttccga

SEQ ID NO: 368 - RNA, 36 nt, synthetic construct
  source 1..36: mol_type = other RNA
cccgcaccttggcgcagcgggttttagagctatgct

SEQ ID NO: 369 - RNA, 37 nt, synthetic construct
  source 1..37: mol_type = other RNA
  modified_base 1: mod_base = OTHER; note = Unlocked nucleic acid
ncccgcaccttggcgcagcgggttttagagctatgct

SEQ ID NO: 370 - RNA, 37 nt, synthetic construct
  source 1..37: mol_type = other RNA
  modified_base 2: mod_base = OTHER; note = Unlocked nucleic acid
cnccgcaccttggcgcagcgggttttagagctatgct

SEQ ID NO: 371 - RNA, 37 nt, synthetic construct
  source 1..37: mol_type = other RNA
  modified_base 3: mod_base = OTHER; note = Unlocked nucleic acid
ccncgcaccttggcgcagcgggttttagagctatgct

SEQ ID NO: 372 - RNA, 37 nt, synthetic construct
  source 1..37: mol_type = other RNA
  modified_base 4: mod_base = OTHER; note = Unlocked nucleic acid
cccngcaccttggcgcagcgggttttagagctatgct

SEQ ID NO: 373 - RNA, 37 nt, synthetic construct
  source 1..37: mol_type = other RNA
  modified_base 5: mod_base = OTHER; note = Unlocked nucleic acid
cccgncaccttggcgcagcgggttttagagctatgct

SEQ ID NO: 374 - RNA, 37 nt, synthetic construct
  source 1..37: mol_type = other RNA
  modified_base 6: mod_base = OTHER; note = Unlocked nucleic acid
cccgcnaccttggcgcagcgggttttagagctatgct

SEQ ID NO: 375 - RNA, 37 nt, synthetic construct
  source 1..37: mol_type = other RNA
  modified_base 7: mod_base = OTHER; note = Unlocked nucleic acid
cccgcanccttggcgcagcgggttttagagctatgct

SEQ ID NO: 376 - RNA, 37 nt, synthetic construct
  source 1..37: mol_type = other RNA
  modified_base 8: mod_base = OTHER; note = Unlocked nucleic acid
cccgcacncttggcgcagcgggttttagagctatgct

SEQ ID NO: 377 - RNA, 37 nt, synthetic construct
  source 1..37: mol_type = other RNA
  modified_base 9: mod_base = OTHER; note = Unlocked nucleic acid
cccgcaccnttggcgcagcgggttttagagctatgct

SEQ ID NO: 378 - RNA, 37 nt, synthetic construct
  source 1..37: mol_type = other RNA
  modified_base 10: mod_base = OTHER; note = Unlocked nucleic acid
cccgcacctntggcgcagcgggttttagagctatgct

SEQ ID NO: 379 - RNA, 37 nt, synthetic construct
  source 1..37: mol_type = other RNA
  modified_base 11: mod_base = OTHER; note = Unlocked nucleic acid
cccgcaccttnggcgcagcgggttttagagctatgct

SEQ ID NO: 380 - RNA, 37 nt, synthetic construct
  source 1..37: mol_type = other RNA
  modified_base 12: mod_base = OTHER; note = Unlocked nucleic acid
cccgcaccttgngcgcagcgggttttagagctatgct

SEQ ID NO: 381 - RNA, 37 nt, synthetic construct
  source 1..37: mol_type = other RNA
  modified_base 13: mod_base = OTHER; note = Unlocked nucleic acid
cccgcaccttggncgcagcgggttttagagctatgct

SEQ ID NO: 382 - RNA, 37 nt, synthetic construct
  source 1..37: mol_type = other RNA
  modified_base 14: mod_base = OTHER; note = Unlocked nucleic acid
cccgcaccttggcngcagcgggttttagagctatgct

SEQ ID NO: 383 - RNA, 37 nt, synthetic construct
  source 1..37: mol_type = other RNA
  modified_base 15: mod_base = OTHER; note = Unlocked nucleic acid
cccgcaccttggcgncagcgggttttagagctatgct

SEQ ID NO: 384 - RNA, 37 nt, synthetic construct
  source 1..37: mol_type = other RNA
  modified_base 16: mod_base = OTHER; note = Unlocked nucleic acid
cccgcaccttggcgcnagcgggttttagagctatgct

SEQ ID NO: 385 - RNA, 37 nt, synthetic construct
  source 1..37: mol_type = other RNA
  modified_base 17: mod_base = OTHER; note = Unlocked nucleic acid
cccgcaccttggcgcangcgggttttagagctatgct

SEQ ID NO: 386 - RNA, 37 nt, synthetic construct
  source 1..37: mol_type = other RNA
  modified_base 18: mod_base = OTHER; note = Unlocked nucleic acid
cccgcaccttggcgcagncgggttttagagctatgct

SEQ ID NO: 387 - RNA, 37 nt, synthetic construct
  source 1..37: mol_type = other RNA
  modified_base 19: mod_base = OTHER; note = Unlocked nucleic acid
cccgcaccttggcgcagcngggttttagagctatgct

SEQ ID NO: 388 - RNA, 37 nt, synthetic construct
  source 1..37: mol_type = other RNA
  modified_base 20: mod_base = OTHER; note = Unlocked nucleic acid
cccgcaccttggcgcagcgnggttttagagctatgct

SEQ ID NO: 389 - RNA, 36 nt, synthetic construct
  source 1..36: mol_type = other RNA
gcggaattgacaagttccgagttttagagctatgct

SEQ ID NO: 390 - RNA, 37 nt, synthetic construct
  source 1..37: mol_type = other RNA
  modified_base 1: mod_base = OTHER; note = Unlocked nucleic acid
ngcggaattgacaagttccgagttttagagctatgct

SEQ ID NO: 391 - RNA, 37 nt, synthetic construct
  source 1..37: mol_type = other RNA
  modified_base 2: mod_base = OTHER; note = Unlocked nucleic acid
gncggaattgacaagttccgagttttagagctatgct

SEQ ID NO: 392 - RNA, 37 nt, synthetic construct
  source 1..37: mol_type = other RNA
  modified_base 3: mod_base = OTHER; note = Unlocked nucleic acid
gcnggaattgacaagttccgagttttagagctatgct

SEQ ID NO: 393 - RNA, 37 nt, synthetic construct
  source 1..37: mol_type = other RNA
  modified_base 4: mod_base = OTHER; note = Unlocked nucleic acid
gcgngaattgacaagttccgagttttagagctatgct

SEQ ID NO: 394 - RNA, 37 nt, synthetic construct
  source 1..37: mol_type = other RNA
  modified_base 5: mod_base = OTHER; note = Unlocked nucleic acid
gcggnaattgacaagttccgagttttagagctatgct

SEQ ID NO: 395 - RNA, 37 nt, synthetic construct
  source 1..37: mol_type = other RNA
  modified_base 6: mod_base = OTHER; note = Unlocked nucleic acid
gcgganattgacaagttccgagttttagagctatgct

SEQ ID NO: 396 - RNA, 37 nt, synthetic construct
  source 1..37: mol_type = other RNA
  modified_base 7: mod_base = OTHER; note = Unlocked nucleic acid
gcggaanttgacaagttccgagttttagagctatgct

SEQ ID NO: 397 - RNA, 37 nt, synthetic construct
  source 1..37: mol_type = other RNA
  modified_base 8: mod_base = OTHER; note = Unlocked nucleic acid
gcggaatntgacaagttccgagttttagagctatgct

SEQ ID NO: 398 - RNA, 37 nt, synthetic construct
  source 1..37: mol_type = other RNA
  modified_base 9: mod_base = OTHER; note = Unlocked nucleic acid
gcggaattngacaagttccgagttttagagctatgct

SEQ ID NO: 399 - RNA, 37 nt, synthetic construct
  source 1..37: mol_type = other RNA
  modified_base 10: mod_base = OTHER; note = Unlocked nucleic acid
gcggaattgnacaagttccgagttttagagctatgct

SEQ ID NO: 400 - RNA, 36 nt, synthetic construct
  source 1..36: mol_type = other RNA
ggcgccctggccagtcgtctgttttagagctatgct

SEQ ID NO: 401 - RNA, 37 nt, synthetic construct
  source 1..37: mol_type = other RNA
  modified_base 1: mod_base = OTHER; note = Unlocked nucleic acid
nggcgccctggccagtcgtctgttttagagctatgct

SEQ ID NO: 402 - RNA, 37 nt, synthetic construct
  source 1..37: mol_type = other RNA
  modified_base 2: mod_base = OTHER; note = Unlocked nucleic acid
gngcgccctggccagtcgtctgttttagagctatgct

SEQ ID NO: 403 - RNA, 37 nt, synthetic construct
  source 1..37: mol_type = other RNA
  modified_base 3: mod_base = OTHER; note = Unlocked nucleic acid
ggncgccctggccagtcgtctgttttagagctatgct

SEQ ID NO: 404 - RNA, 37 nt, synthetic construct
  source 1..37: mol_type = other RNA
  modified_base 4: mod_base = OTHER; note = Unlocked nucleic acid
ggcngccctggccagtcgtctgttttagagctatgct

SEQ ID NO: 405 - RNA, 37 nt, synthetic construct
  source 1..37: mol_type = other RNA
  modified_base 5: mod_base = OTHER; note = Unlocked nucleic acid
ggcgnccctggccagtcgtctgttttagagctatgct

SEQ ID NO: 406 - RNA, 37 nt, synthetic construct
  source 1..37: mol_type = other RNA
  modified_base 6: mod_base = OTHER; note = Unlocked nucleic acid
ggcgcncctggccagtcgtctgttttagagctatgct

SEQ ID NO: 407 - RNA, 37 nt, synthetic construct
  source 1..37: mol_type = other RNA
  modified_base 7: mod_base = OTHER; note = Unlocked nucleic acid
ggcgccnctggccagtcgtctgttttagagctatgct

SEQ ID NO: 408 - RNA, 37 nt, synthetic construct
  source 1..37: mol_type = other RNA
  modified_base 8: mod_base = OTHER; note = Unlocked nucleic acid
ggcgcccntggccagtcgtctgttttagagctatgct

SEQ ID NO: 409 - RNA, 37 nt, synthetic construct
  source 1..37: mol_type = other RNA
  modified_base 9: mod_base = OTHER; note = Unlocked nucleic acid
ggcgccctnggccagtcgtctgttttagagctatgct

SEQ ID NO: 410 - RNA, 37 nt, synthetic construct
  source 1..37: mol_type = other RNA
  modified_base 10: mod_base = OTHER; note = Unlocked nucleic acid
ggcgccctgngccagtcgtctgttttagagctatgct

SEQ ID NO: 411 - RNA, 36 nt, synthetic construct
  source 1..36: mol_type = other RNA
tgtgctagacatgaggtctagttttagagctatgct

SEQ ID NO: 412 - RNA, 37 nt, synthetic construct
  source 1..37: mol_type = other RNA
  modified_base 1: mod_base = OTHER; note = Unlocked nucleic acid
ntgtgctagacatgaggtctagttttagagctatgct

SEQ ID NO: 413 - RNA, 37 nt, synthetic construct
  source 1..37: mol_type = other RNA
  modified_base 2: mod_base = OTHER; note = Unlocked nucleic acid
tngtgctagacatgaggtctagttttagagctatgct

SEQ ID NO: 414 - RNA, 37 nt, synthetic construct
  source 1..37: mol_type = other RNA
  modified_base 3: mod_base = OTHER; note = Unlocked nucleic acid
tgntgctagacatgaggtctagttttagagctatgct

SEQ ID NO: 415 - RNA, 37 nt, synthetic construct
  source 1..37: mol_type = other RNA
  modified_base 4: mod_base = OTHER; note = Unlocked nucleic acid
tgtngctagacatgaggtctagttttagagctatgct

SEQ ID NO: 416 - RNA, 37 nt, synthetic construct
  source 1..37: mol_type = other RNA
  modified_base 5: mod_base = OTHER; note = Unlocked nucleic acid
tgtgnctagacatgaggtctagttttagagctatgct

SEQ ID NO: 417 - RNA, 37 nt, synthetic construct
  source 1..37: mol_type = other RNA
  modified_base 6: mod_base = OTHER; note = Unlocked nucleic acid
tgtgcntagacatgaggtctagttttagagctatgct

SEQ ID NO: 418 - RNA, 37 nt, synthetic construct
  source 1..37: mol_type = other RNA
  modified_base 7: mod_base = OTHER; note = Unlocked nucleic acid
tgtgctnagacatgaggtctagttttagagctatgct

SEQ ID NO: 419 - RNA, 37 nt, synthetic construct
  source 1..37: mol_type = other RNA
  modified_base 8: mod_base = OTHER; note = Unlocked nucleic acid
tgtgctangacatgaggtctagttttagagctatgct

SEQ ID NO: 420 - RNA, 37 nt, synthetic construct
  source 1..37: mol_type = other RNA
  modified_base 9: mod_base = OTHER; note = Unlocked nucleic acid
tgtgctagnacatgaggtctagttttagagctatgct

SEQ ID NO: 421 - RNA, 37 nt, synthetic construct
  source 1..37: mol_type = other RNA
  modified_base 10: mod_base = OTHER; note = Unlocked nucleic acid
tgtgctagancatgaggtctagttttagagctatgct

SEQ ID NO: 422 - RNA, 36 nt, synthetic construct
  source 1..36: mol_type = other RNA
gagcagggctggggagaagggttttagagctatgct

SEQ ID NO: 423 - RNA, 36 nt, synthetic construct; identical to SEQ ID NO: 25
  source 1..36: mol_type = other RNA

SEQ ID NO: 424 - RNA, 36 nt, synthetic construct
  source 1..36: mol_type = other RNA
gctgcagaagggattccatggttttagagctatgct

SEQ ID NO: 425 - RNA, 36 nt, synthetic construct; identical to SEQ ID NO: 1
  source 1..36: mol_type = other RNA

SEQ ID NO: 426 - RNA, 36 nt, synthetic construct
  source 1..36: mol_type = other RNA
cttgccccacagggcagtaagttttagagctatgct

SEQ ID NO: 427 - RNA, 36 nt, synthetic construct; identical to SEQ ID NO: 24
  source 1..36: mol_type = other RNA

SEQ ID NO: 428 - RNA, 36 nt, synthetic construct
  source 1..36: mol_type = other RNA
ggcccagactgagcacgtgagttttagagctatgct

SEQ ID NO: 429 - RNA, 36 nt, synthetic construct
  source 1..36: mol_type = other RNA
aattatggggattactaggagttttagagctatgct

SEQ ID NO: 430 - RNA, 36 nt, synthetic construct
  source 1..36: mol_type = other RNA
cttaccccacttaactatctgttttagagctatgct

SEQ ID NO: 431 - RNA, 36 nt, synthetic construct
  source 1..36: mol_type = other RNA
cggtcaagatggaccagcacgttttagagctatgct

SEQ ID NO: 432 - RNA, 36 nt, synthetic construct
  source 1..36: mol_type = other RNA
ttgggacaacgttgtccagcgttttagagctatgct

SEQ ID NO: 433 - RNA, 36 nt, synthetic construct
  source 1..36: mol_type = other RNA
ctacgaggagcatttgcactgttttagagctatgct

SEQ ID NO: 434 - RNA, 37 nt, synthetic construct
  source 1..37: mol_type = other RNA
  modified_base 1: mod_base = OTHER; note = Unlocked nucleic acid
ngagcagggctggggagaagggttttagagctatgct

SEQ ID NO: 435 - RNA, 37 nt, synthetic construct
  source 1..37: mol_type = other RNA
  modified_base 2: mod_base = OTHER; note = Unlocked nucleic acid
gnagcagggctggggagaagggttttagagctatgct

SEQ ID NO: 436 - RNA, 37 nt, synthetic construct
  source 1..37: mol_type = other RNA
  modified_base 3: mod_base = OTHER; note = Unlocked nucleic acid
gangcagggctggggagaagggttttagagctatgct

SEQ ID NO: 437 - RNA, 37 nt, synthetic construct
  source 1..37: mol_type = other RNA
  modified_base 4: mod_base = OTHER; note = Unlocked nucleic acid
gagncagggctggggagaagggttttagagctatgct

SEQ ID NO: 438 - RNA, 37 nt, synthetic construct
  source 1..37: mol_type = other RNA
  modified_base 5: mod_base = OTHER; note = Unlocked nucleic acid
gagcnagggctggggagaagggttttagagctatgct

SEQ ID NO: 439 - RNA, 37 nt, synthetic construct
  source 1..37: mol_type = other RNA
  modified_base 6: mod_base = OTHER; note = Unlocked nucleic acid
gagcangggctggggagaagggttttagagctatgct

SEQ ID NO: 440 - RNA, 37 nt, synthetic construct
  source 1..37: mol_type = other RNA
  modified_base 7: mod_base = OTHER; note = Unlocked nucleic acid
gagcagnggctggggagaagggttttagagctatgct

SEQ ID NO: 441 - RNA, 37 nt, synthetic construct
  source 1..37: mol_type = other RNA
  modified_base 8: mod_base = OTHER; note = Unlocked nucleic acid
gagcaggngctggggagaagggttttagagctatgct

SEQ ID NO: 442 - RNA, 37 nt, synthetic construct
  source 1..37: mol_type = other RNA
  modified_base 9: mod_base = OTHER; note = Unlocked nucleic acid
gagcagggnctggggagaagggttttagagctatgct

SEQ ID NO: 443 - RNA, 37 nt, synthetic construct
  source 1..37: mol_type = other RNA
  modified_base 10: mod_base = OTHER; note = Unlocked nucleic acid
gagcagggcntggggagaagggttttagagctatgct

SEQ ID NO: 444 - RNA, 37 nt, synthetic construct
  source 1..37: mol_type = other RNA
  modified_base 11: mod_base = OTHER; note = Unlocked nucleic acid
gagcagggctnggggagaagggttttagagctatgct

SEQ ID NO: 445 - RNA, 37 nt, synthetic construct
  source 1..37: mol_type = other RNA
  modified_base 12: mod_base = OTHER; note = Unlocked nucleic acid
gagcagggctgngggagaagggttttagagctatgct

SEQ ID NO: 446 - RNA, 37 nt, synthetic construct
  source 1..37: mol_type = other RNA
  modified_base 13: mod_base = OTHER; note = Unlocked nucleic acid
gagcagggctggnggagaagggttttagagctatgct

SEQ ID NO: 447 - RNA, 37 nt, synthetic construct
  source 1..37: mol_type = other RNA
  modified_base 14: mod_base = OTHER; note = Unlocked nucleic acid
gagcagggctgggngagaagggttttagagctatgct

SEQ ID NO: 448 - RNA, 37 nt, synthetic construct
  source 1..37: mol_type = other RNA
  modified_base 15: mod_base = OTHER; note = Unlocked nucleic acid
gagcagggctggggnagaagggttttagagctatgct

SEQ ID NO: 449 - RNA, 37 nt, synthetic construct
  source 1..37: mol_type = other RNA
  modified_base 16: mod_base = OTHER; note = Unlocked nucleic acid
gagcagggctggggangaagggttttagagctatgct

SEQ ID NO: 450 - RNA, 37 nt, synthetic construct
  source 1..37: mol_type = other RNA
  modified_base 17: mod_base = OTHER; note = Unlocked nucleic acid
gagcagggctggggagnaagggttttagagctatgct

SEQ ID NO: 451 - RNA, 37 nt, synthetic construct
  source 1..37: mol_type = other RNA
  modified_base 18: mod_base = OTHER; note = Unlocked nucleic acid
gagcagggctggggaganagggttttagagctatgct

SEQ ID NO: 452 - RNA, 37 nt, synthetic construct
  source 1..37: mol_type = other RNA
  modified_base 19: mod_base = OTHER; note = Unlocked nucleic acid
gagcagggctggggagaangggttttagagctatgct

SEQ ID NO: 453 - RNA, 37 nt, synthetic construct
  source 1..37: mol_type = other RNA
  modified_base 20: mod_base = OTHER; note = Unlocked nucleic acid
gagcagggctggggagaagnggttttagagctatgct

SEQ ID NO: 454 - RNA, 39 nt, synthetic construct
  source 1..39: mol_type = other RNA
  modified_base 1: mod_base = OTHER; note = Unlocked nucleic acid
tnagaaggctgagatcctggagggttttagagctatgct

SEQ ID NO: 455 - RNA, 37 nt, synthetic construct; identical to SEQ ID NO: 47
  source 1..37: mol_type = other RNA
  modified_base 2: mod_base = OTHER; note = Unlocked nucleic acid

SEQ ID NO: 456 - RNA, 37 nt, synthetic construct; identical to SEQ ID NO: 48
  source 1..37: mol_type = other RNA
  modified_base 3: mod_base = OTHER; note = Unlocked nucleic acid

SEQ ID NO: 457 - RNA, 37 nt, synthetic construct; identical to SEQ ID NO: 49
  source 1..37: mol_type = other RNA
  modified_base 4: mod_base = OTHER; note = Unlocked nucleic acid

SEQ ID NO: 458 - RNA, 37 nt, synthetic construct; identical to SEQ ID NO: 50
  source 1..37: mol_type = other RNA
  modified_base 5: mod_base = OTHER; note = Unlocked nucleic acid

SEQ ID NO: 459 - RNA, 37 nt, synthetic construct; identical to SEQ ID NO: 51
  source 1..37: mol_type = other RNA
  modified_base 6: mod_base = OTHER; note = Unlocked nucleic acid

SEQ ID NO: 460 - RNA, 37 nt, synthetic construct; identical to SEQ ID NO: 52
  source 1..37: mol_type = other RNA
  modified_base 7: mod_base = OTHER; note = Unlocked nucleic acid

SEQ ID NO: 461 - RNA, 37 nt, synthetic construct; identical to SEQ ID NO: 53
  source 1..37: mol_type = other RNA
  modified_base 8: mod_base = OTHER; note = Unlocked nucleic acid

SEQ ID NO: 462 - RNA, 37 nt, synthetic construct; identical to SEQ ID NO: 54
  source 1..37: mol_type = other RNA
  modified_base 9: mod_base = OTHER; note = Unlocked nucleic acid

SEQ ID NO: 463 - RNA, 37 nt, synthetic construct; identical to SEQ ID NO: 55
  source 1..37: mol_type = other RNA
  modified_base 10: mod_base = OTHER; note = Unlocked nucleic acid

SEQ ID NO: 464 - RNA, 37 nt, synthetic construct; identical to SEQ ID NO: 56
  source 1..37: mol_type = other RNA
  modified_base 11: mod_base = OTHER; note = Unlocked nucleic acid

SEQ ID NO: 465 - RNA, 37 nt, synthetic construct; identical to SEQ ID NO: 57
  source 1..37: mol_type = other RNA
  modified_base 12: mod_base = OTHER; note = Unlocked nucleic acid

SEQ ID NO: 466 - RNA, 37 nt, synthetic construct; identical to SEQ ID NO: 58
  source 1..37: mol_type = other RNA
  modified_base 13: mod_base = OTHER; note = Unlocked nucleic acid

SEQ ID NO: 467 - RNA, 37 nt, synthetic construct; identical to SEQ ID NO: 59
  source 1..37: mol_type = other RNA
  modified_base 14: mod_base = OTHER; note = Unlocked nucleic acid

SEQ ID NO: 468 - RNA, 37 nt, synthetic construct; identical to SEQ ID NO: 60
  source 1..37: mol_type = other RNA
  modified_base 15: mod_base = OTHER; note = Unlocked nucleic acid

SEQ ID NO: 469 - RNA, 37 nt, synthetic construct; identical to SEQ ID NO: 61
  source 1..37: mol_type = other RNA
  modified_base 16: mod_base = OTHER; note = Unlocked nucleic acid

SEQ ID NO: 470 - RNA, 37 nt, synthetic construct; identical to SEQ ID NO: 62
  source 1..37: mol_type = other RNA
  modified_base 17: mod_base = OTHER; note = Unlocked nucleic acid

SEQ ID NO: 471 - RNA, 37 nt, synthetic construct; identical to SEQ ID NO: 63
  source 1..37: mol_type = other RNA
  modified_base 18: mod_base = OTHER; note = Unlocked nucleic acid

SEQ ID NO: 472 - RNA, 37 nt, synthetic construct; identical to SEQ ID NO: 64
  source 1..37: mol_type = other RNA
  modified_base 19: mod_base = OTHER; note = Unlocked nucleic acid

SEQ ID NO: 473 - RNA, 37 nt, synthetic construct; identical to SEQ ID NO: 65
  source 1..37: mol_type = other RNA
  modified_base 20: mod_base = OTHER; note = Unlocked nucleic acid

SEQ ID NO: 474 - RNA, 37 nt, synthetic construct
  source 1..37: mol_type = other RNA
  modified_base 1: mod_base = OTHER; note = Unlocked nucleic acid
ngctgcagaagggattccatggttttagagctatgct

SEQ ID NO: 475 - RNA, 37 nt, synthetic construct
  source 1..37: mol_type = other RNA
  modified_base 2: mod_base = OTHER; note = Unlocked nucleic acid
gnctgcagaagggattccatggttttagagctatgct

SEQ ID NO: 476 - RNA, 37 nt, synthetic construct
  source 1..37: mol_type = other RNA
  modified_base 3: mod_base = OTHER; note = Unlocked nucleic acid
gcntgcagaagggattccatggttttagagctatgct

SEQ ID NO: 477 - RNA, 37 nt, synthetic construct
  source 1..37: mol_type = other RNA
  modified_base 4: mod_base = OTHER; note = Unlocked nucleic acid
gctngcagaagggattccatggttttagagctatgct

SEQ ID NO: 478 - RNA, 37 nt, synthetic construct
  source 1..37: mol_type = other RNA
  modified_base 5: mod_base = OTHER; note = Unlocked nucleic acid
gctgncagaagggattccatggttttagagctatgct

SEQ ID NO: 479 - RNA, 37 nt, synthetic construct
  source 1..37: mol_type = other RNA
  modified_base 6: mod_base = OTHER; note = Unlocked nucleic acid
gctgcnagaagggattccatggttttagagctatgct

SEQ ID NO: 480 - RNA, 37 nt, synthetic construct
  source 1..37: mol_type = other RNA
  modified_base 7: mod_base = OTHER; note = Unlocked nucleic acid
gctgcangaagggattccatggttttagagctatgct

SEQ ID NO: 481 - RNA, 37 nt, synthetic construct
  source 1..37: mol_type = other RNA
  modified_base 8: mod_base = OTHER; note = Unlocked nucleic acid
gctgcagnaagggattccatggttttagagctatgct

SEQ ID NO: 482 - RNA, 37 nt, synthetic construct
  source 1..37: mol_type = other RNA
  modified_base 9: mod_base = OTHER; note = Unlocked nucleic acid
gctgcaganagggattccatggttttagagctatgct

SEQ ID NO: 483 - RNA, 37 nt, synthetic construct
  source 1..37: mol_type = other RNA
  modified_base 10: mod_base = OTHER; note = Unlocked nucleic acid
gctgcagaangggattccatggttttagagctatgct

SEQ ID NO: 484 - RNA, 37 nt, synthetic construct
  source 1..37: mol_type = other RNA
  modified_base 11: mod_base = OTHER; note = Unlocked nucleic acid
gctgcagaagnggattccatggttttagagctatgct

SEQ ID NO: 485 - RNA, 37 nt, synthetic construct
  source 1..37: mol_type = other RNA
  modified_base 12: mod_base = OTHER; note = Unlocked nucleic acid
gctgcagaaggngattccatggttttagagctatgct

SEQ ID NO: 486 - RNA, 37 nt, synthetic construct
  source 1..37: mol_type = other RNA
  modified_base 13: mod_base = OTHER; note = Unlocked nucleic acid
gctgcagaagggnattccatggttttagagctatgct

SEQ ID NO: 487 - RNA, 37 nt, synthetic construct
  source 1..37: mol_type = other RNA
  modified_base 14: mod_base = OTHER; note = Unlocked nucleic acid
gctgcagaaggganttccatggttttagagctatgct

SEQ ID NO: 488 - RNA, 37 nt, synthetic construct
  source 1..37: mol_type = other RNA
  modified_base 15: mod_base = OTHER; note = Unlocked nucleic acid
gctgcagaagggatntccatggttttagagctatgct

SEQ ID NO: 489 - RNA, 37 nt, synthetic construct
  source 1..37: mol_type = other RNA
  modified_base 16: mod_base = OTHER; note = Unlocked nucleic acid
gctgcagaagggattnccatggttttagagctatgct

SEQ ID NO: 490 - RNA, 37 nt, synthetic construct
  source 1..37: mol_type = other RNA
  modified_base 17: mod_base = OTHER; note = Unlocked nucleic acid
gctgcagaagggattcncatggttttagagctatgct

SEQ ID NO: 491 - RNA, 37 nt, synthetic construct
  source 1..37: mol_type = other RNA
  modified_base 18: mod_base = OTHER; note = Unlocked nucleic acid
gctgcagaagggattccnatggttttagagctatgct

SEQ ID NO: 492 - RNA, 37 nt, synthetic construct
  source 1..37: mol_type = other RNA
  modified_base 19: mod_base = OTHER; note = Unlocked nucleic acid
gctgcagaagggattccantggttttagagctatgct

SEQ ID NO: 493 - RNA, 37 nt, synthetic construct
  source 1..37: mol_type = other RNA
  modified_base 20: mod_base = OTHER; note = Unlocked nucleic acid
gctgcagaagggattccatnggttttagagctatgct

SEQ ID NO: 494 - RNA, 37 nt, synthetic construct; identical to SEQ ID NO: 4
  source 1..37: mol_type = other RNA
  modified_base 1: mod_base = OTHER; note = Unlocked nucleic acid

SEQ ID NO: 495 - RNA, 37 nt, synthetic construct; identical to SEQ ID NO: 5
  source 1..37: mol_type = other RNA
  modified_base 2: mod_base = OTHER; note = Unlocked nucleic acid

SEQ ID NO: 496 - RNA, 37 nt, synthetic construct; identical to SEQ ID NO: 6
  source 1..37: mol_type = other RNA
  modified_base 3: mod_base = OTHER; note = Unlocked nucleic acid

SEQ ID NO: 497 - RNA, 37 nt, synthetic construct; identical to SEQ ID NO: 7
  source 1..37: mol_type = other RNA
  modified_base 4: mod_base = OTHER; note = Unlocked nucleic acid

SEQ ID NO: 498 - RNA, 37 nt, synthetic construct; identical to SEQ ID NO: 8
  source 1..37: mol_type = other RNA
  modified_base 5: mod_base = OTHER; note = Unlocked nucleic acid

SEQ ID NO: 499 - RNA, 37 nt, synthetic construct; identical to SEQ ID NO: 9
  source 1..37: mol_type = other RNA
  modified_base 6: mod_base = OTHER; note = Unlocked nucleic acid

SEQ ID NO: 500 - RNA, 37 nt, synthetic construct; identical to SEQ ID NO: 10
  source 1..37: mol_type = other RNA
  modified_base 7: mod_base = OTHER; note = Unlocked nucleic acid

SEQ ID NO: 501 - RNA, 37 nt, synthetic construct; identical to SEQ ID NO: 11
  source 1..37: mol_type = other RNA
  modified_base 8: mod_base = OTHER; note = Unlocked nucleic acid

SEQ ID NO: 502 - RNA, 37 nt, synthetic construct; identical to SEQ ID NO: 12
  source 1..37: mol_type = other RNA
  modified_base 9: mod_base = OTHER; note = Unlocked nucleic acid

SEQ ID NO: 503 - RNA, 37 nt, synthetic construct; identical to SEQ ID NO: 13
  source 1..37: mol_type = other RNA
  modified_base 10: mod_base = OTHER; note = Unlocked nucleic acid

SEQ ID NO: 504 - RNA, 37 nt, synthetic construct; identical to SEQ ID NO: 14
  source 1..37: mol_type = other RNA
  modified_base 11: mod_base = OTHER; note = Unlocked nucleic acid

SEQ ID NO: 505 - RNA, 37 nt, synthetic construct; identical to SEQ ID NO: 15
  source 1..37: mol_type = other RNA
  modified_base 12: mod_base = OTHER; note = Unlocked nucleic acid

SEQ ID NO: 506 - RNA, 37 nt, synthetic construct; identical to SEQ ID NO: 16
  source 1..37: mol_type = other RNA
  modified_base 13: mod_base = OTHER; note = Unlocked nucleic acid

SEQ ID NO: 507 - RNA, 37 nt, synthetic construct; identical to SEQ ID NO: 17
  source 1..37: mol_type = other RNA
  modified_base 14: mod_base = OTHER; note = Unlocked nucleic acid

SEQ ID NO: 508 - RNA, 37 nt, synthetic construct; identical to SEQ ID NO: 18
  source 1..37: mol_type = other RNA
  modified_base 15: mod_base = OTHER; note = Unlocked nucleic acid

SEQ ID NO: 509 - RNA, 37 nt, synthetic construct; identical to SEQ ID NO: 19
  source 1..37: mol_type = other RNA
  modified_base 16: mod_base = OTHER; note = Unlocked nucleic acid

SEQ ID NO: 510 - RNA, 37 nt, synthetic construct; identical to SEQ ID NO: 20
  source 1..37: mol_type = other RNA
  modified_base 17: mod_base = OTHER; note = Unlocked nucleic acid

SEQ ID NO: 511 - RNA, 37 nt, synthetic construct; identical to SEQ ID NO: 21
  source 1..37: mol_type = other RNA
  modified_base 18: mod_base = OTHER; note = Unlocked nucleic acid

SEQ ID NO: 512 - RNA, 37 nt, synthetic construct; identical to SEQ ID NO: 22
  source 1..37: mol_type = other RNA
  modified_base 19: mod_base = OTHER; note = Unlocked nucleic acid

SEQ ID NO: 513 - RNA, 37 nt, synthetic construct; identical to SEQ ID NO: 23
  source 1..37: mol_type = other RNA
  modified_base 20: mod_base = OTHER; note = Unlocked nucleic acid

SEQ ID NO: 514 - RNA, 37 nt, synthetic construct
  source 1..37: mol_type = other RNA
  modified_base 1: mod_base = OTHER; note = Unlocked nucleic acid
ncttgccccacagggcagtaagttttagagctatgct

SEQ ID NO: 515 - RNA, 37 nt, synthetic construct
  source 1..37: mol_type = other RNA
  modified_base 2: mod_base = OTHER; note = Unlocked nucleic acid
cnttgccccacagggcagtaagttttagagctatgct

SEQ ID NO: 516 - RNA, 37 nt, synthetic construct
  source 1..37: mol_type = other RNA
  modified_base 3: mod_base = OTHER; note = Unlocked nucleic acid
ctntgccccacagggcagtaagttttagagctatgct

SEQ ID NO: 517 - RNA, 37 nt, synthetic construct
  source 1..37: mol_type = other RNA
  modified_base 4: mod_base = OTHER; note = Unlocked nucleic acid
cttngccccacagggcagtaagttttagagctatgct

SEQ ID NO: 518 - RNA, 37 nt, synthetic construct
  source 1..37: mol_type = other RNA
  modified_base 5: mod_base = OTHER; note = Unlocked nucleic acid
cttgnccccacagggcagtaagttttagagctatgct

SEQ ID NO: 519 - RNA, 37 nt, synthetic construct
  source 1..37: mol_type = other RNA
  modified_base 6: mod_base = OTHER; note = Unlocked nucleic acid
cttgcncccacagggcagtaagttttagagctatgct

SEQ ID NO: 520 - RNA, 37 nt, synthetic construct
  source 1..37: mol_type = other RNA
  modified_base 7: mod_base = OTHER; note = Unlocked nucleic acid
cttgccnccacagggcagtaagttttagagctatgct

SEQ ID NO: 521 - RNA, 37 nt, synthetic construct
  source 1..37: mol_type = other RNA
  modified_base 8: mod_base = OTHER; note = Unlocked nucleic acid
cttgcccncacagggcagtaagttttagagctatgct

SEQ ID NO: 522 - RNA, 37 nt, synthetic construct
  source 1..37: mol_type = other RNA
  modified_base 9: mod_base = OTHER; note = Unlocked nucleic acid
cttgccccnacagggcagtaagttttagagctatgct

SEQ ID NO: 523 - RNA, 37 nt, synthetic construct
  source 1..37: mol_type = other RNA
  modified_base 10: mod_base = OTHER; note = Unlocked nucleic acid
cttgccccancagggcagtaagttttagagctatgct

SEQ ID NO: 524 - RNA, 37 nt, synthetic construct
  source 1..37: mol_type = other RNA
  modified_base 11: mod_base = OTHER; note = Unlocked nucleic acid
cttgccccacnagggcagtaagttttagagctatgct

SEQ ID NO: 525 - RNA, 37 nt, synthetic construct
  source 1..37: mol_type = other RNA
  modified_base 12: mod_base = OTHER; note = Unlocked nucleic acid
cttgccccacangggcagtaagttttagagctatgct

SEQ ID NO: 526 - RNA, 37 nt, synthetic construct
  source 1..37: mol_type = other RNA
  modified_base 13: mod_base = OTHER; note = Unlocked nucleic acid
cttgccccacagnggcagtaagttttagagctatgct

SEQ ID NO: 527 - RNA, 37 nt, synthetic construct
  source 1..37: mol_type = other RNA
  modified_base 14: mod_base = OTHER; note = Unlocked nucleic acid
cttgccccacaggngcagtaagttttagagctatgct

SEQ ID NO: 528 - RNA, 37 nt, synthetic construct
  source 1..37: mol_type = other RNA
  modified_base 15: mod_base = OTHER; note = Unlocked nucleic acid
cttgccccacagggncagtaagttttagagctatgct

SEQ ID NO: 529 - RNA, 37 nt, synthetic construct
  source 1..37: mol_type = other RNA
  modified_base 16: mod_base = OTHER; note = Unlocked nucleic acid
cttgccccacagggcnagtaagttttagagctatgct

SEQ ID NO: 530 - RNA, 37 nt, synthetic construct
  source 1..37: mol_type = other RNA
  modified_base 17: mod_base = OTHER; note = Unlocked nucleic acid
cttgccccacagggcangtaagttttagagctatgct

SEQ ID NO: 531 - RNA, 37 nt, synthetic construct
  source 1..37: mol_type = other RNA
  modified_base 18: mod_base = OTHER; note = Unlocked nucleic acid
cttgccccacagggcagntaagttttagagctatgct

SEQ ID NO: 532 - RNA, 37 nt, synthetic construct
  source 1..37: mol_type = other RNA
  modified_base 19: mod_base = OTHER; note = Unlocked nucleic acid
cttgccccacagggcagtnaagttttagagctatgct

SEQ ID NO: 533 - RNA, 37 nt, synthetic construct
  source 1..37: mol_type = other RNA
  modified_base 20: mod_base = OTHER; note = Unlocked nucleic acid
cttgccccacagggcagtanagttttagagctatgct

SEQ ID NO: 534 - RNA, 37 nt, synthetic construct; identical to SEQ ID NO: 26
  source 1..37: mol_type = other RNA
  modified_base 1: mod_base = OTHER; note = Unlocked nucleic acid

SEQ ID NO: 535 - RNA, 37 nt, synthetic construct; identical to SEQ ID NO: 27
  source 1..37: mol_type = other RNA
  modified_base 2: mod_base = OTHER; note = Unlocked nucleic acid

SEQ ID NO: 536 - RNA, 37 nt, synthetic construct; identical to SEQ ID NO: 28
  source 1..37: mol_type = other RNA
  modified_base 3: mod_base = OTHER; note = Unlocked nucleic acid

SEQ ID NO: 537 - RNA, 37 nt, synthetic construct; identical to SEQ ID NO: 29
  source 1..37: mol_type = other RNA
  modified_base 4: mod_base = OTHER; note = Unlocked nucleic acid

SEQ ID NO: 538 - RNA, 37 nt, synthetic construct; identical to SEQ ID NO: 30
  source 1..37: mol_type = other RNA
  modified_base 5: mod_base = OTHER; note = Unlocked nucleic acid

SEQ ID NO: 539 - RNA, 37 nt, synthetic construct; identical to SEQ ID NO: 31
  source 1..37: mol_type = other RNA
  modified_base 6: mod_base = OTHER; note = Unlocked nucleic acid

SEQ ID NO: 540 - RNA, 37 nt, synthetic construct; identical to SEQ ID NO: 32
  source 1..37: mol_type = other RNA
  modified_base 7: mod_base = OTHER; note = Unlocked nucleic acid

SEQ ID NO: 541 - RNA, 37 nt, synthetic construct; identical to SEQ ID NO: 33
  source 1..37: mol_type = other RNA
  modified_base 8: mod_base = OTHER; note = Unlocked nucleic acid

SEQ ID NO: 542 - RNA, 37 nt, synthetic construct; identical to SEQ ID NO: 34
  source 1..37: mol_type = other RNA
  modified_base 9: mod_base = OTHER; note = Unlocked nucleic acid

SEQ ID NO: 543 - RNA, 37 nt, synthetic construct; identical to SEQ ID NO: 35
  source 1..37: mol_type = other RNA
  modified_base 10: mod_base = OTHER; note = Unlocked nucleic acid

SEQ ID NO: 544 - RNA, 37 nt, synthetic construct; identical to SEQ ID NO: 36
  source 1..37: mol_type = other RNA
  modified_base 11: mod_base = OTHER; note = Unlocked nucleic acid

SEQ ID NO: 545 - RNA, 37 nt, synthetic construct; identical to SEQ ID NO: 37
  source 1..37: mol_type = other RNA
  modified_base 12: mod_base = OTHER; note = Unlocked nucleic acid

SEQ ID NO: 546 - RNA, 37 nt, synthetic construct; identical to SEQ ID NO: 38
  source 1..37: mol_type = other RNA
  modified_base 13: mod_base = OTHER; note = Unlocked nucleic acid

SEQ ID NO: 547 - RNA, 37 nt, synthetic construct; identical to SEQ ID NO: 39
  source 1..37: mol_type = other RNA
  modified_base 14: mod_base = OTHER; note = Unlocked nucleic acid

SEQ ID NO: 548 - RNA, 37 nt, synthetic construct; identical to SEQ ID NO: 40
  source 1..37: mol_type = other RNA
  modified_base 15: mod_base = OTHER; note = Unlocked nucleic acid

SEQ ID NO: 549 - RNA, 37 nt, synthetic construct; identical to SEQ ID NO: 41
  source 1..37: mol_type = other RNA
  modified_base 16: mod_base = OTHER; note = Unlocked nucleic acid

SEQ ID NO: 550 - RNA, 37 nt, synthetic construct; identical to SEQ ID NO: 42
  source 1..37: mol_type = other RNA
  modified_base 17: mod_base = OTHER; note = Unlocked nucleic acid

SEQ ID NO: 551 - RNA, 37 nt, synthetic construct; identical to SEQ ID NO: 43
  source 1..37: mol_type = other RNA
  modified_base 18: mod_base = OTHER; note = Unlocked nucleic acid

SEQ ID NO: 552 - RNA, 37 nt, synthetic construct; identical to SEQ ID NO: 44
  source 1..37: mol_type = other RNA
  modified_base 19: mod_base = OTHER; note = Unlocked nucleic acid

SEQ ID NO: 553 - RNA, 37 nt, synthetic construct; identical to SEQ ID NO: 45
  source 1..37: mol_type = other RNA
  modified_base 20: mod_base = OTHER; note = Unlocked nucleic acid

SEQ ID NO: 554 - RNA, 37 nt, synthetic construct
  source 1..37: mol_type = other RNA
  modified_base 1: mod_base = OTHER; note = Unlocked nucleic acid
nggcccagactgagcacgtgagttttagagctatgct

SEQ ID NO: 555 - RNA, 37 nt, synthetic construct
  source 1..37: mol_type = other RNA
  modified_base 2: mod_base = OTHER; note = Unlocked nucleic acid
gngcccagactgagcacgtgagttttagagctatgct

SEQ ID NO: 556 - RNA, 37 nt, synthetic construct
  source 1..37: mol_type = other RNA
  modified_base 3: mod_base = OTHER; note = Unlocked nucleic acid
ggncccagactgagcacgtgagttttagagctatgct

SEQ ID NO: 557 - RNA, 37 nt, synthetic construct
  source 1..37: mol_type = other RNA
  modified_base 4: mod_base = OTHER; note = Unlocked nucleic acid
ggcnccagactgagcacgtgagttttagagctatgct

SEQ ID NO: 558 - RNA, 37 nt, synthetic construct
  source 1..37: mol_type = other RNA
  modified_base 5: mod_base = OTHER; note = Unlocked nucleic acid
ggccncagactgagcacgtgagttttagagctatgct

SEQ ID NO: 559 - RNA, 37 nt, synthetic construct
  source 1..37: mol_type = other RNA
  modified_base 6: mod_base = OTHER; note = Unlocked nucleic acid
ggcccnagactgagcacgtgagttttagagctatgct

SEQ ID NO: 560 - RNA, 37 nt, synthetic construct
  source 1..37: mol_type = other RNA
  modified_base 7: mod_base = OTHER; note = Unlocked nucleic acid
ggcccangactgagcacgtgagttttagagctatgct

SEQ ID NO: 561 - RNA, 37 nt, synthetic construct
  source 1..37: mol_type = other RNA
  modified_base 8: mod_base = OTHER; note = Unlocked nucleic acid
ggcccagnactgagcacgtgagttttagagctatgct

SEQ ID NO: 562 - RNA, 37 nt, synthetic construct
  source 1..37: mol_type = other RNA
  modified_base 9: mod_base = OTHER; note = Unlocked nucleic acid
ggcccaganctgagcacgtgagttttagagctatgct

SEQ ID NO: 563 - RNA, 37 nt, synthetic construct
  source 1..37: mol_type = other RNA
  modified_base 10: mod_base = OTHER; note = Unlocked nucleic acid
ggcccagacntgagcacgtgagttttagagctatgct

SEQ ID NO: 564 - RNA, 37 nt, synthetic construct
  source 1..37: mol_type = other RNA
  modified_base 11: mod_base = OTHER; note = Unlocked nucleic acid
ggcccagactngagcacgtgagttttagagctatgct

SEQ ID NO: 565 - RNA, 37 nt, synthetic construct
  source 1..37: mol_type = other RNA
  modified_base 12: mod_base = OTHER; note = Unlocked nucleic acid
ggcccagactgnagcacgtgagttttagagctatgct

SEQ ID NO: 566 - RNA, 37 nt, synthetic construct
  source 1..37: mol_type = other RNA
  modified_base 13: mod_base = OTHER; note = Unlocked nucleic acid
ggcccagactgangcacgtgagttttagagctatgct

SEQ ID NO: 567 - RNA, 37 nt, synthetic construct
  source 1..37: mol_type = other RNA
  modified_base 14: mod_base = OTHER; note = Unlocked nucleic acid
ggcccagactgagncacgtgagttttagagctatgct

SEQ ID NO: 568 - RNA, 37 nt, synthetic construct
  source 1..37: mol_type = other RNA
  modified_base 15: mod_base = OTHER; note = Unlocked nucleic acid
ggcccagactgagcnacgtgagttttagagctatgct

SEQ ID NO: 569 - RNA, 37 nt, synthetic construct
  source 1..37: mol_type = other RNA
  modified_base 16: mod_base = OTHER; note = Unlocked nucleic acid
ggcccagactgagcancgtgagttttagagctatgct

SEQ ID NO: 570 - RNA, 37 nt, synthetic construct
  source 1..37: mol_type = other RNA
  modified_base 17: mod_base = OTHER; note = Unlocked nucleic acid
ggcccagactgagcacngtgagttttagagctatgct

SEQ ID NO: 571 - RNA, 37 nt, synthetic construct
  source 1..37: mol_type = other RNA
  modified_base 18: mod_base = OTHER; note = Unlocked nucleic acid
ggcccagactgagcacgntgagttttagagctatgct

SEQ ID NO: 572 - RNA, 37 nt, synthetic construct
  source 1..37: mol_type = other RNA
  modified_base 19: mod_base = OTHER; note = Unlocked nucleic acid
ggcccagactgagcacgtngagttttagagctatgct

SEQ ID NO: 573 - RNA, 37 nt, synthetic construct
  source 1..37: mol_type = other RNA
  modified_base 20: mod_base = OTHER; note = Unlocked nucleic acid
ggcccagactgagcacgtgnagttttagagctatgct

SEQ ID NO: 574 - RNA, 37 nt, synthetic construct
  source 1..37: mol_type = other RNA
  modified_base 1: mod_base = OTHER; note = Unlocked nucleic acid
naattatggggattactaggagttttagagctatgct

SEQ ID NO: 575 - RNA, 37 nt, synthetic construct
  source 1..37: mol_type = other RNA
  modified_base 2: mod_base = OTHER; note = Unlocked nucleic acid
anattatggggattactaggagttttagagctatgct

SEQ ID NO: 576 - RNA, 37 nt, synthetic construct
  source 1..37: mol_type = other RNA
  modified_base 3: mod_base = OTHER; note = Unlocked nucleic acid
aanttatggggattactaggagttttagagctatgct

SEQ ID NO: 577 - RNA, 37 nt, synthetic construct
  source 1..37: mol_type = other RNA
  modified_base 4: mod_base = OTHER; note = Unlocked nucleic acid
aatntatggggattactaggagttttagagctatgct

SEQ ID NO: 578 - RNA, 37 nt, synthetic construct
  source 1..37: mol_type = other RNA
  modified_base 5: mod_base = OTHER; note = Unlocked nucleic acid
aattnatggggattactaggagttttagagctatgct

SEQ ID NO: 579 - RNA, 37 nt, synthetic construct
  source 1..37: mol_type = other RNA
  modified_base 6: mod_base = OTHER; note = Unlocked nucleic acid
aattantggggattactaggagttttagagctatgct

SEQ ID NO: 580 - RNA, 37 nt, synthetic construct
  source 1..37: mol_type = other RNA
  modified_base 7: mod_base = OTHER; note = Unlocked nucleic acid
aattatnggggattactaggagttttagagctatgct

SEQ ID NO: 581 - RNA, 37 nt, synthetic construct
  source 1..37: mol_type = other RNA
  modified_base 8: mod_base = OTHER; note = Unlocked nucleic acid
aattatgngggattactaggagttttagagctatgct

SEQ ID NO: 582 - RNA, 37 nt, synthetic construct
  source 1..37: mol_type = other RNA
  modified_base 9: mod_base = OTHER; note = Unlocked nucleic acid
aattatggnggattactaggagttttagagctatgct

SEQ ID NO: 583 - RNA, 37 nt, synthetic construct
  source 1..37: mol_type = other RNA
  modified_base 10: mod_base = OTHER; note = Unlocked nucleic acid
aattatgggngattactaggagttttagagctatgct

SEQ ID NO: 584 - RNA, 37 nt, synthetic construct
  source 1..37: mol_type = other RNA
  modified_base 11: mod_base = OTHER; note = Unlocked nucleic acid
aattatggggnattactaggagttttagagctatgct

SEQ ID NO: 585 - RNA, 37 nt, synthetic construct
  source 1..37: mol_type = other RNA
  modified_base 12: mod_base = OTHER; note = Unlocked nucleic acid
aattatgggganttactaggagttttagagctatgct

SEQ ID NO: 586 - RNA, 37 nt, synthetic construct
  source 1..37: mol_type = other RNA
  modified_base 13: mod_base = OTHER; note = Unlocked nucleic acid
aattatggggatntactaggagttttagagctatgct

SEQ ID NO: 587 - RNA, 37 nt, synthetic construct
  source 1..37: mol_type = other RNA
  modified_base 14: mod_base = OTHER; note = Unlocked nucleic acid
aattatggggattnactaggagttttagagctatgct

SEQ ID NO: 588 - RNA, 37 nt, synthetic construct
  source 1..37: mol_type = other RNA
  modified_base 15: mod_base = OTHER; note = Unlocked nucleic acid
aattatggggattanctaggagttttagagctatgct

SEQ ID NO: 589 - RNA, 37 nt, synthetic construct
  source 1..37: mol_type = other RNA
  modified_base 16: mod_base = OTHER; note = Unlocked nucleic acid
aattatggggattacntaggagttttagagctatgct

SEQ ID NO: 590 - RNA, 37 nt, synthetic construct
  source 1..37: mol_type = other RNA
  modified_base 17: mod_base = OTHER; note = Unlocked nucleic acid
aattatggggattactnaggagttttagagctatgct

SEQ ID NO: 591 - RNA, 37 nt, synthetic construct
  source 1..37: mol_type = other RNA
  modified_base 18: mod_base = OTHER; note = Unlocked nucleic acid
aattatggggattactanggagttttagagctatgct

SEQ ID NO: 592 - RNA, 37 nt, synthetic construct
  source 1..37: mol_type = other RNA
  modified_base 19: mod_base = OTHER; note = Unlocked nucleic acid
aattatggggattactagngagttttagagctatgct

SEQ ID NO: 593 - RNA, 37 nt, synthetic construct
  source 1..37: mol_type = other RNA
  modified_base 20: mod_base = OTHER; note = Unlocked nucleic acid
aattatggggattactaggnagttttagagctatgct

SEQ ID NO: 594 - RNA, 37 nt, synthetic construct
  source 1..37: mol_type = other RNA
  modified_base 1: mod_base = OTHER; note = Unlocked nucleic acid
ncttaccccacttaactatctgttttagagctatgct

SEQ ID NO: 595 - RNA, 37 nt, synthetic construct
  source 1..37: mol_type = other RNA
  modified_base 2: mod_base = OTHER; note = Unlocked nucleic acid
cnttaccccacttaactatctgttttagagctatgct

SEQ ID NO: 596 - RNA, 37 nt, synthetic construct
  source 1..37: mol_type = other RNA
  modified_base 3: mod_base = OTHER; note = Unlocked nucleic acid
ctntaccccacttaactatctgttttagagctatgct

SEQ ID NO: 597 - RNA, 37 nt, synthetic construct
  source 1..37: mol_type = other RNA
  modified_base 4: mod_base = OTHER; note = Unlocked nucleic acid
cttnaccccacttaactatctgttttagagctatgct

SEQ ID NO: 598 - RNA, 37 nt, synthetic construct
  source 1..37: mol_type = other RNA
  modified_base 5: mod_base = OTHER; note = Unlocked nucleic acid
cttanccccacttaactatctgttttagagctatgct

SEQ ID NO: 599 - RNA, 37 nt, synthetic construct
  source 1..37: mol_type = other RNA
  modified_base 6: mod_base = OTHER; note = Unlocked nucleic acid
cttacncccacttaactatctgttttagagctatgct

SEQ ID NO: 600 - RNA, 37 nt, synthetic construct
  source 1..37: mol_type = other RNA
  modified_base 7: mod_base = OTHER; note = Unlocked nucleic acid
cttaccnccacttaactatctgttttagagctatgct

SEQ ID NO: 601 - RNA, 37 nt, synthetic construct
  source 1..37: mol_type = other RNA
  modified_base 8: mod_base = OTHER; note = Unlocked nucleic acid
cttacccncacttaactatctgttttagagctatgct

SEQ ID NO: 602 - RNA, 37 nt, synthetic construct
  source 1..37: mol_type = other RNA
  modified_base 9: mod_base = OTHER; note = Unlocked nucleic acid
cttaccccnacttaactatctgttttagagctatgct

SEQ ID NO: 603 - RNA, 37 nt, synthetic construct
  source 1..37: mol_type = other RNA
  modified_base 10: mod_base = OTHER; note = Unlocked nucleic acid
cttaccccancttaactatctgttttagagctatgct

SEQ ID NO: 604 - RNA, 37 nt, synthetic construct
  source 1..37: mol_type = other RNA
  modified_base 11: mod_base = OTHER; note = Unlocked nucleic acid
cttaccccacnttaactatctgttttagagctatgct

SEQ ID NO: 605 - RNA, 37 nt, synthetic construct
  source 1..37: mol_type = other RNA
  modified_base 12: mod_base = OTHER; note = Unlocked nucleic acid
cttaccccactntaactatctgttttagagctatgct

SEQ ID NO: 606 - RNA, 37 nt, synthetic construct
  source 1..37: mol_type = other RNA
  modified_base 13: mod_base = OTHER; note = Unlocked nucleic acid
cttaccccacttnaactatctgttttagagctatgct

SEQ ID NO: 607 - RNA, 37 nt, synthetic construct
  source 1..37: mol_type = other RNA
  modified_base 14: mod_base = OTHER; note = Unlocked nucleic acid
cttaccccacttanactatctgttttagagctatgct

SEQ ID NO: 608 - RNA, 37 nt, synthetic construct
  source 1..37: mol_type = other RNA
  modified_base 15: mod_base = OTHER; note = Unlocked nucleic acid
cttaccccacttaanctatctgttttagagctatgct

SEQ ID NO: 609 - RNA, 37 nt, synthetic construct
  source 1..37: mol_type = other RNA
  modified_base 16: mod_base = OTHER; note = Unlocked nucleic acid
cttaccccacttaacntatctgttttagagctatgct

SEQ ID NO: 610 - RNA, 37 nt, synthetic construct
  source 1..37: mol_type = other RNA
  modified_base 17: mod_base = OTHER; note = Unlocked nucleic acid
cttaccccacttaactnatctgttttagagctatgct

SEQ ID NO: 611 - RNA, 37 nt, synthetic construct
  source 1..37: mol_type = other RNA
  modified_base 18: mod_base = OTHER; note = Unlocked nucleic acid
cttaccccacttaactantctgttttagagctatgct

SEQ ID NO: 612 - RNA, 37 nt, synthetic construct
  source 1..37: mol_type = other RNA
  modified_base 19: mod_base = OTHER; note = Unlocked nucleic acid
cttaccccacttaactatnctgttttagagctatgct

SEQ ID NO: 613 - RNA, 37 nt, synthetic construct
  source 1..37: mol_type = other RNA
  modified_base 20: mod_base = OTHER; note = Unlocked nucleic acid
cttaccccacttaactatcntgttttagagctatgct

SEQ ID NO: 614 - RNA, 37 nt, synthetic construct
  source 1..37: mol_type = other RNA
  modified_base 1: mod_base = OTHER; note = Unlocked nucleic acid
ncggtcaagatggaccagcacgttttagagctatgct

SEQ ID NO: 615 - RNA, 37 nt, synthetic construct
  source 1..37: mol_type = other RNA
  modified_base 2: mod_base = OTHER; note = Unlocked nucleic acid
cnggtcaagatggaccagcacgttttagagctatgct

SEQ ID NO: 616 - RNA, 37 nt, synthetic construct
  source 1..37: mol_type = other RNA
  modified_base 3: mod_base = OTHER; note = Unlocked nucleic acid
cgngtcaagatggaccagcacgttttagagctatgct

SEQ ID NO: 617 - RNA, 37 nt, synthetic construct
  source 1..37: mol_type = other RNA
  modified_base 4: mod_base = OTHER; note = Unlocked nucleic acid
cggntcaagatggaccagcacgttttagagctatgct

SEQ ID NO: 618 - RNA, 37 nt, synthetic construct
  source 1..37: mol_type = other RNA
  modified_base 5: mod_base = OTHER; note = Unlocked nucleic acid
cggtncaagatggaccagcacgttttagagctatgct

SEQ ID NO: 619 - RNA, 37 nt, synthetic construct
  source 1..37: mol_type = other RNA
  modified_base 6: mod_base = OTHER; note = Unlocked nucleic acid
cggtcnaagatggaccagcacgttttagagctatgct

SEQ ID NO: 620 - RNA, 37 nt, synthetic construct
  source 1..37: mol_type = other RNA
  modified_base 7: mod_base = OTHER; note = Unlocked nucleic acid
cggtcanagatggaccagcacgttttagagctatgct

SEQ ID NO: 621 - RNA, 37 nt, synthetic construct
  source 1..37: mol_type = other RNA
  modified_base 8: mod_base = OTHER; note = Unlocked nucleic acid
cggtcaangatggaccagcacgttttagagctatgct

SEQ ID NO: 622 - RNA, 37 nt, synthetic construct
  source 1..37: mol_type = other RNA
  modified_base 9: mod_base = OTHER; note = Unlocked nucleic acid
cggtcaagnatggaccagcacgttttagagctatgct

SEQ ID NO: 623 - RNA, 37 nt, synthetic construct
  source 1..37: mol_type = other RNA
  modified_base 10: mod_base = OTHER; note = Unlocked nucleic acid
cggtcaagantggaccagcacgttttagagctatgct

SEQ ID NO: 624 - RNA, 37 nt, synthetic construct
  source 1..37: mol_type = other RNA
  modified_base 1: mod_base = OTHER; note = Unlocked nucleic acid
nttgggacaacgttgtccagcgttttagagctatgct

SEQ ID NO: 625 - RNA, 37 nt, synthetic construct
  source 1..37: mol_type = other RNA
  modified_base 2: mod_base = OTHER; note = Unlocked nucleic acid
tntgggacaacgttgtccagcgttttagagctatgct

SEQ ID NO: 626 - RNA, 37 nt, synthetic construct
  source 1..37: mol_type = other RNA
  modified_base 3: mod_base = OTHER; note = Unlocked nucleic acid
ttngggacaacgttgtccagcgttttagagctatgct

SEQ ID NO: 627 - RNA, 37 nt, synthetic construct
  source 1..37: mol_type = other RNA
  modified_base 4: mod_base = OTHER; note = Unlocked nucleic acid
ttgnggacaacgttgtccagcgttttagagctatgct

SEQ ID NO: 628 - RNA, 37 nt, synthetic construct
  source 1..37: mol_type = other RNA
  modified_base 5: mod_base = OTHER; note = Unlocked nucleic acid
ttggngacaacgttgtccagcgttttagagctatgct

SEQ ID NO: 629 - RNA, 37 nt, synthetic construct
  source 1..37: mol_type = other RNA
  modified_base 6: mod_base = OTHER; note = Unlocked nucleic acid
ttgggnacaacgttgtccagcgttttagagctatgct

SEQ ID NO: 630 - RNA, 37 nt, synthetic construct
  source 1..37: mol_type = other RNA
  modified_base 7: mod_base = OTHER; note = Unlocked nucleic acid
ttgggancaacgttgtccagcgttttagagctatgct

SEQ ID NO: 631 - RNA, 37 nt, synthetic construct
  source 1..37: mol_type = other RNA
  modified_base 8: mod_base = OTHER; note = Unlocked nucleic acid
ttgggacnaacgttgtccagcgttttagagctatgct

SEQ ID NO: 632 - RNA, 37 nt, synthetic construct
  source 1..37: mol_type = other RNA
  modified_base 9: mod_base = OTHER; note = Unlocked nucleic acid
ttgggacanacgttgtccagcgttttagagctatgct

SEQ ID NO: 633 - RNA, 37 nt, synthetic construct
  source 1..37: mol_type = other RNA
  modified_base 10: mod_base = OTHER; note = Unlocked nucleic acid
ttgggacaancgttgtccagcgttttagagctatgct

SEQ ID NO: 634 - RNA, 37 nt, synthetic construct
  source 1..37: mol_type = other RNA
  modified_base 1: mod_base = OTHER; note = Unlocked nucleic acid
nctacgaggagcatttgcactgttttagagctatgct

SEQ ID NO: 635 - RNA, 37 nt, synthetic construct
  source 1..37: mol_type = other RNA
  modified_base 2: mod_base = OTHER; note = Unlocked nucleic acid
cntacgaggagcatttgcactgttttagagctatgct

SEQ ID NO: 636 - RNA, 37 nt, synthetic construct
  source 1..37: mol_type = other RNA
  modified_base 3: mod_base = OTHER; note = Unlocked nucleic acid
ctnacgaggagcatttgcactgttttagagctatgct

SEQ ID NO: 637 - RNA, 37 nt, synthetic construct
  source 1..37: mol_type = other RNA
  modified_base 4: mod_base = OTHER; note = Unlocked nucleic acid
ctancgaggagcatttgcactgttttagagctatgct

SEQ ID NO: 638 - RNA, 37 nt, synthetic construct
  source 1..37: mol_type = other RNA
  modified_base 5: mod_base = OTHER; note = Unlocked nucleic acid
ctacngaggagcatttgcactgttttagagctatgct

SEQ ID NO: 639 - RNA, 37 nt, synthetic construct
  source 1..37: mol_type = other RNA
  modified_base 6: mod_base = OTHER; note = Unlocked nucleic acid
ctacgnaggagcatttgcactgttttagagctatgct

SEQ ID NO: 640 - RNA, 37 nt, synthetic construct
  source 1..37: mol_type = other RNA
  modified_base 7: mod_base = OTHER; note = Unlocked nucleic acid
ctacganggagcatttgcactgttttagagctatgct

SEQ ID NO: 641 - RNA, 37 nt, synthetic construct
  source 1..37: mol_type = other RNA
  modified_base 8: mod_base = OTHER; note = Unlocked nucleic acid
ctacgagngagcatttgcactgttttagagctatgct

SEQ ID NO: 642 - RNA, 37 nt, synthetic construct
  source 1..37: mol_type = other RNA
  modified_base 9: mod_base = OTHER; note = Unlocked nucleic acid
ctacgaggnagcatttgcactgttttagagctatgct

SEQ ID NO: 643 - RNA, 37 nt, synthetic construct
  source 1..37: mol_type = other RNA
  modified_base 10: mod_base = OTHER; note = Unlocked nucleic acid
ctacgaggangcatttgcactgttttagagctatgct

SEQ ID NO: 644 - RNA, 100 nt, synthetic construct
  source 1..100: mol_type = other RNA
  modified_base order(1..3,97..99): mod_base = OTHER; note = 2-Prime-O-methyl nucleotides
  modified_base order(1..4,97..100): mod_base = OTHER; note = Phosphorothioate linkage
gagtccgagcagaagaagaagttttagagctagaaatagcaagttaaaataaggctagtccgttatcaacttgaaaaagtggcaccgagtcggtgctttt

SEQ ID NO: 645 - RNA, 100 nt, synthetic construct
  source 1..100: mol_type = other RNA
  modified_base order(1..4,97..100): mod_base = OTHER; note = Phosphorothioate linkage
  modified_base order(1..3,97..99): mod_base = OTHER; note = 2-Prime-O-methyl nucleotides
ggggccactagggacaggatgttttagagctagaaatagcaagttaaaataaggctagtccgttatcaacttgaaaaagtggcaccgagtcggtgctttt

SEQ ID NO: 646 - RNA, 101 nt, synthetic construct
  source 1..101: mol_type = other RNA
  modified_base 3: mod_base = OTHER; note = Unlocked nucleic acid
  modified_base order(1..2,98..100): mod_base = OTHER; note = 2-Prime-O-methyl nucleotides
  modified_base order(1..3,4^5,98..101): mod_base = OTHER; note = Phosphorothioate linkage
gangtccgagcagaagaagaagttttagagctagaaatagcaagttaaaataaggctagtccgttatcaacttgaaaaagtggcaccgagtcggtgctttt

SEQ ID NO: 647 - RNA, 101 nt, synthetic construct
  source 1..101: mol_type = other RNA
  modified_base 3: mod_base = OTHER; note = Unlocked nucleic acid
  modified_base order(1..2,98..100): mod_base = OTHER; note = 2-Prime-O-methyl nucleotides
  modified_base order(1..3,4^5,98..101): mod_base = OTHER; note = Phosphorothioate linkage
ggnggccactagggacaggatgttttagagctagaaatagcaagttaaaataaggctagtccgttatcaacttgaaaaagtggcaccgagtcggtgctttt

SEQ ID NO: 648 - RNA, 23 nt, synthetic construct
  source 1..23: mol_type = other RNA
gagtccgagcagaagaagaaggg

SEQ ID NO: 649 - RNA, 25 nt, synthetic construct
  source 1..25: mol_type = other RNA
  modified_base 23..25: mod_base = OTHER; note = Phosphorothioate linkage
  modified_base 23..24: mod_base = OTHER; note = Locked nucleic acid
  modified_base order(22,25): mod_base = OTHER; note = 2-Prime-O-methyl nucleotides
  misc_feature 1: note = U at location 1 is joined to another nucleic acid U via a C3 spacer
tgagtccaggttttagagctatgct

SEQ ID NO: 650 - RNA, 24 nt, synthetic construct
  source 1..24: mol_type = other RNA
  modified_base 22..24: mod_base = OTHER; note = Phosphorothioate linkage
  modified_base 22..23: mod_base = OTHER; note = Locked nucleic acid
  modified_base order(21,24): mod_base = OTHER; note = 2-Prime-O-methyl nucleotides
  misc_feature 1: note = G at location 1 is joined to another nucleic acid C via a C3 spacer
gagtccaggttttagagctatgct

SEQ ID NO: 651 - RNA, 23 nt, synthetic construct
  source 1..23: mol_type = other RNA
  modified_base 21..23: mod_base = OTHER; note = Phosphorothioate linkage
  modified_base 21..22: mod_base = OTHER; note = Locked nucleic acid
  modified_base order(20,23): mod_base = OTHER; note = 2-Prime-O-methyl nucleotides
  misc_feature 1: note = A at location 1 is joined to another nucleic acid U via a C3 spacer
agtccaggttttagagctatgct

SEQ ID NO: 652 - RNA, 22 nt, synthetic construct
  source 1..22: mol_type = other RNA
  modified_base 20..22: mod_base = OTHER; note = Phosphorothioate linkage
  modified_base 20..21: mod_base = OTHER; note = Locked nucleic acid
  modified_base order(19,22): mod_base = OTHER; note = 2-Prime-O-methyl nucleotides
  misc_feature 1: note = G at location 1 is joined to another nucleic acid G via a C3 spacer
gtccaggttttagagctatgct

SEQ ID NO: 653 - RNA, 21 nt, synthetic construct
  source 1..21: mol_type = other RNA
  modified_base 19..21: mod_base = OTHER; note = Phosphorothioate linkage
  modified_base 19..20: mod_base = OTHER; note = Locked nucleic acid
  modified_base order(18,21): mod_base = OTHER; note = 2-Prime-O-methyl nucleotides
  misc_feature 1: note = U at location 1 is joined to another nucleic acid A via a C3 spacer
tccaggttttagagctatgct

SEQ ID NO: 654 - RNA, 20 nt, synthetic construct
  source 1..20: mol_type = other RNA
  modified_base 18..20: mod_base = OTHER; note = Phosphorothioate linkage
  modified_base 18..19: mod_base = OTHER; note = Locked nucleic acid
  modified_base order(17,20): mod_base = OTHER; note = 2-Prime-O-methyl nucleotides
  misc_feature 1: note = C at location 1 is joined to another nucleic acid G via a C3 spacer
ccaggttttagagctatgct

SEQ ID NO: 655 - RNA, 19 nt, synthetic construct
  source 1..19: mol_type = other RNA
  modified_base 17..19: mod_base = OTHER; note = Phosphorothioate linkage
  modified_base 17..18: mod_base = OTHER; note = Locked nucleic acid
  modified_base order(16,19): mod_base = OTHER; note = 2-Prime-O-methyl nucleotides
  misc_feature 1: note = C at location 1 is joined to another nucleic acid U via a C3 spacer
caggttttagagctatgct

SEQ ID NO: 656 - RNA, 18 nt, synthetic construct
  source 1..18: mol_type = other RNA
  modified_base 16..18: mod_base = OTHER; note = Phosphorothioate linkage
  modified_base 16..17: mod_base = OTHER; note = Locked nucleic acid
  modified_base order(15,18): mod_base = OTHER; note = 2-Prime-O-methyl nucleotides
  misc_feature 1: note = A at location 1 is joined to another nucleic acid C via a C3 spacer
aggttttagagctatgct

SEQ ID NO: 657 - RNA, 17 nt, synthetic construct
  source 1..17: mol_type = other RNA
  modified_base 15..17: mod_base = OTHER; note = Phosphorothioate linkage
  modified_base 15..16: mod_base = OTHER; note = Locked nucleic acid
  modified_base order(14,17): mod_base = OTHER; note = 2-Prime-O-methyl nucleotides
  misc_feature 1: note = G at location 1 is joined to another nucleic acid C via a C3 spacer
ggttttagagctatgct

SEQ ID NO: 658 - RNA, 16 nt, synthetic construct
  source 1..16: mol_type = other RNA
  modified_base 14..16: mod_base = OTHER; note = Phosphorothioate linkage
  modified_base 14..15: mod_base = OTHER; note = Locked nucleic acid
  modified_base order(13,16): mod_base = OTHER; note = 2-Prime-O-methyl nucleotides
  misc_feature 1: note = G at location 1 is joined to another nucleic acid A via a C3 spacer
gttttagagctatgct

SEQ ID NO: 659 - RNA, 25 nt, synthetic construct
  source 1..25: mol_type = other RNA
  modified_base 23..25: mod_base = OTHER; note = Phosphorothioate linkage
  modified_base 23..24: mod_base = OTHER; note = Locked nucleic acid
  modified_base order(22,25): mod_base = OTHER; note = 2-Prime-O-methyl nucleotides
  misc_feature 1: note = U at location 1 is joined to another nucleic acid G via a C3 spacer
tcctggagggttttagagctatgct

SEQ ID NO: 660 - RNA, 24 nt, synthetic construct
  source 1..24: mol_type = other RNA
  modified_base 22..24: mod_base = OTHER; note = Phosphorothioate linkage
  modified_base 22..23: mod_base = OTHER; note = Locked nucleic acid
  modified_base order(21,24): mod_base = OTHER; note = 2-Prime-O-methyl nucleotides
  misc_feature 1: note = C at location 1 is joined to another nucleic acid A via a C3 spacer
cctggagggttttagagctatgct

SEQ ID NO: 661 - RNA, 23 nt, synthetic construct
  source 1..23: mol_type = other RNA
  modified_base 21..23: mod_base = OTHER; note = Phosphorothioate linkage
  modified_base 21..22: mod_base = OTHER; note = Locked nucleic acid
  modified_base order(20,23): mod_base = OTHER; note = 2-Prime-O-methyl nucleotides
  misc_feature 1: note = C at location 1 is joined to another nucleic acid U via a C3 spacer
ctggagggttttagagctatgct

SEQ ID NO: 662 - RNA, 22 nt, synthetic construct
  source 1..22: mol_type = other RNA
  modified_base 20..22: mod_base = OTHER; note = Phosphorothioate linkage
  modified_base 20..21: mod_base = OTHER; note = Locked nucleic acid
  modified_base order(19,22): mod_base = OTHER; note = 2-Prime-O-methyl nucleotides
  misc_feature 1: note = U at location 1 is joined to another nucleic acid C via a C3 spacer
tggagggttttagagctatgct

SEQ ID NO: 663 - RNA, 21 nt, synthetic construct
  source 1..21: mol_type = other RNA
  modified_base 19..21: mod_base = OTHER; note = Phosphorothioate linkage
  modified_base 19..20: mod_base = OTHER; note = Locked nucleic acid
  modified_base order(18,21): mod_base = OTHER; note = 2-Prime-O-methyl nucleotides
  misc_feature 1: note = G at location 1 is joined to another nucleic acid C via a C3 spacer
ggagggttttagagctatgct

SEQ ID NO: 664 - RNA, 20 nt, synthetic construct
  source 1..20: mol_type = other RNA
  modified_base 18..20: mod_base = OTHER; note = Phosphorothioate linkage
  modified_base 18..19: mod_base = OTHER; note = Locked nucleic acid
  modified_base order(17,20): mod_base = OTHER; note = 2-Prime-O-methyl nucleotides
  misc_feature 1: note = G at location 1 is joined to another nucleic acid U via a C3 spacer
gagggttttagagctatgct

SEQ ID NO: 665 - RNA, 19 nt, synthetic construct
  source 1..19: mol_type = other RNA
  modified_base 17..19: mod_base = OTHER; note = Phosphorothioate linkage
  modified_base 17..18: mod_base = OTHER; note = Locked nucleic acid
  modified_base order(16,19): mod_base = OTHER; note = 2-Prime-O-methyl nucleotides
  misc_feature 1: note = A at location 1 is joined to another nucleic acid G via a C3 spacer
agggttttagagctatgct

SEQ ID NO: 666 - RNA, 18 nt, synthetic construct
  source 1..18: mol_type = other RNA
  modified_base 16..18: mod_base = OTHER; note = Phosphorothioate linkage
  modified_base 16..17: mod_base = OTHER; note = Locked nucleic acid
  modified_base order(15,18): mod_base = OTHER; note = 2-Prime-O-methyl nucleotides
  misc_feature 1: note = G at location 1 is joined to another nucleic acid G via a C3 spacer
gggttttagagctatgct

SEQ ID NO: 667 - RNA, 17 nt, synthetic construct
  source 1..17: mol_type = other RNA
  modified_base 15..17: mod_base = OTHER; note = Phosphorothioate linkage
  modified_base 15..16: mod_base = OTHER; note = Locked nucleic acid
  modified_base order(14,17): mod_base = OTHER; note = 2-Prime-O-methyl nucleotides
  misc_feature 1: note = G at location 1 is joined to another nucleic acid A via a C3 spacer
ggttttagagctatgct

SEQ ID NO: 668 - RNA, 16 nt, synthetic construct
  source 1..16: mol_type = other RNA
  modified_base 14..16: mod_base = OTHER; note = Phosphorothioate linkage
  modified_base 14..15: mod_base = OTHER; note = Locked nucleic acid
  modified_base order(13,16): mod_base = OTHER; note = 2-Prime-O-methyl nucleotides
  misc_feature 1: note = G at location 1 is joined to another nucleic acid G via a C3 spacer
gttttagagctatgct

SEQ ID NO: 669 - RNA, 25 nt, synthetic construct
  source 1..25: mol_type = other RNA
  modified_base 23..25: mod_base = OTHER; note = Phosphorothioate linkage
  modified_base 23..24: mod_base = OTHER; note = Locked nucleic acid
  modified_base order(22,25): mod_base = OTHER; note = 2-Prime-O-methyl nucleotides
  misc_feature 1: note = G at location 1 is joined to another nucleic acid C via a C3 spacer
gaagaagaagttttagagctatgct

SEQ ID NO: 670 - RNA, 24 nt, synthetic construct
  source 1..24: mol_type = other RNA
  modified_base 22..24: mod_base = OTHER; note = Phosphorothioate linkage
  modified_base 22..23: mod_base = OTHER; note = Locked nucleic acid
  modified_base order(21,24): mod_base = OTHER; note = 2-Prime-O-methyl nucleotides
  misc_feature 1: note = A at location 1 is joined to another nucleic acid A via a C3 spacer
aagaagaagttttagagctatgct

SEQ ID NO: 671 - RNA, 23 nt, synthetic construct
  source 1..23: mol_type = other RNA
  modified_base 21..23: mod_base = OTHER; note = Phosphorothioate linkage
  modified_base 21..22: mod_base = OTHER; note = Locked nucleic acid
  modified_base order(20,23): mod_base = OTHER; note = 2-Prime-O-methyl nucleotides
  misc_feature 1: note = A at location 1 is joined to another nucleic acid G via a C3 spacer
agaagaagttttagagctatgct

SEQ ID NO: 672 - RNA, 22 nt, synthetic construct
  source 1..22: mol_type = other RNA
  modified_base 20..22: mod_base = OTHER; note = Phosphorothioate linkage
  modified_base 20..21: mod_base = OTHER; note = Locked nucleic acid
  modified_base order(19,22): mod_base = OTHER; note = 2-Prime-O-methyl nucleotides
  misc_feature 1: note = G at location 1 is joined to another nucleic acid A via a C3 spacer
gaagaagttttagagctatgct

SEQ ID NO: 673 - RNA, 21 nt, synthetic construct
  source 1..21: mol_type = other RNA
  modified_base 19..21: mod_base = OTHER; note = Phosphorothioate linkage
  modified_base 19..20: mod_base = OTHER; note = Locked nucleic acid
  modified_base order(18,21): mod_base = OTHER; note = 2-Prime-O-methyl nucleotides
  misc_feature 1: note = A at location 1 is joined to another nucleic acid A via a C3 spacer
aagaagttttagagctatgct

SEQ ID NO: 674 - RNA, 20 nt, synthetic construct
  source 1..20: mol_type = other RNA
  modified_base 18..20: mod_base = OTHER; note = Phosphorothioate linkage
  modified_base 18..19: mod_base = OTHER; note = Locked nucleic acid
  modified_base order(17,20): mod_base = OTHER; note = 2-Prime-O-methyl nucleotides
  misc_feature 1: note = A at location 1 is joined to another nucleic acid G via a C3 spacer
agaagttttagagctatgct

SEQ ID NO: 675 - RNA, 19 nt, synthetic construct
  source 1..19: mol_type = other RNA
  modified_base 17..19: mod_base = OTHER; note = Phosphorothioate linkage
  modified_base 17..18: mod_base = OTHER; note = Locked nucleic acid
  modified_base order(16,19): mod_base = OTHER; note = 2-Prime-O-methyl nucleotides
  misc_feature 1: note = G at location 1 is joined to another nucleic acid A via a C3 spacer
gaagttttagagctatgct

SEQ ID NO: 676 - RNA, 18 nt, synthetic construct
  source 1..18: mol_type = other RNA
  modified_base 16..18: mod_base = OTHER; note = Phosphorothioate linkage
  modified_base 16..17: mod_base = OTHER; note = Locked nucleic acid
  modified_base order(15,18): mod_base = OTHER; note = 2-Prime-O-methyl nucleotides
  misc_feature 1: note = A at location 1 is joined to another nucleic acid A via a C3 spacer
aagttttagagctatgct

SEQ ID NO: 677 - RNA, 17 nt, synthetic construct
  source 1..17: mol_type = other RNA
  modified_base 15..17: mod_base = OTHER; note = Phosphorothioate linkage
  modified_base 15..16: mod_base = OTHER; note = Locked nucleic acid
  modified_base order(14,17): mod_base = OTHER; note = 2-Prime-O-methyl nucleotides
  misc_feature 1: note = A at location 1 is joined to another nucleic acid G via a C3 spacer
agttttagagctatgct

(19) United States
(12) Patent Application Publication
(10) Pub. No.: US 2026/0218173 A1
(43) Pub. Date: Jul. 30, 2026

(54) MODIFIED GUIDE RNA FOR REDUCING OFF-TARGET EDITING

(71) Applicant: Integrated DNA Technologies, Inc., Coralville, IA (US)

(72) Inventors: Kyle KINNEY, Coralville, IA (US); Garrett R. RETTIG, Coralville, IA (US); Karthik MURUGAN, Coralville, IA (US)

(73) Assignee: Integrated DNA Technologies, Inc., Coralville, IA (US)

(21) Appl. No.: 19/419,399

(22) Filed: Dec. 15, 2025

Related U.S. Application Data
(60) Provisional application No. 63/735,995, filed on Dec. 19, 2024.
(60) Provisional application No. 63/929,023, filed on Dec. 2, 2025.

(51) Int. Cl. C12N 15/11

(52) U.S. Cl. CPC C12N 15/11; C12N 2310/20; C12N 2310/322; C12N 2310/3231; C12N 2310/344; C12N 2320/52

(57) ABSTRACT

Modified synthetic guide RNAs which can be used to broadly reduce off-target editing while retaining on-target editing efficiency

This application claims the benefit of U.S. Ser. No. 63/735,995, filed Dec. 19, 2024, and U.S. Ser. No. 63/929,023, filed Dec. 2, 2025 the entireties of which are incorporated herein by reference.

### REFERENCE TO AN ELECTRONIC SEQUENCE LISTING

A Sequence Listing is provided herewith as a Sequence Listing XML, “6391-0012WO01” is 1,414,205 bytes in size and was created on Dec. 12, 2025. The contents of the Sequence Listing XML are incorporated by reference herein in their entirety.

### BACKGROUND

The CRISPR-Cas9 system has fundamentally transformed genetic engineering and molecular biology by enabling precise and efficient genome editing. Originally derived from a natural adaptive immune mechanism in bacteria, CRISPR-Cas9 allows researchers to make targeted modifications to the DNA of various organisms, offering unprecedented potential for applications in medicine, agriculture, and biotechnology (1). The system comprises two main components: the Cas9 nuclease, which acts as molecular scissors to cleave DNA, and the guide RNA (gRNA), which directs the Cas9 to specific DNA sequences for editing (1).

However, despite its precision, CRISPR-Cas9 is not without its challenges. One of the most significant issues is the occurrence of off-target effects (OTEs), where unintended genomic regions similar to the target sequence are also edited (2, 3). These off-target edits can lead to unwanted genetic changes that can disrupt the function of essential genes, leading to adverse effects on cellular function, viability, development of secondary diseases, or exacerbate existing conditions that pose risks in therapeutic applications.

Another significant issue affecting Cas9 is its cleavage efficiency. Several factors have been linked to editing efficiency at the intended target which include GC content, chromatin state, and gRNA structure². Depending on target application, the intended target's design constraints may impede optimal gRNA design, which could lead to reduced activity or an increased risk of off-target effects. Therefore, enhancing the specificity of gRNAs while either maintaining or increasing editing efficiency is crucial for improving the efficacy, safety, and reliability of CRISPR-Cas based genome editing.

Efforts to mitigate off-target effects primarily focus on optimizing the design and modification of the gRNA. Enhancing the specificity of gRNA without sacrificing its efficiency is crucial for improving the safety and reliability of CRISPR-Cas9-based genome editing. To mitigate off-target effects and modulate editing efficiency, researchers have developed several strategies focused on improving gRNA design and modifying the CRISPR-Cas9 system. These strategies include rationale gRNA design, chemical modifications of the gRNA, truncated gRNAs, high-fidelity Cas-nuclease variants (HiFi), and CRISPR hybrid RNA-DNA (chRDNA) gRNAs (4-6, 8). All of these strategies have known trade-offs.

For example, gRNA design is essential to get the best editing outcome; however, it might not be possible to design a gRNA without potential off-targets while also keeping high on-target editing efficiency. This is further exacerbated when designing base editing therapeutics where disease-causing SNPs limit the design of gRNAs to specific regions. Additionally, current chemical modifications such as the 2′-O-Methyl and phosphorothioates do not broadly eliminate off-target editing.

On the other hand, high-fidelity Cas nucleases broadly reduce off-target editing but can also reduce on-target editing in a target-dependent manner. The current best strategy to mitigate off-targets while retaining on-target activity is the use of chRDNA gRNAs. The RNA:DNA chimeric nature of these gRNAs distorts the structure of the heteroduplex, slowing the Cas9 cleavage rate and promotes dissociation of the off-target substrate (8). The downside to this type of gRNA is that the number and location of the DNAs in the spacer is unique to each target.

Therefore, one would have to screen potentially hundreds to thousands of gRNA designs to optimize the on-/off-target editing ratio. The screening of hundreds to thousands of gRNAs is only tenable for therapeutic labs. Notably, none of the chRDNAs increased editing efficiency compared to RNA only gRNAs. Moreover, none of the chRDNAs were tested with gRNAs that exhibited poor on-target editing performance.

This creates the need for a more universal strategy that can broadly reduce OTEs while retaining on-target activity and improve editing performance across various metrics and editing modalities.

This disclosure provides potential modification patterns with single base modifications of the gRNA spacer region that reduce off-target editing while retaining on-target activity. Also provided are UNA locations within the gRNA spacer region that either improved on-target editing efficiency for multiple targets or showed strong reductions in off-target editing while retaining on-target activity.

All references cited herein are incorporated herein by reference in their entireties.

### BRIEF SUMMARY

In exemplary embodiments the disclosure provides a synthetic guide RNA (“sgRNA”) comprising: (i) a first nucleotide sequence comprising at least one modified nucleotide, wherein the first nucleotide is partially or completely complementary to a target nucleic acid; and (ii) a second nucleic acid sequence which interacts with a CRISPR-associated protein (Cas) polypeptide, wherein the synthetic guide RNA guides the Cas polypeptide to the target nucleic acid, and wherein the synthetic guide RNA exhibits a reduced off-target editing relative to an unmodified gRNA. The disclosure provides a synthetic guide RNA wherein the sgRNA exhibits an enhanced on-target activity. The disclosure provides a synthetic guide RNA wherein at least one modified nucleotide is selected from the group consisting of unlocked nucleic acid (UNA), locked nucleic acid (LNA), 2′fluoro, C3 spacer, dSpacer, and combinations thereof. The disclosure provides a synthetic guide RNA wherein the first nucleic acid and second nucleic acid are a single nucleic acid strand. The disclosure provides a synthetic guide RNA wherein the first nucleic acid and second nucleic acid are two separate nucleic acid strands. The disclosure provides a synthetic guide RNA wherein the first nucleotide sequence is about 14-25 nucleotides in length. The disclosure provides a synthetic guide RNA wherein the at least one modified nucleotide is present at a position selected from the group consisting of nucleotide 1, nucleotide 2, nucleotide 3, nucleotide 4, nucleotide 5, nucleotide 6, nucleotide 7, nucleotide 8, nucleotide 9, nucleotide 10, nucleotide 11, nucleotide 12, nucleotide 13, nucleotide 14, nucleotide 15, nucleotide 16, nucleotide 17, nucleotide 18, nucleotide 19, and nucleotide 20, wherein the nucleotides are numbered from the first nucleotide of the 5′ end of the first nucleic acid. The disclosure provides a synthetic guide RNA wherein off-target editing relative to an unmodified gRNA is reduced by at least an amount selected from the group consisting of about 50%, 60%, 70%, 80%, 85%, 90%, 95%, and 99%. The disclosure provides a synthetic guide RNA wherein the first nucleic acid comprises a 3′ modification. The disclosure provides a synthetic guide RNA wherein the first nucleic acid comprises a 5′ modification. The disclosure provides a synthetic guide RNA wherein the at least one modified nucleotide alters base-pairing thermostability. The disclosure provides a synthetic guide RNA wherein said at least one modified nucleotide enhances base-pairing thermostability. The disclosure provides a synthetic guide RNA wherein said at least one modified nucleotide decreases base-pairing thermostability. The disclosure provides a synthetic guide RNA wherein the at least one modified nucleotide is a specificity-altering modification. The disclosure provides a synthetic guide RNA wherein the specificity-altering at least one modified nucleotide is located in the guide sequence. The disclosure provides a synthetic guide RNA wherein at least two nucleotides in the first nucleotide sequence are modified nucleotides. The disclosure provides a synthetic guide RNA wherein one or more modified nucleotides are located within five nucleotides from the 5′-end of the first nucleotide sequence. The disclosure provides a synthetic guide RNA wherein from about 5% to about 30% of the nucleotides in the first nucleotide sequence are modified nucleotides. The disclosure provides a synthetic guide RNA wherein the at least one modified nucleotide is located within five nucleotides from the 3′-end of the second nucleotide sequence. The disclosure provides a synthetic guide RNA wherein the modified sgRNA comprises one, two, or three consecutive or non-consecutive modified nucleotides at or near the 5′-end of the first nucleotide sequence and one, two, or three consecutive or non-consecutive modified nucleotides at or near the 3′-end of the second nucleotide sequence. The disclosure provides a synthetic guide RNA wherein the modified sgRNA comprises three consecutive modified nucleotides at the 5′-end of the first nucleotide sequence and three consecutive modified nucleotides at the 3′-end of the second nucleotide sequence. The disclosure provides a synthetic guide RNA wherein the modified sgRNA is chemically synthesized.

The disclosure provides a set or library of RNA molecules comprising two or more synthetic guide RNAs as disclosed herein. The disclosure provides a kit comprising the synthetic guide RNA as disclosed herein. The disclosure provides an array of RNA molecules comprising two or more synthetic guide RNAs as disclosed herein.

The disclosure provides a method for reducing off-target effect in a cell, the method comprising: introducing into the cell: (a) synthetic guide RNA comprising: (i) a first nucleotide sequence comprising at least one modified nucleotide, wherein the first nucleotide sequence is partially or completely complementary to a target sequence; and (ii) a second nucleic acid sequence which interacts with a CRISPR-associated protein (Cas) polypeptide, wherein the synthetic guide RNA guides the Cas polypeptide to the target nucleic acid, and wherein the synthetic guide RNA exhibits a reduced off-target effect relative to an unmodified gRNA, (b) a Cas polypeptide, an mRNA encoding a Cas polypeptide, or a recombinant expression vector comprising a nucleotide sequence encoding a Cas polypeptide, wherein the synthetic guide RNA guides the Cas polypeptide to the target nucleic acid, and wherein the synthetic guide RNA induces a gene regulation of the target nucleic acid with an enhanced activity relative to a corresponding unmodified gRNA. The disclosure provides a method for reducing off-target effect, wherein the sgRNA exhibits an enhanced on-target activity. The disclosure provides a method for reducing off-target effect in a cell wherein the at least one modified nucleotide is selected from the group consisting of unlocked nucleic acid (UNA), locked nucleic acid (LNA), 2′fluoro, C3 spacer, dSpacer, and combinations thereof. The disclosure provides a method for inducing gene regulation of a target nucleic acid in a cell wherein the first nucleic acid and second nucleic acid are a single nucleic acid strand. The disclosure provides a method for inducing gene regulation of a target nucleic acid in a cell wherein the first nucleic acid and second nucleic acid are two separate nucleic acid strands. The disclosure provides a method for inducing gene regulation of a target nucleic acid in a cell wherein the first nucleotide sequence is about 20 nucleotides in length. The disclosure provides a method for inducing gene regulation of a target nucleic acid in a cell wherein the at least one modified nucleotide is present at a position selected from the group consisting of nucleotide 1, nucleotide 2, nucleotide 3, nucleotide 4, nucleotide 5, nucleotide 6, nucleotide 7, nucleotide 8, nucleotide 9, nucleotide 10, nucleotide 11, nucleotide 12, nucleotide 13, nucleotide 14, nucleotide 15, nucleotide 16, nucleotide 17, nucleotide 18, nucleotide 19, and nucleotide 20, wherein the nucleotides are numbered from the first nucleotide of the 5′ end of the first nucleic acid. The disclosure provides a method for reducing off-target effect in a cell wherein the off-target effect of the synthetic guide RNA relative to an unmodified gRNA is reduced by at least an amount selected from the group consisting of 50%, 60%, 70%, 80%, 85%, 90%, 95%, and 99%. The disclosure provides a method for reducing off-target effect in a cell wherein the first nucleic acid comprises a 3′ modification. The disclosure provides a method for inducing gene regulation of a target nucleic acid in a cell wherein the first nucleic acid comprises a 5′ modification. The disclosure provides a method for inducing gene regulation of a target nucleic acid in a cell wherein the at least one modified nucleotide alters base-pairing thermostability. The disclosure provides a method for inducing gene regulation of a target nucleic acid in a cell wherein said at least one modified nucleotide enhances base-pairing thermostability. The disclosure provides a method for inducing gene regulation of a target nucleic acid in a cell wherein said at least one modified nucleotide decreases base-pairing thermostability. The disclosure provides a method for inducing gene regulation of a target nucleic acid in a cell wherein the at least one modified nucleotide is a specificity-altering modification. The disclosure provides a method for inducing gene regulation of a target nucleic acid in a cell wherein the specificity-altering at least one modified nucleotide is located in the guide sequence. The disclosure provides a method for inducing gene regulation of a target nucleic acid in a cell wherein at least two nucleotides in the first nucleotide sequence are modified nucleotides. The disclosure provides a method for inducing gene regulation of a target nucleic acid in a cell wherein one or more modified nucleotides are located within five nucleotides from the 5′-end of the first nucleotide sequence. The disclosure provides a method for inducing gene regulation of a target nucleic acid in a cell wherein from about 5% to about 30% of the nucleotides in the first nucleotide sequence are modified nucleotides. The disclosure provides a method for inducing gene regulation of a target nucleic acid in a cell wherein the at least one modified nucleotide is located within five nucleotides from the 3′-end of the second nucleotide sequence. The disclosure provides a method for inducing gene regulation of a target nucleic acid in a cell wherein the modified sgRNA comprises one, two, or three consecutive or non-consecutive modified nucleotides at or near the 5′-end of the first nucleotide sequence and one, two, or three consecutive or non-consecutive modified nucleotides at or near the 3′-end of the second nucleotide sequence. The disclosure provides a method for inducing gene regulation of a target nucleic acid in a cell wherein the modified sgRNA comprises three consecutive modified nucleotides at the 5′-end of the first nucleotide sequence and three consecutive modified nucleotides at the 3′-end of the second nucleotide sequence. The disclosure provides a method for inducing gene regulation of a target nucleic acid in a cell wherein the modified sgRNA is chemically synthesized. The disclosure provides a method for inducing gene regulation of a target nucleic acid in a cell wherein the target nucleic acid comprises a target DNA or a target RNA. The disclosure provides a method for inducing gene regulation of a target nucleic acid in a cell wherein the gene regulation comprises genome editing of the target DNA. The disclosure provides a method for inducing gene regulation of a target nucleic acid in a cell wherein the genome editing comprises homologous-directed repair (HDR) or nonhomologous end joining (NHEJ) of the target DNA. The disclosure provides a method for inducing gene regulation of a target nucleic acid in a cell further comprising introducing a recombinant donor repair template into the cell. The disclosure provides a method for inducing gene regulation of a target nucleic acid in a cell wherein the modified sgRNA in (a) and the Cas polypeptide in (b) are introduced into the cell in a ribonucleoprotein (RNP) complex. The disclosure provides a method for inducing gene regulation of a target nucleic acid in a cell wherein the gene regulation induced by the introduction of (a) and (b) is stable in the cell for at least 24 hours. The disclosure provides a method for inducing gene regulation of a target nucleic acid in a cell wherein the modified sgRNA in (a) and the Cas polypeptide in (b) are introduced into the cell in a lipofection reagent. The disclosure provides a method for inducing gene regulation of a target nucleic acid in a cell wherein the modified sgRNA in (a) and the Cas polypeptide in (b) are introduced into the cell via exosomes. The disclosure provides a method for inducing gene regulation of a target nucleic acid in a cell wherein the modified sgRNA in (a) and the Cas polypeptide in (b) are introduced into the cell via lipid nanoparticles. The disclosure provides a method for inducing gene regulation of a target nucleic acid in a cell wherein the modified sgRNA in (a) and the Cas polypeptide in (b) are introduced into the cell via viral vector.

### BRIEF DESCRIPTION OF SEVERAL VIEWS OF THE DRAWINGS

The invention will be described in conjunction with the following drawings in which like reference numerals designate like elements and wherein:

FIG. 1 is a chart showing the chemical structures of nucleic acid modifications.

FIG. 2 (SEQ ID Nos: 335-338) is a chart showing Single UNA modifications within the gRNA spacer reduce editing of off-targets. On- and off-target editing as determined by RHAMPSEQ NGS for EMX1. Low to high indel formation is indicated by a white to black heat map. Individual values for indel formation are indicated in each cell. The top three edited off-targets are shown with mismatches relative to the on-target underlined, all other off-targets exhibited the same reduction in editing as the top three OTE sites.

FIG. 3A (SEQ ID Nos: 339-342) Single UNA modifications within the gRNA spacer reduce editing of off-targets for multiple targets. On- and off-target editing as determined by RHAMPSEQ NGS for AR. Low to high indel formation is indicated by a white to black heat map. The top three edited off-targets are shown with mismatches relative to the on-target underlined for each gRNA.

FIG. 3B (SEQ ID Nos: 343-346) Single UNA modifications within the gRNA spacer reduce editing of off-targets for multiple targets. On- and off-target editing as determined by RHAMPSEQ NGS for LAG3. Low to high indel formation is indicated by a white to black heat map. The top three edited off-targets are shown with mismatches relative to the on-target underlined for each gRNA.

FIG. 4A (SEQ ID Nos: 335-338) Single UNA modifications within the gRNA spacer reduce editing of off-targets when delivered by RNP. On- and off-target editing as determined by RHAMPSEQ NGS for EMX1. Low to high indel formation is indicated by a white to black heat map. The top three edited off-targets are shown with mismatches relative to the on-target underlined for each gRNA.

FIG. 4B (SEQ ID Nos: 339-342) Single UNA modifications within the gRNA spacer reduce editing of off-targets when delivered by WT Cas9 RNP. On- and off-target editing as determined by RHAMPSEQ NGS for AR. Low to high indel formation is indicated by a white to black heat map. AR had no off-target editing above 0.5%, only the first five OTEs are shown. The top three edited off-targets are shown with mismatches relative to the on-target underlined for each gRNA.

FIG. 4C (SEQ ID Nos: 343-346) Single UNA modifications within the gRNA spacer reduce editing of off-targets when delivered by RNP. On- and off-target editing as determined by RHAMPSEQ NGS for LAG3. Low to high indel formation is indicated by a white to black heat map. Only OTEs with editing above 1% are shown for LAG3. The top three edited off-targets are shown with mismatches relative to the on-target underlined for each gRNA.

FIG. 5A (SEQ ID Nos: 335-338) Single LNA modifications within the gRNA spacer reduce editing of off-targets. On- and off-target editing as determined by RHAMPSEQ NGS for LNA with EMX1. Low to high indel formation is indicated by a white to black heat map. Individual values for indel formation are indicated in each cell. The top three edited off-targets are shown with mismatches relative to the on-target underlined for each gRNA.

FIG. 5B (SEQ ID Nos: 335-338) Single 2′Fluoro modifications within the gRNA spacer reduce editing of off-targets. On- and off-target editing as determined by RHAMPSEQ NGS for 2′fluoro with EMX1. Low to high indel formation is indicated by a white to black heat map. Individual values for indel formation are indicated in each cell. The top three edited off-targets are shown with mismatches relative to the on-target underlined for each gRNA.

FIG. 5C (SEQ ID Nos: 339-342) Single LNA modifications within the gRNA spacer reduce editing of off-targets. On- and off-target editing as determined by RHAMPSEQ NGS for LNA with AR. Low to high indel formation is indicated by a white to black heat map. The top three edited off-targets are shown with mismatches relative to the on-target underlined for each gRNA.

FIG. 5D (SEQ ID Nos: 339-342) Single 2′Fluoro modifications within the gRNA spacer reduce editing of off-targets. On- and off-target editing as determined by RHAMPSEQ NGS for 2′Fluoro with AR. Low to high indel formation is indicated by a white to black heat map. The top three edited off-targets are shown with mismatches relative to the on-target underlined for each gRNA.

FIG. 5E (SEQ ID Nos: 343-346) Single LNA modifications within the gRNA spacer reduce editing of off-targets. On- and off-target editing as determined by RHAMPSEQ NGS for LNA with LAG3. Low to high indel formation is indicated by a white to black heat map. The top three edited off-targets are shown with mismatches relative to the on-target underlined for each gRNA.

FIG. 5F (SEQ ID Nos: 343-346) Single 2′Fluoro modifications within the gRNA spacer reduce editing of off-targets. On- and off-target editing as determined by RHAMPSEQ NGS for 2′Fluoro with LAG3. Low to high indel formation is indicated by a white to black heat map The top three edited off-targets are shown with mismatches relative to the on-target underlined for each gRNA.

FIG. 6A (SEQ ID Nos: 335-338) Single LNA modifications within the gRNA spacer reduce editing of off-targets when delivered by RNP. On- and off-target editing as determined by RHAMPSEQ NGS for LNA with EMX1. Low to high indel formation is indicated by a white to black heat map. Individual values for indel formation are indicated in each cell. The top three edited off-targets are shown with mismatches relative to the on-target underlined for each gRNA.

FIG. 6B (SEQ ID Nos: 335-338) Single 2′Fluoro modifications within the gRNA spacer reduce editing of off-targets when delivered by RNP. On- and off-target editing as determined by RHAMPSEQ NGS for 2′Fluoro with EMX1. Low to high indel formation is indicated by a white to black heat map. Individual values for indel formation are indicated in each cell. The top three edited off-targets are shown with mismatches relative to the on-target underlined for each gRNA.

FIG. 6C (SEQ ID Nos: 339-342) Single LNA modifications within the gRNA spacer reduce editing of off-targets when delivered by RNP. On- and off-target editing as determined by RHAMPSEQ NGS for LNA with AR. Low to high indel formation is indicated by a white to black heat map. Individual values for indel formation are indicated in each cell. AR had no off-target editing above 0.5%, only the first five OTEs are shown. The top three edited off-targets are shown with mismatches relative to the on-target underlined for each gRNA.

FIG. 6D (SEQ ID Nos: 339-342) Single 2′Fluoro modifications within the gRNA spacer reduce editing of off-targets when delivered by RNP. On- and off-target editing as determined by RHAMPSEQ NGS for 2′Fluoro with AR. Low to high indel formation is indicated by a white to black heat map. Individual values for indel formation are indicated in each cell. AR had no off-target editing above 0.5%, only the first five OTEs are shown. The top three edited off-targets are shown with mismatches relative to the on-target underlined for each gRNA.

FIG. 6E (SEQ ID Nos: 343-346) Single LNA modifications within the gRNA spacer reduce editing of off-targets when delivered by RNP. On- and off-target editing as determined by RHAMPSEQ NGS for LNA with LAG3. Low to high indel formation is indicated by a white to black heat map. Individual values for indel formation are indicated in each cell. Only OTEs with editing above 1% are shown for LAG3. The top three edited off-targets are shown with mismatches relative to the on-target underlined for each gRNA.

FIG. 6F (SEQ ID Nos: 343-346) Single 2′Fluoro modifications within the gRNA spacer reduce editing of off-targets when delivered by RNP. On- and off-target editing as determined by RHAMPSEQ NGS for 2′Fluoro with LAG3. Low to high indel formation is indicated by a white to black heat map. Individual values for indel formation are indicated in each cell. Only OTEs with editing above 1% are shown for LAG3. The top three edited off-targets are shown with mismatches relative to the on-target underlined for each gRNA.

FIG. 7A (SEQ ID Nos: 335-338) Single C3 spacer modifications within the gRNA spacer reduce editing of off-targets. On- and off-target editing as determined by RHAMPSEQ NGS for C3 spacer with EMX1. Low to high indel formation is indicated by a white to black heat map. Individual values for indel formation are indicated in each cell. The top three edited off-targets are shown with mismatches relative to the on-target underlined for each gRNA.

FIG. 7B (SEQ ID Nos: 335-338) Single dSpacer modifications within the gRNA spacer reduce editing of off-targets. On- and off-target editing as determined by RHAMPSEQ NGS for dSpacer with EMX1. Low to high indel formation is indicated by a white to black heat map. Individual values for indel formation are indicated in each cell. The top three edited off-targets are shown with mismatches relative to the on-target underlined for each gRNA.

FIG. 7C (SEQ ID Nos: 339-342) Single C3 spacer modifications within the gRNA spacer reduce editing of off-targets. On- and off-target editing as determined by RHAMPSEQ NGS for C3 spacer with AR. Low to high indel formation is indicated by a white to black heat map. The top three edited off-targets are shown with mismatches relative to the on-target underlined for each gRNA.

FIG. 7D (SEQ ID Nos: 339-342) Single dSpacer modifications within the gRNA spacer reduce editing of off-targets. On- and off-target editing as determined by RHAMPSEQ NGS for dSpacer with AR. Low to high indel formation is indicated by a white to black heat map. The top three edited off-targets are shown with mismatches relative to the on-target underlined for each gRNA.

FIG. 7E (SEQ ID Nos: 343-346) Single C3 spacer modifications within the gRNA spacer reduce editing of off-targets. On- and off-target editing as determined by RHAMPSEQ NGS for C3 spacer with LAG3. Low to high indel formation is indicated by a white to black heat map. The top three edited off-targets are shown with mismatches relative to the on-target underlined for each gRNA.

FIG. 7F (SEQ ID Nos: 343-346) Single dSpacer modifications within the gRNA spacer reduce editing of off-targets. On- and off-target editing as determined by RHAMPSEQ NGS for dSpacer with LAG3. Low to high indel formation is indicated by a white to black heat map. The top three edited off-targets are shown with mismatches relative to the on-target underlined for each gRNA.

FIG. 8A (SEQ ID Nos: 335-337) are charts showing a single UNA modification at spacer position 18 reduces off-target editing similar to HiFi-Cas9 in various cell types and nuclease delivery formats; RNP dose titration in K562s with EMX1.

FIG. 8B (SEQ ID Nos: 335-337) are charts showing a single UNA modification at spacer position 18 reduces off-target editing similar to HiFi-Cas9 in various cell types and nuclease delivery formats; RNP dose titration in iPSCs with EMX1

FIG. 8C (SEQ ID Nos: 347-349) are charts showing a single UNA modification at spacer position 18 reduces off-target editing similar to HiFi-Cas9 in various cell types and nuclease delivery formats; RNP dose titration in iPSCs with AAVS1

FIG. 8D are charts showing a single UNA modification at spacer position 18 reduces off-target editing similar to HiFi-Cas9 in various cell types and nuclease delivery formats; Editing with EMX1 and AAVS1 in iPSCs with delivery of Cas-nuclease via mRNA. On- and off-target editing as determined by RHAMPSEQ NGS for EMX1 and AAVS1. The top two edited off-target targets are shown with mismatches underlined relative to the on-target. All other off-targets had <1% indel. Data is representative of three biological replicates.

FIG. 9 is a chart showing single UNA modifications within the gRNA spacer increase on-target editing efficiency. On-target editing as determined by RHAMPSEQ NGS.

FIG. 10A is a chart showing Single UNA modifications within the gRNA spacer increase on-target editing efficiency with stable-Cas9 genomic expression.

FIG. 10B is a chart showing single UNA modifications within the gRNA spacer increase on-target editing efficiency RNP Cas9 delivery. On-target editing as determined by RHAMPSEQ NGS.

FIG. 11 is a chart showing UNCOVERseq gRNA editing specificity scores. Specificity Score=(On-target UMI Reads)/(On and off target UMI Reads).

FIG. 12A is a chart showing placement of a single UNA within the gRNA spacer modulates CRISPR-Cas editing efficiency. Editing for UNA placement in each spacer location for all sixteen targets (Table 8; SEQ ID NO: 352 to SEQ ID NO: 367) is normalized to the editing of the RNA only spacer region crRNAs for the on-target site and the top edited off-target site with stable Cas9 expression. Data is represented as the median±95% CI.

FIG. 12B is a chart showing placement of a single UNA within the gRNA spacer modulates CRISPR-Cas editing efficiency. Editing for UNA placement in each spacer location for all sixteen targets (Table 8; SEQ ID NO: 352 to SEQ ID NO: 367) is normalized to the editing of the RNA only spacer region crRNAs for the on-target site and the top edited off-target site with RNP. Data is represented as the median±95% CI.

FIG. 13A is a chart showing optimally placed UNA modified gRNAs increase editing specificity. A) Comparison of editing frequencies between ALT-R crRNAs and UNA modified crRNAs. On-target sites are solid black points; off-targets are grey points.

FIG. 13B is a chart showing Tukey box plot showing fold change of all on-targets and off-targets between UNA modified crRNAs and ALT-R crRNAs. Results are from editing rates from on- and off-target sites for each gRNA. UNA modified gRNAs used in comparisons are as follows: SEQ ID NO: 372, SEQ ID NO: 392, SEQ ID NO: 401, SEQ ID NO: 418, SEQ ID NO: 440, SEQ ID NO: 462, SEQ ID NO: 484, SEQ ID NO: 496, SEQ ID NO: 517, SEQ ID NO: 539, SEQ ID NO: 560, SEQ ID NO: 576, SEQ ID NO: 602, SEQ ID NO: 614, SEQ ID NO: 624, SEQ ID NO: 634. Statistical significance was determined using Mann-Whitney test. B) ****P<0.0001.

FIG. 14A and FIG. 14B. UNA modifications within the gRNA spacer improve editing specificity with sgRNAs. On/off-target editing as determined by RHAMPSEQ NGS. EMX1 ALT-R sgRNA: SEQ_ID_644, EMX1 UNA modified sgRNA: SEQ_ID_646, AAVS1 ALT-R sgRNA: SEQ_ID_645, AAVS1 UNA modified sgRNA: SEQ_ID_647. EMX1 On-target: SEQ_ID_335, OTE1: SEQ_ID_336, OTE2: SEQ_ID_337. AAVS1 On-target: SEQ_ID_347, OTE1: SEQ_ID_348, OTE2: SEQ_ID_349.

FIG. 15. (SEQ ID NO: 648) Labeling scheme for placement of nucleic acid modifications. Cas9 target site example, EMX1, where the gRNA spacer region is underlined and numbered from 1-20 starting at the PAM adjacent base. The PAM is indicated by bold letters.

### DETAILED DESCRIPTION

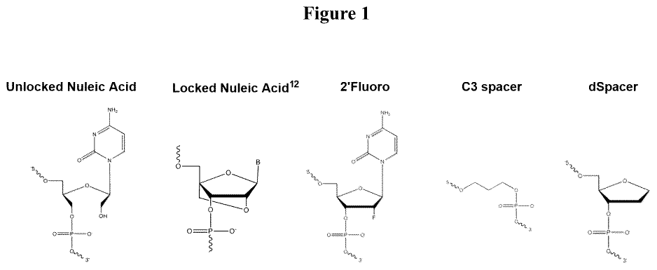

The current disclosure provides novel designs of modified synthetic guide RNAs (sgRNAs”) for CRISPR systems, which have reduced off-target editing, relative to conventional gRNAs, while retaining on-target editing efficiency. Utilizing the same working mechanism of heteroduplex distortion to decrease off-target editing, a list of five nucleic acid modifications with different structural properties was generated as potential gRNA spacer modifications candidates: unlocked nucleic acid (UNA), locked nucleic acid (LNA), 2′fluoro, C3 spacer, and dSpacer (See FIG. 1). UNAs are acyclic RNA mimics that have a highly flexible structure due to the missing bond between C2′ and C3′ atoms of the ribose ring (9). UNAs, depending on their position in the duplex, can either increase or decrease mismatch discrimination against RNA/DNA target strands while simultaneously decreasing the thermodynamic stability of the duplex (9). These properties make it the ideal test modification for gRNA duplex distortion to reduce off-target editing. Contrastingly, LNAs are in many ways the polar opposites of UNAs. LNAs contain a methylene bridge that connects the 2′-oxygen of ribose with the 4′-carbon (10). This bridge results in a locked confirmation, reducing the conformational flexibility of the ribose and increases the local organization of the phosphate backbone (10). Furthermore, LNAs have an increased affinity for complementary RNA/DNA making the modification ideal for testing comparisons with UNAs. In addition to UNAs and LNAs, the 2′fluoro has been studied for therapeutic applications due to its unique properties of small size and high electronegativity. 2′fluoro nucleotides replace the 2′-hydrooxyl group in a RNA monomer with a fluorine molecule and have increased binding affinity and nuclease resistance while retaining similar structural properties to standard RNA bases meaning it may have a subtler effect on duplex distortion (11). Lastly, abasic modifications such as the C3 spacer and dSpacer offer two different chemical structures (flexible—C3 spacer, standard deoxyribose sugar phosphate backbone—dSpacer) while also providing a universal mismatch with no nucleotide base being present. The disclosure provides a list of potential modification patterns with, for example, single base modifications of the gRNA spacer region that reduces off-target editing while retaining on-target activity.

The modified sgRNAs as disclosed herein can be used to broadly reduce off-target editing while retaining on-target editing efficiency with, for example, a WT-Cas9 enzyme. This may satisfy known gaps in editing efficiencies of HiFi Cas nuclease systems where the HiFi enzyme reduces both on/off-target editing. Furthermore, the screening required to optimize placement of individual gRNA spacer modifications to enhance the on/off-target editing ratio is dramatically lower than what is required for optimization of chRDNAs. These results open the door for these modified sgRNAs to be used in a wider context compared to chRDNAs, including, for example, phenotypic screens, cell line engineering, therapeutic development, etc.

The modified sgRNAs as disclosed herein have the advantage of increasing WT Cas9 gRNA specificity while retaining on-target cleavage efficiency with the use of a single modified base in the gRNA spacer region. The modified sgRNAs as disclosed herein have the further advantage of decreasing the amount of gRNA screening necessary to find top performing gRNA, i.e., gRNAs with high on-target/low off-target activity.

The term “nucleic acid” refers to a nucleotide polymer, and unless otherwise limited, includes analogs of natural nucleotides that can function in a similar manner (e.g., hybridize) to naturally occurring nucleotides. The term “nucleic acid” encompasses multi-stranded, as well as single-stranded molecules. In double- or triple-stranded nucleic acids, the nucleic acid strands need not be coextensive (i.e., a double-stranded nucleic acid need not be double-stranded along the entire length of both strands). Nucleic acid templates described herein may be any size depending on the sample (from small cell-free DNA fragments to entire genomes), including but not limited to 50-300 bases, 100-2000 bases, 100-750 bases, 170-500 bases, 100-5000 bases, 50-10,000 bases, or 50-2000 bases in length. In some instances, templates are at least 50, 100, 200, 500, 1000, 2000, 5000, 10,000, 20,000 50,000, 100,000, 200,000, 500,000, 1,000,000 or more than 1,000,000 bases in length. Methods described herein provide for the amplification of nucleic acids, such as nucleic acid templates. Methods described herein additionally provide for the generation of isolated and at least partially purified nucleic acids and libraries of nucleic acids. Nucleic acids include but are not limited to those comprising DNA, RNA, circular RNA, cfDNA (cell free DNA), cfRNA (cell free RNA), siRNA (small interfering RNA), cffDNA (cell free fetal DNA), mRNA, tRNA, rRNA, miRNA (microRNA), synthetic polynucleotides, polynucleotide analogues, any other nucleic acid consistent with the specification, or any combinations thereof. The length of polynucleotides, when provided, are described as the number of bases and abbreviated, such as nt (nucleotides), bp (bases), kb (kilobases), or Gb (gigabases).

The term nucleic acid includes any form of DNA or RNA, including, for example, genomic DNA; complementary DNA (cDNA), which is a DNA representation of mRNA, usually obtained by reverse transcription of messenger RNA (mRNA) or by amplification; DNA molecules produced synthetically or by amplification; mRNA; and non-coding RNA.

The term nucleic acid encompasses double- or triple-stranded nucleic acid complexes, as well as single-stranded molecules. In double- or triple-stranded nucleic acid complexes, the nucleic acid strands need not be coextensive (i.e., a double-stranded nucleic acid need not be double-stranded along the entire length of both strands).

The term nucleic acid also encompasses any modifications thereof, such as by methylation and/or by capping. Nucleic acid modifications can include addition of chemical groups that incorporate additional charge, polarizability, hydrogen bonding, electrostatic interaction, and functionality to the individual nucleic acid bases or to the nucleic acid as a whole. Such modifications may include base modifications such as 2′-position sugar modifications, 5-position pyrimidine modifications, 8-position purine modifications, modifications at cytosine exocyclic amines, substitutions of 5-bromo-uracil, sugar-phosphate backbone modifications, unusual base pairing combinations such as the isobases isocytidine and isoguanidine, and the like. More particularly, in some embodiments, nucleic acids, can include polydeoxyribonucleotides (containing 2-deoxy-D-ribose), polyribonucleotides (containing D-ribose), and any other type of nucleic acid that is an N- or C-glycoside of a purine or pyrimidine base, as well as other polymers containing nonnucleotidic backbones, for example, polyamide (e.g., peptide nucleic acids (PNAs)) and polymorpholino polymers (see, e.g., Summerton and Weller (1997) “Morpholino Antisense Oligomers: Design, Preparation, and Properties,” Antisense & Nucleic Acid Drug Dev. 7:1817-195; Okamoto et al. (20020) “Development of electrochemically gene-analyzing method using DNA-modified electrodes,” Nucleic Acids Res. Supplement No. 2:171-172), and other synthetic sequence-specific nucleic acid polymers providing that the polymers contain nucleobases in a configuration which allows for base pairing and base stacking, such as is found in DNA and RNA. The term nucleic acid also encompasses locked nucleic acids (LNAs).

The nucleic acid(s) can be derived from a completely chemical synthesis process, such as a solid phase-mediated chemical synthesis, from a biological source, such as through isolation from any species that produces nucleic acid, or from processes that involve the manipulation of nucleic acids by molecular biology tools, such as DNA replication, PCR amplification, reverse transcription, or from a combination of those processes.

As used herein, the term “complementary” refers to the capacity for precise pairing between two nucleotides, i.e., if a nucleotide at a given position of a nucleic acid is capable of hydrogen bonding with a nucleotide of another nucleic acid to form a canonical base pair, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. The degree of complementarity between nucleic acid strands has significant effects on the efficiency and strength of hybridization between nucleic acid strands.

The term “oligonucleotide” is used to refer to a nucleic acid that is relatively short, generally shorter than 200 nucleotides, more particularly, shorter than 100 nucleotides, most particularly, shorter than 50 nucleotides. Typically, oligonucleotides are single-stranded DNA molecules. The term “oligonucleotide,” as used herein, refer to polydeoxyribonucleotides (containing 2-deoxy-D-ribose), polyribonucleotides (containing D-ribose), and to any other type of polynucleotide which is an N glycoside of a purine or pyrimidine base (a single nucleotide is also referred to as a “base” or “residue”). There is no intended distinction in length between the terms “nucleic acid”, “oligonucleotide” and “polynucleotide”, and these terms can be used interchangeably. These terms refer only to the primary structure of the molecule. Thus, these terms include double- and single-stranded DNA, as well as double- and single-stranded RNA. For use in the present invention, an oligonucleotide also can comprise nucleotide analogs in which the base, sugar or phosphate backbone is modified as well as non-purine or non-pyrimidine nucleotide analogs. An oligonucleotide may comprise ribonucleotides, deoxyribonucleotides, modified nucleotides (e.g., nucleotides with 2′ modifications, synthetic base analogs, etc.) or combinations thereof.

The term “ribonucleotide” encompasses natural and synthetic, unmodified and modified ribonucleotides. Modifications include changes to the sugar moiety, to the base moiety and/or to the linkages between ribonucleotides in the oligonucleotide.

The term “polypeptide” refers to any linear or branched peptide comprising more than one amino acid. Polypeptide includes protein or fragment thereof or fusion thereof, provided such protein, fragment or fusion retains a useful biochemical or biological activity.

Next Generation Sequencing (NGS) allows rapid and high-throughput sequencing of DNA and RNA. Unlike earlier methods such as Sanger sequencing, which sequences one DNA fragment at a time, NGS enables the simultaneous sequencing of millions of DNA fragments, making it much faster, cheaper, and more efficient. In NGS, a DNA or RNA from the sample is extracted and fragmented into smaller pieces. These fragments are then attached to short synthetic DNA sequences called adapters, which are needed for binding to the sequencing platform. The DNA fragments with adapters are amplified (copied many times) to create a “library” of DNA fragments. This increases the amount of DNA available for sequencing. Most NGS platforms, like Illumina, use a method called “sequencing by synthesis.” Each fragment is attached to a solid surface and copied in place. Fluorescently-labeled nucleotides (A, T, C, and G) are added one by one. As they bind to the complementary strand, the machine detects the fluorescent signal, allowing the sequence of bases to be read. The massive amount of sequencing data is analyzed using bioinformatics tools. The overlapping DNA fragments are assembled back into their original sequence by aligning them to a reference genome or constructing new genomes (de novo sequencing). NGS allows for High Throughput, since millions to billions of DNA fragments can be sequenced in parallel, producing vast amounts of data, is cost-effective, and can sequence entire genomes or large sets of genes in days, making it much faster than older sequencing methods.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example, “1, 2 and/or 3” is equivalent to “1, 2, 3, 1 and 2, 1 and 3, 2 and 3, or 1, 2, and 3”.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition, in a description of a method, or in a description of elements of a device, is understood to encompass those compositions, methods, or devices consisting essentially of and consisting of the recited components or elements, optionally in addition to other components or elements. The disclosure as illustratively described herein suitably may be practiced in the absence of any element, elements, limitation, or limitations which is not specifically disclosed herein.

As used herein and in the appended claims, the singular forms “a”, “an”, and “the” include plural referents unless the context clearly dictates otherwise. Thus, for example, reference to “a method” includes a plurality of such methods and reference to “the nanoparticle” includes reference to one or more nanoparticles and equivalents thereof known to those skilled in the art, and so forth. The terms and expressions which have been employed are used as terms of description and not of limitation, and there is no intention in the use of such terms and expressions of excluding any equivalents of the features shown and described or portions thereof, but it is recognized that various modifications are possible within the scope as disclosed herein claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

As used herein, the term “about” when used in conjunction with a stated numerical value or range has the meaning reasonably ascribed to it by a person skilled in the art, i.e., denoting somewhat more or somewhat less than the stated value or range.

As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is an elderly human. In another embodiment, the subject is a human adult. In another embodiment, the subject is a human child. In yet another embodiment, the subject is a human infant.

CRISPR-associated (Cas) proteins useful in certain embodiments as disclosed herein may include: Cas9: The most well-known CRISPR protein, primarily from Streptococcus pyogenes (SpCas9), which cuts double-stranded DNA with high precision using a single-guide RNA (sgRNA); SaCas9 (Staphylococcus aureus)—smaller than SpCas9, useful for viral delivery; NmCas9 (Neisseria meningitidis)—recognizes a different protospacer adjacent motif (PAM) and offers alternative targeting sites; St1Cas9 (Streptococcus thermophilus)—used for organisms with specific PAM requirements; Cas9 Nickase Variants (Cas9n); Cas9 modified to create single-strand cuts (nicks) instead of double-strand breaks; HiFi Cas9 was developed as an alternative to Cas9 to create an enzyme that maintained potent on-target editing activity but had reduced off-target editing activity; Dead Cas9 (dCas9), A catalytically inactive form of Cas9. Used for gene regulation and visualization, as it can bind to DNA without cutting it; Cas12 (Cpf1), Alternative to Cas9, derived from Francisella novicida (FnCpf1) and Acidaminococcus (AsCpf1), Creates staggered (sticky) ends rather than blunt ends; Cas12a—recognizes a T-rich PAM, useful for AT-rich genomes; Cas12b—smaller Cas12 variant, suitable for viral delivery systems; Cas12f (Cpf1 Mini), a Smaller variant useful for gene-editing applications with size constraints; AsCas12a Ultra is an enhanced variant of the original AsCas12a, a CRISPR-associated protein from the Cas12a family (formerly known as Cpf1) derived from Acidaminococcus species; MAD7 is a CRISPR-associated protein that belongs to the Cas12 family (a Type V CRISPR system). Developed by the company Inscripta; Cas13, Targets RNA instead of DNA, useful for RNA interference and detection; Cas13a (formerly C2c2)—cleaves RNA and has been used in diagnostics; Cas13b different RNA cleavage specificity and applications in gene silencing; Cas13d—smaller version of Cas13, enabling delivery via compact vectors; Cas3, Known for its processive degradation of DNA; Csf1, Type III (RNA-Targeting): Csm and Cmr Complexes, Type III CRISPR systems target RNA with Csm and Cmr protein complexes, Useful for viral RNA degradation in bacterial immunity.

CRISPR guide RNA (gRNA) directs the Cas9 enzyme to a specific location in the genome where it needs to make a cut. The gRNA is designed to match a target DNA sequence, ensuring the CRISPR-Cas9 system edits only the intended site. The guide RNA is made up of two main parts: CRISPR RNA (crRNA) which is a sequence of about 20 nucleotides that is complementary to the target DNA sequence. Its primary function is to guide the Cas9 protein to the exact location in the genome where the DNA cut should be made; and Trans-activating CRISPR RNA (tracrRNA) which aids in forming a stable complex with the Cas9 enzyme. It's necessary for the activation of the Cas9 protein, enabling it to perform its function as molecular scissors.

A CRISPR eukaryotic expression cassette typically consists of several elements that together allow the CRISPR system to function efficiently within eukaryotic cells. These elements include the necessary components for gene editing, such as the Cas protein (usually Cas9) and the guide RNA (gRNA), such as the sgRNA as disclosed herein, system. In certain embodiments as disclosed herein, additional components for a eukaryotic CRISPR expression cassette may include a promoter for Cas Protein Expression, such as CMV (Cytomegalovirus) promoter, EF1α (Elongation factor-1α) promoter, Ubiquitin C (UbC) promoter, or Tissue-specific promoters for targeted Cas9 expression, e.g., neuron-specific promoters like Synapsin (Syn) or liver-specific like Albumin promoter. In certain embodiments as disclosed herein, additional components for a eukaryotic CRISPR expression cassette may include Cas Protein Coding Sequence, such as HiFi Cas, or SpCas9, Cpf1/Cas12a, SaCas9, or other Cas9 variants. In certain embodiments as disclosed herein, additional components for a eukaryotic CRISPR expression cassette may include Nuclear Localization Signal (NLS) to ensure proper transport of the Cas9 protein into the nucleus of the eukaryotic cell. In certain embodiments as disclosed herein, additional components for a eukaryotic CRISPR expression cassette may include a Promoter for gRNA Expression, such as a U6 promoter, an H1 promoter, or a Tissue-specific Pol II promoters. In certain embodiments as disclosed herein, additional components for a eukaryotic CRISPR expression cassette may include a Guide RNA (gRNA) Expression Unit, such as gRNA scaffold, or Multiplexing gRNAs. In certain embodiments as disclosed herein, additional components for a eukaryotic CRISPR expression cassette may include a Polyadenylation Signal (pA), or a Selectable Marker such as Antibiotic resistance genes, or Fluorescent markers. In certain embodiments as disclosed herein the expression cassette may include Viral Vector Elements, such as Lentiviral vectors, AAV (Adeno-associated virus) vectors, or Self-inactivating (SIN) elements. In certain embodiments as disclosed herein the expression cassette may include Inducible Systems, such as Tet-On/Tet-Off systems or CRISPRa/i systems, or Insulator Sequences such as cHS4 insulators.

A Cas NLS (Nuclear Localization Signal) is a short peptide sequence that is added to CRISPR-associated (Cas) proteins, like Cas9, to help them enter the nucleus of a eukaryotic cell. Since gene-editing processes like CRISPR-Cas9 target DNA, which is located in the cell's nucleus, it's essential that Cas proteins efficiently reach this compartment. The Nuclear Localization Signal (NLS) is a specific sequence of amino acids that is recognized by the cell's transport machinery. This sequence acts like a “tag” that signals the cell to transport the Cas protein into the nucleus. The NLS binds to nuclear import proteins, which then facilitate the passage of the Cas protein through nuclear pores, channels that regulate movement between the cytoplasm and the nucleus. By attaching an NLS to Cas proteins, scientists ensure that these proteins reach the nucleus quickly and efficiently, enabling precise and effective gene editing within the target DNA.

There are several effective strategies for introducing the sgRNAs and/or CRISPR components (like plasmids, ribonucleoprotein complexes, or mRNA) as disclosed herein as disclosed herein into target cells. Exemplary embodiments as disclosed herein include Viral Vectors, such as Adeno-Associated Virus (AAV) which are widely used for CRISPR delivery because they are generally safe, induce minimal immune response, and have been approved in some gene therapy applications. However, their small packaging capacity (around 4.7 kb) limits the size of CRISPR systems they can carry, so they work best for smaller Cas proteins (like Cas9 variants or Cas12a); Lentivirus and Retrovirus: Lentiviral vectors have a larger capacity than AAV and can integrate the CRISPR components into the host genome, allowing for stable, long-term expression. However, this integration can cause insertional mutagenesis; Adenovirus: Adenovirus vectors can carry larger payloads, including the standard SpCas9 and multiple gRNAs. They are non-integrating, but they can induce stronger immune responses, which may limit their use in some settings.

Additional methods for introducing the sgRNAs and/or CRISPR proteins as disclosed herein into target cells includes, for example, Lipid Nanoparticles (LNPs) which are commonly used for delivering RNA-based therapies, including mRNA for Cas proteins and gRNA complexes. They are a non-viral delivery method that is scalable and relatively low-risk, with minimal immune response and no genomic integration. LNPs are currently used in clinical applications and are effective for delivery in vivo, especially in the liver and other tissues with good blood flow; Electroporation, which involves applying an electrical field to create temporary pores in the cell membrane, allowing the sgRNAs and/or CRISPR components (like plasmids, ribonucleoprotein complexes, or mRNA) as disclosed herein to enter the cell. It is especially effective for cell lines, primary cells, and immune cells such as T-cells. This method is efficient but can be harsh on sensitive cells, leading to higher cell mortality. Additional methods for introducing the sgRNAs and/or CRISPR components (like plasmids, ribonucleoprotein complexes, or mRNA) as disclosed herein into target cells includes, for example, Ribonucleoprotein (RNP) Complexes which involves directly delivering the Cas9 protein pre-complexed with guide RNA, such as the sgRNA as disclosed hereininto cells, usually via electroporation or lipid-based transfection. This approach has advantages: it minimizes the risk of off-target effects, reduces immune response, and is transient, avoiding genomic integration. Lipid-Based Transfection Agents (lipofection) uses lipid-based reagents to encapsulate CRISPR plasmids or RNP complexes and facilitate their uptake by cells. This is straightforward and widely used for cell lines, but its efficiency can vary across cell types and is generally less effective for primary or difficult-to-transfect cells.

Other methods for introducing the sgRNAs and/or CRISPR components (for example, plasmids, ribonucleoprotein complexes, or mRNA) as disclosed herein into target cells includes, for example, physical methods such as Microinjection, which directly injects the sgRNAs and/or CRISPR components (for example, plasmids, ribonucleoprotein complexes, or mRNA) as disclosed herein into cells, typically used in single-cell embryos or zygotes for generating transgenic animals. This is a precise but labor-intensive approach; Nanoneedles and Microfluidics: Emerging physical methods like nanoneedles or microfluidic devices can introduce the sgRNAs and/or CRISPR components (for example, plasmids, ribonucleoprotein complexes, or mRNA) as disclosed herein with minimal damage to cells. They're promising for in vitro applications and high-throughput settings but are still being developed. Exosome-Mediated Delivery, which can be engineered to carry the sgRNAs and/or CRISPR components (for example, plasmids, ribonucleoprotein complexes, or mRNA) as disclosed herein and target them to specific cells. This is a promising, non-viral, cell-derived delivery method that may allow for targeted delivery with minimal immune response.

Next Generation Sequencing (NGS) allows rapid and high-throughput sequencing of DNA and RNA. Unlike earlier methods such as Sanger sequencing, which sequences one DNA fragment at a time, NGS enables the simultaneous sequencing of millions of DNA fragments, making it much faster, cheaper, and more efficient. In NGS, a DNA or RNA from the sample is extracted and fragmented into smaller pieces. These fragments are then attached to short synthetic DNA sequences called adapters, which are needed for binding to the sequencing platform. The DNA fragments with adapters are amplified (copied many times) to create a “library” of DNA fragments. This increases the amount of DNA available for sequencing. Most NGS platforms, like Illumina, use a method called “sequencing by synthesis.” Each fragment is attached to a solid surface and copied in place. Fluorescently-labeled nucleotides (A, T, C, and G) are added one by one. As they bind to the complementary strand, the machine detects the fluorescent signal, allowing the sequence of bases to be read. The massive amount of sequencing data is analyzed using bioinformatics tools. The overlapping DNA fragments are assembled back into their original sequence by aligning them to a reference genome or constructing new genomes (de novo sequencing). NGS allows for High Throughput, since millions to billions of DNA fragments can be sequenced in parallel, producing vast amounts of data, is cost-effective, and can sequence entire genomes or large sets of genes in days, making it much faster than older sequencing methods.

All references throughout this application, for example patent documents, including issued or granted patents or equivalents and patent application publications, and non-patent literature documents or other source material are hereby incorporated by reference herein in their entireties, as though individually incorporated by reference. None is admitted to be prior art.

The invention will be illustrated in more detail with reference to the following Examples, but it should be understood that the present invention is not deemed to be limited thereto.

### EXAMPLES

### Example 1. Assessment of UNA Modifications on Cas9 Editing

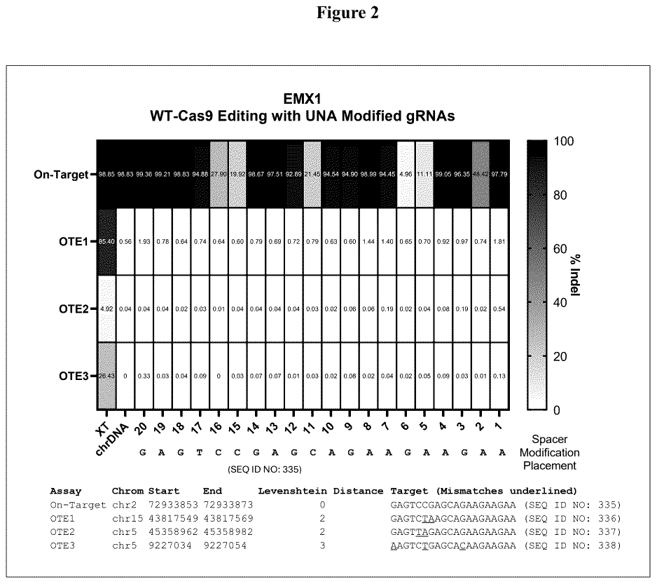

crRNAs for the guide EMX1 were designed and produced with UNA modifications that were placed sequentially along the gRNA spacer (SEQ ID NO: 4 to SEQ ID NO: 23) to determine what effect UNAs would have on Cas9 editing. The crRNAs retained the 3′ modifications of IDT's standard crRNA XT to ensure proper annealing to the tracrRNA; however, the 2′-O-Methyls and phosphorothioate linkages on the 5′ end of the crRNA were removed to confirm that any reduction in editing was due solely to the single UNA modification. These modified sgRNAs were compared to IDT's standard crRNA XT as well as the published EMX1 chRDNA design (SEQ ID NO: 1, SEQ ID NO: 2) (11). All XT and modified sgRNAs used throughout the invention disclosure were annealed to IDT's tracrRNA except for the published EMX1 chRDNA, which uses a published RNA:DNA tracrRNA (SEQ ID NO: 2, SEQ ID NO: 3) (8). Cas9 editing was assessed with HEK293 cells constitutively expressing WT Cas9 (ATCC HEK293-Cas9) in order to maximize the number of OTEs. In brief, HEK293-Cas9 cells were nucleofected with modified sgRNAs (10 μM), incubated for 72 hrs., gDNA was collected with QuickExtract, known editing sites were prepped for NGS using RHAMPSEQ, and analyzed using CRISPy (Table 1, 2). Nearly all UNA placements along the spacer yielded significant decreases in off-target editing while retaining on-target editing efficiency as compared to the standard XT modified gRNA (FIG. 2). These results were comparable to the reduction seen in off-target editing with the chRDNA control gRNA. Only UNAs placed in positions 16, 15, 11, 6, 5, and 2 saw reduction in on-target editing. Sequence mismatches within the OTE target did not seem to influence the broad reduction of editing (FIG. 2). For example, position 15 had mismatches for all 3 OTEs relative to the on-target; however, editing was reduced for all sites including the on-target. This creates indirect evidence that the structural features of the UNA modified gRNA-gDNA heteroduplex within the Cas9 enzyme maybe affecting enzyme kinetics. These results were the first instance that a single UNA modification in the gRNA spacer region can broadly reduce OTEs.

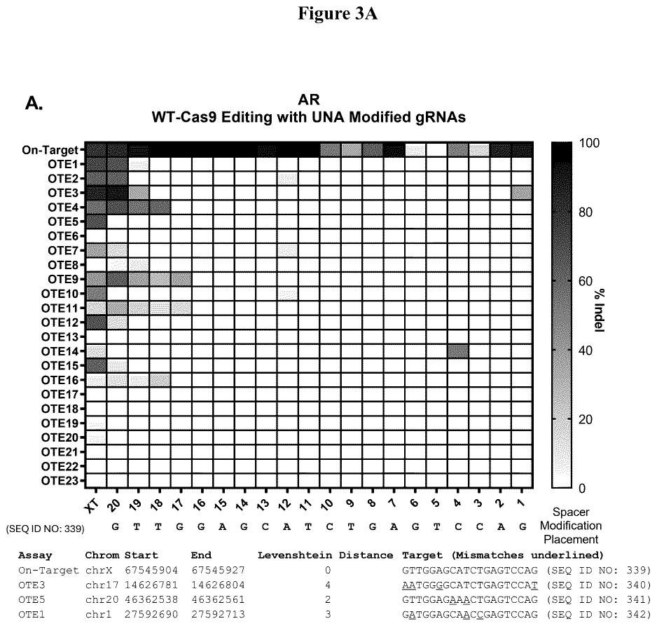

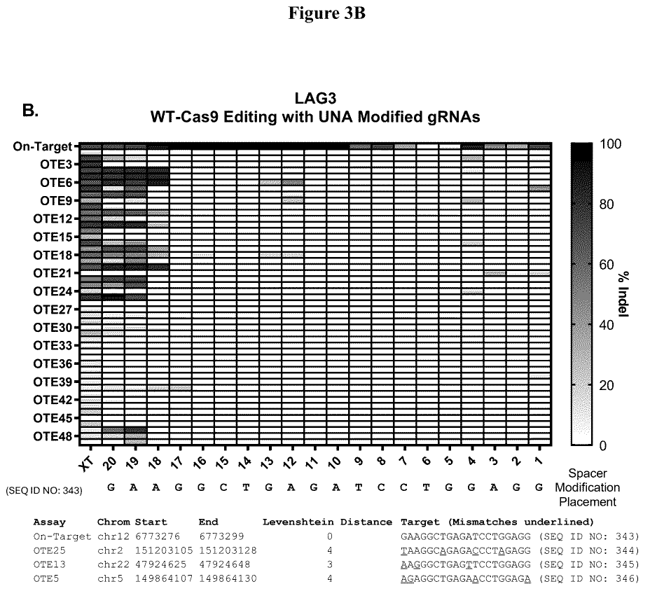

Following the results of UNA-modified EMX1 gRNAs, additional guides were ordered for targets AR and LAG3 (SEQ ID NO: 24 to SEQ ID NO: 65). UNAs were placed within the spacer in a similar manner as the EMX1 guides described above. However, the crRNA XT 5′ modifications. 2′-O-Methyl and phosphorothioates, were also added back into the design to test whether the UNA modification could combine with these modifications while still providing a functional gRNA. The UNA-modified sgRNAs were tested for editing capabilities similarly to the EMX1 gRNAs described above with previously built RHAMPSEQ panels that included the highest edited OTEs for each target (Table 3, 4). For both AR and LAG3, single UNAs placed along the gRNA spacer reduced off-target editing while retaining on-target editing (FIG. 3A and FIG. 3B). Similar to EMX1, UNAs placed in positions 12-14 broadly reduced off-target editing, creating the possibility that a UNA base could be placed in a singular location for most gRNAs to reduce OTE editing. However, UNAs placed at certain locations did show increases in OTE editing, which highlights that there may be important sequence context to how the UNA modification affects editing.

TABLE 1
EMX1 HiFi 32plex RHAMPSEQ Panel-RHC.W1014A901B0294D

| | | | | | % Indel in | % Indel in |
|---|---|---|---|---|---|---|
| | | | | | HEK293-Cas9 | unedited |
| rhAmpSeq | | | | | cells with XT | HEK293-Cas9 |
| Assay ID | Chrom | Start | End | Strand | 2-part gRNA | cells |
| EMX1_iGS_1R | chr2 | 72933853 | 72933873 | + | 98.85 | 0.35 |
| H.D049F13291 | | | | | | |
| 284F7Z0Z | | | | | | |
| EMX1_iGS_2R | chr15 | 43817549 | 43817569 | + | 85.4 | 0.62 |
| H.0F2DC25AE | | | | | | |
| 6F8476Z0Z | | | | | | |
| EMX1_iGS_6R | chr5 | 9227034 | 9227054 | + | 26.43 | 0.02 |
| H.D0DD1BA39 | | | | | | |
| 4D745BZ0Z | | | | | | |
| EMX1_iGS_3R | chr5 | 45358962 | 45358982 | + | 4.92 | 0.02 |
| H.0209198AA9 | | | | | | |
| 7B4B0Z0Z | | | | | | |
| EMX1_iGS_5R | chr8 | 127788996 | 127789016 | + | 4.82 | 0.06 |
| H.DE5AE16BF | | | | | | |
| 5A94BCZ0Z | | | | | | |
| H.3FF9CD566E | | | | | | |
| EMX1_iGS_7R | chr3 | 4989913 | 4989933 | + | 2.9 | 0.04 |
| H.3FF9CD566E | | | | | | |
| B2454Z0Z | | | | | | |
| EMX1_iGS_9R | chr3 | 34001466 | 34001486 | + | 2.51 | 0.11 |
| H.F5E73CF629 | | | | | | |
| 85480Z0Z | | | | | | |
| EMX1_picks_3 | chr4 | 25059120 | 25059142 | + | 9.96 | 7.69 |
| RH.EDC43F229 | | | | | | |
| 18D423Z0Z | | | | | | |
| EMX1_iGS_4R | chr2 | 218980334 | 218980354 | + | 1.95 | 0 |
| H.5961DD885E | | | | | | |
| 8A438Z0Z | | | | | | |
| EMX1_picks_1 | chr6 | 110170195 | 110170217 | + | 1.33 | 0.69 |
| RH.4C7CC5E7 | | | | | | |
| B805479Z0Z | | | | | | |
| EMX1_iGS_13 | chr12 | 63795301 | 63795321 | + | 78.32 | 77.75 |
| RH.73ECEF96 | | | | | | |
| CF324F0Z0Z | | | | | | |
| EMX1_iGS_10 | chr6 | 9118563 | 9118583 | + | 0.45 | 0.03 |
| RH.A4994D64E | | | | | | |
| 98547CZ0Z | | | | | | |
| EMX1_iGS_12 | chr11 | 43726382 | 43726402 | + | 0.35 | 0.02 |
| RH.3369C9084 | | | | | | |
| 5F64ACZ0Z | | | | | | |
| EMX1_iGS_20 | chr22 | 29209831 | 29209851 | + | 0.31 | 0.05 |
| RH.5AECBF55 | | | | | | |
| 6499472Z0Z | | | | | | |
| EMX1_aGS_2R | chrX | 53440758 | 53440781 | + | 0.27 | 0.02 |
| H.5DC785865B | | | | | | |
| 8E4BDZ0Z | | | | | | |
| EMX1_iGS_11 | chr1 | 234357116 | 234357136 | + | 0.29 | 0.05 |
| RH.0575AAFD | | | | | | |
| B47F4B4Z0Z | | | | | | |
| EMX1_picks_2 | chr11 | 6265868 | 6265890 | + | 1.8 | 1.64 |
| RH.A15AB0DB | | | | | | |
| 1005440Z0Z | | | | | | |
| EMX1_iGS_19 | chr4 | 53164820 | 53164840 | + | 1.89 | 1.76 |
| RH.9E7531076 | | | | | | |
| 76E454Z0Z | | | | | | |
| EMX1_picks_8 | chr10 | 128309468 | 128309490 | + | 0.32 | 0.24 |
| RH.7D16F71AF | | | | | | |
| 1724BEZ0Z | | | | | | |
| EMX1_iGS_18 | chr3 | 63482435 | 63482455 | + | 0.15 | 0.07 |
| RH.1414AACB | | | | | | |
| AF3D4F2Z0Z | | | | | | |
| EMX1_aGS_1R | chr1 | 23394119 | 23394142 | + | 0.07 | 0.01 |
| H.269461AAFC | | | | | | |
| 394BEZ0Z | | | | | | |
| EMX1_iGS_17 | chr12 | 121970230 | 121970250 | + | 0.07 | 0.01 |
| RH.2037479DF | | | | | | |
| B2E4B4Z0Z | | | | | | |
| EMX1_picks_6 | chr2 | 202842752 | 202842774 | + | 0.1 | 0.07 |
| RH.F64EE96D4 | | | | | | |
| 445444Z0Z | | | | | | |
| EMX1_aGS_3R | chr5 | 147453621 | 147453644 | + | 0.08 | 0.05 |
| H.9492583992F | | | | | | |
| A405Z0Z | | | | | | |
| EMX1_iGS_8R | chr16 | 56150160 | 56150180 | + | 0.11 | 0.11 |
| H.AE575FCOEF | | | | | | |
| 1B470Z0Z | | | | | | |
| EMX1_picks_7 | chr11 | 30280257 | 30280279 | + | 0.01 | 0.01 |
| RH.B8A71E4B | | | | | | |
| 6B474AAZ0Z | | | | | | |
| EMX1_iGS_15 | chr7 | 73900798 | 73900818 | + | 0.03 | 0.04 |
| RH.042CD79E7 | | | | | | |
| BFA4F2Z0Z | | | | | | |
| EMX1_picks_9 | chr14 | 42687599 | 42687621 | + | 0.37 | 0.38 |
| RH.CDF7A091 | | | | | | |
| 3EB7433Z0Z0 | | | | | | |
| RH.9D8FEE22 | | | | | | |
| EMX1_picks_10 | chr2 | 217513380 | 217513402 | + | 0.06 | 0.09 |
| RH.9D8FEE22 | | | | | | |
| EAE845EZ0Z | | | | | | |
| EMX1_picks_4 | chr7 | 140837898 | 140837920 | + | 0 | 0.03 |
| RH.53B8E1C96 | | | | | | |
| 01745AZ0Z | | | | | | |
| EMX1_iGS_14 | chr1 | 35353288 | 35353308 | + | 0.02 | 0.06 |
| RH.3779C1B5D | | | | | | |
| 4224D6Z0Z | | | | | | |
| EMX1_picks_5 | chr4 | 86335534 | 86335556 | + | 2.29 | 3.14 |
| RH.B009628D8 | | | | | | |
| 63040DZ0Z | | | | | | |

TABLE 2
EMX1_Top10v2 RHAMPSEQ Panel-RHC.F3644FF7B16242C

| | | | | | % Indel in | |
|---|---|---|---|---|---|---|
| | | | | | HEK293-Cas9 | % Indel in |
| | | | | | cells with | unedited |
| rhAmpSeq | | | | | XT 2-part | HEK293-Cas9 |
| Assay ID | Chrom | Start | End | Strand | gRNA | cells |
| EMX1OnTRH.1 | chr2 | 72933852 | 72933875 | + | 98.45 | 0.39 |
| 7730AC8CEF4 | | | | | | |
| 4C5Z0Z | | | | | | |
| EMX1OTE001 | chr15 | 43817548 | 43817571 | + | 90.14 | 0.68 |
| RH.9DDB49A8 | | | | | | |
| 2C8C440Z0Z | | | | | | |
| EMX1OTE002 | chr5 | 45358958 | 45358981 | - | 89.81 | 0.07 |
| RH.2CA5E0423 | | | | | | |
| 5C34DFZ0Z | | | | | | |
| EMX1OTE003 | chr5 | 9227033 | 9227056 | + | 34.35 | 0.04 |
| RH.96864F3D2 | | | | | | |
| 166494Z0Z | | | | | | |
| EMX1OTE004 | chr8 | 127788995 | 127789018 | + | 11.07 | 0.09 |
| RH.4280E82040 | | | | | | |
| AD43FZ0Z | | | | | | |
| EMX1OTE005 | chrX | 53440757 | 53440780 | - | 4.19 | 0.01 |
| RH.D532A8C0 | | | | | | |
| 428444DZ0Z | | | | | | |
| EMX1OTE006 | chr2 | 218980333 | 218980356 | + | 2.59 | 0 |
| RH.3027735429 | | | | | | |
| F444AZ0Z | | | | | | |
| EMX1OTE007 | chr3 | 4989912 | 4989935 | + | 2.08 | 0.16 |
| RH.68CE2561A | | | | | | |
| 33A4E0Z0Z | | | | | | |
| EMX1OTE008 | chr3 | 34001465 | 34001487 | + | 4.14 | 0.06 |
| RH.6737976A5 | | | | | | |
| 2694DEZ0Z | | | | | | |
| EMX1OTE009 | chr6 | 9118559 | 9118582 | - | 1.27 | 0.07 |
| RH.83FDB76D | | | | | | |
| 02914D1Z0Z | | | | | | |

TABLE 3
AR_Top25 RHAMPSEQ Panel-RHC.639E9BFE3C594AC

| | | | | | % Indel in | |
|---|---|---|---|---|---|---|
| | | | | | HEK293-Cas9 | % Indel in |
| | | | | | cells with | unedited |
| rhAmpSeq | | | | | XT 2-part | HEK293-Cas9 |
| Assay ID | Chrom | Start | End | Strand | gRNA | cells |
| ARall_001RH.7 | chrX | 67545904 | 67545927 | + | 82.33 | 0.225 |
| 5FE149A32D54 | | | | | | |
| B1Z0Z | | | | | | |
| ARall_002RH.8 | chr1 | 27592690 | 27592713 | - | 56.88 | 0.035 |
| 72507C6EA824 | | | | | | |
| 02Z0Z | | | | | | |
| ARall_003RH.8 | chr7 | 22126332 | 22126355 | - | 47.49 | 0.165 |
| CB5A0C40F20 | | | | | | |
| 449Z0Z | | | | | | |
| ARall_004RH.6 | chr17 | 14626781 | 14626804 | - | 74.17 | 0.095 |
| 3C10B389D134 | | | | | | |
| B2Z0Z | | | | | | |
| ARall_006RH.3 | chr12 | 122113355 | 122113378 | - | 44.095 | 0.1 |
| 9A2EBF9F2B5 | | | | | | |
| 4B5Z0Z | | | | | | |
| ARall_007RH. | chr20 | 46362538 | 46362561 | - | 58.045 | 0.015 |
| A42E04C6C9F | | | | | | |
| E402Z0Z | | | | | | |
| DD4A477DA2 | | | | | | |
| ARall_008RH. | chr6 | 150412433 | 150412455 | - | 1.5 | 0.01 |
| DD4A477DA2 | | | | | | |
| BE484Z0Z | | | | | | |
| ARall_009RH.E | chr8 | 70014900 | 70014923 | - | 25.13 | 0.12 |
| 7C332E7797B4 | | | | | | |
| D1Z0Z | | | | | | |
| ARall_010RH.7 | chr18 | 26782003 | 26782026 | + | 2.985 | 0.06 |
| 7B9DAB45DE7 | | | | | | |
| 493Z0Z | | | | | | |
| ARall_011RH.B | chr10 | 75605945 | 75605968 | - | 27.365 | 0.055 |
| 2361525E71D4 | | | | | | |
| 72Z0Z | | | | | | |
| ARall_012RH.C | chr15 | 32101670 | 32101693 | - | 42.175 | 0.21 |
| 1142667FCD24 | | | | | | |
| FAZ0Z | | | | | | |
| ARall_013RH.0 | chr6 | 110986340 | 110986364 | + | 9.845 | 0.08 |
| 6AD4BD86962 | | | | | | |
| 4B4Z0Z | | | | | | |
| ARall_014RH.0 | chr1 | 195970288 | 195970311 | - | 53.82 | 0.16 |
| F19F82DA9BE | | | | | | |
| 474Z0Z | | | | | | |
| ARall_015RH.F | chr19 | 39394095 | 39394118 | - | 1.04 | 0.025 |
| BA89781A3834 | | | | | | |
| 52Z0Z | | | | | | |
| ARall_016RH.7 | chr2 | 99538778 | 99538801 | + | 9.875 | 0.12 |
| 5BA692504684 | | | | | | |
| E1Z0Z | | | | | | |
| ARall_017RH. | chr4 | 33488669 | 33488692 | + | 45.61 | 0.165 |
| A23BC0BFC3F | | | | | | |
| 24ADZ0Z | | | | | | |
| ARall_018RH.9 | chr6 | 111617768 | 111617791 | + | 5.2 | 0.54 |
| 0EB6C97E22B | | | | | | |
| 44FZ0Z | | | | | | |
| ARall_019RH.7 | chr17 | 41631349 | 41631371 | - | 0.185 | 0.105 |
| 970A6B4B83F4 | | | | | | |
| 79Z0Z | | | | | | |
| ARall_020RH.E | chr17 | 16212979 | 16213002 | + | 0.81 | 0.015 |
| 1B19AC4C2C2 | | | | | | |
| 40FZ0Z | | | | | | |
| ARall_021RH.7 | chr17 | 80396918 | 80396939 | - | 3.075 | 0.07 |
| C5D69D0C906 | | | | | | |
| 497Z0Z | | | | | | |
| ARall_022RH.9 | chr9 | 129737563 | 129737586 | - | 3.51 | 0.125 |
| D7B9B43CF60 | | | | | | |
| 4F6Z0Z | | | | | | |
| ARall_023RH.7 | chr10 | 116741323 | 116741346 | - | 2.565 | 0 |
| 8F3FBC3F5864 | | | | | | |
| 6BZ0Z | | | | | | |
| ARall_024RH.0 | chr6 | 43246482 | 43246503 | + | 0.37 | 0.19 |
| 9480DEF1ED9 | | | | | | |
| 4CEZ0Z | | | | | | |
| ARall_026RH.3 | chr7 | 155641558 | 155641581 | - | 0.795 | 0.12 |
| 95B3BFE67834 | | | | | | |
| 78Z0Z | | | | | | |
| ARGSi_375RH. | chr12 | 1819410 | 1819434 | + | 0.06 | 0.19 |
| 7DBF9C142C0 | | | | | | |
| E459Z0Z | | | | | | |

TABLE 4
RHAMPSEQ Panel-RHC.CF702EBACB4E415

| | | | | | % Indel in | |
|---|---|---|---|---|---|---|
| | | | | | HEK293-Cas9 | % Indel |
| | | | | | cells with | in unedited |
| rhAmpSeq | | | | | XT 2-part | HEK293-Cas9 |
| Assay ID | Chrom | Start | End | Strand | gRNA | cells |
| LAG3_site_9_0 | chr12 | 6773276 | 6773299 | - | 72.365 | 0.33 |
| 01RH.22F7C9E | | | | | | |
| 936284EBZ0Z | | | | | | |
| LAG3_site_9_0 | chr8 | 1944116 | 1944139 | - | 10.565 | 0.39 |
| 02RH.81494F08 | | | | | | |
| 8811466Z0Z | | | | | | |
| 03RH.773DE39 | | | | | | |
| LAG3_site_9_0 | chr1 | 64133480 | 64133503 | + | 67.115 | 0.13 |
| 03RH.773DE39 | | | | | | |
| 66F1A425Z0Z | | | | | | |
| LAG3_site_9_0 | chr18 | 23732318 | 23732341 | - | 76.19 | 1.99 |
| 04RH.2DAA13 | | | | | | |
| 6F92E4418Z0Z | | | | | | |
| LAG3_site_9_0 | chr14 | 90593844 | 90593867 | - | 69.79 | 0.215 |
| 05RH.AA877E6 | | | | | | |
| FA6A24D1Z0Z | | | | | | |
| LAG3_site_9_0 | chr5 | 149864107 | 149864130 | - | 76.275 | 0.065 |
| 06RH.42A5229 | | | | | | |
| 000A84E1Z0Z | | | | | | |
| LAG3_site_9_0 | chr3 | 67021410 | 67021433 | - | 61.765 | 0.065 |
| 07RH.FFAF111 | | | | | | |
| 8A8B0446Z0Z | | | | | | |
| LAG3_site_9_0 | chr2 | 217789960 | 217789983 | + | 62.935 | 0.09 |
| 08RH.2AAFE2 | | | | | | |
| 0057E848FZ0Z | | | | | | |
| LAG3_site_9_0 | chr15 | 69319847 | 69319870 | - | 53.38 | 0.055 |
| 09RH.E62D0B3 | | | | | | |
| DOD5A441Z0Z | | | | | | |
| LAG3_site_9_0 | chr8 | 102730074 | 102730097 | - | 24.86 | 0 |
| 10RH.30C85EB | | | | | | |
| E71D2458Z0Z | | | | | | |
| LAG3_site_9_0 | chr10 | 23362361 | 23362384 | - | 46.45 | 0.16 |
| 11RH.577D19B | | | | | | |
| C5F2D4E6Z0Z | | | | | | |
| LAG3_site_9_0 | chr7 | 101283211 | 101283234 | - | 58.37 | 0.045 |
| 12RH.D669B4 | | | | | | |
| ACA170426Z0Z | | | | | | |
| LAG3_site_9_0 | chr16 | 75111804 | 75111827 | + | 37.275 | 0.1 |
| 13RH.096A095 | | | | | | |
| 24D584DCZ0Z | | | | | | |
| LAG3_site_9_0 | chr22 | 47924625 | 47924648 | + | 78.11 | 0.23 |
| 14RH.EBEB4A | | | | | | |
| 10DF6A476Z0Z | | | | | | |
| LAG3_site_9_0 | chr1 | 202586267 | 202586290 | + | 38.505 | 0.11 |
| 15RH.05731F31 | | | | | | |
| 83CE49FZ0Z | | | | | | |
| LAG3_site_9_0 | chr3 | 10338273 | 10338296 | - | 18.62 | 0 |
| 16RH.94FCDF2 | | | | | | |
| 4B2744C1Z0Z | | | | | | |
| LAG3_site_9_0 | chr3 | 42907094 | 42907117 | - | 64.49 | 0 |
| 17RH.69928A1 | | | | | | |
| C36EA400Z0Z | | | | | | |
| LAG3_site_9_0 | chr8 | 29646304 | 29646327 | - | 28.745 | 0.16 |
| 18RH.F8988587 | | | | | | |
| 91CA4E9Z0Z | | | | | | |
| LAG3_site_9_0 | chr7 | 4961802 | 4961825 | - | 37.645 | 0.07 |
| 19RH.5971518 | | | | | | |
| BF622404Z0Z | | | | | | |
| LAG3_site_9_0 | chr3 | 10473746 | 10473769 | + | 32.595 | 0.21 |
| 20RH.71FCD97 | | | | | | |
| C250849FZ0Z | | | | | | |
| LAG3_site_9_0 | chrX | 109930511 | 109930534 | - | 44.76 | 0.19 |
| 21RH.E1C04B4 | | | | | | |
| 20B954EFZ0Z | | | | | | |
| LAG3_site_9_0 | chr5 | 173295131 | 173295154 | + | 3.23 | 0.045 |
| 22RH.9545B4F | | | | | | |
| 17F384FAZ0Z | | | | | | |
| LAG3_site_9_0 | chr1 | 179806581 | 179806604 | + | 35.485 | 0.085 |
| 23RH.6E61C68 | | | | | | |
| 111A04E3Z0Z | | | | | | |
| LAG3_site_9_0 | chr1 | 43279798 | 43279821 | - | 29.105 | 0.16 |
| 24RH.8D87AF | | | | | | |
| COF87B498Z0Z | | | | | | |
| 25RH.F28C401 | | | | | | |
| LAG3_site_9_0 | chr20 | 57124384 | 57124407 | + | 10.395 | 0 |
| 25RH.F28C401 | | | | | | |
| CEC594E2Z0Z | | | | | | |
| LAG3_site_9_0 | chr2 | 151203105 | 151203128 | - | 81.325 | 0 |
| 26RH.F61CF56 | | | | | | |
| 7366B4B4Z0Z | | | | | | |
| LAG3_site_9_0 | chr17 | 5976298 | 5976321 | - | 0.95 | 0.285 |
| 27RH.9A8922F | | | | | | |
| 8978E459Z0Z | | | | | | |
| LAG3_site_9_0 | chr12 | 120684762 | 120684785 | - | 5.905 | 0.08 |
| 28RH.4758D12 | | | | | | |
| 43C474CEZ0Z | | | | | | |
| LAG3_site_9_0 | chr7 | 50764042 | 50764065 | - | 8.075 | 0.145 |
| 29RH.5E87643 | | | | | | |
| 3ABDA48AZ0Z | | | | | | |
| LAG3_site_9_0 | chr9 | 133804569 | 133804592 | - | 10.73 | 0.065 |
| 30RH.65F088E | | | | | | |
| 456544B6Z0Z | | | | | | |
| LAG3_site_9_0 | chr11 | 17496696 | 17496719 | + | 4.63 | 0.015 |
| 31RH.CE5A756 | | | | | | |
| BD29246DZ0Z | | | | | | |
| LAG3_site_9_0 | chr16 | 55491673 | 55491697 | - | 18.38 | 0.05 |
| 32RH.D536E45 | | | | | | |
| A63054B1Z0Z | | | | | | |
| LAG3_site_9_0 | chr17 | 63474374 | 63474397 | - | 7.1 | 0 |
| 33RH.47A3C43 | | | | | | |
| 0F91F4F1Z0Z | | | | | | |
| LAG3_site_9_0 | chr5 | 113119765 | 113119788 | - | 5.025 | 0.08 |
| 34RH.BFD9C6 | | | | | | |
| 8929F74B7Z0Z | | | | | | |
| LAG3_site_9_0 | chr16 | 51868589 | 51868612 | - | 3.74 | 0.105 |
| 35RH.A0E5EA | | | | | | |
| COB38B494Z0Z | | | | | | |
| LAG3_site_9_0 | chr18 | 77403690 | 77403713 | + | 2.315 | 0.295 |
| 36RH.9EA0AF | | | | | | |
| DC49C1441Z07 | | | | | | |
| LAG3_site_9_0 | chr7 | 129460833 | 129460856 | + | 6.29 | 0.01 |
| 37RH.5B57CF9 | | | | | | |
| 2561343AZ0Z | | | | | | |
| LAG3_site_9_0 | chr6 | 27632534 | 27632557 | - | 4.55 | 0.07 |
| 38RH.E7505CA | | | | | | |
| DE877479Z0Z | | | | | | |
| LAG3 site 9_0 | chr1 | 184664107 | 184664130 | - | 7.155 | 0.05 |
| 39RH.75F1D6D | | | | | | |
| 3A92B4F1Z0Z | | | | | | |
| LAG3_site_9_0 | chr22 | 50224159 | 50224183 | - | 5.56 | 0.19 |
| 40RH.6A33D94 | | | | | | |
| DE87244AZ0Z | | | | | | |
| LAG3_site_9_0 | chr7 | 157032941 | 157032964 | + | 6.835 | 0.04 |
| 41RH.3C16D84 | | | | | | |
| DF842480Z0Z | | | | | | |
| LAG3 site_9_0 | chr6 | 123513131 | 123513154 | - | 4.645 | 0.195 |
| 42RH.4CE38D0 | | | | | | |
| F6FED475Z0Z | | | | | | |
| LAG3_site_9_0 | chr5 | 86093859 | 86093882 | - | 9.235 | 0.155 |
| 43RH.2370197 | | | | | | |
| ECA9046EZ0Z | | | | | | |
| LAG3_site_9_0 | chr2 | 106630587 | 106630611 | + | 6.115 | 0.03 |
| 44RH.FACAAE | | | | | | |
| 5EF874494Z0Z | | | | | | |
| LAG3 site 9_0 | chr1 | 77338615 | 77338638 | + | 11.98 | 0.025 |
| 45RH.043F18D | | | | | | |
| 3D32B45BZ0Z | | | | | | |
| LAG3_site_9_0 | chr3 | 171739147 | 171739170 | - | 2.33 | 0.155 |
| 46RH.094A283 | | | | | | |
| 8661C445Z0Z | | | | | | |
| 47RH.61AB514 | | | | | | |
| LAG3_site_9_0 | chr21 | 19492741 | 19492764 | + | 12.18 | 0.025 |
| 47RH.61AB514 | | | | | | |
| C7E9344EZ0Z | | | | | | |
| LAG3 site 9_0 | chr2 | 68706891 | 68706914 | - | 5.825 | 0.14 |
| 48RH.2E6B147 | | | | | | |
| 6100D41FZ0Z | | | | | | |
| LAG3_site_9_0 | chr8 | 24745109 | 24745132 | - | 5.28 | 0.095 |
| 49RH.D5C63D | | | | | | |
| 1E118241BZ0Z | | | | | | |
| LAG3_site_9_0 | chr9 | 126674774 | 126674797 | - | 2.165 | 0.165 |
| 50RH.0B2507D | | | | | | |
| 048C5483Z0Z | | | | | | |

### Example 2. RNP Delivery of UNA-Modified sgRNAs at Subsaturating Doses

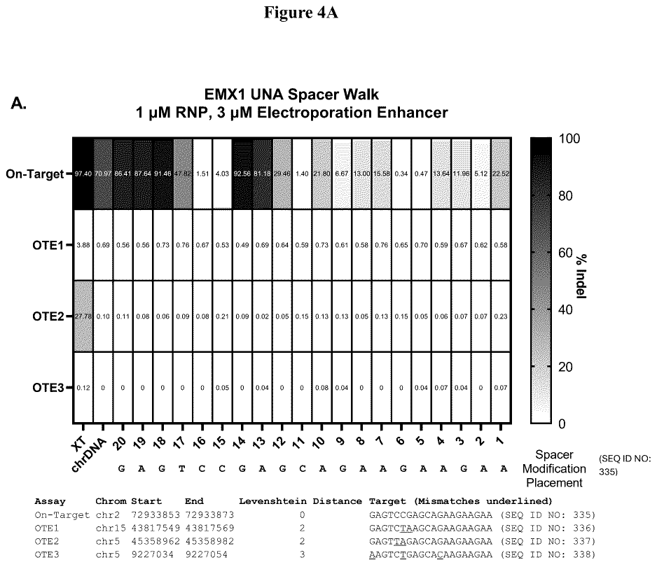

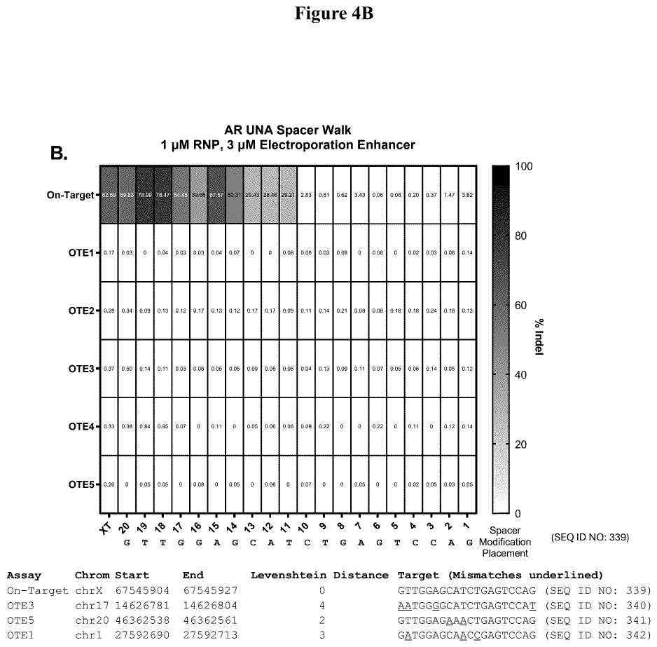

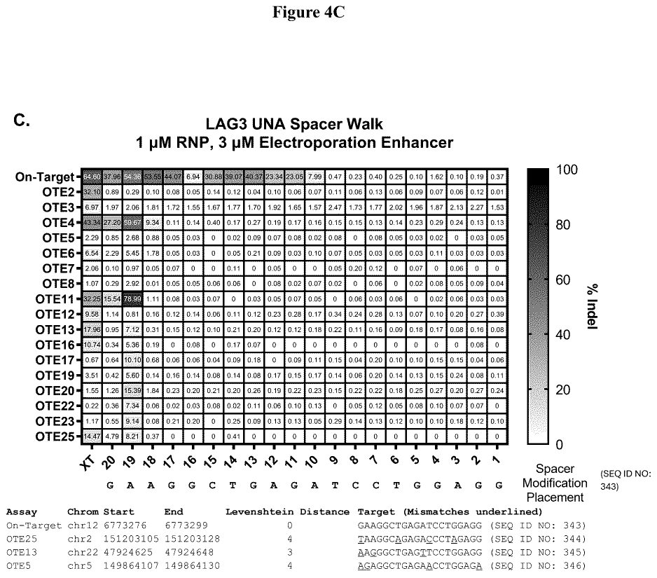

To ensure that UNA-modified sgRNAs were not affecting overall editing of the gRNA due to the saturating conditions of the stable Cas9 expression system, these gRNAs (SEQ ID NO: 4 to SEQ ID NO: 65) were used with non-saturating doses of RNP (1 μM RNP, WT-Cas9 V3, with 3 μM electroporation enhancer) in HEK293 cells. Library prep and RHAMPSEQ panels were prepared as described above. For all targets, if a UNA was placed in the seed region (positions 1-10), there was a dramatic decrease in on-target editing (FIG. 4A-FIG. 4C). This stands in contrast to the mod walk performed in the stable expression cell line where UNAs could be placed in the seed region and retain on-target editing. These results illustrate that depending on the expression/delivery of Cas9, the placement of the UNA maybe fine-tuned to elicit the desired on-/off-target editing ratio. Additionally, placement of the UNA in position 18 (SEQ ID NO: 6, SEQ ID NO: 28, SEQ ID NO: 48) showed the greatest retention of on-target editing and reduction of off-target editing when delivering Cas9 as RNP showing that UNAs are not lowering off-target editing thru reduction in total editing and that there is possibly a target agnostic location for UNA modifications.

### Example 3. Assessment of LNA and 2′Fluoro Modifications on Cas9 Editing

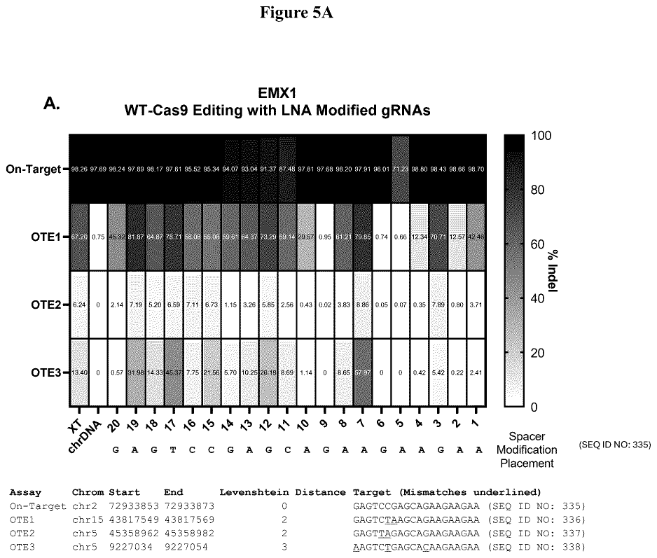

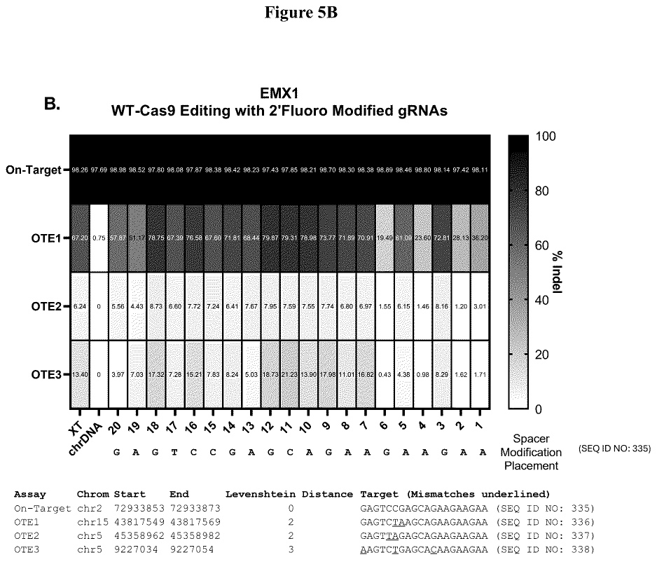

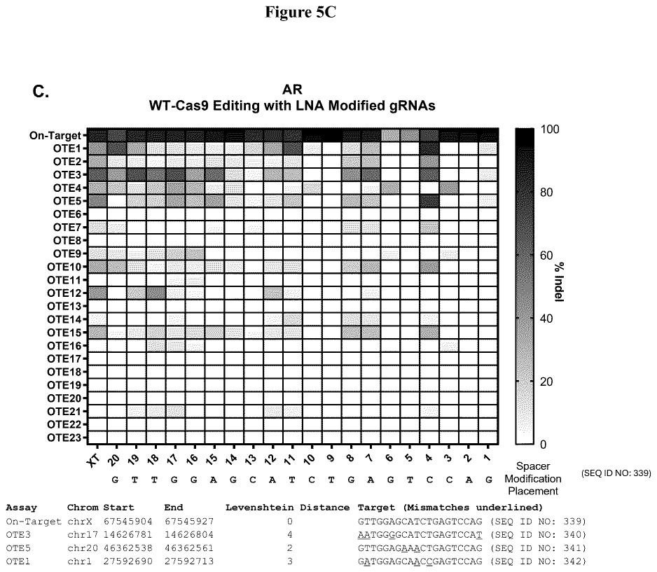

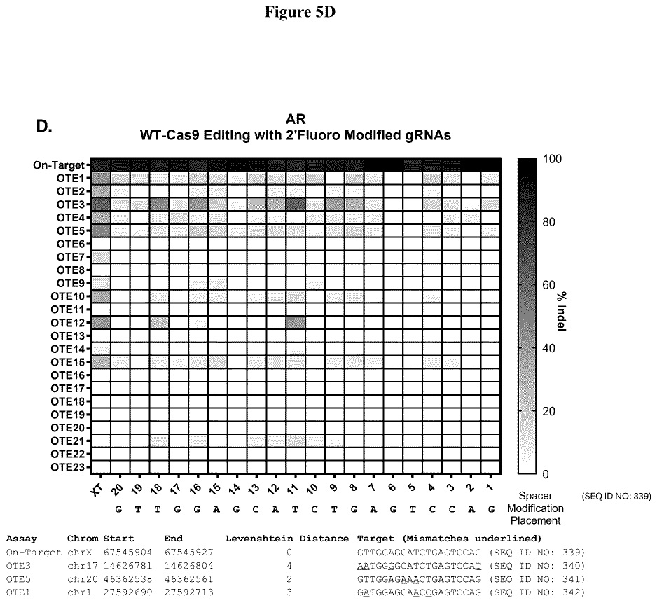

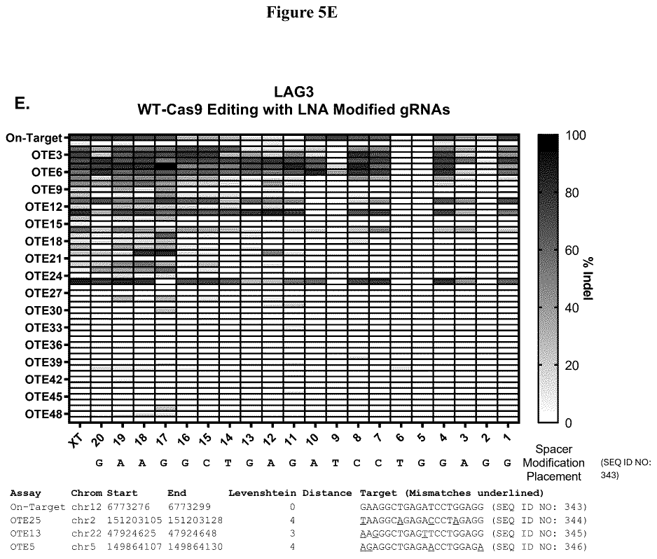

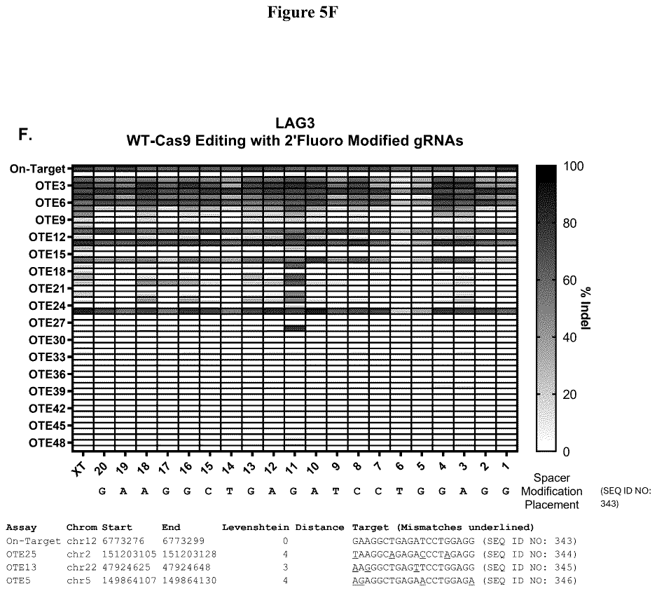

Following the results of UNA modifications to Cas9 editing additional modifications including the LNA and 2′fluoro were tested for their effect on Cas9 editing. gRNAs with a single modification placed along each base of the gRNA spacer were ordered for three targets: EMX1, AR, and LAG3 (SEQ ID NO: 66 to SEQ ID NO: 185). These gRNAs were tested in the same manner previously described using the HEK293-Cas9 stable expression system with saturating conditions along with non-saturating conditions with RNP delivery. Under saturating conditions with Cas9 stable expression, both the LNA and 2′fluoro modified sgRNAs reduced off-target editing (FIG. 5A-FIG. 5F). 2′fluoro modifications, however; did not yield as dramatic of a reduction in off-target editing as the UNA or LNA modifications possibly due to the fact the chemical structure is not as markedly different from a standard RNA. Correspondingly, the 2′fluoro retained on-target editing when placed at nearly all locations along the gRNA spacer for each target. Interestingly, the placement of the modification that yielded the best on/off-target editing ratio was unique to each modification within the seed region of the gRNA (position 9 for the LNA [SEQ ID NO: 77, SEQ ID NO: 117, SEQ ID NO: 137,], position 6 for 2′fluoro [SEQ ID NO: 100, SEQ ID NO: 160, SEQ ID NO: 180], which provides further evidence that the divergent chemical structure of these modifications is what is influencing nuclease editing. Similarly to the UNA, neither the LNA nor 2′fluoro showed strong evidence that mismatch discrimination is the primary reason for reduction in off-target editing. In contrast, placement of the LNA in several locations near the 5′ end of the gRNA significantly increased off-target editing with similar findings for 2′fluoro placement in positions 8-12.

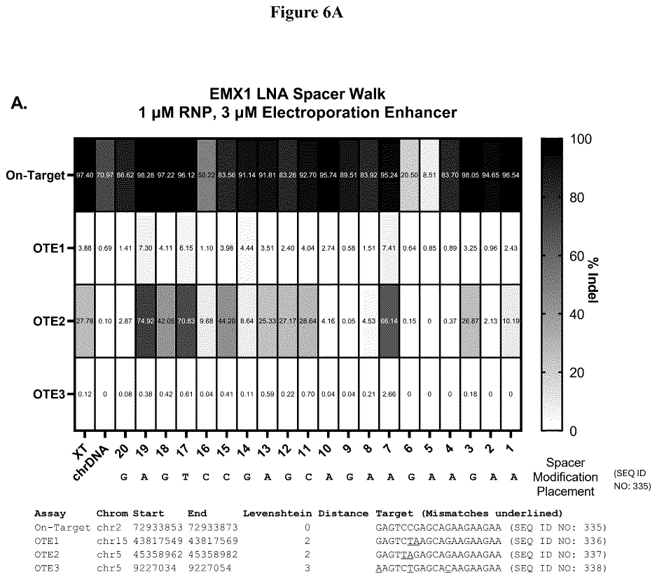

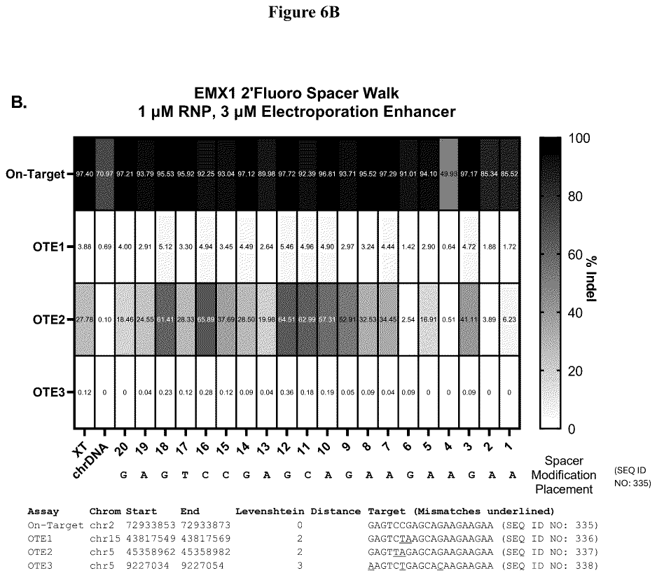

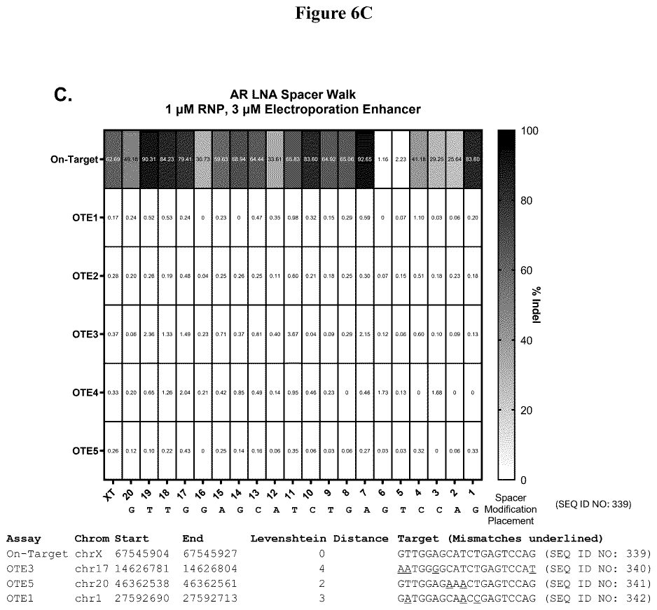

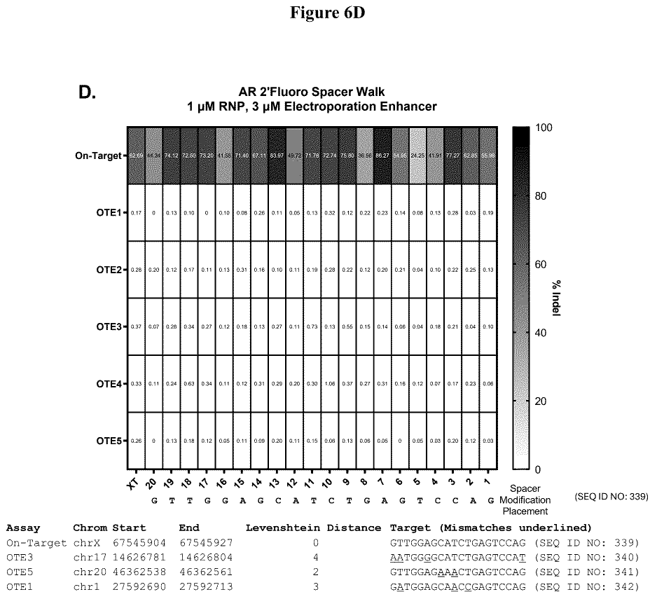

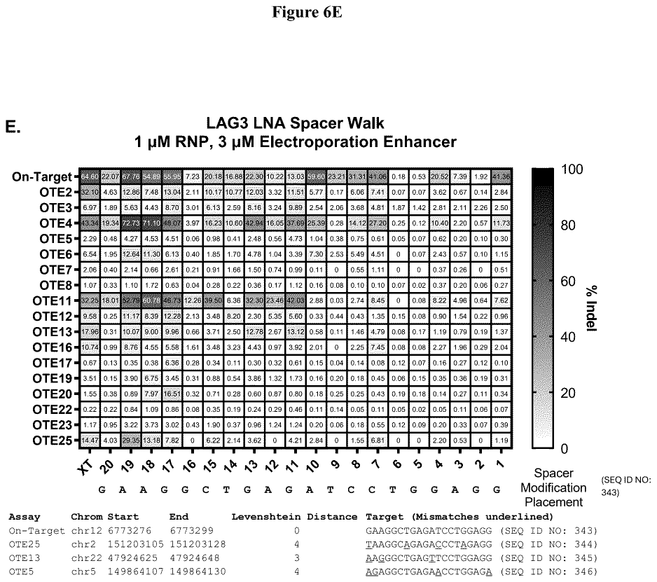

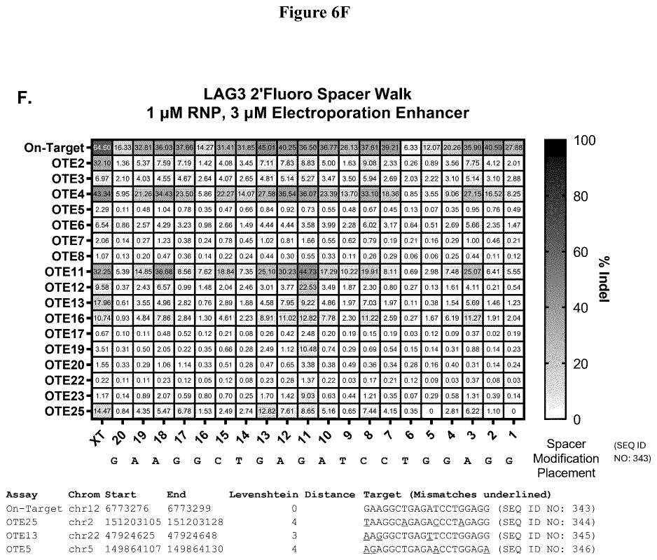

RNP delivery at non-saturating RNP concentrations was used to ascertain whether the modifications were lowering off-target editing by reducing the overall editing efficiency of the Cas nuclease. LNA modifications showed a target dependent retention in on-target editing efficiency (FIG. 6A-FIG. 6F). For targets EMX1 and AR, LNA placement at positions 5 and 6 had the greatest impact on on-target editing, whereas most LNA placements along the spacer for LAG3 led to total editing reduction. Intriguingly, LNA placement at position 9 (SEQ ID NO: 77, SEQ ID NO: 117) exhibited the same reduction in off-target editing while retaining on-target editing as was seen in the stable expression Cas9 cell line. Impact of 2′fluoro modifications on editing outcomes were likewise influenced in a target dependent manner with RNP delivery. For EMX1, on-target activity was broadly maintained with 2′fluoro placement throughout the spacer region though reduction in off-target editing was only appreciably seen with placement at positions 1, 2, 6 (SEQ ID NO: 100, SEQ ID NO: 104, SEQ ID NO: 105). Likewise, AR saw retention in on-target activity with multiple 2′fluoro placements throughout the spacer, however; little off-target activity was seen in any gRNA used. LAG3 exhibited a universal reduction in on-target editing with 2′fluoro modifications and very little reduction in off-target edits showing the 2′fluoro modification cannot be used for gRNA with this amount of off-target editing. Additionally, there is a high likelihood that sequence context of the duplex matters for the effectiveness of the modifications to influence editing. A highly diverse set of gRNAs will be needed to tease the mechanistic underpinnings that effect duplex distortion and editing rate. Overall, both the LNA and 2′fluoro lowered off-target editing while maintaining on-target editing efficiency dependent on target and nuclease delivery/expression.

### Example 4. Assessment of Cas9 Editing with Abasic Site gRNAs

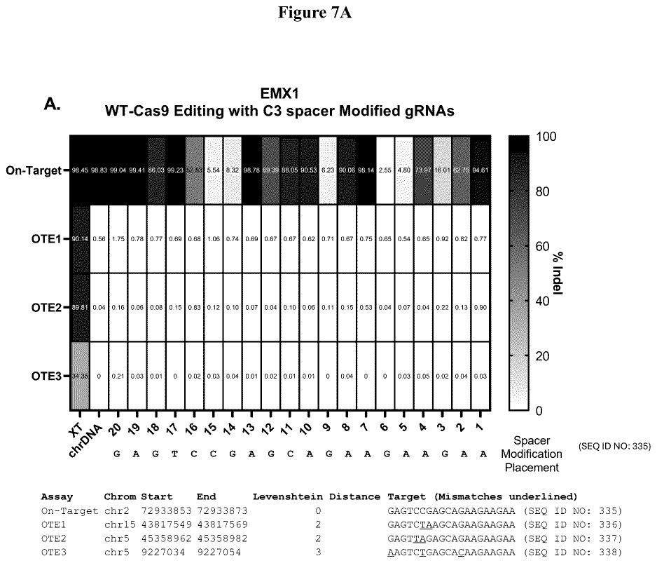

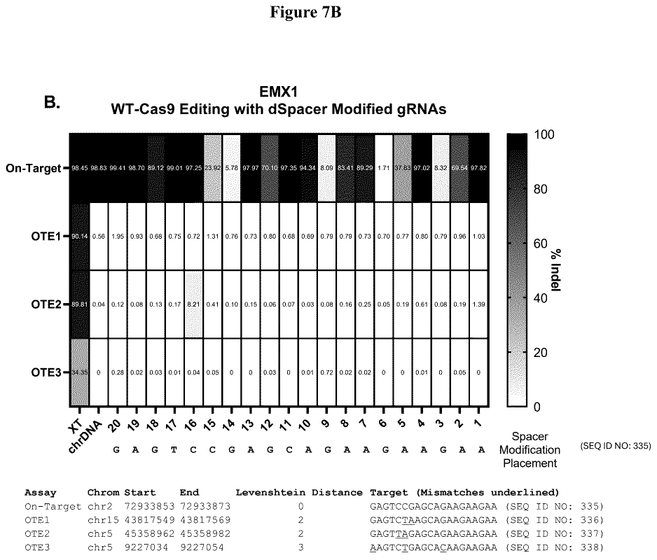

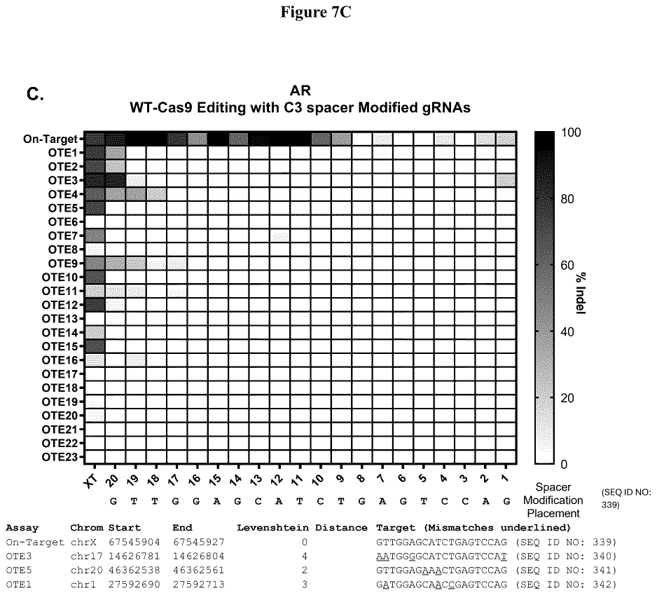

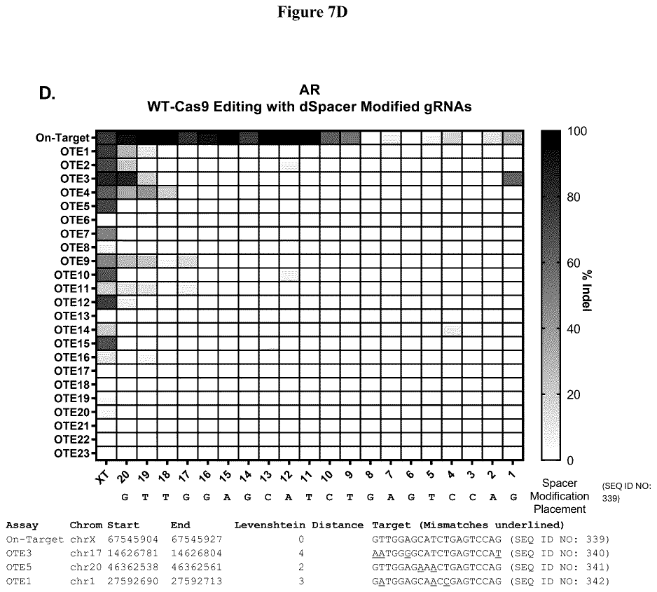

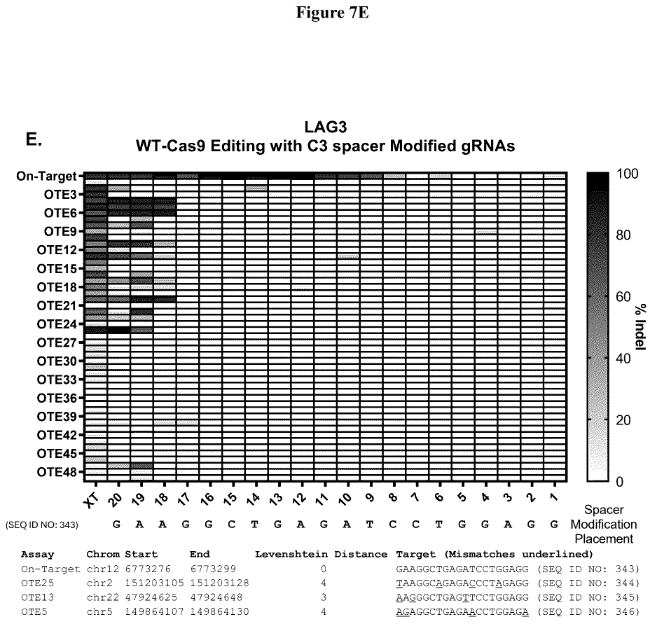

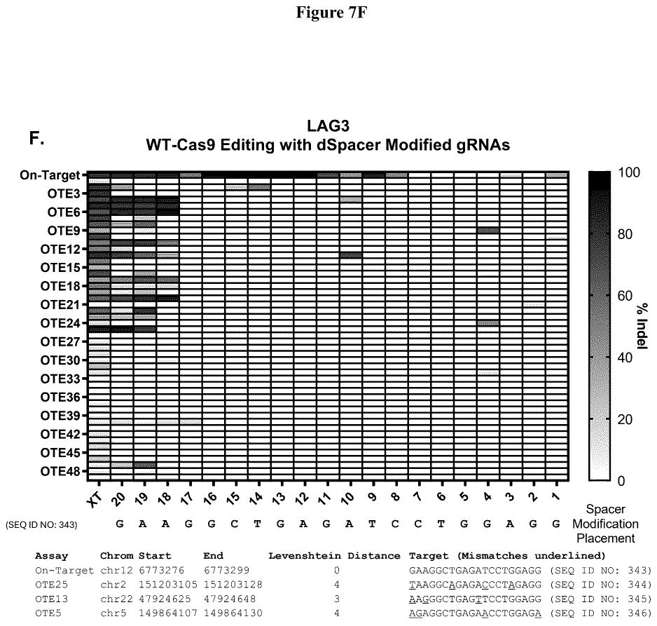

The last set of modifications to be tested for their effect on Cas nuclease editing were the abasic modifications: C3 spacer and dSpacer. gRNAs with a single modification placed along each base of the gRNA spacer were ordered for three targets: EMX1, AR, and LAG3 (SEQ ID NO: 186 to SEQ ID NO: 305). These gRNAs were tested in the same manner previously described using the HEK293-Cas9 stable expression system with saturating conditions. For all targets, placement of the abasic site modifications lowered off-target editing (FIG. 7A-FIG. 7F). The reduction in off-target editing was most pronounced with abasic modifications placed in positions 11-17. In contrast, modification placement in the seed region (1-10) led to significant reduction in total editing for both the C3 spacer and dSpacer. Overall, there was no major on/off-target editing differences in modification placement between the C3 spacer and dSpacer, indicating that the structural differences between these two modifications is not the main reason for off-target editing reduction; lending support to the hypothesis that the lack of base pairing at the abasic site alters the duplex structure to affect nuclease editing.

### Example 5. Comparison of UNA Modified gRNAs with HiFi-Cas9

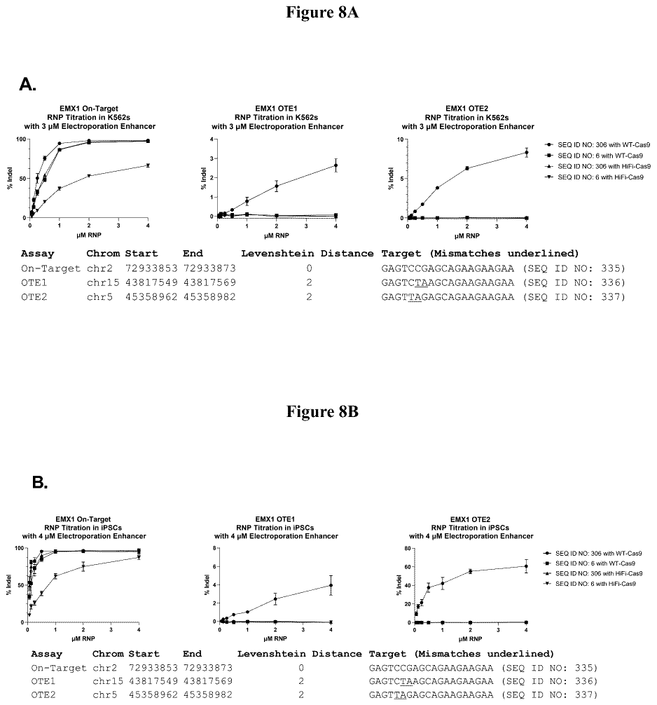

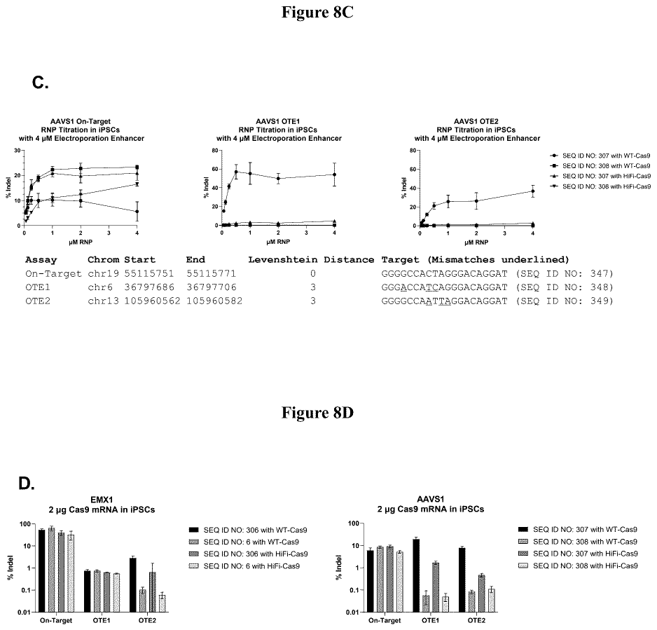

A RNP dose titration in K562s was used to assess how modified gRNAs influenced editing at lower RNP doses compared to HiFi-Cas9. A RNP dilution series (0.0625-4 μM) was utilized with a gRNA with the UNA modification in position 18 on the spacer and a standard end-blocking AltR modified crRNA with either WT-Cas9 or HiFi-Cas9 (SEQ ID NO: 06, SEQ ID NO: 306). The RNP complex was nucleofected into K562s with 3 μM electroporation enhancer and assessed for editing using RHAMPSEQ. The UNA modified gRNA complexed with WT-Cas9 showed a similar dose-response curve as a standard gRNA complexed with HiFi-Cas9 for on-target editing (FIG. 8A-FIG. 8D). In addition, the off-target editing for UNA gRNAs mirrored the reduction in off-target editing using HiFi. Interestingly, when combining the UNA gRNA with HiFi-Cas9, the editing at the on-target was reduced and is comparable to what was previously shown with chrDNAs and HiFi nucleases (8). To show that UNA modified gRNAs could impact editing in clinically relevant cell types, a RNP dose titration was used with two target sites (EMX1 and AAVS1) in induced pluripotent stem cells (iPSCs). In iPSCs, the EMX1 UNA modified gRNA mirrored what was previously seen in K562s with retention of on-target editing while reducing off-target editing similarly to HiFi-Cas9 (FIG. 8A-FIG. 8D). For the AAVS1 target site, on-target editing increased when using UNA modified gRNAs or HiFi-Cas9 compared to standard gRNAs and WT-Cas9 while simultaneously lowering the off-target editing. Lastly, to show that the reduction in editing is independent of the delivery format of the Cas-nuclease, Cas9 mRNA (WT and HiFi) was used in combination with standard or UNA modified gRNAs for the targets EMX1 and AAVS1. Both sites show comparable on/off-target editing ratios as was seen with RNP delivery of Cas9. These results highlight that UNA modified gRNAs can perform similar to HiFi systems and can provide therapeutic utility with their reduction of off-target editing.

### Example 6. Assessment of UNA Modifications on Cas9 On-Target Editing

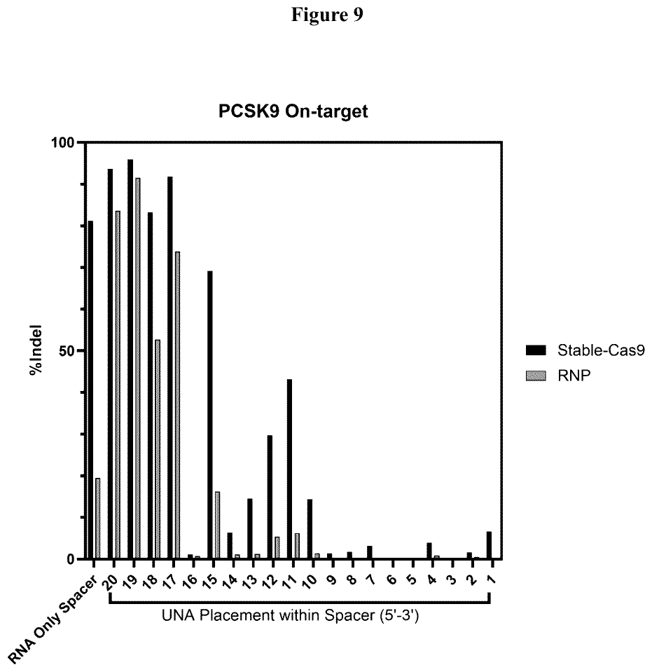

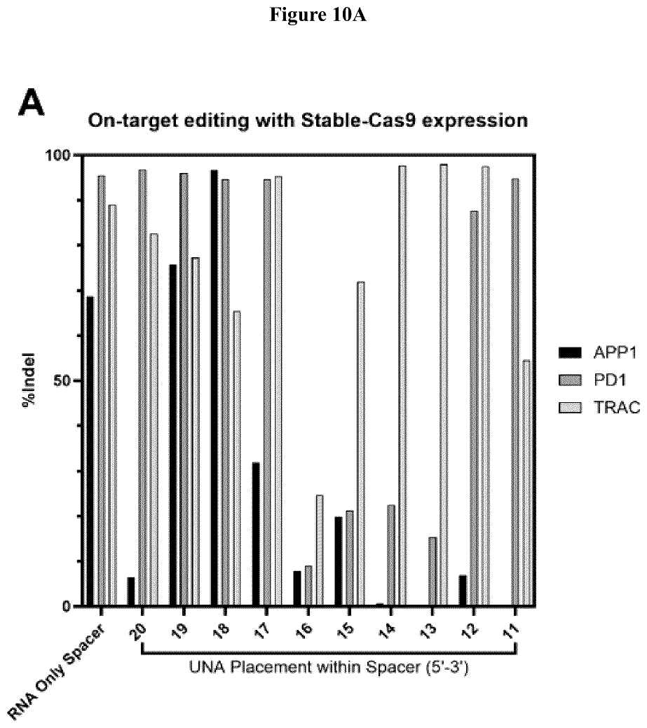

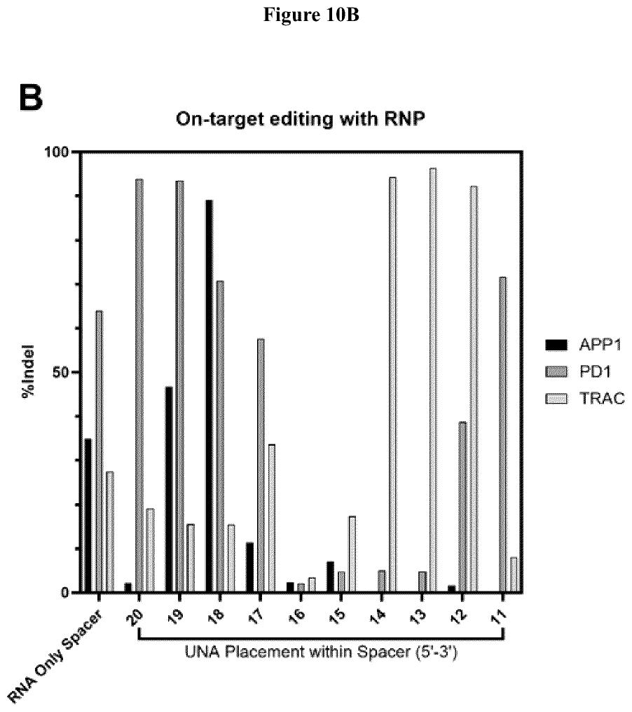

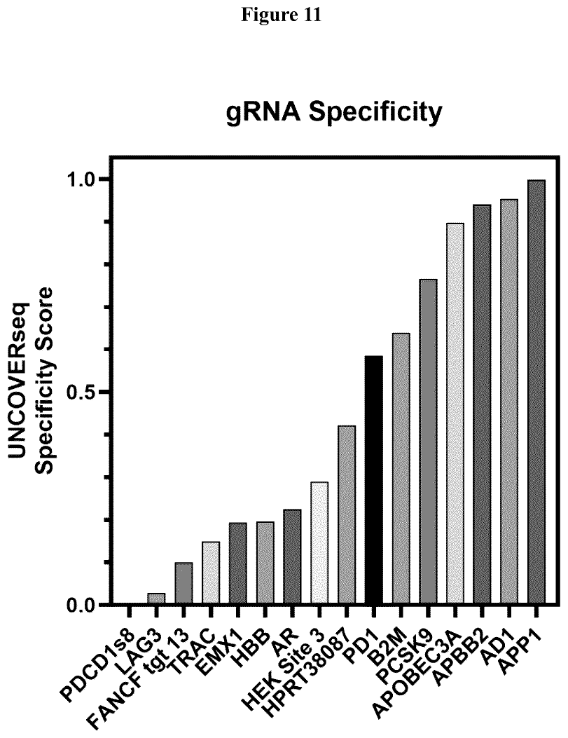

crRNAs for the guide PCSK9 were ordered with UNA modifications that were placed sequentially along the gRNA spacer (SEQ ID NO: 367-SEQ ID NO: 388) to see what effect UNAs would have on Cas9 on-target editing. These modified gRNAs were compared to IDT's standard AltR™ crRNA (SEQ ID NO; 368). All gRNAs used throughout the invention disclosure were annealed to IDT's tracrRNA. Cas9 editing was assessed with HEK293 cells constitutively expressing WT Cas9 (ATCC HEK293-Cas9) or with WT-Cas9 RNP delivery. In brief, HEK293-Cas9 stably expressing Cas9 were nucleofected (Lonza) with 5 μM of modified gRNAs. Non-stably expressed cells were nucleofected with 4 μM RNP, WT-Cas9 V3 (IDT), with 3 μM electroporation enhancer (IDT). All samples were incubated for 72 hrs., gDNA collected with QuickExtract, the on-target editing site was amplified and prepped for NGS using RHAMPSEQ, and analyzed using CRISPAltRations as previously described¹⁰. crRNAs with UNAs placed at positions 20-17 (SEQ ID NO: 369-SEQ ID NO: 372) increased editing compared to an RNA only crRNA with the PCSK9 target site with both stable Cas9 expression and RNP (FIG. 9). To ensure that this effect wasn't limited to a single target site, additional sites were chosen for on-target editing analysis (SEQ ID NO: 389-SEQ ID NO: 415) and measured in a similar manner as described above except for a shortened UNA modification walk through the spacer (Positions 20-11). Once again, increased on-target editing efficiency was seen with each target site; however, each target site had a differing pattern for optimal placement of the UNA within the spacer region, demonstrating the influence of the target site sequence on the UNA's effectiveness for editing modulation (FIG. 10). These results were the first instance that a single UNA modification in the gRNA spacer region can broadly increase on-target editing.

In some embodiments, in a spacer sequence, positions are numbered from the 5′ to the 3′ end of the spacer sequence, wherein Position 20 being the first nucleotide at the 5′ end and Position 1 being the last nucleotide at the 3′end for a typical 20 nt spacer. For truncated gRNAs, a spacer sequence can be modified to remove one, two, or three nucleotides from the 5′ end (e.g., truncated gRNAs having a spacer with 19 nucleotides has nucleotide positions 19 to 1, instead of positions 20 to 1).

### Example 7. Assessment of UNA Modifications on Cas9 On/Off-Target Editing

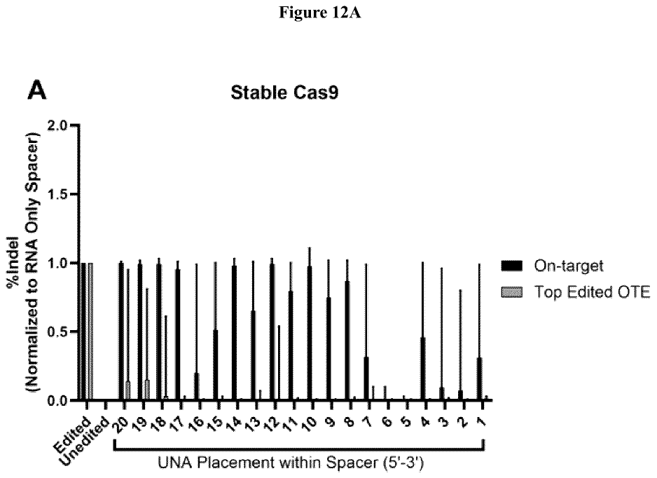

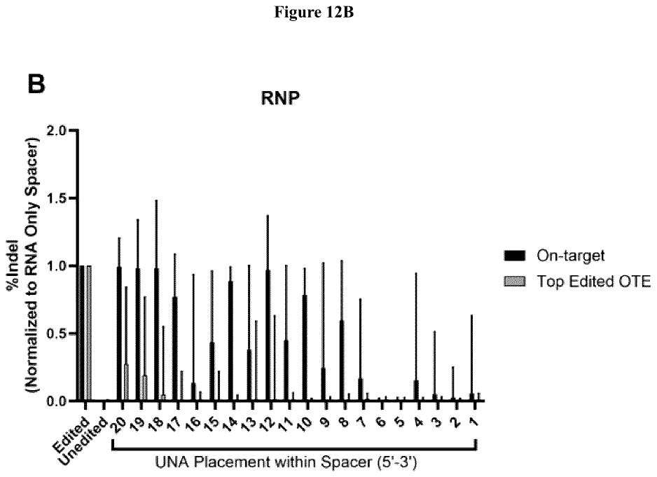

To more broadly assess the effect of UNA modifications on Cas9 editing, sixteen gRNAs were chosen to sequentially place the UNA within the gRNA spacer based on their wide ranging editing specificities as described previously (See Kinney reference at FIG. 3, Table 1; and SEQ ID NO: 369 to SEQ ID NO: 388, SEQ ID NO: 390 to SEQ ID NO: 399, SEQ ID NO: 401 to SEQ ID NO: 410, SEQ ID NO: 412 to SEQ ID NO: 421, SEQ ID NO: 434 to SEQ ID NO: 643)¹¹. As described herein, all UNA modified gRNAs were compared to IDT's standard AltR™ crRNAs (SEQ ID NO: 367, SEQ ID NO: 389, SEQ ID NO: 300, SEQ ID NO: 411, SEQ ID NOs: 422-433), delivered in stably expressing Cas9 cells and RNP delivery, gDNA extracted, and editing assessed with RHAMPSEQ. As was seen previously, each target had a unique pattern of optimally placed UNAs within the spacer. To compare UNA placement across multiple target sites, editing at each UNA placement within the spacer across the sixteen target sites was normalized to the editing of either the on-target or top edited off-target site of the standard AltR™ crRNA for the corresponding site (FIG. 12). Normalizing the data in this way allowed trends to emerge where specific locations within the spacer region had a higher probability of either A) increasing editing compared to an unmodified spacer B) retention of on-target editing while lowering off-targeting editing C) partial decrease in on-target editing or D) complete elimination of editing (FIG. 4; Table 9). Across all gRNAs placement of the UNA in Tier 1 positions 20 and 19 had the greatest probability of retaining on-target editing; however, off-target editing could also be retained for a few target sites indicating these UNA locations would work best for high specificity gRNAs with already low numbers of off-targets (Table 9). Similarly to positions 20 and 19, UNAs placed at locations 18, 17, 14, 12, and 10 had a high on-target editing retention probability and were coupled with a higher rate of decreasing off-target editing compared to positions 20 and 19. This makes these positions a good choice for gRNAs with a lower specificity that may have some concerning off-targets that cannot be avoided with gRNA design considerations. Positions 16, 15, 13, 11, 9, 8, 7, 4 had a highly variable performance for on-target editing retention meaning that the target sequence highly influences the impact of UNAs on CRISPR-Cas editing. Due to their lower probability of being the ideal placement of UNAs within the spacer, we designated them as Tier 2 sites, which should be tested if Tier 1 sites do not yield desired editing levels (Table 9). Lastly, Tier 3 sites (6, 5, 3, 2, 1) knocked down editing levels for nearly all gRNAs tested with UNAs located in positions 6 and 5 knocking down editing levels comparable to unedited samples (FIG. 12; Table 9).

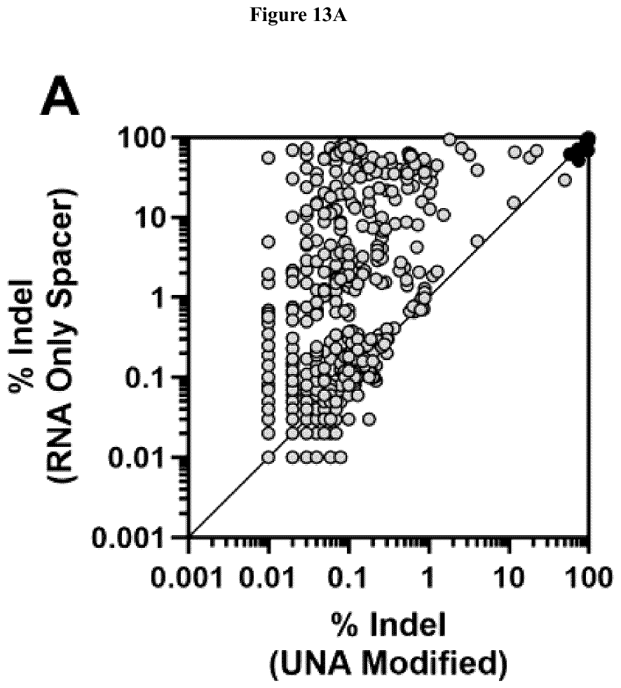

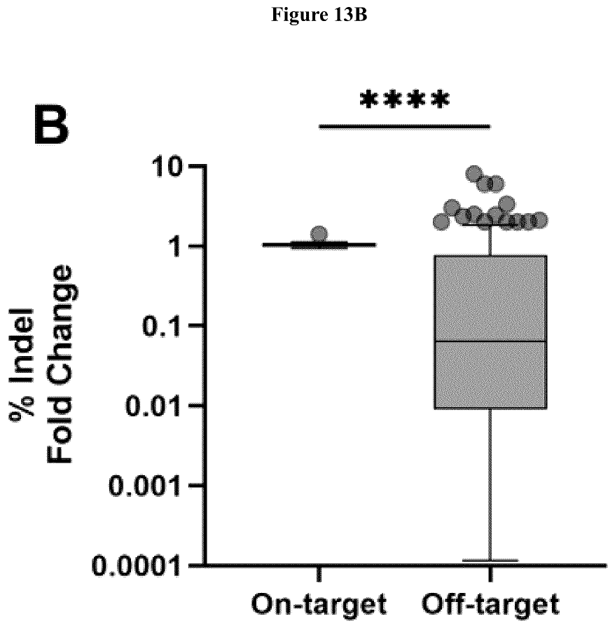

To highlight the impact of UNAs placed in optimized positions compared to standard AltR™ crRNAs, we picked UNA locations for each of the sixteen gRNAs that satisfied the ability to either increase or retain on-target editing efficiency while simultaneously lowering off-target editing. UNA locations for each of the gRNAs that satisfied these conditions were as follows: PDCD1s8-14, LAG3-12, FANCF tgt 13-10, TRAC-14, EMX1-18, HBB-17, AR-15, HEK Site 3-14, HPRT 38087-18, PD1-20, B2M-12, PCSK9-17, APOBEC3A-20, APBB2-20, AD1-20, APP1-18 (SEQ ID NO: 372, SEQ ID NO: 392, SEQ ID NO: 401, SEQ ID NO: 418, SEQ ID NO: 440, SEQ ID NO: 462, SEQ ID NO: 484, SEQ ID NO: 496, SEQ ID NO: 517, SEQ ID NO: 539, SEQ ID NO: 560, SEQ ID NO: 576, SEQ ID NO: 602, SEQ ID NO: 614, SEQ ID NO: 624, SEQ ID NO: 634.). Of the sixteen gRNAs, 94% of gRNAs had UNAs placed in Tier 1 locations with many sites having multiple Tier 1 and 2 locations that satisfied our optimized position criteria (Table 9). When comparing editing at all on/off-target sites for all gRNAs between standard AltR™ and optimally placed UNA modified crRNAs with stable Cas9 expression, large decreases in off-target editing (median fold change=0.06; ~15-fold decrease in editing) with corresponding retention in on-target editing (median fold change=1.01) was observed (FIG. 13A and FIG. 13B). This demonstrates that UNAs increase the specificity of CRISPR-Cas editing systems and can provide utility for translational and therapeutic applications.

TABLE 5
AAVS1 30plex RHAMPSEQ Panel - RHC.4C1B1FCBAB3C4CF

| rhAmpSeq Assay ID | Chrom | Start | End | Strand |
|---|---|---|---|---|
| AAVS1_10RH.5276F0443F0947DZ0Z | chr11 | 61343764 | 61343784 | + |
| AAVS1_11RH.326B01606E5349AZ0Z | chr8 | 22778070 | 22778090 | + |
| AAVS1_12RH.D732DA5AB2CE425Z0Z | chr10 | 130782551 | 130782571 | + |
| AAVS1_13RH.591A8C2B5F804E0Z0Z | chr22 | 44303220 | 44303240 | + |
| AAVS1_14RH.4915BB09183E47BZ0Z | chr11 | 118846778 | 118846798 | + |
| AAVS1_15RH.0BEF35537BC04CEZ0Z | chr7 | 2104507 | 2104527 | + |
| AAVS1_16RH.845BF9B0C9BE4DEZ0Z | chr8 | 143802949 | 143802969 | + |
| AAVS1_17RH.E32167A541C549BZ0Z | chr11 | 27064833 | 27064853 | + |
| AAVS1_18RH.71A673C6AE994B9Z0Z | chr17 | 75895457 | 75895477 | + |
| AAVS1_19RH.237C51E0EE8842FZ0Z | chr7 | 74106811 | 74106831 | + |
| AAVS1_1RH.65D655F066094DDZ0Z | chr12 | 107092486 | 107092506 | + |
| AAVS1_20RH.693B74B4EB6247FZ0Z | chr7 | 51607198 | 51607218 | + |
| AAVS1_21RH.53DE392E0E5B424Z0Z | chr2 | 87285027 | 87285047 | + |
| AAVS1_22RH.D5A48E6C8D3641CZ0Z | chr12 | 108187901 | 108187921 | + |
| AAVS1_23RH.92CED1A8F57E4BCZ0Z | chr22 | 22573603 | 22573623 | + |
| AAVS1_24RH.199456172B464BEZ0Z | chr6 | 53335292 | 53335312 | + |
| AAVS1_25RH.3C2FD4ABC63C405Z0Z | chr10 | 113944803 | 113944823 | + |
| AAVS1_26RH.62CF0917A576466Z0Z | chr6 | 70199680 | 70199700 | + |
| AAVS1_27RH.69E4AAC76E13468Z0Z | chr2 | 111576560 | 111576580 | + |
| AAVS1_28RH.75E60456C358483Z0Z | chr16 | 32025810 | 32025830 | + |
| AAVS1_29RH.EDD5F0DB80F5425Z0Z | chr9 | 97300272 | 97300292 | + |
| AAVS1_2RH.E7465CE90E094D9Z0Z | chr6 | 36797686 | 36797706 | + |
| AAVS1_30RH.FCEA75769A4041CZ0Z | chr20 | 53642954 | 53642974 | + |
| AAVS1_3RH.6020640C45F04A2Z0Z | chr19 | 55115751 | 55115771 | + |
| AAVS1_4RH.3B9DEA095A3B4B0Z0Z | chr19 | 16064179 | 16064199 | + |
| AAVS1_5RH.41ABDA09EC8842FZ0Z | chr21 | 41521017 | 41521037 | + |
| AAVS1_6RH.84A59E6D3C6C408Z0Z | chr15 | 89933458 | 89933478 | + |
| AAVS1_7RH.3CB73ACAEC0B48DZ0Z | chr18 | 48222163 | 48222183 | + |
| AAVS1_8RH.6A976D8D5655470Z0Z | chr2 | 204531403 | 204531423 | + |
| AAVS1_9RH.C363E2AF2FCA423Z0Z | chr13 | 105960562 | 105960582 | + |

TABLE 6
Oligos-(m denotes 2′-O-Methyl, * denotes phosphorothioate linkage, + denotes LNA base, iSpC3 denotes C3 spacer, i2FC
denotes 2′Fluoro, idSp denotes dSpacer.)

| Sequence ID | Name | Sequence | Description |
|---|---|---|---|
| SEQ ID NO: 1 | EMX1 XT | mG*mA*mGrUrCrCrGrArGrCrArGr ArArGrArArGrArA | crRNA with IDT′s XT Modification. |
| | | rGrUrUrUrUrArGrArGrCrUrAmU+G*+C*mU | |
| SEQ ID NO: 2 | EMX1_chRDNA | rGAGrUCCrGArGCrAGrArArGrAArGArArGrUrUrUrU | Chimeric RNA:DNA crRNA |
| | | rArGrArGrGrArUrUrGrCrU | |
| SEQ ID NO: 3 | Cariou_chACR | AGCrArArUrCrCrArArGrUrUrArArArArUrArArGrGrC | Chimeric RNA:DNA tracrRNA |
| | | rUrArGrUrCrCrGrUrUrArUrCrArArCrUrUrGrArArArAr | |
| | | ArGrUrGrGCACCrGrArGrUrCGGTGrCrUrU | |
| SEQ ID NO: 4 | EMX1_UNA_20 | /5UNA-rG/rArGrUrCrCrGrArGrCrArGrArArGrArArGr | crRNA with a single UNA base. |
| | | ArArGrUrUrUrUrArGrArGrCrUrAmU+G*+C*mU | |
| SEQ ID NO: 5 | EMX1_UNA_19 | rG/iUNA-rA/rGrUrCrCrGrArGrCrArGrArArGrArArGrA | crRNA with a single UNA base. |
| | | rArGrUrUrUrUrArGrArGrCrUrAmU+G*+C*mU | |
| SEQ ID NO: 6 | EMX1_UNA_18 | rGrA/iUNA-rG/rUrCrCrGrArGrCrArGrArArGrArArGrA | crRNA with a single UNA base. |
| | | rArGrUrUrUrUrArGrArGrCrUrAmU+G*+C*mU | |
| SEQ ID NO: 7 | EMX1_UNA_17 | rGrArG/iUNA-rU/rCrCrGrArGrCrArGrArArGrArArGrA | crRNA with a single UNA base. |
| | | rArGrUrUrUrUrArGrArGrCrUrAmU+G*+C*mU | |
| SEQ ID NO: 8 | EMX1_UNA_16 | rGrArGrU/iUNA-rC/rCrGrArGrCrArGrArArGrArArGrA | crRNA with a single UNA base. |
| | | rArGrUrUrUrUrArGrArGrCrUrAmU+G*+C*mU | |
| SEQ ID NO: 9 | EMX1_UNA_15 | rGrArGrUrC/iUNA-rC/rGrArGrCrArGrArArGrArArGrA | crRNA with a single UNA base. |
| | | rArGrUrUrUrUrArGrArGrCrUrAmU+G*+C*mU | |
| SEQ ID NO: 10 | EMX1_UNA_14 | rGrArGrUrCrC/iUNA-rG/rArGrCrArGrArArGrArArGrA | crRNA with a single UNA base. |
| | | rArGrUrUrUrUrArGrArGrCrUrAmU+G*+C*mU | |
| SEQ ID NO: 11 | EMX1_UNA_13 | rGrArGrUrCrCrG/iUNA-rA/rGrCrArGrArArGrArArGrA | crRNA with a single UNA base. |
| | | rArGrUrUrUrUrArGrArGrCrUrAmU+G*+C*mU | |
| SEQ ID NO: 12 | EMX1_UNA_12 | rGrArGrUrCrCrGrA/iUNA-rG/rCrArGrArArGrArArGrA | crRNA with a single UNA base. |
| | | rArGrUrUrUrUrArGrArGrCrUrAmU+G*+C*mU | |
| SEQ ID NO: 13 | EMX1_UNA_11 | rGrArGrUrCrCrGrArG/iUNA-rC/rArGrArArGrArArGrA | crRNA with a single UNA base. |
| | | rArGrUrUrUrUrArGrArGrCrUrAmU+G*+C*mU | |
| SEQ ID NO: 14 | EMX1_UNA_10 | rGrArGrUrCrCrGrArGrC/iUNA-rA/rGrArArGrArArGrA | crRNA with a single UNA base. |
| | | rArGrUrUrUrUrArGrArGrCrUrAmU+G*+C*mU | |
| SEQ ID NO: 15 | EMX1_UNA_9 | rGrArGrUrCrCrGrArGrCrA/iUNA-rG/rArArGrArArGrA | crRNA with a single UNA base. |
| | | rArGrUrUrUrUrArGrArGrCrUrAmU+G*+C*mU | |
| SEQ ID NO: 16 | EMX1_UNA_8 | rGrArGrUrCrCrGrArGrCrArG/iUNA-rA/rArGrArArGrA | crRNA with a single UNA base. |
| | | rArGrUrUrUrUrArGrArGrCrUrAmU+G*+C*mU | |
| SEQ ID NO: 17 | EMX1_UNA_7 | rGrArGrUrCrCrGrArGrCrArGrA/iUNA-rA/rGrArArGrA | crRNA with a single UNA base. |
| | | rArGrUrUrUrUrArGrArGrCrUrAmU+G*+C*mU | |
| SEQ ID NO: 18 | EMX1_UNA_6 | rGrArGrUrCrCrGrArGrCrArGrArA/iUNA-rG/rArArGrA | crRNA with a single UNA base. |
| | | rArGrUrUrUrUrArGrArGrCrUrAmU+G*+C*mU | |
| SEQ ID NO: 19 | EMX1_UNA_5 | rGrArGrUrCrCrGrArGrCrArGrArArG/iUNA-rA/rArGrA | crRNA with a single UNA base. |
| | | rArGrUrUrUrUrArGrArGrCrUrAmU+G*+C*mU | |
| SEQ ID NO: 20 | EMX1_UNA_4 | rGrArGrUrCrCrGrArGrCrArGrArArGrA/iUNA-rA/rGrA | crRNA with a single UNA base. |
| | | rArGrUrUrUrUrArGrArGrCrUrAmU+G*+C*mU | |
| SEQ ID NO: 21 | EMX1_UNA_3 | rGrArGrUrCrCrGrArGrCrArGrArArGrArA/iUNA-rG/rA | crRNA with a single UNA base. |
| | | rArGrUrUrUrUrArGrArGrCrUrAmU+G*+C*mU | |
| SEQ ID NO: 22 | EMX1_UNA_2 | rGrArGrUrCrCrGrArGrCrArGrArArGrArArG/iUNA-rA/ | crRNA with a single UNA base. |
| | | rArGrUrUrUrUrArGrArGrCrUrAmU+G*+C*mU | |
| SEQ ID NO: 23 | EMX1_UNA_1 | rGrArGrUrCrCrGrArGrCrArGrArArGrArArGrA/iUNA-r | crRNA with a single UNA base. |
| | | A/rGrUrUrUrUrArGrArGrCrUrAmU+G*+C*mU | |
| SEQ ID NO: 24 | AR XT | mG*mU*mUrGrGrArGrCrArUrCrUrGrArGrUrCrCrArG | crRNA with IDT′s XT Modification. |
| | | rGrUrUrUrUrArGrArGrCrUrAmU+G*+C*mU | |
| SEQ ID NO: 25 | LAG3 Site 9 XT | mG*mA*mArGrGrCrUrGrArGrArUrCrCrUrGrGrArGrG | crRNA with IDT′s XT Modification. |
| | | rGrUrUrUrUrArGrArGrCrUrAmU+G*+C*mU | |
| SEQ ID NO: 26 | AR_UNA_20 | /5UNA-rG/*mU*mUrGrGrArGrCrArUrCrUrGrArGrUrC | crRNA with a single UNA base. |
| | | rCrArGrGrUrUrUrUrArGrArGrCrUrAmU+G*+C*mU | |
| SEQ ID NO: 27 | AR_UNA_19 | mG*/iUNA-rU/*mUrGrGrArGrCrArUrCrUrGrArGrUrCr | crRNA with a single UNA base. |
| | | CrArGrGrUrUrUrUrArGrArGrCrUrAmU+G*+C*mU | |
| SEQ ID NO: 28 | AR_UNA_18 | mG*mU*/iUNA-rU/rGrGrArGrCrArUrCrUrGrArGrUrCr | crRNA with a single UNA base. |
| | | CrArGrGrUrUrUrUrArGrArGrCrUrAmU+G*+C*mU | |
| SEQ ID NO: 29 | AR_UNA_17 | mG*mU*mU/iUNA-rG/rGrArGrCrArUrCrUrGrArGrUr | crRNA with a single UNA base. |
| | | CrCrArGrGrUrUrUrUrArGrArGrCrUrAmU+G*+C*mU | |
| SEQ ID NO: 30 | AR_UNA_16 | mG*mU*mUrG/iUNA-rG/rArGrCrArUrCrUrGrArGrUr | crRNA with a single UNA base. |
| | | CrCrArGrGrUrUrUrUrArGrArGrCrUrAmU+G*+C*mU | |
| SEQ ID NO: 31 | AR_UNA_15 | mG*mU*mUrGrG/iUNA-rA/rGrCrArUrCrUrGrArGrUr | crRNA with a single UNA base. |
| | | CrCrArGrGrUrUrUrUrArGrArGrCrUrAmU+G*+C*mU | |
| SEQ ID NO: 32 | AR_UNA_14 | mG*mU*mUrGrGrA/iUNA-rG/rCrArUrCrUrGrArGrUr | crRNA with a single UNA base. |
| | | CrCrArGrGrUrUrUrUrArGrArGrCrUrAmU+G*+C*mU | |
| SEQ ID NO: 33 | AR_UNA_13 | mG*mU*mUrGrGrArG/iUNA-rC/rArUrCrUrGrArGrUr | crRNA with a single UNA base. |
| | | CrCrArGrGrUrUrUrUrArGrArGrCrUrAmU+G*+C*mU | |
| SEQ ID NO: 34 | AR_UNA_12 | mG*mU*mUrGrGrArGrC/iUNA-rA/rUrCrUrGrArGrUr | crRNA with a single UNA base. |
| | | CrCrArGrGrUrUrUrUrArGrArGrCrUrAmU+G*+C*mU | |
| SEQ ID NO: 35 | AR_UNA_11 | mG*mU*mUrGrGrArGrCrA/iUNA-rU/rCrUrGrArGrUr | crRNA with a single UNA base. |
| | | CrCrArGrGrUrUrUrUrArGrArGrCrUrAmU+G*+C*mU | |
| SEQ ID NO: 36 | AR_UNA_10 | mG*mU*mUrGrGrArGrCrArU/iUNA-rC/rUrGrArGrUr | crRNA with a single UNA base. |
| | | CrCrArGrGrUrUrUrUrArGrArGrCrUrAmU+G*+C*mU | |
| SEQ ID NO: 37 | AR_UNA_9 | mG*mU*mUrGrGrArGrCrArUrC/iUNA-rU/rGrArGrUr | crRNA with a single UNA base. |
| | | CrCrArGrGrUrUrUrUrArGrArGrCrUrAmU+G*+C*mU | |
| SEQ ID NO: 38 | AR_UNA_8 | mG*mU*mUrGrGrArGrCrArUrCrU/iUNA-rG/rArGrUr | crRNA with a single UNA base. |
| | | CrCrArGrGrUrUrUrUrArGrArGrCrUrAmU+G*+C*mU | |
| SEQ ID NO: 39 | AR_UNA_7 | mG*mU*mUrGrGrArGrCrArUrCrUrG/iUNA-rA/rGrUr | crRNA with a single UNA base. |
| | | CrCrArGrGrUrUrUrUrArGrArGrCrUrAmU+G*+C*mU | |
| SEQ ID NO: 40 | AR_UNA_6 | mG*mU*mUrGrGrArGrCrArUrCrUrGrA/iUNA-rG/rUr | crRNA with a single UNA base. |
| | | CrCrArGrGrUrUrUrUrArGrArGrCrUrAmU+G*+C*mU | |
| SEQ ID NO: 41 | AR_UNA_5 | mG*mU*mUrGrGrArGrCrArUrCrUrGrArG/iUNA-rU/ | crRNA with a single UNA base. |
| | | rCrCrArGrGrUrUrUrUrArGrArGrCrUrAmU+G*+C*mU | |
| SEQ ID NO: 42 | AR_UNA_4 | mG*mU*mUrGrGrArGrCrArUrCrUrGrArGrU/iUNA-rC | crRNA with a single UNA base. |
| | | /rCrArGrGrUrUrUrUrArGrArGrCrUrAmU+G*+C*mU | |
| SEQ ID NO: 43 | AR_UNA_3 | mG*mU*mUrGrGrArGrCrArUrCrUrGrArGrUrC/iUNA- | crRNA with a single UNA base. |
| | | rC/rArGrGrUrUrUrUrArGrArGrCrUrAmU+G*+C*mU | |
| SEQ ID NO: 44 | AR_UNA_2 | mG*mU*mUrGrGrArGrCrArUrCrUrGrArGrUrCrC/ | crRNA with a single UNA base. |
| | | iUNA-rA/rGrGrUrUrUrUrArGrArGrCrUrAmU+G*+C*mU | |
| SEQ ID NO: 45 | AR_UNA_1 | mG*mU*mUrGrGrArGrCrArUrCrUrGrArGrUrCrCrA/ | crRNA with a single UNA base. |
| | | iUNA-rG/rGrUrUrUrUrArGrArGrCrUrAmU+G*+C*mU | |
| SEQ ID NO: 46 | LAG3s9_UNA_20 | /5UNA-rG/*mA*mArGrGrCrUrGrArGrArUrCrCrUrGrG | crRNA with a single UNA base. |
| | | rArGrGrGrUrUrUrUrArGrArGrCrUrAmU+G*+C*mU | |
| SEQ ID NO: 47 | LAG3s9_UNA_19 | mG*/iUNA-rA/*mAmGrGrCrUrGrArGrArUrCrCrUrGr | crRNA with a single UNA base. |
| | | GrArGrGrGrUrUrUrUrArGrArGrCrUrAmU+G*+C*mU | |
| SEQ ID NO: 48 | LAG3s9_UNA_18 | mG*mA*/iUNA-rA/rGrGrCrUrGrArGrArUrCrCrUrGrGr | crRNA with a single UNA base. |
| | | ArGrGrGrUrUrUrUrArGrArGrCrUrAmU+G*+C*mU | |
| SEQ ID NO: 49 | LAG3s9_UNA_17 | mG*mA*mA/iUNA-rG/rGrCrUrGrArGrArUrCrCrUrGr | crRNA with a single UNA base. |
| | | GrArGrGrGrUrUrUrUrArGrArGrCrUrAmU+G*+C*mU | |
| SEQ ID NO: 50 | LAG3s9_UNA_16 | mG*mA*mArG/iUNA-rG/rCrUrGrArGrArUrCrCrUrGr | crRNA with a single UNA base. |
| | | GrArGrGrGrUrUrUrUrArGrArGrCrUrAmU+G*+C*mU | |
| SEQ ID NO: 51 | LAG3s9_UNA_15 | mG*mA*mArGrG/iUNA-rC/rUrGrArGrArUrCrCrUrGr | crRNA with a single UNA base. |
| | | GrArGrGrGrUrUrUrUrArGrArGrCrUrAmU+G*+C*mU | |
| SEQ ID NO: 52 | LAG3s9_UNA_14 | mG*mA*mArGrGrC/iUNA-rU/rGrArGrArUrCrCrUrGr | crRNA with a single UNA base. |
| | | GrArGrGrGrUrUrUrUrArGrArGrCrUrAmU+G*+C*mU | |
| SEQ ID NO: 53 | LAG3s9_UNA_13 | mG*mA*mArGrGrCrU/iUNA-rG/rArGrArUrCrCrUrGr | crRNA with a single UNA base. |
| | | GrArGrGrGrUrUrUrUrArGrArGrCrUrAmU+G*+C*mU | |
| SEQ ID NO: 54 | LAG3s9_UNA_12 | mG*mA*mArGrGrCrUrG/iUNA-rA/rGrArUrCrCrUrGr | crRNA with a single UNA base. |
| | | GrArGrGrGrUrUrUrUrArGrArGrCrUrAmU+G*+C*mU | |
| SEQ ID NO: 55 | LAG3s9_UNA_11 | mG*mA*mArGrGrCrUrGrA/iUNA-rG/rArUrCrCrUrGr | crRNA with a single UNA base. |
| | | GrArGrGrGrUrUrUrUrArGrArGrCrUrAmU+G*+C*mU | |
| SEQ ID NO: 56 | LAG3s9_UNA_10 | mG*mA*mArGrGrCrUrGrArG/iUNA-rA/rUrCrCrUrGr | crRNA with a single UNA base. |
| | | GrArGrGrGrUrUrUrUrArGrArGrCrUrAmU+G*+C*mU | |
| SEQ ID NO: 57 | LAG3s9_UNA_9 | mG*mA*mArGrGrCrUrGrArGrA/iUNA-rU/rCrCrUrGr | crRNA with a single UNA base. |
| | | GrArGrGrGrUrUrUrUrArGrArGrCrUrAmU+G*+C*mU | |
| SEQ ID NO: 58 | LAG3s9_UNA_8 | mG*mA*mArGrGrCrUrGrArGrArU/iUNA-rC/rCrUrGr | crRNA with a single UNA base. |
| | | GrArGrGrGrUrUrUrUrArGrArGrCrUrAmU+G*+C*mU | |
| SEQ ID NO: 59 | LAG3s9_UNA_7 | mG*mA*mArGrGrCrUrGrArGrArUrC/iUNA-rC/rUrGr | crRNA with a single UNA base. |
| | | GrArGrGrGrUrUrUrUrArGrArGrCrUrAmU+G*+C*mU | |
| SEQ ID NO: 60 | LAG3s9_UNA_6 | mG*mA*mArGrGrCrUrGrArGrArUrCrC/iUNA-rU/rGr | crRNA with a single UNA base. |
| | | GrArGrGrGrUrUrUrUrArGrArGrCrUrAmU+G*+C*mU | |
| SEQ ID NO: 61 | LAG3s9_UNA_5 | mG*mA*mArGrGrCrUrGrArGrArUrCrCrU/iUNA-rG/r | crRNA with a single UNA base. |
| | | GrArGrGrGrUrUrUrUrArGrArGrCrUrAmU+G*+C*mU | |
| SEQ ID NO: 62 | LAG3s9_UNA_4 | mG*mA*mArGrGrCrUrGrArGrArUrCrCrUrG/iUNA-rG | crRNA with a single UNA base. |
| | | /rArGrGrGrUrUrUrUrArGrArGrCrUrAmU+G*+C*mU | |
| SEQ ID NO: 63 | LAG3s9_UNA_3 | mG*mA*mArGrGrCrUrGrArGrArUrCrCrUrGrG/iUNA- | crRNA with a single UNA base. |
| | | rA/rGrGrGrUrUrUrUrArGrArGrCrUrAmU+G*+C*mU | |
| SEQ ID NO: 64 | LAG3s9_UNA_2 | mG*mA*mArGrGrCrUrGrArGrArUrCrCrUrGrGrA/ | crRNA with a single UNA base. |
| | | iUNA-rG/rGrGrUrUrUrUrArGrArGrCrUrAmU+G*+C*mU | |
| SEQ ID NO: 65 | LAG3s9_UNA_1 | mG*mA*mArGrGrCrUrGrArGrArUrCrCrUrGrGrArG/ | crRNA with a single UNA base. |
| | | iUNA-rG/rGrUrUrUrUrArGrArGrCrUrAmU+G*+C*mU | |
| SEQ ID NO: 66 | EMX1_LNA_20 | +GrArGrUrCrCrGrArGrCrArGrArArGrArArGrArArGrU | crRNA with a single LNA base. |
| | | rUrUrUrArGrArGrCrUrAmU+G*+C*mU | |
| SEQ ID NO: 67 | EMX1_LNA_19 | rG+ArGrUrCrCrGrArGrCrArGrArArGrArArGrArArGrU | crRNA with a single LNA base. |
| | | rUrUrUrArGrArGrCrUrAmU+G*+C*mU | |
| SEQ ID NO: 68 | EMX1_LNA_18 | rGrA+GrUrCrCrGrArGrCrArGrArArGrArArGrArArGrU | crRNA with a single LNA base. |
| | | rUrUrUrArGrArGrCrUrAmU+G*+C*mU | |
| SEQ ID NO: 69 | EMX1_LNA_17 | rGrArG+TrCrCrGrArGrCrArGrArArGrArArGrArArGrU | crRNA with a single LNA base. |
| | | rUrUrUrArGrArGrCrUrAmU+G*+C*mU | |
| SEQ ID NO: 70 | EMX1_LNA_16 | rGrArGrU+CrCrGrArGrCrArGrArArGrArArGrArArGrU | crRNA with a single LNA base. |
| | | rUrUrUrArGrArGrCrUrAmU+G*+C*mU | |
| SEQ ID NO: 71 | EMX1_LNA_15 | rGrArGrUrC+CrGrArGrCrArGrArArGrArArGrArArGrU | crRNA with a single LNA base. |
| | | rUrUrUrArGrArGrCrUrAmU+G*+C*mU | |
| SEQ ID NO: 72 | EMX1_LNA_14 | rGrArGrUrCrC+GrArGrCrArGrArArGrArArGrArArGrU | crRNA with a single LNA base. |
| | | rUrUrUrArGrArGrCrUrAmU+G*+C*mU | |
| SEQ ID NO: 73 | EMX1_LNA_13 | rGrArGrUrCrCrG+ArGrCrArGrArArGrArArGrArArGrU | crRNA with a single LNA base. |
| | | rUrUrUrArGrArGrCrUrAmU+G*+C*mU | |
| SEQ ID NO: 74 | EMX1_LNA_12 | rGrArGrUrCrCrGrA+GrCrArGrArArGrArArGrArArGrU | crRNA with a single LNA base. |
| | | rUrUrUrArGrArGrCrUrAmU+G*+C*mU | |
| SEQ ID NO: 75 | EMX1_LNA_11 | rGrArGrUrCrCrGrArG+CrArGrArArGrArArGrArArGrU | crRNA with a single LNA base. |
| | | rUrUrUrArGrArGrCrUrAmU+G*+C*mU | |
| SEQ ID NO: 76 | EMX1_LNA_10 | rGrArGrUrCrCrGrArGrC+ArGrArArGrArArGrArArGrU | crRNA with a single LNA base. |
| | | rUrUrUrArGrArGrCrUrAmU+G*+C*mU | |
| SEQ ID NO: 77 | EMX1_LNA_9 | rGrArGrUrCrCrGrArGrCrA+GrArArGrArArGrArArGrU | crRNA with a single LNA base. |
| | | rUrUrUrArGrArGrCrUrAmU+G*+C*mU | |
| SEQ ID NO: 78 | EMX1_LNA_8 | rGrArGrUrCrCrGrArGrCrArG+ArArGrArArGrArArGrU | crRNA with a single LNA base. |
| | | rUrUrUrArGrArGrCrUrAmU+G*+C*mU | |
| SEQ ID NO: 79 | EMX1_LNA_7 | rGrArGrUrCrCrGrArGrCrArGrA+ArGrArArGrArArGrU | crRNA with a single LNA base. |
| | | rUrUrUrArGrArGrCrUrAmU+G*+C*mU | |
| SEQ ID NO: 80 | EMX1_LNA_6 | rGrArGrUrCrCrGrArGrCrArGrArA+GrArArGrArArGrU | crRNA with a single LNA base. |
| | | rUrUrUrArGrArGrCrUrAmU+G*+C*mU | |
| SEQ ID NO: 81 | EMX1_LNA_5 | rGrArGrUrCrCrGrArGrCrArGrArArG+ArArGrArArGrU | crRNA with a single LNA base. |
| | | rUrUrUrArGrArGrCrUrAmU+G*+C*mU | |
| SEQ ID NO: 82 | EMX1_LNA_4 | rGrArGrUrCrCrGrArGrCrArGrArArGrA+ArGrArArGrU | crRNA with a single LNA base. |
| | | rUrUrUrArGrArGrCrUrAmU+G*+C*mU | |
| SEQ ID NO: 83 | EMX1_LNA_3 | rGrArGrUrCrCrGrArGrCrArGrArArGrArA+GrArArGrU | crRNA with a single LNA base. |
| | | rUrUrUrArGrArGrCrUrAmU+G*+C*mU | |
| SEQ ID NO: 84 | EMX1_LNA_2 | rGrArGrUrCrCrGrArGrCrArGrArArGrArArG+ArArGrU | crRNA with a single LNA base. |
| | | rUrUrUrArGrArGrCrUrAmU+G*+C*mU | |
| SEQ ID NO: 85 | EMX1_LNA_1 | rGrArGrUrCrCrGrArGrCrArGrArArGrArArGrA+ArGrU | crRNA with a single LNA base. |
| | | rUrUrUrArGrArGrCrUrAmU+G*+C*mU | |
| SEQ ID NO: 86 | EMX1_2Fluor_20 | /52FG/rArGrUrCrCrGrArGrCrArGrArArGrArArGrArAr | crRNA with a single 2′Fluoro |
| | | GrUrUrUrUrArGrArGrCrUrAmU+G*+C*mU | modification. |
| SEQ ID NO: 87 | EMX1_2Fluor_19 | rG/i2FA/rGrUrCrCrGrArGrCrArGrArArGrArArGrArAr | crRNA with a single 2′Fluoro |
| | | GrUrUrUrUrArGrArGrCrUrAmU+G*+C*mU | modification. |
| SEQ ID NO: 88 | EMX1_2Fluor_18 | rGrA/i2FG/rUrCrCrGrArGrCrArGrArArGrArArGrArAr | crRNA with a single 2′Fluoro |
| | | GrUrUrUrUrArGrArGrCrUrAmU+G*+C*mU | modification. |
| SEQ ID NO: 89 | EMX1_2Fluor_17 | rGrArG/i2FU/rCrCrGrArGrCrArGrArArGrArArGrArAr | crRNA with a single 2′Fluoro |
| | | GrUrUrUrUrArGrArGrCrUrAmU+G*+C*mU | modification. |
| SEQ ID NO: 90 | EMX1_2Fluor_16 | rGrArGrU/i2FC/rCrGrArGrCrArGrArArGrArArGrArAr | crRNA with a single 2′Fluoro |
| | | GrUrUrUrUrArGrArGrCrUrAmU+G*+C*mU | modification. |
| SEQ ID NO: 91 | EMX1_2Fluor_15 | rGrArGrUrC/i2FC/rGrArGrCrArGrArArGrArArGrArAr | crRNA with a single 2′Fluoro |
| | | GrUrUrUrUrArGrArGrCrUrAmU+G*+C*mU | modification. |
| SEQ ID NO: 92 | EMX1_2Fluor_14 | rGrArGrUrCrC/i2FG/rArGrCrArGrArArGrArArGrArAr | crRNA with a single 2′Fluoro |
| | | GrUrUrUrUrArGrArGrCrUrAmU+G*+C*mU | modification. |
| SEQ ID NO: 93 | EMX1_2Fluor_13 | rGrArGrUrCrCrG/i2FA/rGrCrArGrArArGrArArGrArAr | crRNA with a single 2′Fluoro |
| | | GrUrUrUrUrArGrArGrCrUrAmU+G*+C*mU | modification. |
| SEQ ID NO: 94 | EMX1_2Fluor_12 | rGrArGrUrCrCrGrA/i2FG/rCrArGrArArGrArArGrArAr | crRNA with a single 2′Fluoro |
| | | GrUrUrUrUrArGrArGrCrUrAmU+G*+C*mU | modification. |
| SEQ ID NO: 95 | EMX1_2Fluor_11 | rGrArGrUrCrCrGrArG/i2FC/rArGrArArGrArArGrArAr | crRNA with a single 2′Fluoro |
| | | GrUrUrUrUrArGrArGrCrUrAmU+G*+C*mU | modification. |
| SEQ ID NO: 96 | EMX1_2Fluor_10 | rGrArGrUrCrCrGrArGrC/i2FA/rGrArArGrArArGrArAr | crRNA with a single 2′Fluoro |
| | | GrUrUrUrUrArGrArGrCrUrAmU+G*+C*mU | modification. |
| SEQ ID NO: 97 | EMX1_2Fluor_9 | rGrArGrUrCrCrGrArGrCrA/i2FG/rArArGrArArGrArAr | crRNA with a single 2′Fluoro |
| | | GrUrUrUrUrArGrArGrCrUrAmU+G*+C*mU | modification. |
| SEQ ID NO: 98 | EMX1_2Fluor_8 | rGrArGrUrCrCrGrArGrCrArG/i2FA/rArGrArArGrArAr | crRNA with a single 2′Fluoro |
| | | GrUrUrUrUrArGrArGrCrUrAmU+G*+C*mU | modification. |
| SEQ ID NO: 99 | EMX1_2Fluor_7 | rGrArGrUrCrCrGrArGrCrArGrA/i2FA/rGrArArGrArAr | crRNA with a single 2′Fluoro |
| | | GrUrUrUrUrArGrArGrCrUrAmU+G*+C*mU | modification. |
| SEQ ID NO: 100 | EMX1_2Fluor_6 | rGrArGrUrCrCrGrArGrCrArGrArA/i2FG/rArArGrArAr | crRNA with a single 2′Fluoro |
| | | GrUrUrUrUrArGrArGrCrUrAmU+G*+C*mU | modification. |
| SEQ ID NO: 101 | EMX1_2Fluor_5 | rGrArGrUrCrCrGrArGrCrArGrArArG/i2FA/rArGrArAr | crRNA with a single 2′Fluoro |
| | | GrUrUrUrUrArGrArGrCrUrAmU+G*+C*mU | modification. |
| SEQ ID NO: 102 | EMX1_2Fluor_4 | rGrArGrUrCrCrGrArGrCrArGrArArGrA/i2FA/rGrArAr | crRNA with a single 2′Fluoro |
| | | GrUrUrUrUrArGrArGrCrUrAmU+G*+C*mU | modification. |
| SEQ ID NO: 103 | EMX1_2Fluor_3 | rGrArGrUrCrCrGrArGrCrArGrArArGrArA/i2FG/rArAr | crRNA with a single 2′Fluoro |
| | | GrUrUrUrUrArGrArGrCrUrAmU+G*+C*mU | modification. |
| SEQ ID NO: 104 | EMX1_2Fluor_2 | rGrArGrUrCrCrGrArGrCrArGrArArGrArArG/i2FA/rAr | crRNA with a single 2′Fluoro |
| | | GrUrUrUrUrArGrArGrCrUrAmU+G*+C*mU | modification. |
| SEQ ID NO: 105 | EMX1_2Fluor_1 | rGrArGrUrCrCrGrArGrCrArGrArArGrArArGrA/i2FA/r | crRNA with a single 2′Fluoro |
| | | GrUrUrUrUrArGrArGrCrUrAmU+G*+C*mU | modification. |
| SEQ ID NO: 106 | AR_LNA_20 | +GrUrUrGrGrArGrCrArUrCrUrGrArGrUrCrCrArGrGrU | crRNA with a single LNA base. |
| | | rUrUrUrArGrArGrCrUrAmU+G*+C*mU | |
| SEQ ID NO: 107 | AR_LNA_19 | rG+TrUrGrGrArGrCrArUrCrUrGrArGrUrCrCrArGrGrU | crRNA with a single LNA base. |
| | | rUrUrUrArGrArGrCrUrAmU+G*+C*mU | |
| SEQ ID NO: 108 | AR_LNA_18 | rGrU+TrGrGrArGrCrArUrCrUrGrArGrUrCrCrArGrGrU | crRNA with a single LNA base. |
| | | rUrUrUrArGrArGrCrUrAmU+G*+C*mU | |
| SEQ ID NO: 109 | AR_LNA_17 | rGrUrU+GrGrArGrCrArUrCrUrGrArGrUrCrCrArGrGrU | crRNA with a single LNA base. |
| | | rUrUrUrArGrArGrCrUrAmU+G*+C*mU | |
| SEQ ID NO: | AR_LNA_16 | rGrUrUrG+GrArGrCrArUrCrUrGrArGrUrCrCrArGrGrU | crRNA with a single LNA base. |
| 110 | | rUrUrUrArGrArGrCrUrAmU+G*+C*mU | |
| SEQ ID NO: | AR_LNA_15 | rGrUrUrGrG+ArGrCrArUrCrUrGrArGrUrCrCrArGrGrU | crRNA with a single LNA base. |
| 111 | | rUrUrUrArGrArGrCrUrAmU+G*+C*mU | |
| SEQ ID NO: | AR_LNA_14 | rGrUrUrGrGrA+GrCrArUrCrUrGrArGrUrCrCrArGrGrU | crRNA with a single LNA base. |
| 112 | | rUrUrUrArGrArGrCrUrAmU+G*+C*mU | |
| SEQ ID NO: | AR_LNA_13 | rGrUrUrGrGrArG+CrArUrCrUrGrArGrUrCrCrArGrGrU | crRNA with a single LNA base. |
| 113 | | rUrUrUrArGrArGrCrUrAmU+G*+C*mU | |
| SEQ ID NO: | AR_LNA_12 | rGrUrUrGrGrArGrC+ArUrCrUrGrArGrUrCrCrArGrGrU | crRNA with a single LNA base. |
| 114 | | rUrUrUrArGrArGrCrUrAmU+G*+C*mU | |
| SEQ ID NO: | AR_LNA_11 | rGrUrUrGrGrArGrCrA+TrCrUrGrArGrUrCrCrArGrGrU | crRNA with a single LNA base. |
| 115 | | rUrUrUrArGrArGrCrUrAmU+G*+C*mU | |
| SEQ ID NO: | AR_LNA_10 | rGrUrUrGrGrArGrCrArU+CrUrGrArGrUrCrCrArGrGrU | crRNA with a single LNA base. |
| 116 | | rUrUrUrArGrArGrCrUrAmU+G*+C*mU | |
| SEQ ID NO: | AR_LNA_9 | rGrUrUrGrGrArGrCrArUrC+TrGrArGrUrCrCrArGrGrU | crRNA with a single LNA base. |
| 117 | | rUrUrUrArGrArGrCrUrAmU+G*+C*mU | |
| SEQ ID NO: | AR_LNA_8 | rGrUrUrGrGrArGrCrArUrCrU+GrArGrUrCrCrArGrGrU | crRNA with a single LNA base. |
| 118 | | rUrUrUrArGrArGrCrUrAmU+G*+C*mU | |
| SEQ ID NO: | AR_LNA_7 | rGrUrUrGrGrArGrCrArUrCrUrG+ArGrUrCrCrArGrGrU | crRNA with a single LNA base. |
| 119 | | rUrUrUrArGrArGrCrUrAmU+G*+C*mU | |
| SEQ ID NO: | AR_LNA_6 | rGrUrUrGrGrArGrCrArUrCrUrGrA+GrUrCrCrArGrGrU | crRNA with a single LNA base. |
| 120 | | rUrUrUrArGrArGrCrUrAmU+G*+C*mU | |
| SEQ ID NO: | AR_LNA_5 | rGrUrUrGrGrArGrCrArUrCrUrGrArG+TrCrCrArGrGrU | crRNA with a single LNA base. |
| 121 | | rUrUrUrArGrArGrCrUrAmU+G*+C*mU | |
| SEQ ID NO: 122 | AR_LNA_4 | rGrUrUrGrGrArGrCrArUrCrUrGrArGrU+CrCrArGrGrU | crRNA with a single LNA base. |
| | | rUrUrUrArGrArGrCrUrAmU+G*+C*mU | |
| SEQ ID NO: | AR_LNA_3 | rGrUrUrGrGrArGrCrArUrCrUrGrArGrUrC+CrArGrGrU | crRNA with a single LNA base. |
| 123 | | rUrUrUrArGrArGrCrUrAmU+G*+C*mU | |
| SEQ ID NO: 124 | AR_LNA_2 | rGrUrUrGrGrArGrCrArUrCrUrGrArGrUrCrC+ArGrGrU | crRNA with a single LNA base. |
| | | rUrUrUrArGrArGrCrUrAmU+G*+C*mU | |
| SEQ ID NO: | AR_LNA_1 | rGrUrUrGrGrArGrCrArUrCrUrGrArGrUrCrCrA+GrGrU | crRNA with a single LNA base. |
| 125 | | rUrUrUrArGrArGrCrUrAmU+G*+C*mU | |
| SEQ ID NO: | LAG3_LNA_20 | +GrArArGrGrCrUrGrArGrArUrCrCrUrGrGrArGrGrGrU | crRNA with a single LNA base. |
| 126 | | rUrUrUrArGrArGrCrUrAmU+G*+C*mU | |
| SEQ ID NO: | LAG3_LNA_19 | rG+ArArGrGrCrUrGrArGrArUrCrCrUrGrGrArGrGrGrU | crRNA with a single LNA base. |
| 127 | | rUrUrUrArGrArGrCrUrAmU+G*+C*mU | |
| SEQ ID NO: | LAG3_LNA_18 | rGrA+ArGrGrCrUrGrArGrArUrCrCrUrGrGrArGrGrGrU | crRNA with a single LNA base. |
| 128 | | rUrUrUrArGrArGrCrUrAmU+G*+C*mU | |
| SEQ ID NO: 129 | LAG3_LNA_17 | rGrArA+GrGrCrUrGrArGrArUrCrCrUrGrGrArGrGrGrU | crRNA with a single LNA base. |
| | | rUrUrUrArGrArGrCrUrAmU+G*+C*mU | |
| SEQ ID NO: | LAG3_LNA_16 | rGrArArG+GrCrUrGrArGrArUrCrCrUrGrGrArGrGrGrU | crRNA with a single LNA base. |
| 130 | | rUrUrUrArGrArGrCrUrAmU+G*+C*mU | |
| SEQ ID NO: 131 | LAG3_LNA_15 | rGrArArGrG+CrUrGrArGrArUrCrCrUrGrGrArGrGrGrU | crRNA with a single LNA base. |
| | | rUrUrUrArGrArGrCrUrAmU+G*+C*mU | |
| SEQ ID NO: 132 | LAG3_LNA_14 | rGrArArGrGrC+TrGrArGrArUrCrCrUrGrGrArGrGrGrU | crRNA with a single LNA base. |
| | | rUrUrUrArGrArGrCrUrAmU+G*+C*mU | |
| SEQ ID NO: | LAG3_LNA_13 | rGrArArGrGrCrU+GrArGrArUrCrCrUrGrGrArGrGrGrU | crRNA with a single LNA base. |
| 133 | | rUrUrUrArGrArGrCrUrAmU+G*+C*mU | |
| SEQ ID NO: | LAG3_LNA_12 | rGrArArGrGrCrUrG+ArGrArUrCrCrUrGrGrArGrGrGrU | crRNA with a single LNA base. |
| 134 | | rUrUrUrArGrArGrCrUrAmU+G*+C*mU | |
| SEQ ID NO: | LAG3_LNA_11 | rGrArArGrGrCrUrGrA+GrArUrCrCrUrGrGrArGrGrGrU | crRNA with a single LNA base. |
| 135 | | rUrUrUrArGrArGrCrUrAmU+G*+C*mU | |
| SEQ ID NO: | LAG3_LNA_10 | rGrArArGrGrCrUrGrArG+ArUrCrCrUrGrGrArGrGrGrU | crRNA with a single LNA base. |
| 136 | | rUrUrUrArGrArGrCrUrAmU+G*+C*mU | |
| SEQ ID NO: | LAG3_LNA_9 | rGrArArGrGrCrUrGrArGrA+TrCrCrUrGrGrArGrGrGrU | crRNA with a single LNA base. |
| 137 | | rUrUrUrArGrArGrCrUrAmU+G*+C*mU | |
| SEQ ID NO: 138 | LAG3_LNA_8 | rGrArArGrGrCrUrGrArGrArU+CrCrUrGrGrArGrGrGrU | crRNA with a single LNA base. |
| | | rUrUrUrArGrArGrCrUrAmU+G*+C*mU | |
| SEQ ID NO: | LAG3_LNA_7 | rGrArArGrGrCrUrGrArGrArUrC+CrUrGrGrArGrGrGrU | crRNA with a single LNA base. |
| 139 | | rUrUrUrArGrArGrCrUrAmU+G*+C*mU | |
| SEQ ID NO: 140 | LAG3_LNA_6 | rGrArArGrGrCrUrGrArGrArUrCrC+TrGrGrArGrGrGrU | crRNA with a single LNA base. |
| | | rUrUrUrArGrArGrCrUrAmU+G*+C*mU | |
| SEQ ID NO: | LAG3_LNA_5 | rGrArArGrGrCrUrGrArGrArUrCrCrU+GrGrArGrGrGrU | crRNA with a single LNA base. |
| 141 | | rUrUrUrArGrArGrCrUrAmU+G*+C*mU | |
| SEQ ID NO: | LAG3_LNA_4 | rGrArArGrGrCrUrGrArGrArUrCrCrUrG+GrArGrGrGrU | crRNA with a single LNA base. |
| 142 | | rUrUrUrArGrArGrCrUrAmU+G*+C*mU | |
| SEQ ID NO: | LAG3_LNA_3 | rGrArArGrGrCrUrGrArGrArUrCrCrUrGrG+ArGrGrGrU | crRNA with a single LNA base. |
| 143 | | rUrUrUrArGrArGrCrUrAmU+G*+C*mU | |
| SEQ ID NO: | LAG3_LNA_2 | rGrArArGrGrCrUrGrArGrArUrCrCrUrGrGrA+GrGrGrU | crRNA with a single LNA base. |
| 144 | | rUrUrUrArGrArGrCrUrAmU+G*+C*mU | |
| SEQ ID NO: | LAG3_LNA_1 | rGrArArGrGrCrUrGrArGrArUrCrCrUrGrGrArG+GrGrU | crRNA with a single LNA base. |
| 145 | | rUrUrUrArGrArGrCrUrAmU+G*+C*mU | |
| SEQ ID NO: | AR_2Fluoro_20 | /52FG/rUrUrGrGrArGrCrArUrCrUrGrArGrUrCrCrArGr | crRNA with a single 2′Fluoro |
| 146 | | GrUrUrUrUrArGrArGrCrUrAmU+G*+C*mU | modification. |
| SEQ ID NO: | AR_2Fluoro_19 | rG/i2FU/rUrGrGrArGrCrArUrCrUrGrArGrUrCrCrArGr | crRNA with a single 2′Fluoro |
| 147 | | GrUrUrUrUrArGrArGrCrUrAmU+G*+C*mU | modification. |
| SEQ ID NO: | AR_2Fluoro_18 | rGrU/i2FU/rGrGrArGrCrArUrCrUrGrArGrUrCrCrArGr | crRNA with a single 2′Fluoro |
| 148 | | GrUrUrUrUrArGrArGrCrUrAmU+G*+C*mU | modification. |
| SEQ ID NO: | AR_2Fluoro_17 | rGrUrU/i2FG/rGrArGrCrArUrCrUrGrArGrUrCrCrArGr | crRNA with a single 2′Fluoro |
| 149 | | GrUrUrUrUrArGrArGrCrUrAmU+G*+C*mU | modification. |
| SEQ ID NO: | AR_2Fluoro_16 | rGrUrUrG/i2FG/rArGrCrArUrCrUrGrArGrUrCrCrArGr | crRNA with a single 2′Fluoro |
| 150 | | GrUrUrUrUrArGrArGrCrUrAmU+G*+C*mU | modification. |
| SEQ ID NO: | AR_2Fluoro_15 | rGrUrUrGrG/i2FA/rGrCrArUrCrUrGrArGrUrCrCrArGr | crRNA with a single 2′Fluoro |
| 151 | | GrUrUrUrUrArGrArGrCrUrAmU+G*+C*mU | modification. |
| SEQ ID NO: | AR_2Fluoro_14 | rGrUrUrGrGrA/i2FG/rCrArUrCrUrGrArGrUrCrCrArGr | crRNA with a single 2′Fluoro |
| 152 | | GrUrUrUrUrArGrArGrCrUrAmU+G*+C*mU | modification. |
| SEQ ID NO: | AR_2Fluoro_13 | rGrUrUrGrGrArG/i2FC/rArUrCrUrGrArGrUrCrCrArGr | crRNA with a single 2′Fluoro |
| 153 | | GrUrUrUrUrArGrArGrCrUrAmU+G*+C*mU | modification. |
| SEQ ID NO: 154 | AR_2Fluoro_12 | rGrUrUrGrGrArGrC/i2FA/rUrCrUrGrArGrUrCrCrArGr | crRNA with a single 2′Fluoro |
| | | GrUrUrUrUrArGrArGrCrUrAmU+G*+C*mU | modification. |
| SEQ ID NO: 155 | AR_2Fluoro_11 | rGrUrUrGrGrArGrCrA/i2FU/rCrUrGrArGrUrCrCrArGr | crRNA with a single 2′Fluoro |
| | | GrUrUrUrUrArGrArGrCrUrAmU+G*+C*mU | modification. |
| SEQ ID NO: 156 | AR_2Fluoro_10 | rGrUrUrGrGrArGrCrArU/i2FC/rUrGrArGrUrCrCrArGr | crRNA with a single 2′Fluoro |
| | | GrUrUrUrUrArGrArGrCrUrAmU+G*+C*mU | modification. |
| SEQ ID NO: | AR_2Fluoro_9 | rGrUrUrGrGrArGrCrArUrC/i2FU/rGrArGrUrCrCrArGr | crRNA with a single 2′Fluoro |
| 157 | | GrUrUrUrUrArGrArGrCrUrAmU+G*+C*mU | modification. |
| SEQ ID NO: 158 | AR_2Fluoro_8 | rGrUrUrGrGrArGrCrArUrCrU/i2FG/rArGrUrCrCrArGr | crRNA with a single 2′Fluoro |
| | | GrUrUrUrUrArGrArGrCrUrAmU+G*+C*mU | modification. |
| SEQ ID NO: 159 | AR_2Fluoro_7 | rGrUrUrGrGrArGrCrArUrCrUrG/i2FA/rGrUrCrCrArGr | crRNA with a single 2′Fluoro |
| | | GrUrUrUrUrArGrArGrCrUrAmU+G*+C*mU | modification. |
| SEQ ID NO: 160 | AR_2Fluoro_6 | rGrUrUrGrGrArGrCrArUrCrUrGrA/i2FG/rUrCrCrArGr | crRNA with a single 2′Fluoro |
| | | GrUrUrUrUrArGrArGrCrUrAmU+G*+C*mU | modification. |
| SEQ ID NO: 161 | AR_2Fluoro_5 | rGrUrUrGrGrArGrCrArUrCrUrGrArG/i2FU/rCrCrArGr | crRNA with a single 2′Fluoro |
| | | GrUrUrUrUrArGrArGrCrUrAmU+G*+C*mU | modification. |
| SEQ ID NO: 162 | AR_2Fluoro_4 | rGrUrUrGrGrArGrCrArUrCrUrGrArGrU/i2FC/rCrArGr | crRNA with a single 2′Fluoro |
| | | GrUrUrUrUrArGrArGrCrUrAmU+G*+C*mU | modification. |
| SEQ ID NO: 163 | AR_2Fluoro_3 | rGrUrUrGrGrArGrCrArUrCrUrGrArGrUrC/i2FC/rArGr | crRNA with a single 2′Fluoro |
| | | GrUrUrUrUrArGrArGrCrUrAmU+G*+C*mU | modification. |
| SEQ ID NO: 164 | AR_2Fluoro_2 | rGrUrUrGrGrArGrCrArUrCrUrGrArGrUrCrC/i2FA/rGr | crRNA with a single 2′Fluoro |
| | | GrUrUrUrUrArGrArGrCrUrAmU+G*+C*mU | modification. |
| SEQ ID NO: 165 | AR_2Fluoro_1 | rGrUrUrGrGrArGrCrArUrCrUrGrArGrUrCrCrA/i2FG/r | crRNA with a single 2′Fluoro |
| | | GrUrUrUrUrArGrArGrCrUrAmU+G*+C*mU | modification. |
| SEQ ID NO: | LAG3_2Fluoro_20 | /52FG/rArArGrGrCrUrGrArGrArUrCrCrUrGrGrArGrGr | crRNA with a single 2′Fluoro |
| 166 | | GrUrUrUrUrArGrArGrCrUrAmU+G*+C*mU | modification |
| SEQ ID NO: 167 | LAG3_2Fluoro_19 | rG/i2FA/rArGrGrCrUrGrArGrArUrCrCrUrGrGrArGrGr | crRNA with a single 2′Fluoro |
| | | GrUrUrUrUrArGrArGrCrUrAmU+G*+C*mU | modification. |
| SEQ ID NO: 168 | LAG3_2Fluoro_18 | rGrA/i2FA/rGrGrCrUrGrArGrArUrCrCrUrGrGrArGrGr | crRNA with a single 2′Fluoro |
| | | GrUrUrUrUrArGrArGrCrUrAmU+G*+C*mU | modification. |
| SEQ ID NO: | LAG3_2Fluoro_17 | rGrArA/i2FG/rGrCrUrGrArGrArUrCrCrUrGrGrArGrGr | crRNA with a single 2′Fluoro |
| 169 | | GrUrUrUrUrArGrArGrCrUrAmU+G*+C*mU | modification. |
| SEQ ID NO: 170 | LAG3_2Fluoro_16 | rGrArArG/i2FG/rCrUrGrArGrArUrCrCrUrGrGrArGrGr | crRNA with a single 2′Fluoro |
| | | GrUrUrUrUrArGrArGrCrUrAmU+G*+C*mU | modification. |
| SEQ ID NO: 171 | LAG3_2Fluoro_15 | rGrArArGrG/i2FC/rUrGrArGrArUrCrCrUrGrGrArGrGr | crRNA with a single 2′Fluoro |
| | | GrUrUrUrUrArGrArGrCrUrAmU+G*+C*mU | modification. |
| SEQ ID NO: 172 | LAG3_2Fluoro_14 | rGrArArGrGrC/i2FU/rGrArGrArUrCrCrUrGrGrArGrGr | crRNA with a single 2′Fluoro |
| | | GrUrUrUrUrArGrArGrCrUrAmU+G*+C*mU | modification. |
| SEQ ID NO: 173 | LAG3_2Fluoro_13 | rGrArArGrGrCrU/i2FG/rArGrArUrCrCrUrGrGrArGrGr | crRNA with a single 2′Fluoro |
| | | GrUrUrUrUrArGrArGrCrUrAmU+G*+C*mU | modification. |
| SEQ ID NO: 174 | LAG3_2Fluoro_12 | rGrArArGrGrCrUrG/i2FA/rGrArUrCrCrUrGrGrArGrGr | crRNA with a single 2′Fluoro |
| | | GrUrUrUrUrArGrArGrCrUrAmU+G*+C*mU | modification. |
| SEQ ID NO: 175 | LAG3_2Fluoro_11 | rGrArArGrGrCrUrGrA/i2FG/rArUrCrCrUrGrGrArGrGr | crRNA with a single 2′Fluoro |
| | | GrUrUrUrUrArGrArGrCrUrAmU+G*+C*mU | modification. |
| SEQ ID NO: 176 | LAG3_2Fluoro_10 | rGrArArGrGrCrUrGrArG/i2FA/rUrCrCrUrGrGrArGrGr | crRNA with a single 2′Fluoro |
| | | GrUrUrUrUrArGrArGrCrUrAmU+G*+C*mU | modification. |
| SEQ ID NO: 177 | LAG3_2Fluoro_9 | rGrArArGrGrCrUrGrArGrA/i2FU/rCrCrUrGrGrArGrGr | crRNA with a single 2′Fluoro |
| | | GrUrUrUrUrArGrArGrCrUrAmU+G*+C*mU | modification. |
| SEQ ID NO: 178 | LAG3_2Fluoro_8 | rGrArArGrGrCrUrGrArGrArU/i2FC/rCrUrGrGrArGrGr | crRNA with a single 2′Fluoro |
| | | GrUrUrUrUrArGrArGrCrUrAmU+G*+C*mU | modification. |
| SEQ ID NO: | LAG3_2Fluoro_7 | rGrArArGrGrCrUrGrArGrArUrC/i2FC/rUrGrGrArGrGr | crRNA with a single 2′Fluoro |
| 179 | | GrUrUrUrUrArGrArGrCrUrAmU+G*+C*mU | modification. |
| SEQ ID NO: 180 | LAG3_2Fluoro_6 | rGrArArGrGrCrUrGrArGrArUrCrC/i2FU/rGrGrArGrGr | crRNA with a single 2′Fluoro |
| | | GrUrUrUrUrArGrArGrCrUrAmU+G*+C*mU | modification. |
| SEQ ID NO: 181 | LAG3_2Fluoro_5 | rGrArArGrGrCrUrGrArGrArUrCrCrU/i2FG/rGrArGrGr | crRNA with a single 2′Fluoro |
| | | GrUrUrUrUrArGrArGrCrUrAmU+G*+C*mU | modification. |
| SEQ ID NO: | LAG3_2Fluoro_4 | rGrArArGrGrCrUrGrArGrArUrCrCrUrG/i2FG/rArGrGr | crRNA with a single 2′Fluoro |
| 182 | | GrUrUrUrUrArGrArGrCrUrAmU+G*+C*mU | modification. |
| SEQ ID NO: 183 | LAG3_2Fluoro_3 | rGrArArGrGrCrUrGrArGrArUrCrCrUrGrG/i2FA/rGrGr | crRNA with a single 2′Fluoro |
| | | GrUrUrUrUrArGrArGrCrUrAmU+G*+C*mU | modification. |
| SEQ ID NO: 184 | LAG3_2Fluoro_2 | rGrArArGrGrCrUrGrArGrArUrCrCrUrGrGrA/i2FG/rGr | crRNA with a single 2′Fluoro |
| | | GrUrUrUrUrArGrArGrCrUrAmU+G*+C*mU | modification. |
| SEQ ID NO: 185 | LAG3_2Fluoro_1 | rGrArArGrGrCrUrGrArGrArUrCrCrUrGrGrArG/12FG/r | crRNA with a single 2′Fluoro |
| | | GrUrUrUrUrArGrArGrCrUrAmU+G*+C*mU | modification. |
| SEQ ID NO: 186 | AR_c3_20 | /5SpC3/*mU*mUrGrGrArGrCrArUrCrUrGrArGrUrCrCr | crRNA with a single C3 spacer |
| | | ArGrGrUrUrUrUrArGrArGrCrUrAmU+G*+C*mU (SEQ | modification. |
| | | ID NO: 186) | |
| SEQ ID NO: 187 | AR_c3_19 | mG*/iSpC3/*mUrGrGrArGrCrArUrCrUrGrArGrUrCrCr | crRNA with a single C3 spacer |
| | | ArGrGrUrUrUrUrArGrArGrCrUrAmU+G*+C*mU (SEQ | modification. |
| | | ID NO: 187) | |
| SEQ ID NO: 188 | AR_c3_18 | mG*mU*/iSpC3/rGrGrArGrCrArUrCrUrGrArGrUrCrCr | crRNA with a single C3 spacer |
| | | ArGrGrUrUrUrUrArGrArGrCrUrAmU+G*+C*mU (SEQ | modification. |
| | | ID NO: 188) | |
| SEQ ID NO: 189 | AR_c3_17 | mG*mU*mU/iSpC3/rGrArGrCrArUrCrUrGrArGrUrCrC | crRNA with a single C3 spacer |
| | | rArGrGrUrUrUrUrArGrArGrCrUrAmU+G*+C*mU | modification. |
| | | (SEQ ID NO: 189) | |
| SEQ ID NO: 190 | AR_c3_16 | mG*mU*mUrG/iSpC3/rArGrCrArUrCrUrGrArGrUrCrC | crRNA with a single C3 spacer |
| | | rArGrGrUrUrUrUrArGrArGrCrUrAmU+G*+C*mU | modification. |
| | | (SEQ ID NO: 190) | |
| SEQ ID NO: 191 | AR_c3_15 | mG*mU*mUrGrG/iSpC3/rGrCrArUrCrUrGrArGrUrCrC | crRNA with a single C3 spacer |
| | | rArGrGrUrUrUrUrArGrArGrCrUrAmU+G*+C*mU | modification. |
| | | (SEQ ID NO: 191) | |
| SEQ ID NO: 192 | AR_c3_14 | mG*mU*mUrGrGrA/iSpC3/rCrArUrCrUrGrArGrUrCrC | crRNA with a single C3 spacer |
| | | rArGrGrUrUrUrUrArGrArGrCrUrAmU+G*+C*mU | modification. |
| | | (SEQ ID NO: 192) | |
| SEQ ID NO: 193 | AR_c3_13 | mG*mU*mUrGrGrArG/iSpC3/rArUrCrUrGrArGrUrCrC | crRNA with a single C3 spacer |
| | | rArGrGrUrUrUrUrArGrArGrCrUrAmU+G*+C*mU | modification. |
| | | (SEQ ID NO: 193) | |
| SEQ ID NO: 194 | AR_c3_12 | mG*mU*mUrGrGrArGrC/iSpC3/rUrCrUrGrArGrUrCrC | crRNA with a single C3 spacer |
| | | rArGrGrUrUrUrUrArGrArGrCrUrAmU+G*+C*mU | modification. |
| | | (SEQ ID NO: 194) | |
| SEQ ID NO: | AR_c3_11 | mG*mU*mUrGrGrArGrCrA/iSpC3/rCrUrGrArGrUrCrC | crRNA with a single C3 spacer |
| 195 | | rArGrGrUrUrUrUrArGrArGrCrUrAmU+G*+C*mU | modification. |
| | | (SEQ ID NO: 195) | |
| SEQ ID NO: 196 | AR_c3_10 | mG*mU*mUrGrGrArGrCrArU (SEQ ID NO: 196) | crRNA with a single C3 spacer |
| | | /iSpC3/rUrGrArGrUrCrCrArGrGrUrUrUrUrArGrArGrCr | modification. |
| | | UrAmU+G*+C*mU (SEQ ID NO: 649) | |
| SEQ ID NO: 197 | AR_c3_9 | mG*mU*mUrGrGrArGrCrArUrC (SEQ ID NO: | crRNA with a single C3 spacer |
| | | 197)/iSpC3/rGrArGrUrCrCrArGrGrUrUrUrUrArGrArGr | modification. |
| | | CrUrAmU+G*+C*mU (SEQ ID NO: 650) | |
| SEQ ID NO: 198 | AR_c3_8 | mG*mU*mUrGrGrArGrCrArUrCrU (SEQ ID NO: | crRNA with a single C3 spacer |
| | | 198)/iSpC3/rArGrUrCrCrArGrGrUrUrUrUrArGrArGrCr | modification. |
| | | UrAmU+G*+C*mU (SEQ ID NO: 651) | |
| SEQ ID NO: 199 | AR_c3_7 | mG*mU*mUrGrGrArGrCrArUrCrUrG (SEQ ID NO: | crRNA with a single C3 spacer |
| | | 199)/iSpC3/rGrUrCrCrArGrGrUrUrUrUrArGrArGrCrUr | modification. |
| | | AmU+G*+C*mU (SEQ ID NO: 652) | |
| SEQ ID NO: 200 | AR_c3_6 | mG*mU*mUrGrGrArGrCrArUrCrUrGrA (SEQ ID NO: | crRNA with a single C3 spacer |
| | | 200)/iSpC3/rUrCrCrArGrGrUrUrUrUrArGrArGrCrUrA | modification. |
| | | mU+G*+C*mU (SEQ ID NO: 653) | |
| SEQ ID NO: 201 | AR_c3_5 | mG*mU*mUrGrGrArGrCrArUrCrUrGrArG (SEQ ID | crRNA with a single C3 spacer |
| | | NO: 201)/ | modification. |
| | | iSpC3/rCrCrArGrGrUrUrUrUrArGrArGrCrUrAmU | |
| | | +G*+C*mU (SEQ ID NO: 654) | |
| SEQ ID NO: | AR_c3_4 | mG*mU*mUrGrGrArGrCrArUrCrUrGrArGrU (SEQ ID | crRNA with a single C3 spacer |
| 202 | | NO: 202)/ | modification. |
| | | iSpC3/rCrArGrGrUrUrUrUrArGrArGrCrUrAmU+G | |
| | | *+C*mU (SEQ ID NO: 655) | |
| SEQ ID NO: | AR_c3_3 | mG*mU*mUrGrGrArGrCrArUrCrUrGrArGrUrC (SEQ | crRNA with a single C3 spacer |
| 203 | | ID NO: 203)/ | modification. |
| | | iSpC3/rArGrGrUrUrUrUrArGrArGrCrUrAmU+G* | |
| | | +C*mU (SEQ ID NO: 656) | |
| SEQ ID NO: | AR_c3_2 | mG*mU*mUrGrGrArGrCrArUrCrUrGrArGrUrCrC (SEQ | crRNA with a single C3 spacer |
| 204 | | ID NO: 204)/ | modification. |
| | | iSpC3/rGrGrUrUrUrUrArGrArGrCrUrAmU+G*+C | |
| | | *mU (SEQ ID NO: 657) | |
| SEQ ID NO: | AR_c3_1 | mG*mU*mUrGrGrArGrCrArUrCrUrGrArGrUrCrCrA | crRNA with a single C3 spacer |
| 205 | | (SEQ ID NO: 205)/ | modification. |
| | | iSpC3/rGrUrUrUrUrArGrArGrCrUrAmU+G*+C*mU | |
| | | (SEQ ID NO: 658) | |
| SEQ ID NO: | LAG3s9_c3_20 | /5SpC3/*mA*mArGrGrCrUrGrArGrArUrCrCrUrGrGrAr | crRNA with a single C3 spacer |
| 206 | | GrGrGrUrUrUrUrArGrArGrCrUrAmU+G*+C*mU (SEQ | modification. |
| | | ID NO: 206) | |
| SEQ ID NO: | LAG3s9_c3_19 | mG*/iSpC3/*mAmGrGrCrUrGrArGrArUrCrCrUrGrGrA | crRNA with a single C3 spacer |
| 207 | | rGrGrGrUrUrUrUrArGrArGrCrUrAmU+G*+C*mU | modification. |
| | | (SEQ ID NO: 207) | |
| SEQ ID NO: | LAG3s9_c3_18 | mG*mA*/iSpC3/rGrGrCrUrGrArGrArUrCrCrUrGrGrAr | crRNA with a single C3 spacer |
| 208 | | GrGrGrUrUrUrUrArGrArGrCrUrAmU+G*+C*mU (SEQ | modification. |
| | | ID NO: 208) | |
| SEQ ID NO: | LAG3s9_c3_17 | mG*mA*mA/iSpC3/rGrCrUrGrArGrArUrCrCrUrGrGrA | crRNA with a single C3 spacer |
| 209 | | rGrGrGrUrUrUrUrArGrArGrCrUrAmU+G*+C*mU | modification. |
| | | (SEQ ID NO: 209) | |
| SEQ ID NO: | LAG3s9_c3_16 | mG*mA*mArG/iSpC3/rCrUrGrArGrArUrCrCrUrGrGrA | crRNA with a single C3 spacer |
| 210 | | rGrGrGrUrUrUrUrArGrArGrCrUrAmU+G*+C*mU | modification. |
| | | (SEQ ID NO: 210) | |
| SEQ ID NO: | LAG3s9_c3_15 | mG*mA*mArGrG/iSpC3/rUrGrArGrArUrCrCrUrGrGrA | crRNA with a single C3 spacer |
| 211 | | rGrGrGrUrUrUrUrArGrArGrCrUrAmU+G*+C*mU | modification. |
| | | (SEQ ID NO: 211) | |
| SEQ ID NO: | LAG3s9_c3_14 | mG*mA*mArGrGrC/iSpC3/rGrArGrArUrCrCrUrGrGrA | crRNA with a single C3 spacer |
| 212 | | rGrGrGrUrUrUrUrArGrArGrCrUrAmU+G*+C*mU | modification. |
| | | (SEQ ID NO: 212) | |
| SEQ ID NO: | LAG3s9_c3_13 | mG*mA*mArGrGrCrU/iSpC3/rArGrArUrCrCrUrGrGrA | crRNA with a single C3 spacer |
| 213 | | rGrGrGrUrUrUrUrArGrArGrCrUrAmU+G*+C*mU | modification. |
| | | (SEQ ID NO: 213) | |
| SEQ ID NO: | LAG3s9_c3_12 | mG*mA*mArGrGrCrUrG/iSpC3/rGrArUrCrCrUrGrGrA | crRNA with a single C3 spacer |
| 214 | | rGrGrGrUrUrUrUrArGrArGrCrUrAmU+G*+C*mU | modification. |
| | | (SEQ ID NO: 214) | |
| SEQ ID NO: | LAG3s9_c3_11 | mG*mA*mArGrGrCrUrGrA/iSpC3/rArUrCrCrUrGrGrA | crRNA with a single C3 spacer |
| 215 | | rGrGrGrUrUrUrUrArGrArGrCrUrAmU+G*+C*mU | modification. |
| | | (SEQ ID NO: 215) | |
| SEQ ID NO: | LAG3s9_c3_10 | mG*mA*mArGrGrCrUrGrArG (SEQ ID NO: | crRNA with a single C3 spacer |
| 216 | | 216)/iSpC3/rUrCrCrUrGrGrArGrGrGrUrUrUrUrArGrAr | modification. |
| | | GrCrUrAmU+G*+C*mU (SEQ ID NO: 659) | |
| SEQ ID NO: | LAG3s9_c3_9 | mG*mA*mArGrGrCrUrGrArGrA (SEQ ID NO: | crRNA with a single C3 spacer |
| 217 | | 217)/iSpC3/rCrCrUrGrGrArGrGrGrUrUrUrUrArGrArGr | modification. |
| | | CrUrAmU+G*+C*mU (SEQ ID NO: 660) | |
| SEQ ID NO: | LAG3s9_c3_8 | mG*mA*mArGrGrCrUrGrArGrArU (SEQ ID NO: | crRNA with a single C3 spacer |
| 218 | | 218)/iSpC3/rCrUrGrGrArGrGrGrUrUrUrUrArGrArGrCr | modification. |
| | | UrAmU+G*+C*mU (SEQ ID NO: 661) | |
| SEQ ID NO: | LAG3s9_c3_7 | mG*mA*mArGrGrCrUrGrArGrArUrC (SEQ ID NO: | crRNA with a single C3 spacer |
| 219 | | 219)/iSpC3/rUrGrGrArGrGrGrUrUrUrUrArGrArGrCrUr | modification. |
| | | AmU+G*+C*mU (SEQ ID NO: 662) | |
| SEQ ID NO: | LAG3s9_c3_6 | mG*mA*mArGrGrCrUrGrArGrArUrCrC (SEQ ID NO: | crRNA with a single C3 spacer |
| 220 | | 220)/iSpC3/rGrGrArGrGrGrUrUrUrUrArGrArGrCrUrA | modification. |
| | | mU+G*+C*mU (SEQ ID NO: 663) | |
| SEQ ID NO: 221 | LAG3s9_c3_5 | mG*mA*mArGrGrCrUrGrArGrArUrCrCrU (SEQ ID NO: | crRNA with a single C3 spacer |
| | | 221)/iSpC3/rGrArGrGrGrUrUrUrUrArGrArGrCrUrAmU | modification. |
| | | +G*+C*mU (SEQ ID NO: 664) | |
| SEQ ID NO: 222 | LAG3s9_c3_4 | mG*mA*mArGrGrCrUrGrArGrArUrCrCrUrG (SEQ ID | crRNA with a single C3 spacer |
| | | NO: 222)/ | modification. |
| | | iSpC3/rArGrGrGrUrUrUrUrArGrArGrCrUrAmU+ | |
| | | G*+C*mU (SEQ ID NO: 665) | |
| SEQ ID NO: 223 | LAG3s9_c3_3 | mG*mA*mArGrGrCrUrGrArGrArUrCrCrUrGrG (SEQ | crRNA with a single C3 spacer |
| | | ID NO: | modification. |
| | | 223)/iSpC3/rGrGrGrUrUrUrUrArGrArGrCrUrAmU+G* | |
| | | +C*mU (SEQ ID NO: 666) | |
| SEQ ID NO: 224 | LAG3s9_c3_2 | mG*mA*mArGrGrCrUrGrArGrArUrCrCrUrGrGrA (SEQ | crRNA with a single C3 spacer |
| | | ID NO: | modification. |
| | | 224)/iSpC3/rGrGrUrUrUrUrArGrArGrCrUrAmU+G*+C | |
| | | *mU (SEQ ID NO: 667) | |
| SEQ ID NO: 225 | LAG3s9_c3_1 | mG*mA*mArGrGrCrUrGrArGrArUrCrCrUrGrGrArG | crRNA with a single C3 spacer |
| | | (SEQ ID NO: | modification. |
| | | 225)/iSpC3/rGrUrUrUrUrArGrArGrCrUrAmU+G*+C*m | |
| | | U (SEQ ID NO: 668) | |
| SEQ ID NO: | EMX1_c3_20 | /5SpC3/*mA*mGrUrCrCrGrArGrCrArGrArArGrArArGr | crRNA with a single C3 spacer |
| 226 | | ArArGrUrUrUrUrArGrArGrCrUrAmU+G*+C*mU(SEQ | modification. |
| | | ID No: 226) | |
| SEQ ID NO: | EMX1_c3_19 | mG*/iSpC3/*mGrUrCrCrGrArGrCrArGrArArGrArArGr | crRNA with a single C3 spacer |
| 227 | | ArArGrUrUrUrUrArGrArGrCrUrAmU+G*+C*mU (SEQ | modification. |
| | | ID No: 227) | |
| SEQ ID NO: | EMX1_c3_18 | mG*mA*/iSpC3/rUrCrCrGrArGrCrArGrArArGrArArGr | crRNA with a single C3 spacer |
| 228 | | ArArGrUrUrUrUrArGrArGrCrUrAmU+G*+C*mU (SEQ | modification. |
| | | ID No: 228) | |
| SEQ ID NO: | EMX1_c3_17 | mG*mA*mG/iSpC3/rCrCrGrArGrCrArGrArArGrArArG | crRNA with a single C3 spacer |
| 229 | | ArArGrUrUrUrUrArGrArGrCrUrAmU+G*+C*mU | modification. |
| | | (SEQ ID No: 229) | |
| SEQ ID NO: | EMX1_c3_16 | mG*mA*mGrU/iSpC3/rCrGrArGrCrArGrArArGrArArG | crRNA with a single C3 spacer |
| 230 | | rArArGrUrUrUrUrArGrArGrCrUrAmU+G*+C*mU | modification. |
| | | (SEQ ID No: 230) | |
| SEQ ID NO: | EMX1_c3_15 | mG*mA*mGrUrC/iSpC3/rGrArGrCrArGrArArGrArArG | crRNA with a single C3 spacer |
| 231 | | rArArGrUrUrUrUrArGrArGrCrUrAmU+G*+C*mU | modification. |
| | | (SEQ ID No: 231) | |
| SEQ ID NO: | EMX1_c3_14 | mG*mA*mGrUrCrC/iSpC3/rArGrCrArGrArArGrArArG | crRNA with a single C3 spacer |
| 232 | | rArArGrUrUrUrUrArGrArGrCrUrAmU+G*+C*mU | modification. |
| | | (SEQ ID No: 232) | |
| SEQ ID NO: | EMX1_c3_13 | mG*mA*mGrUrCrCrG/iSpC3/rGrCrArGrArArGrArArG | crRNA with a single C3 spacer |
| 233 | | rArArGrUrUrUrUrArGrArGrCrUrAmU+G*+C*mU | modification. |
| | | (SEQ ID No: 233) | |
| SEQ ID NO: | EMX1_c3_12 | mG*mA*mGrUrCrCrGrA/iSpC3/rCrArGrArArGrArArG | crRNA with a single C3 spacer |
| 234 | | rArArGrUrUrUrUrArGrArGrCrUrAmU+G*+C*mU | modification. |
| | | (SEQ ID No: 234) | |
| SEQ ID NO: | EMX1_c3_11 | mG*mA*mGrUrCrCrGrArG/iSpC3/rArGrArArGrArArG | crRNA with a single C3 spacer |
| 235 | | rArArGrUrUrUrUrArGrArGrCrUrAmU+G*+C*mU | modification. |
| | | (SEQ ID No: 235) | |
| SEQ ID NO: | EMX1_c3_10 | mG*mA*mGrUrCrCrGrArGrC (SEQ ID NO: | crRNA with a single C3 spacer |
| 236 | | 236)/iSpC3/rGrArArGrArArGrArArGrUrUrUrUrArGrAr | modification. |
| | | GrCrUrAmU+G*+C*mU (SEQ ID NO: 669) | |
| SEQ ID NO: | EMX1_c3_9 | mG*mA*mGrUrCrCrGrArGrCrA (SEQ ID NO: | crRNA with a single C3 spacer |
| 237 | | 237)/iSpC3/rArArGrArArGrArArGrUrUrUrUrArGrArGr | modification. |
| | | CrUrAmU+G*+C*mU (SEQ ID NO: 670) | |
| SEQ ID NO: | EMX1_c3_8 | mG*mA*mGrUrCrCrGrArGrCrArG (SEQ ID NO: | crRNA with a single C3 spacer |
| 238 | | 238)/iSpC3/rArGrArArGrArArGrUrUrUrUrArGrArGrCr | modification. |
| | | UrAmU+G*+C*mU (SEQ ID NO: 671) | |
| SEQ ID NO: | EMX1_c3_7 | mG*mA*mGrUrCrCrGrArGrCrArGrA (SEQ ID NO: | crRNA with a single C3 spacer |
| 239 | | 239)/iSpC3/rGrArArGrArArGrUrUrUrUrArGrArGrCrUr | modification. |
| | | AmU+G*+C*mU (SEQ ID NO:672) | |
| SEQ ID NO: | EMX1_c3_6 | mG*mA*mGrUrCrCrGrArGrCrArGrArA (SEQ ID NO: | crRNA with a single C3 spacer |
| 240 | | 240)/iSpC3/rArArGrArArGrUrUrUrUrArGrArGrCrUrA | modification. |
| | | mU+G*+C*mU (SEQ ID NO: 673) | |
| SEQ ID NO: | EMX1_c3_5 | mG*mA*mGrUrCrCrGrArGrCrArGrArArG (SEQ ID NO: | crRNA with a single C3 spacer |
| 241 | | 241)/iSpC3/rArGrArArGrUrUrUrUrArGrArGrCrUrAmU | modification. |
| | | +G*+C*mU (SEQ ID NO: 674) | |
| SEQ ID NO: | EMX1_c3_4 | mG*mA*mGrUrCrCrGrArGrCrArGrArArGrA (SEQ ID | crRNA with a single C3 spacer |
| 242 | | NO: | modification. |
| | | 242)/iSpC3/rGrArArGrUrUrUrUrArGrArGrCrUrAmU+ | |
| | | G*+C*mU (SEQ ID NO: 675) | |
| SEQ ID NO: 243 | EMX1_c3_3 | mG*mA*mGrUrCrCrGrArGrCrArGrArArGrArA (SEQ | crRNA with a single C3 spacer |
| | | ID NO: | modification. |
| | | 243)/iSpC3/rArArGrUrUrUrUrArGrArGrCrUrAmU+G* | |
| | | +C*mU (SEQ ID NO: 676) | |
| SEQ ID NO: 244 | EMX1_c3_2 | mG*mA*mGrUrCrCrGrArGrCrArGrArArGrArArG (SEQ | crRNA with a single C3 spacer |
| | | ID NO: | modification. |
| | | 244)/iSpC3/rArGrUrUrUrUrArGrArGrCrUrAmU+G*+C | |
| | | *mU (SEQ ID NO: 677) | |
| SEQ ID NO: 245 | EMX1_c3_1 | mG*mA*mGrUrCrCrGrArGrCrArGrArArGrArArGrA | crRNA with a single C3 spacer |
| | | (SEQ ID NO: | modification. |
| | | 245)/iSpC3/rGrUrUrUrUrArGrArGrCrUrAmU+G*+C*m | |
| | | U (SEQ ID NO: 658) | |
| SEQ ID NO: 246 | AR_dSpacer_20 | /5dSp/*mU*mUrGrGrArGrCrArUrCrUrGrArGrUrCrCrA | crRNA with a single dSpacer |
| | | rGrGrUrUrUrUrArGrArGrCrUrAmU+G*+C*mU | modification. |
| SEQ ID NO: 247 | AR_dSpacer_19 | mG*/idSp/*mUrGrGrArGrCrArUrCrUrGrArGrUrCrCrA | crRNA with a single dSpacer |
| | | rGrGrUrUrUrUrArGrArGrCrUrAmU+G*+C*mU | modification. |
| SEQ ID NO: 248 | AR_dSpacer_18_00 | mG*mU*/idSp/rGrGrArGrCrArUrCrUrGrArGrUrCrCrA | crRNA with a single dSpacer |
| | | rGrGrUrUrUrUrArGrArGrCrUrAmU+G*+C*mU | modification. |
| SEQ ID NO: 249 | AR_dSpacer_17 | mG*mU*mU/idSp/rGrArGrCrArUrCrUrGrArGrUrCrCr | crRNA with a single dSpacer |
| | | ArGrGrUrUrUrUrArGrArGrCrUrAmU+G*+C*mU | modification. |
| SEQ ID NO: 250 | AR_dSpacer_16 | mG*mU*mUrG/idSp/rArGrCrArUrCrUrGrArGrUrCrCr | crRNA with a single dSpacer |
| | | ArGrGrUrUrUrUrArGrArGrCrUrAmU+G*+C*mU | modification. |
| SEQ ID NO: 251 | AR_dSpacer_15 | mG*mU*mUrGrG/idSp/rGrCrArUrCrUrGrArGrUrCrCr | crRNA with a single dSpacer |
| | | ArGrGrUrUrUrUrArGrArGrCrUrAmU+G*+C*mU | modification. |
| SEQ ID NO: 252 | AR_dSpacer_14 | mG*mU*mUrGrGrA/idSp/rCrArUrCrUrGrArGrUrCrCr | crRNA with a single dSpacer |
| | | ArGrGrUrUrUrUrArGrArGrCrUrAmU+G*+C*mU | modification. |
| SEQ ID NO: 253 | AR_dSpacer_13 | mG*mU*mUrGrGrArG/idSp/rArUrCrUrGrArGrUrCrCr | crRNA with a single dSpacer |
| | | ArGrGrUrUrUrUrArGrArGrCrUrAmU+G*+C*mU | modification. |
| SEQ ID NO: 254 | AR_dSpacer_12 | mG*mU*mUrGrGrArGrC/idSp/rUrCrUrGrArGrUrCrCr | crRNA with a single dSpacer |
| | | ArGrGrUrUrUrUrArGrArGrCrUrAmU+G*+C*mU | modification. |
| SEQ ID NO: 255 | AR_dSpacer_11 | mG*mU*mUrGrGrArGrCrA/idSp/rCrUrGrArGrUrCrCr | crRNA with a single dSpacer |
| | | ArGrGrUrUrUrUrArGrArGrCrUrAmU+G*+C*mU | modification. |
| SEQ ID NO: 256 | AR_dSpacer_10 | mG*mU*mUrGrGrArGrCrArU/idSp/rUrGrArGrUrCrCr | crRNA with a single dSpacer |
| | | ArGrGrUrUrUrUrArGrArGrCrUrAmU+G*+C*mU | modification. |
| SEQ ID NO: 257 | AR_dSpacer_9 | mG*mU*mUrGrGrArGrCrArUrC/idSp/rGrArGrUrCrCr | crRNA with a single dSpacer |
| | | ArGrGrUrUrUrUrArGrArGrCrUrAmU+G*+C*mU | modification. |
| SEQ ID NO: 258 | AR_dSpacer_8 | mG*mU*mUrGrGrArGrCrArUrCrU/idSp/rArGrUrCrCr | crRNA with a single dSpacer |
| | | ArGrGrUrUrUrUrArGrArGrCrUrAmU+G*+C*mU | modification. |
| SEQ ID NO: 259 | AR_dSpacer_7 | mG*mU*mUrGrGrArGrCrArUrCrUrG/idSp/rGrUrCrCr | crRNA with a single dSpacer |
| | | ArGrGrUrUrUrUrArGrArGrCrUrAmU+G*+C*mU | modification. |
| SEQ ID NO: 260 | AR_dSpacer_6 | mG*mU*mUrGrGrArGrCrArUrCrUrGrA/idSp/rUrCrCr | crRNA with a single dSpacer |
| | | ArGrGrUrUrUrUrArGrArGrCrUrAmU+G*+C*mU | modification. |
| SEQ ID NO: 261 | AR_dSpacer_5 | mG*mU*mUrGrGrArGrCrArUrCrUrGrArG/idSp/rCrCr | crRNA with a single dSpacer |
| | | ArGrGrUrUrUrUrArGrArGrCrUrAmU+G*+C*mU | modification. |
| SEQ ID NO: 262 | AR_dSpacer_4 | mG*mU*mUrGrGrArGrCrArUrCrUrGrArGrU/idSp/rCr | crRNA with a single dSpacer |
| | | ArGrGrUrUrUrUrArGrArGrCrUrAmU+G*+C*mU | modification. |
| SEQ ID NO: 263 | AR_dSpacer_3 | mG*mU*mUrGrGrArGrCrArUrCrUrGrArGrUrC/idSp/r | crRNA with a single dSpacer |
| | | ArGrGrUrUrUrUrArGrArGrCrUrAmU+G*+C*mU | modification. |
| SEQ ID NO: 264 | AR_dSpacer_2 | mG*mU*mUrGrGrArGrCrArUrCrUrGrArGrUrCrC/idSp | crRNA with a single dSpacer |
| | | /rGrGrUrUrUrUrArGrArGrCrUrAmU+G*+C*mU | modification. |
| SEQ ID NO: 265 | AR_dSpacer_1 | mG*mU*mUrGrGrArGrCrArUrCrUrGrArGrUrCrCrA/ | crRNA with a single dSpacer |
| | | idSp/rGrUrUrUrUrArGrArGrCrUrAmU+G*+C*mU | modification. |
| SEQ ID NO: 266 | LAG3s9_dSpacer_20 | /5dSp/*mA*mArGrGrCrUrGrArGrArUrCrCrUrGrGrArG | crRNA with a single dSpacer |
| | | rGrGrUrUrUrUrArGrArGrCrUrAmU+G*+C*mU | modification. |
| SEQ ID NO: 267 | LAG3s9_dSpacer_19 | mG*/idSp/*mAmGrGrCrUrGrArGrArUrCrCrUrGrGrAr | crRNA with a single dSpacer |
| | | GrGrGrUrUrUrUrArGrArGrCrUrAmU+G*+C*mU | modification. |
| SEQ ID NO: 268 | LAG3s9_dSpacer_18 | mG*mA*/idSp/rGrGrCrUrGrArGrArUrCrCrUrGrGrArG | crRNA with a single dSpacer |
| | | rGrGrUrUrUrUrArGrArGrCrUrAmU+G*+C*mU | modification. |
| SEQ ID NO: 269 | LAG3s9_dSpacer_17 | mG*mA*mA/idSp/rGrCrUrGrArGrArUrCrCrUrGrGrAr | crRNA with a single dSpacer |
| | | GrGrGrUrUrUrUrArGrArGrCrUrAmU+G*+C*mU | modification. |
| SEQ ID NO: 270 | LAG3s9_dSpacer_16 | mG*mA*mArG/idSp/rCrUrGrArGrArUrCrCrUrGrGrAr | crRNA with a single dSpacer |
| | | GrGrGrUrUrUrUrArGrArGrCrUrAmU+G*+C*mU | modification. |
| SEQ ID NO: 271 | LAG3s9_dSpacer_15 | mG*mA*mArGrG/idSp/rUrGrArGrArUrCrCrUrGrGrAr | crRNA with a single dSpacer |
| | | GrGrGrUrUrUrUrArGrArGrCrUrAmU+G*+C*mU | modification. |
| SEQ ID NO: 272 | LAG3s9_dSpacer_14 | mG*mA*mArGrGrC/idSp/rGrArGrArUrCrCrUrGrGrAr | crRNA with a single dSpacer |
| | | GrGrGrUrUrUrUrArGrArGrCrUrAmU+G*+C*mU | modification. |
| SEQ ID NO: 273 | LAG3s9_dSpacer_13 | mG*mA*mArGrGrCrU/idSp/rArGrArUrCrCrUrGrGrAr | crRNA with a single dSpacer |
| | | GrGrGrUrUrUrUrArGrArGrCrUrAmU+G*+C*mU | modification. |
| SEQ ID NO: 274 | LAG3s9_dSpacer_12 | mG*mA*mArGrGrCrUrG/idSp/rGrArUrCrCrUrGrGrAr | crRNA with a single dSpacer |
| | | GrGrGrUrUrUrUrArGrArGrCrUrAmU+G*+C*mU | modification. |
| SEQ ID NO: 275 | LAG3s9_dSpacer_11 | mG*mA*mArGrGrCrUrGrA/idSp/rArUrCrCrUrGrGrAr | crRNA with a single dSpacer |
| | | GrGrGrUrUrUrUrArGrArGrCrUrAmU+G*+C*mU | modification. |
| SEQ ID NO: 276 | LAG3s9_dSpacer_10 | mG*mA*mArGrGrCrUrGrArG/idSp/rUrCrCrUrGrGrAr | crRNA with a single dSpacer |
| | | GrGrGrUrUrUrUrArGrArGrCrUrAmU+G*+C*mU | modification. |
| SEQ ID NO: 277 | LAG3s9_dSpacer_9 | mG*mA*mArGrGrCrUrGrArGrA/idSp/rCrCrUrGrGrAr | crRNA with a single dSpacer |
| | | GrGrGrUrUrUrUrArGrArGrCrUrAmU+G*+C*mU | modification. |
| SEQ ID NO: 278 | LAG3s9_dSpacer_8 | mG*mA*mArGrGrCrUrGrArGrArU/idSp/rCrUrGrGrAr | crRNA with a single dSpacer |
| | | GrGrGrUrUrUrUrArGrArGrCrUrAmU+G*+C*mU | modification. |
| SEQ ID NO: 279 | LAG3s9_dSpacer_7 | mG*mA*mArGrGrCrUrGrArGrArUrC/idSp/rUrGrGrAr | crRNA with a single dSpacer |
| | | GrGrGrUrUrUrUrArGrArGrCrUrAmU+G*+C*mU | modification. |
| SEQ ID NO: 280 | LAG3s9_dSpacer_6 | mG*mA*mArGrGrCrUrGrArGrArUrCrC/idSp/rGrGrAr | crRNA with a single dSpacer |
| | | GrGrGrUrUrUrUrArGrArGrCrUrAmU+G*+C*mU | modification. |
| SEQ ID NO: 281 | LAG3s9_dSpacer_5 | mG*mA*mArGrGrCrUrGrArGrArUrCrCrU/idSp/rGrAr | crRNA with a single dSpacer |
| | | GrGrGrUrUrUrUrArGrArGrCrUrAmU+G*+C*mU | modification. |
| SEQ ID NO: 282 | LAG3s9_dSpacer_4 | mG*mA*mArGrGrCrUrGrArGrArUrCrCrUrG/idSp/rAr | crRNA with a single dSpacer |
| | | GrGrGrUrUrUrUrArGrArGrCrUrAmU+G*+C*mU | modification. |
| SEQ ID NO: 283 | LAG3s9_dSpacer_3 | mG*mA*mArGrGrCrUrGrArGrArUrCrCrUrGrG/idSp/r | crRNA with a single dSpacer |
| | | GrGrGrUrUrUrUrArGrArGrCrUrAmU+G*+C*mU | modification. |
| SEQ ID NO: 284 | LAG3s9_dSpacer_2 | mG*mA*mArGrGrCrUrGrArGrArUrCrCrUrGrGrA/idSp | crRNA with a single dSpacer |
| | | /rGrGrUrUrUrUrArGrArGrCrUrAmU+G*+C*mU | modification. |
| SEQ ID NO: 285 | LAG3s9_dSpacer_1 | mG*mA*mArGrGrCrUrGrArGrArUrCrCrUrGrGrArG/ | crRNA with a single dSpacer |
| | | idSp/rGrUrUrUrUrArGrArGrCrUrAmU+G*+C*mU | modification. |
| SEQ ID NO: 286 | EMX1_dSpacer_20 | /5dSp/*mA*mGrUrCrCrGrArGrCrArGrArArGrArArGrA | crRNA with a single dSpacer |
| | | rArGrUrUrUrUrArGrArGrCrUrAmU+G*+C*mU | modification. |
| SEQ ID NO: 287 | EMX1_dSpacer_19 | mG*/idSp/*mGrUrCrCrGrArGrCrArGrArArGrArArGrA | crRNA with a single dSpacer |
| | | rArGrUrUrUrUrArGrArGrCrUrAmU+G*+C*mU | modification. |
| SEQ ID NO: 288 | EMX1_dSpacer_18 | mG*mA*/idSp/rUrCrCrGrArGrCrArGrArArGrArArGrA | crRNA with a single dSpacer |
| | | rArGrUrUrUrUrArGrArGrCrUrAmU+G*+C*mU | modification. |
| SEQ ID NO: 289 | EMX1_dSpacer_17 | mG*mA*mG/idSp/rCrCrGrArGrCrArGrArArGrArArGr | crRNA with a single dSpacer |
| | | ArArGrUrUrUrUrArGrArGrCrUrAmU+G*+C*mU | modification. |
| SEQ ID NO: 290 | EMX1_dSpacer_16 | mG*mA*mGrU/idSp/rCrGrArGrCrArGrArArGrArArGr | crRNA with a single dSpacer |
| | | ArArGrUrUrUrUrArGrArGrCrUrAmU+G*+C*mU | modification. |
| SEQ ID NO: 291 | EMX1_dSpacer_15 | mG*mA*mGrUrC/idSp/rGrArGrCrArGrArArGrArArGr | crRNA with a single dSpacer |
| | | ArArGrUrUrUrUrArGrArGrCrUrAmU+G*+C*mU | modification. |
| SEQ ID NO: 292 | EMX1_dSpacer_14 | mG*mA*mGrUrCrC/idSp/rArGrCrArGrArArGrArArGr | crRNA with a single dSpacer |
| | | ArArGrUrUrUrUrArGrArGrCrUrAmU+G*+C*mU | modification. |
| SEQ ID NO: 293 | EMX1_dSpacer_13 | mG*mA*mGrUrCrCrG/idSp/rGrCrArGrArArGrArArGr | crRNA with a single dSpacer |
| | | ArArGrUrUrUrUrArGrArGrCrUrAmU+G*+C*mU | modification. |
| SEQ ID NO: 294 | EMX1_dSpacer_12 | mG*mA*mGrUrCrCrGrA/idSp/rCrArGrArArGrArArGr | crRNA with a single dSpacer |
| | | ArArGrUrUrUrUrArGrArGrCrUrAmU+G*+C*mU | modification. |
| SEQ ID NO: 295 | EMX1_dSpacer_11 | mG*mA*mGrUrCrCrGrArG/idSp/rArGrArArGrArArGr | crRNA with a single dSpacer |
| | | ArArGrUrUrUrUrArGrArGrCrUrAmU+G*+C*mU | modification. |
| SEQ ID NO: 296 | EMX1_dSpacer_10 | mG*mA*mGrUrCrCrGrArGrC/idSp/rGrArArGrArArGr | crRNA with a single dSpacer |
| | | ArArGrUrUrUrUrArGrArGrCrUrAmU+G*+C*mU | modification. |
| SEQ ID NO: 297 | EMX1_dSpacer_9 | mG*mA*mGrUrCrCrGrArGrCrA/idSp/rArArGrArArGr | crRNA with a single dSpacer |
| | | ArArGrUrUrUrUrArGrArGrCrUrAmU+G*+C*mU | modification. |
| SEQ ID NO: 298 | EMX1_dSpacer_8 | mG*mA*mGrUrCrCrGrArGrCrArG/idSp/rArGrArArGr | crRNA with a single dSpacer |
| | | ArArGrUrUrUrUrArGrArGrCrUrAmU+G*+C*mU | modification. |
| SEQ ID NO: 299 | EMX1_dSpacer_7 | mG*mA*mGrUrCrCrGrArGrCrArGrA/idSp/rGrArArGr | crRNA with a single dSpacer |
| | | ArArGrUrUrUrUrArGrArGrCrUrAmU+G*+C*mU | modification. |
| SEQ ID NO: 300 | EMX1_dSpacer_6 | mG*mA*mGrUrCrCrGrArGrCrArGrArA/idSp/rArArGr | crRNA with a single dSpacer |
| | | ArArGrUrUrUrUrArGrArGrCrUrAmU+G*+C*mU | modification. |
| SEQ ID NO: 301 | EMX1_dSpacer_5 | mG*mA*mGrUrCrCrGrArGrCrArGrArArG/idSp/rArGr | crRNA with a single dSpacer |
| | | ArArGrUrUrUrUrArGrArGrCrUrAmU+G*+C*mU | modification. |
| SEQ ID NO: 302 | EMX1_dSpacer_4 | mG*mA*mGrUrCrCrGrArGrCrArGrArArGrA/idSp/rGr | crRNA with a single dSpacer |
| | | ArArGrUrUrUrUrArGrArGrCrUrAmU+G*+C*mU | modification. |
| SEQ ID NO: 303 | EMX1_dSpacer_3 | mG*mA*mGrUrCrCrGrArGrCrArGrArArGrArA/idSp/r | crRNA with a single dSpacer |
| | | ArArGrUrUrUrUrArGrArGrCrUrAmU+G*+C*mU | modification. |
| SEQ ID NO: 304 | EMX1_dSpacer_2 | mG*mA*mGrUrCrCrGrArGrCrArGrArArGrArArG/idSp | crRNA with a single dSpacer |
| | | /rArGrUrUrUrUrArGrArGrCrUrAmU+G*+C*mU | modification. |
| SEQ ID NO: 305 | EMX1_dSpacer_1 | mG*mA*mGrUrCrCrGrArGrCrArGrArArGrArArGrA/ | crRNA with a single dSpacer |
| | | idSp/rGrUrUrUrUrArGrArGrCrUrAmU+G*+C*mU | modification. |
| SEQ ID NO: 306 | EMX1 crRNA | /AltR1/rGrArGrUrCrCrGrArGrCrArGrArArGrArArGrAr | crRNA with IDT AltR modifications |
| | | ArGrUrUrUrUrArGrArGrCrUrArUrGrCrU/AltR2/ | |
| SEQ ID NO: 307 | AAVS1 crRNA | /AltR1/rGrGrGrGrCrCrArCrUrArGrGrGrArCrArGrGrAr | crRNA with IDT AltR modifications |
| | | UrGrUrUrUrUrArGrArGrCrUrArUrGrCrU/AltR2/ | |
| SEQ ID NO: 308 | AAVS1_UNAaltR_18 | /AltR1/rGrG/iUNA- | crRNA with a single UNA modification. |
| | | rG/rGrCrCrArCrUrArGrGrGrArCrArGrGrArUrGrUrUrU | |
| | | rUrArGrArGrCrUrArUrGrCrU/AltR2/ | |

TABLE 7
Top Modification Placement

| | Top Placement of single |
|---|---|
| Modification | modifications within gRNA Spacer |
| LNA | 9 |
| 2′Fluoro | 6 |
| C3 spacer | 11-18 |
| dSpacer | 11-18 |

TABLE 8
gRNA region sequences

| Target | | |
|---|---|---|
| Site | Sequence (5′-3) | SEQ_ID |
| PDCD1s8 | GAGCAGGGCTGGGGAGAAGG | SEQ ID NO: |
| | | 352 |
| LAG3 | GAAGGCTGAGATCCTGGAGG | SEQ ID |
| | | No: 353 |
| FANCF | GCTGCAGAAGGGATTCCATG | SEQ ID NO: |
| tgt 13 | | 354 |
| TRAC | TGTGCTAGACATGAGGTCTA | SEQ ID NO: |
| | | 355 |
| EMX1 | GAGTCCGAGCAGAAGAAGAA | SEQ ID NO: |
| | | 356 |
| HBB | CTTGCCCCACAGGGCAGTAA | SEQ ID NO: |
| | | 357 |
| AR | GTTGGAGCATCTGAGTCCAG | SEQ ID NO: |
| | | 358 |
| HEK | GGCCCAGACTGAGCACGTGA | SEQ ID NO: |
| Site 3 | | 359 |
| HPRT | AATTATGGGGATTACTAGGA | SEQ ID NO: |
| 38087 | | 360 |
| PD1 | GGCGCCCTGGCCAGTCGTCT | SEQ ID NO: |
| | | 361 |
| B2M | CTTACCCCACTTAACTATCT | SEQ ID NO: |
| | | 362 |
| PCSK9 | CCCGCACCTTGGCGCAGCGG | SEQ ID NO: |
| | | 363 |
| APOBEC3A | CGGTCAAGATGGACCAGCAC | SEQ ID NO: |
| | | 364 |
| APBB2 | TTGGGACAACGTTGTCCAGC | SEQ ID NO: |
| | | 365 |
| AD1 | CTACGAGGAGCATTTGCACT | SEQ ID NO: |
| | | 366 |
| APP1 | GCGGAATTGACAAGTTCCGA | SEQ ID NO: |
| | | 367 |

TABLE 9
Optimal placements for UNA modifications
within the gRNA spacer region

| | UNA Position* | |
|---|---|---|
| Tier | in Spacer | Description |
| 1 | 20, 19, 18, | Highest probability of either |
| | 17, 14, 12, 10 | increasing on-target editing |
| | | and/or retaining on-target |
| | | editing efficiency and |
| | | reducing off-target editing |
| 2 | 16, 15, 13, | High target variability of |
| | 11, 9, 8, 7, 4 | retention of on-target editing |
| | | efficiency |
| 3 | 6, 5, 3, 2, 1 | Disrupts editing efficiency |

*UNA positions are numbered from the 5′ to the 3′ end of the spacer sequence, wherein Position 20 being the first nucleotide at the 5′ end and Position 1 being the last nucleotide at the 3′end.

TABLE 10
Oligos

| Sequence ID | Name | Sequence | Description |
|---|---|---|---|
| SEQ ID NO: 368 | PCSK9s1 | /AltR1/rCrCrCrGrCrArCrCrUrUrGrGrCrGrCrArGrCrGrGrGr | crRNA |
| | | UrUrUrUrArGrArGrCrUrArUrGrCrU/AltR2/ | |
| SEQ ID NO: 369 | PCSK9_UNA20 | /AltR1//iUNA- | crRNA with single UNA base |
| | | rC/rCrCrGrCrArCrCrUrUrGrGrCrGrCrArGrCrGrGrGrUrUrU | |
| | | rUrArGrArGrCrUrArUrGrCrU/AltR2/ | |
| SEQ ID NO: 370 | PCSK9_UNA19 | /AltR1/rC/iUNA- | crRNA with single UNA base |
| | | rC/rCrGrCrArCrCrUrUrGrGrCrGrCrArGrCrGrGrGrUrUrUrU | |
| | | rArGrArGrCrUrArUrGrCrU/AltR2/ | |
| SEQ ID NO: 371 | PCSK9_UNA18 | /AltR1/rCrC/iUNA- | crRNA with single UNA base |
| | | rC/rGrCrArCrCrUrUrGrGrCrGrCrArGrCrGrGrGrUrUrUrUrA | |
| | | rGrArGrCrUrArUrGrCrU/AltR2/ | |
| SEQ ID NO: 372 | PCSK9_UNA17 | /AltR1/rCrCrC/iUNA- | crRNA with single UNA base |
| | | rG/rCrArCrCrUrUrGrGrCrGrCrArGrCrGrGrGrUrUrUrUrAr | |
| | | GrArGrCrUrArUrGrCrU/AltR2/ | |
| SEQ ID NO: 373 | PCSK9_UNA16 | /AltR1/rCrCrCrG/iUNA- | crRNA with single UNA base |
| | | rC/rArCrCrUrUrGrGrCrGrCrArGrCrGrGrGrUrUrUrUrArGr | |
| | | ArGrCrUrArUrGrCrU/AltR2/ | |
| SEQ ID NO: 374 | PCSK9_UNA15 | /AltR1/rCrCrCrGrC/iUNA- | crRNA with single UNA base |
| | | rA/rCrCrUrUrGrGrCrGrCrArGrCrGrGrGrUrUrUrUrArGrAr | |
| | | GrCrUrArUrGrCrU/AltR2/ | |
| SEQ ID NO: 375 | PCSK9_UNA14 | /AltR1/rCrCrCrGrCrA/iUNA- | crRNA with single UNA base |
| | | rC/rCrUrUrGrGrCrGrCrArGrCrGrGrGrUrUrUrUrArGrArGr | |
| | | CrUrArUrGrCrU/AltR2/ | |
| SEQ ID NO: 376 | PCSK9_UNA13 | /AltR1/rCrCrCrGrCrArC/iUNA- | crRNA with single UNA base |
| | | rC/rUrUrGrGrCrGrCrArGrCrGrGrGrUrUrUrUrArGrArGrCr | |
| | | UrArUrGrCrU/AltR2/ | |
| SEQ ID NO: 377 | PCSK9_UNA12 | /AltR1/rCrCrCrGrCrArCrC/iUNA- | crRNA with single UNA base |
| | | rU/rUrGrGrCrGrCrArGrCrGrGrGrUrUrUrUrArGrArGrCrUr | |
| | | ArUrGrCrU/AltR2/ | |
| SEQ ID NO: 378 | PCSK9_UNA11 | /AltR1/rCrCrCrGrCrArCrCrU/iUNA- | crRNA with single UNA base |
| | | rU/rGrGrCrGrCrArGrCrGrGrGrUrUrUrUrArGrArGrCrUrAr | |
| | | UrGrCrU/AltR2/ | |
| SEQ ID NO: 379 | PCSK9_UNA10 | /AltR1/rCrCrCrGrCrArCrCrUrU/iUNA- | crRNA with single UNA base |
| | | rG/rGrCrGrCrArGrCrGrGrGrUrUrUrUrArGrArGrCrUrArUr | |
| | | GrCrU/AltR2/ | |
| SEQ ID NO: 380 | PCSK9_UNA9 | /AltR1/rCrCrCrGrCrArCrCrUrUrG/iUNA- | crRNA with single UNA base |
| | | rG/rCrGrCrArGrCrGrGrGrUrUrUrUrArGrArGrCrUrArUrGr | |
| | | CrU/AltR2/ | |
| SEQ ID NO: 381 | PCSK9_UNA8 | /AltR1/rCrCrCrGrCrArCrCrUrUrGrG/iUNA- | crRNA with single UNA base |
| | | rC/rGrCrArGrCrGrGrGrUrUrUrUrArGrArGrCrUrArUrGrCr | |
| | | U/AltR2/ | |
| SEQ ID NO: 382 | PCSK9_UNA7 | /AltR1/rCrCrCrGrCrArCrCrUrUrGrGrC/iUNA- | crRNA with single UNA base |
| | | rG/rCrArGrCrGrGrGrUrUrUrUrArGrArGrCrUrArUrGrCrU/ | |
| | | AltR2/ | |
| SEQ ID NO: 383 | PCSK9_UNA6 | /AltR1/rCrCrCrGrCrArCrCrUrUrGrGrCrG/iUNA- | crRNA with single UNA base |
| | | rC/rArGrCrGrGrGrUrUrUrUrArGrArGrCrUrArUrGrCrU/Alt | |
| | | R2/ | |
| SEQ ID NO: 384 | PCSK9_UNA5 | /AltR1/rCrCrCrGrCrArCrCrUrUrGrGrCrGrC/iUNA- | crRNA with single UNA base |
| | | rA/rGrCrGrGrGrUrUrUrUrArGrArGrCrUrArUrGrCrU/AltR2 | |
| | | / | |
| SEQ ID NO: 385 | PCSK9_UNA4 | /AltR1/rCrCrCrGrCrArCrCrUrUrGrGrCrGrCrA/iUNA- | crRNA with single UNA base |
| | | rG/rCrGrGrGrUrUrUrUrArGrArGrCrUrArUrGrCrU/AltR2/ | |
| SEQ ID NO: 386 | PCSK9_UNA3 | /AltR1/rCrCrCrGrCrArCrCrUrUrGrGrCrGrCrArG/iUNA- | crRNA with single UNA base |
| | | rC/rGrGrGrUrUrUrUrArGrArGrCrUrArUrGrCrU/AltR2/ | |
| SEQ ID NO: 387 | PCSK9_UNA2 | /AltR1/rCrCrCrGrCrArCrCrUrUrGrGrCrGrCrArGrC/iUNA- | crRNA with single UNA base |
| | | rG/rGrGrUrUrUrUrArGrArGrCrUrArUrGrCrU/AltR2/ | |
| SEQ ID NO: 388 | PCSK9_UNA1 | /AltR1/rCrCrCrGrCrArCrCrUrUrGrGrCrGrCrArGrCrG/iUN | crRNA with single UNA base |
| | | A-rG/rGrUrUrUrUrArGrArGrCrUrArUrGrCrU/AltR2/ | |
| SEQ ID NO: 389 | APP1_crRNA | /AltR1/rGrCrGrGrArArUrUrGrArCrArArGrUrUrCrCrGrArG | crRNA |
| | | rUrUrUrUrArGrArGrCrUrArUrGrCrU/AltR2/ | |
| SEQ ID NO: 390 | APP1_UNA20 | /AltR1//iUNA- | crRNA with single UNA base |
| | | rG/rCrGrGrArArUrUrGrArCrArArGrUrUrCrCrGrArGrUrUr | |
| | | UrUrArGrArGrCrUrArUrGrCrU/AltR2/ | |
| SEQ ID NO: 391 | APP1_UNA19 | /AltR1/rG/iUNA- | crRNA with single UNA base |
| | | rC/rGrGrArArUrUrGrArCrArArGrUrUrCrCrGrArGrUrUrUr | |
| | | UrArGrArGrCrUrArUrGrCrU/AltR2/ | |
| SEQ ID NO: 392 | APP1_UNA18 | /AltR1/rGrC/iUNA- | crRNA with single UNA base |
| | | rG/rGrArArUrUrGrArCrArArGrUrUrCrCrGrArGrUrUrUrUr | |
| | | ArGrArGrCrUrArUrGrCrU/AltR2/ | |
| SEQ ID NO: 393 | APP1_UNA17 | /AltR1/rGrCrG/iUNA- | crRNA with single UNA base |
| | | rG/rArArUrUrGrArCrArArGrUrUrCrCrGrArGrUrUrUrUrAr | |
| | | GrArGrCrUrArUrGrCrU/AltR2/ | |
| SEQ ID NO: 394 | APP1_UNA16 | /AltR1/rGrCrGrG/iUNA- | crRNA with single UNA base |
| | | rA/rArUrUrGrArCrArArGrUrUrCrCrGrArGrUrUrUrUrArGr | |
| | | ArGrCrUrArUrGrCrU/AltR2/ | |
| SEQ ID NO: 395 | APP1_UNA15 | /AltR1/rGrCrGrGrA/iUNA- | crRNA with single UNA base |
| | | rA/rUrUrGrArCrArArGrUrUrCrCrGrArGrUrUrUrUrArGrAr | |
| | | GrCrUrArUrGrCrU/AltR2/ | |
| SEQ ID NO: 396 | APP1_UNA14 | /AltR1/rGrCrGrGrArA/iUNA- | crRNA with single UNA base |
| | | rU/rUrGrArCrArArGrUrUrCrCrGrArGrUrUrUrUrArGrArGr | |
| | | CrUrArUrGrCrU/AltR2/ | |
| SEQ ID NO: 397 | APP1_UNA13 | /AltR1/rGrCrGrGrArArU/iUNA- | crRNA with single UNA base |
| | | rU/rGrArCrArArGrUrUrCrCrGrArGrUrUrUrUrArGrArGrCr | |
| | | UrArUrGrCrU/AltR2/ | |
| SEQ ID NO: 398 | APP1_UNA12 | /AltR1/rGrCrGrGrArArUrU/iUNA- | crRNA with single UNA base |
| | | rG/rArCrArArGrUrUrCrCrGrArGrUrUrUrUrArGrArGrCrUr | |
| | | ArUrGrCrU/AltR2/ | |
| SEQ ID NO: 399 | APP1_UNA11 | /AltR1/rGrCrGrGrArArUrUrG/iUNA- | crRNA with single UNA base |
| | | rA/rCrArArGrUrUrCrCrGrArGrUrUrUrUrArGrArGrCrUrAr | |
| | | UrGrCrU/AltR2/ | |
| SEQ ID NO: 400 | PD1_crRNA | /AltR1/rGrGrCrGrCrCrCrUrGrGrCrCrArGrUrCrGrUrCrUrGr | crRNA |
| | | UrUrUrUrArGrArGrCrUrArUrGrCrU/AltR2/ | |
| SEQ ID NO: 401 | PD1_UNA20 | /AltR1//iUNA- | crRNA with single UNA base |
| | | rG/rGrCrGrCrCrCrUrGrGrCrCrArGrUrCrGrUrCrUrGrUrUrU | |
| | | rUrArGrArGrCrUrArUrGrCrU/AltR2/ | |
| SEQ ID NO: 402 | PD1_UNA19 | /AltR1/rG/iUNA- | crRNA with single UNA base |
| | | rG/rCrGrCrCrCrUrGrGrCrCrArGrUrCrGrUrCrUrGrUrUrUrU | |
| | | rArGrArGrCrUrArUrGrCrU/AltR2/ | |
| SEQ ID NO: 403 | PD1_UNA18 | /AltR1/rGrG/iUNA- | crRNA with single UNA base |
| | | rC/rGrCrCrCrUrGrGrCrCrArGrUrCrGrUrCrUrGrUrUrUrUrA | |
| | | rGrArGrCrUrArUrGrCrU/AltR2/ | |
| SEQ ID NO: 404 | PD1_UNA17 | /AltR1/rGrGrC/iUNA- | crRNA with single UNA base |
| | | rG/rCrCrCrUrGrGrCrCrArGrUrCrGrUrCrUrGrUrUrUrUrArG | |
| | | rArGrCrUrArUrGrCrU/AltR2/ | |
| SEQ ID NO: 405 | PD1_UNA16 | /AltR1/rGrGrCrG/iUNA- | crRNA with single UNA base |
| | | rC/rCrCrUrGrGrCrCrArGrUrCrGrUrCrUrGrUrUrUrUrArGrA | |
| | | rGrCrUrArUrGrCrU/AltR2/ | |
| SEQ ID NO: 406 | PD1_UNA15 | /AltR1/rGrGrCrGrC/iUNA- | crRNA with single UNA base |
| | | rC/rCrUrGrGrCrCrArGrUrCrGrUrCrUrGrUrUrUrUrArGrAr | |
| | | GrCrUrArUrGrCrU/AltR2/ | |
| SEQ ID NO: 407 | PD1_UNA14 | /AltR1/rGrGrCrGrCrC/iUNA- | crRNA with single UNA base |
| | | rC/rUrGrGrCrCrArGrUrCrGrUrCrUrGrUrUrUrUrArGrArGr | |
| | | CrUrArUrGrCrU/AltR2/ | |
| SEQ ID NO: 408 | PD1_UNA13 | /AltR1/rGrGrCrGrCrCrC/iUNA- | crRNA with single UNA base |
| | | rU/rGrGrCrCrArGrUrCrGrUrCrUrGrUrUrUrUrArGrArGrCr | |
| | | UrArUrGrCrU/AltR2/ | |
| SEQ ID NO: 409 | PD1_UNA12 | /AltR1/rGrGrCrGrCrCrCrU/iUNA- | crRNA with single UNA base |
| | | rG/rGrCrCrArGrUrCrGrUrCrUrGrUrUrUrUrArGrArGrCrUr | |
| | | ArUrGrCrU/AltR2/ | |
| SEQ ID NO: 410 | PD1_UNA11 | /AltR1/rGrGrCrGrCrCrCrUrG/iUNA- | crRNA with single UNA base |
| | | rG/rCrCrArGrUrCrGrUrCrUrGrUrUrUrUrArGrArGrCrUrAr | |
| | | UrGrCrU/AltR2/ | |
| SEQ ID NO: 411 | TRAC_crRNA | /AltR1/rUrGrUrGrCrUrArGrArCrArUrGrArGrGrUrCrUrArG | crRNA |
| | | rUrUrUrUrArGrArGrCrUrArUrGrCrU/AltR2/ | |
| SEQ ID NO: 412 | TRAC_UNA20 | /AltR1//iUNA- | crRNA with single UNA base |
| | | rU/rGrUrGrCrUrArGrArCrArUrGrArGrGrUrCrUrArGrUrUr | |
| | | UrUrArGrArGrCrUrArUrGrCrU/AltR2/ | |
| SEQ ID NO: 413 | TRAC_UNA19 | /AltR1/rU/iUNA- | crRNA with single UNA base |
| | | rG/rUrGrCrUrArGrArCrArUrGrArGrGrUrCrUrArGrUrUrUr | |
| | | UrArGrArGrCrUrArUrGrCrU/AltR2/ | |
| SEQ ID NO: 414 | TRAC_UNA18 | /AltR1/rUrG/iUNA- | crRNA with single UNA base |
| | | rU/rGrCrUrArGrArCrArUrGrArGrGrUrCrUrArGrUrUrUrUr | |
| | | ArGrArGrCrUrArUrGrCrU/AltR2/ | |
| SEQ ID NO: 415 | TRAC_UNA17 | /AltR1/rUrGrU/iUNA- | crRNA with single UNA base |
| | | rG/rCrUrArGrArCrArUrGrArGrGrUrCrUrArGrUrUrUrUrAr | |
| | | GrArGrCrUrArUrGrCrU/AltR2/ | |
| SEQ ID NO: 416 | TRAC_UNA16 | /AltR1/rUrGrUrG/iUNA- | crRNA with single UNA base |
| | | rC/rUrArGrArCrArUrGrArGrGrUrCrUrArGrUrUrUrUrArGr | |
| | | ArGrCrUrArUrGrCrU/AltR2/ | |
| SEQ ID NO: 417 | TRAC_UNA15 | /AltR1/rUrGrUrGrC/iUNA- | crRNA with single UNA base |
| | | rU/rArGrArCrArUrGrArGrGrUrCrUrArGrUrUrUrUrArGrAr | |
| | | GrCrUrArUrGrCrU/AltR2 | |
| SEQ ID NO: 418 | TRAC_UNA14 | /AltR1/rUrGrUrGrCrU/iUNA- | crRNA with single UNA base |
| | | rA/rGrArCrArUrGrArGrGrUrCrUrArGrUrUrUrUrArGrArGr | |
| | | CrUrArUrGrCrU/AltR2/ | |
| SEQ ID NO: 419 | TRAC_UNA13 | /AltR1/rUrGrUrGrCrUrA/iUNA- | crRNA with single UNA base |
| | | rG/rArCrArUrGrArGrGrUrCrUrArGrUrUrUrUrArGrArGrCr | |
| | | UrArUrGrCrU/AltR2/ | |
| SEQ ID NO: 420 | TRAC_UNA12 | /AltR1/rUrGrUrGrCrUrArG/iUNA- | crRNA with single UNA base |
| | | rA/rCrArUrGrArGrGrUrCrUrArGrUrUrUrUrArGrArGrCrUr | |
| | | ArUrGrCrU/AltR2/ | |
| SEQ ID NO: 421 | TRAC_UNA11 | /AltR1/rUrGrUrGrCrUrArGrA/iUNA- | crRNA with single UNA base |
| | | rC/rArUrGrArGrGrUrCrUrArGrUrUrUrUrArGrArGrCrUrAr | |
| | | UrGrCrU/AltR2/ | |
| SEQ ID NO: 422 | PDCD1s8_crRNA | /AltR1/rGrArGrCrArGrGrGrCrUrGrGrGrGrArGrArArGrGrG | crRNA |
| | | rUrUrUrUrArGrArGrCrUrArUrGrCrU/AltR2/ | |
| SEQ ID NO: 423 | LAG3_crRNA | /AltR1/rGrArArGrGrCrUrGrArGrArUrCrCrUrGrGrArGrGrG | crRNA |
| | | rUrUrUrUrArGrArGrCrUrArUrGrCrU/AltR2/ | |
| SEQ ID NO: 424 | FANCF_tgt13_crRNA | /AltR1/rGrCrUrGrCrArGrArArGrGrGrArUrUrCrCrArUrGrG | crRNA |
| | | rUrUrUrUrArGrArGrCrUrArUrGrCrU/AltR2/ | |
| SEQ ID NO: 425 | EMX1_crRNA | /AltR1/rGrArGrUrCrCrGrArGrCrArGrArArGrArArGrArArG | crRNA |
| | | rUrUrUrUrArGrArGrCrUrArUrGrCrU/AltR2/ | |
| SEQ ID NO: 426 | HBB_crRNA | /AltR1/rCrUrUrGrCrCrCrCrArCrArGrGrGrCrArGrUrArArGr | crRNA |
| | | UrUrUrUrArGrArGrCrUrArUrGrCrU/AltR2/ | |
| SEQ ID NO: 427 | AR_crRNA | /AltR1/rGrUrUrGrGrArGrCrArUrCrUrGrArGrUrCrCrArGrG | crRNA |
| | | rUrUrUrUrArGrArGrCrUrArUrGrCrU/AltR2/ | |
| SEQ ID NO: 428 | HEK_Site_3_crRNA | /AltR1/rGrGrCrCrCrArGrArCrUrGrArGrCrArCrGrUrGrArGr | crRNA |
| | | UrUrUrUrArGrArGrCrUrArUrGrCrU/AltR2/ | |
| SEQ ID NO: 429 | HPRT_38087_crRNA | /AltR1/rArArUrUrArUrGrGrGrGrArUrUrArCrUrArGrGrArG | crRNA |
| | | rUrUrUrUrArGrArGrCrUrArUrGrCrU/AltR2/ | |
| SEQ ID NO: 430 | B2M_crRNA | /AltR1/rCrUrUrArCrCrCrCrArCrUrUrArArCrUrArUrCrUrGr | crRNA |
| | | UrUrUrUrArGrArGrCrUrArUrGrCrU/AltR2/ | |
| SEQ ID NO: 431 | APOBEC3A_crRNA | /AltR1/rCrGrGrUrCrArArGrArUrGrGrArCrCrArGrCrArCrGr | crRNA |
| | | UrUrUrUrArGrArGrCrUrArUrGrCrU/AltR2/ | |
| SEQ ID NO: 432 | APBB2_crRNA | /AltR1/rUrUrGrGrGrArCrArArCrGrUrUrGrUrCrCrArGrCrGr | crRNA |
| | | UrUrUrUrArGrArGrCrUrArUrGrCrU/AltR2/ | |
| SEQ ID NO: 433 | ADI_crRNA | /AltR1/rCrUrArCrGrArGrGrArGrCrArUrUrUrGrCrArCrUrGr | crRNA |
| | | UrUrUrUrArGrArGrCrUrArUrGrCrU/AltR2/ | |
| SEQ ID NO: 434 | PDCD1_8_UNA20 | /AltR1//iUNA- | crRNA with single UNA base |
| | | rG/rArGrCrArGrGrGrCrUrGrGrGrGrArGrArArGrGrGrUrUr | |
| | | UrUrArGrArGrCrUrArUrGrCrU/AltR2/ | |
| SEQ ID NO: 435 | PDCD1_8_UNA19 | /AltR1/rG/iUNA- | crRNA with single UNA base |
| | | rA/rGrCrArGrGrGrCrUrGrGrGrGrArGrArArGrGrGrUrUrUr | |
| | | UrArGrArGrCrUrArUrGrCrU/AltR2/ | |
| SEQ ID NO: 436 | PDCD1_8_UNA18 | /AltR1/rGrA/iUNA- | crRNA with single UNA base |
| | | rG/rCrArGrGrGrCrUrGrGrGrGrArGrArArGrGrGrUrUrUrUr | |
| | | ArGrArGrCrUrArUrGrCrU/AltR2/ | |
| SEQ ID NO: 437 | PDCD1_8_UNA17 | /AltR1/rGrArG/iUNA- | crRNA with single UNA base |
| | | rC/rArGrGrGrCrUrGrGrGrGrArGrArArGrGrGrUrUrUrUrAr | |
| | | GrArGrCrUrArUrGrCrU/AltR2/ | |
| SEQ ID NO: 438 | PDCD1_8_UNA16 | /AltR1/rGrArGrC/iUNA- | crRNA with single UNA base |
| | | rA/rGrGrGrCrUrGrGrGrGrArGrArArGrGrGrUrUrUrUrArGr | |
| | | ArGrCrUrArUrGrCrU/AltR2/ | |
| SEQ ID NO: 439 | PDCD1_8_UNA15 | /AltR1/rGrArGrCrA/iUNA- | crRNA with single UNA base |
| | | rG/rGrGrCrUrGrGrGrGrArGrArArGrGrGrUrUrUrUrArGrAr | |
| | | GrCrUrArUrGrCrU/AltR2/ | |
| SEQ ID NO: 440 | PDCD1_8_UNA14 | /AltR1/rGrArGrCrArG/iUNA- | crRNA with single UNA base |
| | | rG/rGrCrUrGrGrGrGrArGrArArGrGrGrUrUrUrUrArGrArGr | |
| | | CrUrArUrGrCrU/AltR2/ | |
| SEQ ID NO: 441 | PDCD1_8_UNA13 | /AltR1/rGrArGrCrArGrG/iUNA- | crRNA with single UNA base |
| | | rG/rCrUrGrGrGrGrArGrArArGrGrGrUrUrUrUrArGrArGrCr | |
| | | UrArUrGrCrU/AltR2/ | |
| SEQ ID NO: 442 | PDCD1_8_UNA12 | /AltR1/rGrArGrCrArGrGrG/iUNA- | crRNA with single UNA base |
| | | rC/rUrGrGrGrGrArGrArArGrGrGrUrUrUrUrArGrArGrCrUr | |
| | | ArUrGrCrU/AltR2/ | |
| SEQ ID NO: 443 | PDCD1_8_UNA11 | /AltR1/rGrArGrCrArGrGrGrC/iUNA- | crRNA with single UNA base |
| | | rU/rGrGrGrGrArGrArArGrGrGrUrUrUrUrArGrArGrCrUrAr | |
| | | UrGrCrU/AltR2/ | |
| SEQ ID NO: 444 | PDCD1_8_UNA10 | /AltR1/rGrArGrCrArGrGrGrCrU/iUNA- | crRNA with single UNA base |
| | | rG/rGrGrGrArGrArArGrGrGrUrUrUrUrArGrArGrCrUrArUr | |
| | | GrCrU/AltR2/ | |
| SEQ ID NO: 445 | PDCD1_8_UNA9 | /AltR1/rGrArGrCrArGrGrGrCrUrG/iUNA- | crRNA with single UNA base |
| | | rG/rGrGrArGrArArGrGrGrUrUrUrUrArGrArGrCrUrArUrGr | |
| | | CrU/AltR2/ | |
| SEQ ID NO: 446 | PDCD1_8_UNA8 | /AltR1/rGrArGrCrArGrGrGrCrUrGrG/iUNA- | crRNA with single UNA base |
| | | rG/rGrArGrArArGrGrGrUrUrUrUrArGrArGrCrUrArUrGrCr | |
| | | U/AltR2/ | |
| SEQ ID NO: 447 | PDCD1_8_UNA7 | /AltR1/rGrArGrCrArGrGrGrCrUrGrGrG/iUNA- | crRNA with single UNA base |
| | | rG/rArGrArArGrGrGrUrUrUrUrArGrArGrCrUrArUrGrCrU/ | |
| | | AltR2/ | |
| SEQ ID NO: 448 | PDCD1_8_UNA6 | /AltR1/rGrArGrCrArGrGrGrCrUrGrGrGrG/iUNA- | crRNA with single UNA base |
| | | rA/rGrArArGrGrGrUrUrUrUrArGrArGrCrUrArUrGrCrU/Alt | |
| | | R2/ | |
| SEQ ID NO: 449 | PDCD1_8_UNA5 | /AltR1/rGrArGrCrArGrGrGrCrUrGrGrGrGrA/iUNA- | crRNA with single UNA base |
| | | rG/rArArGrGrGrUrUrUrUrArGrArGrCrUrArUrGrCrU/AltR2 | |
| | | / | |
| SEQ ID NO: 450 | PDCD1_8_UNA4 | /AltR1/rGrArGrCrArGrGrGrCrUrGrGrGrGrArG/iUNA- | crRNA with single UNA base |
| | | rA/rArGrGrGrUrUrUrUrArGrArGrCrUrArUrGrCrU/AltR2/ | |
| SEQ ID NO: 451 | PDCD1_8_UNA3 | /AltR1/rGrArGrCrArGrGrGrCrUrGrGrGrGrArGrA/iUNA- | crRNA with single UNA base |
| | | rA/rGrGrGrUrUrUrUrArGrArGrCrUrArUrGrCrU/AltR2/ | |
| SEQ ID NO: 452 | PDCD1_8_UNA2 | /AltR1/rGrArGrCrArGrGrGrCrUrGrGrGrGrArGrArA/iUNA- | crRNA with single UNA base |
| | | rG/rGrGrUrUrUrUrArGrArGrCrUrArUrGrCrU/AltR2/ | |
| SEQ ID NO: 453 | PDCD1_8_UNA1 | /AltR1/rGrArGrCrArGrGrGrCrUrGrGrGrGrArGrArArG/iUN | crRNA with single UNA base |
| | | A-rG/rGrUrUrUrUrArGrArGrCrUrArUrGrCrU/AltR2/ | |
| SEQ ID NO: 454 | LAG3s9_UNA_20 | /AltR1//5UNA- | crRNA with single UNA base |
| | | rG/rArArGrGrCrUrGrArGrArUrCrCrUrGrGrArGrGrGrUrUr | |
| | | UrUrArGrArGrCrUrArUrGrCrU/AltR2/ | |
| SEQ ID NO: 455 | LAG3s9_UNA_19 | /AltR1/rG/iUNA- | crRNA with single UNA base |
| | | rA/rArGrGrCrUrGrArGrArUrCrCrUrGrGrArGrGrGrUrUrUr | |
| | | UrArGrArGrCrUrArUrGrCrU/AltR2/ | |
| SEQ ID NO: 456 | LAG3s9_UNA_18 | /AltR1/rGrA/iUNA- | crRNA with single UNA base |
| | | rA/rGrGrCrUrGrArGrArUrCrCrUrGrGrArGrGrGrUrUrUrUr | |
| | | ArGrArGrCrUrArUrGrCrU/AltR2/ | |
| SEQ ID NO: 457 | LAG3s9_UNA_17 | /AltR1/rGrArA/iUNA- | crRNA with single UNA base |
| | | rG/rGrCrUrGrArGrArUrCrCrUrGrGrArGrGrGrUrUrUrUrAr | |
| | | GrArGrCrUrArUrGrCrU/AltR2/ | |
| SEQ ID NO: 458 | LAG3s9_UNA_16 | /AltR1/rGrArArG/iUNA- | crRNA with single UNA base |
| | | rG/rCrUrGrArGrArUrCrCrUrGrGrArGrGrGrUrUrUrUrArGr | |
| | | ArGrCrUrArUrGrCrU/AltR2/ | |
| SEQ ID NO: 459 | LAG3s9_UNA_15 | /AltR1/rGrArArGrG/iUNA- | crRNA with single UNA base |
| | | rC/rUrGrArGrArUrCrCrUrGrGrArGrGrGrUrUrUrUrArGrAr | |
| | | GrCrUrArUrGrCrU/AltR2/ | |
| SEQ ID NO: 460 | LAG3s9_UNA_14 | /AltR1/rGrArArGrGrC/iUNA- | crRNA with single UNA base |
| | | rU/rGrArGrArUrCrCrUrGrGrArGrGrGrUrUrUrUrArGrArGr | |
| | | CrUrArUrGrCrU/AltR2/ | |
| SEQ ID NO: 461 | LAG3s9_UNA_13 | /AltR1/rGrArArGrGrCrU/iUNA- | crRNA with single UNA base |
| | | rG/rArGrArUrCrCrUrGrGrArGrGrGrUrUrUrUrArGrArGrCr | |
| | | UrArUrGrCrU/AltR2/ | |
| SEQ ID NO: 462 | LAG3s9_UNA_12 | /AltR1/rGrArArGrGrCrUrG/iUNA- | crRNA with single UNA base |
| | | rA/rGrArUrCrCrUrGrGrArGrGrGrUrUrUrUrArGrArGrCrUr | |
| | | ArUrGrCrU/AltR2/ | |
| SEQ ID NO: 463 | LAG3s9_UNA_11 | /AltR1/rGrArArGrGrCrUrGrA/iUNA- | crRNA with single UNA base |
| | | rG/rArUrCrCrUrGrGrArGrGrGrUrUrUrUrArGrArGrCrUrAr | |
| | | UrGrCrU/AltR2/ | |
| SEQ ID NO: 464 | LAG3s9_UNA_10 | /AltR1/rGrArArGrGrCrUrGrArG/iUNA- | crRNA with single UNA base |
| | | rA/rUrCrCrUrGrGrArGrGrGrUrUrUrUrArGrArGrCrUrArUr | |
| | | GrCrU/AltR2/ | |
| SEQ ID NO: 465 | LAG3s9_UNA_9 | /AltR1/rGrArArGrGrCrUrGrArGrA/iUNA- | crRNA with single UNA base |
| | | rU/rCrCrUrGrGrArGrGrGrUrUrUrUrArGrArGrCrUrArUrGr | |
| | | CrU/AltR2/ | |
| SEQ ID NO: 466 | LAG3s9_UNA_8 | /AltR1/rGrArArGrGrCrUrGrArGrArU/iUNA- | crRNA with single UNA base |
| | | rC/rCrUrGrGrArGrGrGrUrUrUrUrArGrArGrCrUrArUrGrCr | |
| | | U/AltR2/ | |
| SEQ ID NO: 467 | LAG3s9_UNA_7 | /AltR1/rGrArArGrGrCrUrGrArGrArUrC/iUNA- | crRNA with single UNA base |
| | | rC/rUrGrGrArGrGrGrUrUrUrUrArGrArGrCrUrArUrGrCrU/ | |
| | | AltR2/ | |
| SEQ ID NO: 468 | LAG3s9_UNA_6 | /AltR1/rGrArArGrGrCrUrGrArGrArUrCrC/iUNA- | crRNA with single UNA base |
| | | rU/rGrGrArGrGrGrUrUrUrUrArGrArGrCrUrArUrGrCrU/Alt | |
| | | R2/ | |
| SEQ ID NO: 469 | LAG3s9_UNA_5 | /AltR1/rGrArArGrGrCrUrGrArGrArUrCrCrU/iUNA- | crRNA with single UNA base |
| | | rG/rGrArGrGrGrUrUrUrUrArGrArGrCrUrArUrGrCrU/ | |
| | | AltR2/ | |
| SEQ ID NO: 470 | LAG3s9_UNA_4 | /AltR1/rGrArArGrGrCrUrGrArGrArUrCrCrUrG/iUNA- | crRNA with single UNA base |
| | | rG/rArGrGrGrUrUrUrUrArGrArGrCrUrArUrGrCrU/AltR2/ | |
| SEQ ID NO: 471 | LAG3s9_UNA_3 | /AltR1/rGrArArGrGrCrUrGrArGrArUrCrCrUrGrG/iUNA- | crRNA with single UNA base |
| | | rA/rGrGrGrUrUrUrUrArGrArGrCrUrArUrGrCrU/AltR2/ | |
| SEQ ID NO: 472 | LAG3s9_UNA_2 | /AltR1/rGrArArGrGrCrUrGrArGrArUrCrCrUrGrGrA/iUNA- | crRNA with single UNA base |
| | | rG/rGrGrUrUrUrUrArGrArGrCrUrArUrGrCrU/AltR2/ | |
| SEQ ID NO: 473 | LAG3s9_UNA_1 | /AltR1/rGrArArGrGrCrUrGrArGrArUrCrCrUrGrGrArG/iUN | crRNA with single UNA base |
| | | A-rG/rGrUrUrUrUrArGrArGrCrUrArUrGrCrU/AltR2/ | |
| SEQ ID NO: 474 | FANCF_tgt13_UNA20 | /AltR1//iUNA- | crRNA with single UNA base |
| | | rG/rCrUrGrCrArGrArArGrGrGrArUrUrCrCrArUrGrGrUrUr | |
| | | UrUrArGrArGrCrUrArUrGrCrU/AltR2/ | |
| SEQ ID NO: 475 | FANCF_tgt13_UNA19 | /AltR1/rG/iUNA- | crRNA with single UNA base |
| | | rC/rUrGrCrArGrArArGrGrGrArUrUrCrCrArUrGrGrUrUrUr | |
| | | UrArGrArGrCrUrArUrGrCrU/AltR2/ | |
| SEQ ID NO: 476 | FANCF_tgt13_UNA18 | /AltR1/rGrC/iUNA- | crRNA with single UNA base |
| | | rU/rGrCrArGrArArGrGrGrArUrUrCrCrArUrGrGrUrUrUrUr | |
| | | ArGrArGrCrUrArUrGrCrU/AltR2/ | |
| SEQ ID NO: 477 | FANCF_tgt13_UNA17 | /AltR1/rGrCrU/iUNA- | crRNA with single UNA base |
| | | rG/rCrArGrArArGrGrGrArUrUrCrCrArUrGrGrUrUrUrUrAr | |
| | | GrArGrCrUrArUrGrCrU/AltR2/ | |
| SEQ ID NO: 478 | FANCF_tgt13_UNA16 | /AltR1/rGrCrUrG/iUNA- | crRNA with single UNA base |
| | | rC/rArGrArArGrGrGrArUrUrCrCrArUrGrGrUrUrUrUrArGr | |
| | | ArGrCrUrArUrGrCrU/AltR2/ | |
| SEQ ID NO: 479 | FANCF_tgt13_UNA15 | /AltR1/rGrCrUrGrC/iUNA- | crRNA with single UNA base |
| | | rA/rGrArArGrGrGrArUrUrCrCrArUrGrGrUrUrUrUrArGrAr | |
| | | GrCrUrArUrGrCrU/AltR2/ | |
| SEQ ID NO: 480 | FANCF_tgt13_UNA14 | /AltR1/rGrCrUrGrCrA/iUNA- | crRNA with single UNA base |
| | | rG/rArArGrGrGrArUrUrCrCrArUrGrGrUrUrUrUrArGrArGr | |
| | | CrUrArUrGrCrU/AltR2/ | |
| SEQ ID NO: 481 | FANCF_tgt13_UNA13 | /AltR1/rGrCrUrGrCrArG/iUNA- | crRNA with single UNA base |
| | | rA/rArGrGrGrArUrUrCrCrArUrGrGrUrUrUrUrArGrArGrCr | |
| | | UrArUrGrCrU/AltR2/ | |
| SEQ ID NO: 482 | FANCF_tgt13_UNA12 | /AltR1/rGrCrUrGrCrArGrA/iUNA- | crRNA with single UNA base |
| | | rA/rGrGrGrArUrUrCrCrArUrGrGrUrUrUrUrArGrArGrCrUr | |
| | | ArUrGrCrU/AltR2/ | |
| SEQ ID NO: 483 | FANCF_tgt13_UNA11 | /AltR1/rGrCrUrGrCrArGrArA/iUNA- | crRNA with single UNA base |
| | | rG/rGrGrArUrUrCrCrArUrGrGrUrUrUrUrArGrArGrCrUrAr | |
| | | UrGrCrU/AltR2/ | |
| SEQ ID NO: 484 | FANCF_tgt13_UNA10 | /AltR1/rGrCrUrGrCrArGrArArG/iUNA- | crRNA with single UNA base |
| | | rG/rGrArUrUrCrCrArUrGrGrUrUrUrUrArGrArGrCrUrArUr | |
| | | GrCrU/AltR2/ | |
| SEQ ID NO: 485 | FANCF_tgt13_UNA9 | /AltR1/rGrCrUrGrCrArGrArArGrG/iUNA- | crRNA with single UNA base |
| | | rG/rArUrUrCrCrArUrGrGrUrUrUrUrArGrArGrCrUrArUrGr | |
| | | CrU/AltR2/ | |
| SEQ ID NO: 486 | FANCF_tgt13_UNA8 | /AltR1/rGrCrUrGrCrArGrArArGrGrG/iUNA- | crRNA with single UNA base |
| | | rA/rUrUrCrCrArUrGrGrUrUrUrUrArGrArGrCrUrArUrGrCr | |
| | | U/AltR2/ | |
| SEQ ID NO: 487 | FANCF_tgt13_UNA7 | /AltR1/rGrCrUrGrCrArGrArArGrGrGrA/iUNA- | crRNA with single UNA base |
| | | rU/rUrCrCrArUrGrGrUrUrUrUrArGrArGrCrUrArUrGrCrU/ | |
| | | AltR2/ | |
| SEQ ID NO: 488 | FANCF_tgt13_UNA6 | /AltR1/rGrCrUrGrCrArGrArArGrGrGrArU/iUNA- | crRNA with single UNA base |
| | | rU/rCrCrArUrGrGrUrUrUrUrArGrArGrCrUrArUrGrCrU/Alt | |
| | | R2/ | |
| SEQ ID NO: 489 | FANCF_tgt13_UNA5 | /AltR1/rGrCrUrGrCrArGrArArGrGrGrArUrU/iUNA- | crRNA with single UNA base |
| | | rC/rCrArUrGrGrUrUrUrUrArGrArGrCrUrArUrGrCrU/AltR2/ | |
| SEQ ID NO: 490 | FANCF_tgt13_UNA4 | /AltR1/rGrCrUrGrCrArGrArArGrGrGrArUrUrC/iUNA- | crRNA with single UNA base |
| | | rC/rArUrGrGrUrUrUrUrArGrArGrCrUrArUrGrCrU/AltR2/ | |
| SEQ ID NO: 491 | FANCF_tgt13_UNA3 | /AltR1/rGrCrUrGrCrArGrArArGrGrGrArUrUrCrC/iUNA- | crRNA with single UNA base |
| | | rA/rUrGrGrUrUrUrUrArGrArGrCrUrArUrGrCrU/AltR2/ | |
| SEQ ID NO: 492 | FANCF_tgt13_UNA2 | /AltR1/rGrCrUrGrCrArGrArArGrGrGrArUrUrCrCrA/iUNA- | crRNA with single UNA base |
| | | rU/rGrGrUrUrUrUrArGrArGrCrUrArUrGrCrU/AltR2/ | |
| SEQ ID NO: 493 | FANCF_tgt13_UNA1 | /AltR1/rGrCrUrGrCrArGrArArGrGrGrArUrUrCrCrArU/iUN | crRNA with single UNA base |
| | | A-rG/rGrUrUrUrUrArGrArGrCrUrArUrGrCrU/AltR2/ | |
| SEQ ID NO: 494 | EMX1_UNA20 | /AltR1//iUNA- | crRNA with single UNA base |
| | | rG/rArGrUrCrCrGrArGrCrArGrArArGrArArGrArArGrUrUr | |
| | | UrUrArGrArGrCrUrArUrGrCrU/AltR2/ | |
| SEQ ID NO: 495 | EMX1_UNA19 | /AltR1/rG/iUNA- | crRNA with single UNA base |
| | | rA/rGrUrCrCrGrArGrCrArGrArArGrArArGrArArGrUrUrUr | |
| | | UrArGrArGrCrUrArUrGrCrU/AltR2/ | |
| SEQ ID NO: 496 | EMX1_UNA18 | /AltR1/rGrA/iUNA- | crRNA with single UNA base |
| | | rG/rUrCrCrGrArGrCrArGrArArGrArArGrArArGrUrUrUrUr | |
| | | ArGrArGrCrUrArUrGrCrU/AltR2/ | |
| SEQ ID NO: 497 | EMX1_UNA17 | /AltR1/rGrArG/iUNA- | crRNA with single UNA base |
| | | rU/rCrCrGrArGrCrArGrArArGrArArGrAr ArGrUrUrUrUrAr | |
| | | GrArGrCrUrArUrGrCrU/AltR2/ | |
| SEQ ID NO: 498 | EMX1_UNA16 | /AltR1/rGrArGrU/iUNA- | crRNA with single UNA base |
| | | rC/rCrGrArGrCrArGrArArGrArArGrArArGrUrUrUrUrArGr | |
| | | ArGrCrUrArUrGrCrU/AltR2/ | |
| SEQ ID NO: 499 | EMX1_UNA15 | /AltR1/rGrArGrUrC/iUNA- | crRNA with single UNA base |
| | | rC/rGrArGrCrArGrArArGrArArGrArArGrUrUrUrUrArGrAr | |
| | | GrCrUrArUrGrCrU/AltR2/ | |
| SEQ ID NO: 500 | EMX1_UNA14 | /AltR1/rGrArGrUrCrC/iUNA- | crRNA with single UNA base |
| | | rG/rArGrCrArGrArArGrArArGrArArGrUrUrUrUrArGrArGr | |
| | | CrUrArUrGrCrU/AltR2/ | |
| SEQ ID NO: 501 | EMX1_UNA13 | /AltR1/rGrArGrUrCrCrG/iUNA- | crRNA with single UNA base |
| | | rA/rGrCrArGrArArGrArArGrArArGrUrUrUrUrArGrArGrCr | |
| | | UrArUrGrCrU/AltR2/ | |
| SEQ ID NO: 502 | EMX1_UNA12 | /AltR1/rGrArGrUrCrCrGrA/iUNA- | crRNA with single UNA base |
| | | rG/rCrArGrArArGrArArGrArArGrUrUrUrUrArGrArGrCrUr | |
| | | ArUrGrCrU/AltR2/ | |
| SEQ ID NO: 503 | EMX1_UNA11 | /AltR1/rGrArGrUrCrCrGrArG/iUNA- | crRNA with single UNA base |
| | | rC/rArGrArArGrArArGrArArGrUrUrUrUrArGrArGrCrUrAr | |
| | | UrGrCrU/AltR2/ | |
| SEQ ID NO: 504 | EMX1_UNA10 | /AltR1/rGrArGrUrCrCrGrArGrC/iUNA- | crRNA with single UNA base |
| | | rA/rGrArArGrArArGrArArGrUrUrUrUrArGrArGrCrUrArUr | |
| | | GrCrU/AltR2/ | |
| SEQ ID NO: 505 | EMX1_UNA9 | /AltR1/rGrArGrUrCrCrGrArGrCrA/iUNA- | crRNA with single UNA base |
| | | rG/rArArGrArArGrArArGrUrUrUrUrArGrArGrCrUrArUrGr | |
| | | CrU/AltR2/ | |
| SEQ ID NO: 506 | EMX1_UNA8 | /AltR1/rGrArGrUrCrCrGrArGrCrArG/iUNA- | crRNA with single UNA base |
| | | rA/rArGrArArGrArArGrUrUrUrUrArGrArGrCrUrArUrGrCr | |
| | | U/AltR2/ | |
| SEQ ID NO: 507 | EMX1_UNA7 | /AltR1/rGrArGrUrCrCrGrArGrCrArGrA/iUNA- | crRNA with single UNA base |
| | | rA/rGrArArGrArArGrUrUrUrUrArGrArGrCrUrArUrGrCrU/ | |
| | | AltR2/ | |
| SEQ ID NO: 508 | EMX1_UNA6 | /AltR1/rGrArGrUrCrCrGrArGrCrArGrArA/iUNA- | crRNA with single UNA base |
| | | rG/rArArGrArArGrUrUrUrUrArGrArGrCrUrArUrGrCrU/Alt | |
| | | R2/ | |
| SEQ ID NO: 509 | EMX1_UNA5 | /AltR1/rGrArGrUrCrCrGrArGrCrArGrArArG/iUNA- | crRNA with single UNA base |
| | | rA/rArGrArArGrUrUrUrUrArGrArGrCrUrArUrGrCrU/AltR2 | |
| | | / | |
| SEQ ID NO: 510 | EMX1_UNA4 | /AltR1/rGrArGrUrCrCrGrArGrCrArGrArArGrA/iUNA- | crRNA with single UNA base |
| | | rA/rGrArArGrUrUrUrUrArGrArGrCrUrArUrGrCrU/AltR2/ | |
| SEQ ID NO: 511 | EMX1_UNA3 | /AltR1/rGrArGrUrCrCrGrArGrCrArGrArArGrArA/iUNA- | crRNA with single UNA base |
| | | rG/rArArGrUrUrUrUrArGrArGrCrUrArUrGrCrU/AltR2/ | |
| SEQ ID NO: 512 | EMX1_UNA2 | /AltR1/rGrArGrUrCrCrGrArGrCrArGrArArGrArArG/iUNA- | crRNA with single UNA base |
| | | rA/rArGrUrUrUrUrArGrArGrCrUrArUrGrCrU/AltR2/ | |
| SEQ ID NO: 513 | EMX1_UNA1 | /AltR1/rGrArGrUrCrCrGrArGrCrArGrArArGrArArGrA/iUN | crRNA with single UNA base |
| | | A-rA/rGrUrUrUrUrArGrArGrCrUrArUrGrCrU/AltR2/ | |
| SEQ ID NO: 514 | HBB_UNA20 | /AltR1//iUNA- | crRNA with single UNA base |
| | | rC/rUrUrGrCrCrCrCrArCrArGrGrGrCrArGrUrArArGrUrUrU | |
| | | rUrArGrArGrCrUrArUrGrCrU/AltR2/ | |
| SEQ ID NO: 515 | HBB_UNA19 | /AltR1/rC/iUNA- | crRNA with single UNA base |
| | | rU/rUrGrCrCrCrCrArCrArGrGrGrCrArGrUrArArGrUrUrUr | |
| | | UrArGrArGrCrUrArUrGrCrU/AltR2/ | |
| SEQ ID NO: 516 | HBB_UNA18 | /AltR1/rCrU/iUNA- | crRNA with single UNA base |
| | | rU/rGrCrCrCrCrArCrArGrGrGrCrArGrUrArArGrUrUrUrUr | |
| | | ArGrArGrCrUrArUrGrCrU/AltR2/ | |
| SEQ ID NO: 517 | HBB_UNA17 | /AltR1/rCrUrU/iUNA- | crRNA with single UNA base |
| | | rG/rCrCrCrCrArCrArGrGrGrCrArGrUrArArGrUrUrUrUrAr | |
| | | GrArGrCrUrArUrGrCrU/AltR2/ | |
| SEQ ID NO: 518 | HBB_UNA16 | /AltR1/rCrUrUrG/iUNA- | crRNA with single UNA base |
| | | rC/rCrCrCrArCrArGrGrGrCrArGrUrArArGrUrUrUrUrArGr | |
| | | ArGrCrUrArUrGrCrU/AltR2/ | |
| SEQ ID NO: 519 | HBB_UNA15 | /AltR1/rCrUrUrGrC/iUNA- | crRNA with single UNA base |
| | | rC/rCrCrArCrArGrGrGrCrArGrUrArArGrUrUrUrUrArGrAr | |
| | | GrCrUrArUrGrCrU/AltR2/ | |
| SEQ ID NO: 520 | HBB_UNA14 | /AltR1/rCrUrUrGrCrC/iUNA- | crRNA with single UNA base |
| | | rC/rCrArCrArGrGrGrCrArGrUrArArGrUrUrUrUrArGrArGr | |
| | | CrUrArUrGrCrU/AltR2/ | |
| SEQ ID NO: 521 | HBB_UNA13 | /AltR1/rCrUrUrGrCrCrC/iUNA- | crRNA with single UNA base |
| | | rC/rArCrArGrGrGrCrArGrUrArArGrUrUrUrUrArGrArGrCr | |
| | | UrArUrGrCrU/AltR2/ | |
| SEQ ID NO: 522 | HBB_UNA12 | /AltR1/rCrUrUrGrCrCrCrC/iUNA- | crRNA with single UNA base |
| | | rA/rCrArGrGrGrCrArGrUrArArGrUrUrUrUrArGrArGrCrUr | |
| | | ArUrGrCrU/AltR2/ | |
| SEQ ID NO: 523 | HBB_UNA11 | /AltR1/rCrUrUrGrCrCrCrCrA/iUNA- | crRNA with single UNA base |
| | | rC/rArGrGrGrCrArGrUrArArGrUrUrUrUrArGrArGrCrUrAr | |
| | | UrGrCrU/AltR2/ | |
| SEQ ID NO: 524 | HBB_UNA10 | /AltR1/rCrUrUrGrCrCrCrCrArC/iUNA- | crRNA with single UNA base |
| | | rA/rGrGrGrCrArGrUrArArGrUrUrUrUrArGrArGrCrUrArUr | |
| | | GrCrU/AltR2/ | |
| SEQ ID NO: 525 | HBB_UNA9 | /AltR1/rCrUrUrGrCrCrCrCrArCrA/iUNA- | crRNA with single UNA base |
| | | rG/rGrGrCrArGrUrArArGrUrUrUrUrArGrArGrCrUrArUrGr | |
| | | CrU/AltR2/ | |
| SEQ ID NO: 526 | HBB_UNA8 | /AltR1/rCrUrUrGrCrCrCrCrArCrArG/iUNA- | crRNA with single UNA base |
| | | rG/rGrCrArGrUrArArGrUrUrUrUrArGrArGrCrUrArUrGrCr | |
| | | U/AltR2/ | |
| SEQ ID NO: 527 | HBB_UNA7 | /AltR1/rCrUrUrGrCrCrCrCrArCrArGrG/iUNA- | crRNA with single UNA base |
| | | rG/rCrArGrUrArArGrUrUrUrUrArGrArGrCrUrArUrGrCrU/ | |
| | | AltR2/ | |
| SEQ ID NO: 528 | HBB_UNA6 | /AltR1/rCrUrUrGrCrCrCrCrArCrArGrGrG/iUNA- | crRNA with single UNA base |
| | | rC/rArGrUrArArGrUrUrUrUrArGrArGrCrUrArUrGrCrU/Alt | |
| | | R2/ | |
| SEQ ID NO: 529 | HBB_UNA5 | /AltR1/rCrUrUrGrCrCrCrCrArCrArGrGrGrC/iUNA- | crRNA with single UNA base |
| | | rA/rGrUrArArGrUrUrUrUrArGrArGrCrUrArUrGrCrU/AltR2 | |
| | | / | |
| SEQ ID NO: 530 | HBB_UNA4 | /AltR1/rCrUrUrGrCrCrCrCrArCrArGrGrGrCrA/iUNA- | crRNA with single UNA base |
| | | rG/rUrArArGrUrUrUrUrArGrArGrCrUrArUrGrCrU/AltR2/ | |
| SEQ ID NO: 531 | HBB_UNA3 | /AltR1/rCrUrUrGrCrCrCrCrArCrArGrGrGrCrArG/iUNA- | crRNA with single UNA base |
| | | rU/rArArGrUrUrUrUrArGrArGrCrUrArUrGrCrU/AltR2/ | |
| SEQ ID NO: 532 | HBB_UNA2 | /AltR1/rCrUrUrGrCrCrCrCrArCrArGrGrGrCrArGrU/iUNA- | crRNA with single UNA base |
| | | rA/rArGrUrUrUrUrArGrArGrCrUrArUrGrCrU/AltR2/ | |
| SEQ ID NO: 533 | HBB_UNA1 | /AltR1/rCrUrUrGrCrCrCrCrArCrArGrGrGrCrArGrUrA/iUN | crRNA with single UNA base |
| | | A-rA/rGrUrUrUrUrArGrArGrCrUrArUrGrCrU/AltR2/ | |
| SEQ ID NO: 534 | AR_UNA20 | /AltR1//iUNA- | crRNA with single UNA base |
| | | rG/rUrUrGrGrArGrCrArUrCrUrGrArGrUrCrCrArGrGrUrUr | |
| | | UrUrArGrArGrCrUrArUrGrCrU/AltR2/ | |
| SEQ ID NO: 535 | AR_UNA19 | /AltR1/rG/iUNA- | crRNA with single UNA base |
| | | rU/rUrGrGrArGrCrArUrCrUrGrArGrUrCrCrArGrGrUrUrUr | |
| | | UrArGrArGrCrUrArUrGrCrU/AltR2/ | |
| SEQ ID NO: 536 | AR_UNA18 | /AltR1/rGrU/iUNA- | crRNA with single UNA base |
| | | rU/rGrGrArGrCrArUrCrUrGrArGrUrCrCrArGrGrUrUrUrUr | |
| | | ArGrArGrCrUrArUrGrCrU/AltR2/ | |
| SEQ ID NO: 537 | AR_UNA17 | /AltR1/rGrUrU/iUNA- | crRNA with single UNA base |
| | | rG/rGrArGrCrArUrCrUrGrArGrUrCrCrArGrGrUrUrUrUrAr | |
| | | GrArGrCrUrArUrGrCrU/AltR2/ | |
| SEQ ID NO: 538 | AR_UNA16 | /AltR1/rGrUrUrG/iUNA- | crRNA with single UNA base |
| | | rG/rArGrCrArUrCrUrGrArGrUrCrCrArGrGrUrUrUrUrArGr | |
| | | ArGrCrUrArUrGrCrU/AltR2/ | |
| SEQ ID NO: 539 | AR_UNA15 | /AltR1/rGrUrUrGrG/iUNA- | crRNA with single UNA base |
| | | rA/rGrCrArUrCrUrGrArGrUrCrCrArGrGrUrUrUrUrArGrAr | |
| | | GrCrUrArUrGrCrU/AltR2/ | |
| SEQ ID NO: 540 | AR_UNA14 | /AltR1/rGrUrUrGrGrA/iUNA- | crRNA with single UNA base |
| | | rG/rCrArUrCrUrGrArGrUrCrCrArGrGrUrUrUrUrArGrArGr | |
| | | CrUrArUrGrCrU/AltR2/ | |
| SEQ ID NO: 541 | AR_UNA13 | /AltR1/rGrUrUrGrGrArG/iUNA- | crRNA with single UNA base |
| | | rC/rArUrCrUrGrArGrUrCrCrArGrGrUrUrUrUrArGrArGrCr | |
| | | UrArUrGrCrU/AltR2/ | |
| SEQ ID NO: 542 | AR_UNA12 | /AltR1/rGrUrUrGrGrArGrC/iUNA- | crRNA with single UNA base |
| | | rA/rUrCrUrGrArGrUrCrCrArGrGrUrUrUrUrArGrArGrCrUr | |
| | | ArUrGrCrU/AltR2/ | |
| SEQ ID NO: 543 | AR_UNA11 | /AltR1/rGrUrUrGrGrArGrCrA/iUNA- | crRNA with single UNA base |
| | | rU/rCrUrGrArGrUrCrCrArGrGrUrUrUrUrArGrArGrCrUrAr | |
| | | UrGrCrU/AltR2/ | |
| SEQ ID NO: 544 | AR_UNA10 | /AltR1/rGrUrUrGrGrArGrCrArU/iUNA- | crRNA with single UNA base |
| | | rC/rUrGrArGrUrCrCrArGrGrUrUrUrUrArGrArGrCrUrArUr | |
| | | GrCrU/AltR2/ | |
| SEQ ID NO: 545 | AR_UNA9 | /AltR1/rGrUrUrGrGrArGrCrArUrC/iUNA- | crRNA with single UNA base |
| | | rU/rGrArGrUrCrCrArGrGrUrUrUrUrArGrArGrCrUrArUrGr | |
| | | CrU/AltR2/ | |
| SEQ ID NO: 546 | AR_UNA8 | /AltR1/rGrUrUrGrGrArGrCrArUrCrU/iUNA- | crRNA with single UNA base |
| | | rG/rArGrUrCrCrArGrGrUrUrUrUrArGrArGrCrUrArUrGrCr | |
| | | U/AltR2/ | |
| SEQ ID NO: 547 | AR_UNA7 | /AltR1/rGrUrUrGrGrArGrCrArUrCrUrG/iUNA- | crRNA with single UNA base |
| | | rA/rGrUrCrCrArGrGrUrUrUrUrArGrArGrCrUrArUrGrCrU/ | |
| | | AltR2/ | |
| SEQ ID NO: 548 | AR_UNA6 | /AltR1/rGrUrUrGrGrArGrCrArUrCrUrGrA/iUNA- | crRNA with single UNA base |
| | | rG/rUrCrCrArGrGrUrUrUrUrArGrArGrCrUrArUrGrCrU/Alt | |
| | | R2/ | |
| SEQ ID NO: 549 | AR_UNA5 | /AltR1/rGrUrUrGrGrArGrCrArUrCrUrGrArG/iUNA- | crRNA with single UNA base |
| | | rU/rCrCrArGrGrUrUrUrUrArGrArGrCrUrArUrGrCrU/AltR2/ | |
| SEQ ID NO: 550 | AR_UNA4 | /AltR1/rGrUrUrGrGrArGrCrArUrCrUrGrArGrU/iUNA- | crRNA with single UNA base |
| | | rC/rCrArGrGrUrUrUrUrArGrArGrCrUrArUrGrCrU/AltR2/ | |
| SEQ ID NO: 551 | AR_UNA3 | /AltR1/rGrUrUrGrGrArGrCrArUrCrUrGrArGrUrC/iUNA- | crRNA with single UNA base |
| | | rC/rArGrGrUrUrUrUrArGrArGrCrUrArUrGrCrU/AltR2/ | |
| SEQ ID NO: 552 | AR_UNA2 | /AltR1/rGrUrUrGrGrArGrCrArUrCrUrGrArGrUrCrC/iUNA- | crRNA with single UNA base |
| | | rA/rGrGrUrUrUrUrArGrArGrCrUrArUrGrCrU/AltR2/ | |
| SEQ ID NO: 553 | AR_UNA1 | /AltR1/rGrUrUrGrGrArGrCrArUrCrUrGrArGrUrCrCrA/iUN | crRNA with single UNA base |
| | | A-rG/rGrUrUrUrUrArGrArGrCrUrArUrGrCrU/AltR2/ | |
| SEQ ID NO: 554 | HEKs3_UNA20 | /AltR1//iUNA- | crRNA with single UNA base |
| | | rG/rGrCrCrCrArGrArCrUrGrArGrCrArCrGrUrGrArGrUrUr | |
| | | UrUrArGrArGrCrUrArUrGrCrU/AltR2/ | |
| SEQ ID NO: 555 | HEKs3_UNA19 | /AltR1/rG/iUNA- | crRNA with single UNA base |
| | | rG/rCrCrCrArGrArCrUrGrArGrCrArCrGrUrGrArGrUrUrUr | |
| | | UrArGrArGrCrUrArUrGrCrU/AltR2/ | |
| SEQ ID NO: 556 | HEKs3_UNA18 | /AltR1/rGrG/iUNA- | crRNA with single UNA base |
| | | rC/rCrCrArGrArCrUrGrArGrCrArCrGrUrGrArGrUrUrUrUr | |
| | | ArGrArGrCrUrArUrGrCrU/AltR2/ | |
| SEQ ID NO: 557 | HEKs3_UNA17 | /AltR1/rGrGrC/iUNA- | crRNA with single UNA base |
| | | rC/rCrArGrArCrUrGrArGrCrArCrGrUrGrArGrUrUrUrUrAr | |
| | | GrArGrCrUrArUrGrCrU/AltR2/ | |
| SEQ ID NO: 558 | HEKs3_UNA16 | /AltR1/rGrGrCrC/iUNA- | crRNA with single UNA base |
| | | rC/rArGrArCrUrGrArGrCrArCrGrUrGrArGrUrUrUrUrArGr | |
| | | ArGrCrUrArUrGrCrU/AltR2/ | |
| SEQ ID NO: 559 | HEKs3_UNA15 | /AltR1/rGrGrCrCrC/iUNA- | crRNA with single UNA base |
| | | rA/rGrArCrUrGrArGrCrArCrGrUrGrArGrUrUrUrUrArGrAr | |
| | | GrCrUrArUrGrCrU/AltR2/ | |
| SEQ ID NO: 560 | HEKs3_UNA14 | /AltR1/rGrGrCrCrCrA/iUNA- | crRNA with single UNA base |
| | | rG/rArCrUrGrArGrCrArCrGrUrGrArGrUrUrUrUrArGrArGr | |
| | | CrUrArUrGrCrU/AltR2/ | |
| SEQ ID NO: 561 | HEKs3_UNA13 | /AltR1/rGrGrCrCrCrArG/iUNA- | crRNA with single UNA base |
| | | rA/rCrUrGrArGrCrArCrGrUrGrArGrUrUrUrUrArGrArGrCr | |
| | | UrArUrGrCrU/AltR2/ | |
| SEQ ID NO: 562 | HEKs3_UNA12 | /AltR1/rGrGrCrCrCrArGrA/iUNA- | crRNA with single UNA base |
| | | rC/rUrGrArGrCrArCrGrUrGrArGrUrUrUrUrArGrArGrCrUr | |
| | | ArUrGrCrU/AltR2/ | |
| SEQ ID NO: 563 | HEKs3_UNA11 | /AltR1/rGrGrCrCrCrArGrArC/iUNA- | crRNA with single UNA base |
| | | rU/rGrArGrCrArCrGrUrGrArGrUrUrUrUrArGrArGrCrUrAr | |
| | | UrGrCrU/AltR2/ | |
| SEQ ID NO: 564 | HEKs3_UNA10 | /AltR1/rGrGrCrCrCrArGrArCrU/iUNA- | crRNA with single UNA base |
| | | rG/rArGrCrArCrGrUrGrArGrUrUrUrUrArGrArGrCrUrArUr | |
| | | GrCrU/AltR2 | |
| SEQ ID NO: 565 | HEKs3_UNA9 | /AltR1/rGrGrCrCrCrArGrArCrUrG/iUNA- | crRNA with single UNA base |
| | | rA/rGrCrArCrGrUrGrArGrUrUrUrUrArGrArGrCrUrArUrGr | |
| | | CrU/AltR2/ | |
| SEQ ID NO: 566 | HEKs3_UNA8 | /AltR1/rGrGrCrCrCrArGrArCrUrGrA/iUNA- | crRNA with single UNA base |
| | | rG/rCrArCrGrUrGrArGrUrUrUrUrArGrArGrCrUrArUrGrCr | |
| | | U/AltR2/ | |
| SEQ ID NO: 567 | HEKs3_UNA7 | /AltR1/rGrGrCrCrCrArGrArCrUrGrArG/iUNA- | crRNA with single UNA base |
| | | rC/rArCrGrUrGrArGrUrUrUrUrArGrArGrCrUrArUrGrCrU/ | |
| | | AltR2/ | |
| SEQ ID NO: 568 | HEKs3_UNA6 | /AltR1/rGrGrCrCrCrArGrArCrUrGrArGrC/iUNA- | crRNA with single UNA base |
| | | rA/rCrGrUrGrArGrUrUrUrUrArGrArGrCrUrArUrGrCrU/Alt | |
| | | R2/ | |
| SEQ ID NO: 569 | HEKs3_UNA5 | /AltR1/rGrGrCrCrCrArGrArCrUrGrArGrCrA/iUNA- | crRNA with single UNA base |
| | | rC/rGrUrGrArGrUrUrUrUrArGrArGrCrUrArUrGrCrU/AltR2 | |
| | | / | |
| SEQ ID NO: 570 | HEKs3_UNA4 | /AltR1/rGrGrCrCrCrArGrArCrUrGrArGrCrArC/iUNA- | crRNA with single UNA base |
| | | rG/rUrGrArGrUrUrUrUrArGrArGrCrUrArUrGrCrU/AltR2/ | |
| SEQ ID NO: 571 | HEKs3_UNA3 | /AltR1/rGrGrCrCrCrArGrArCrUrGrArGrCrArCrG/iUNA- | crRNA with single UNA base |
| | | rU/rGrArGrUrUrUrUrArGrArGrCrUrArUrGrCrU/AltR2/ | |
| SEQ ID NO: 572 | HEKs3_UNA2 | /AltR1/rGrGrCrCrCrArGrArCrUrGrArGrCrArCrGrU/iUNA- | crRNA with single UNA base |
| | | rG/rArGrUrUrUrUrArGrArGrCrUrArUrGrCrU/AltR2/ | |
| SEQ ID NO: 573 | HEKs3_UNA1 | /AltR1/rGrGrCrCrCrArGrArCrUrGrArGrCrArCrGrUrG/iUN | crRNA with single UNA base |
| | | A-rA/rGrUrUrUrUrArGrArGrCrUrArUrGrCrU/AltR2/ | |
| SEQ ID NO: 574 | HPRT38087_UNA20 | /AltR1//iUNA- | crRNA with single UNA base |
| | | rA/rArUrUrArUrGrGrGrGrArUrUrArCrUrArGrGrArGrUrUr | |
| | | UrUrArGrArGrCrUrArUrGrCrU/AltR2/ | |
| SEQ ID NO: 575 | HPRT38087_UNA19 | /AltR1/rA/iUNA- | crRNA with single UNA base |
| | | rA/rUrUrArUrGrGrGrGrArUrUrArCrUrArGrGrArGrUrUrUr | |
| | | UrArGrArGrCrUrArUrGrCrU/AltR2/ | |
| SEQ ID NO: 576 | HPRT38087_UNA18 | /AltR1/rArA/iUNA- | crRNA with single UNA base |
| | | rU/rUrArUrGrGrGrGrArUrUrArCrUrArGrGrArGrUrUrUrUr | |
| | | ArGrArGrCrUrArUrGrCrU/AltR2/ | |
| SEQ ID NO: 577 | HPRT38087_UNA17 | /AltR1/rArArU/iUNA- | crRNA with single UNA base |
| | | rU/rArUrGrGrGrGrArUrUrArCrUrArGrGrArGrUrUrUrUrAr | |
| | | GrArGrCrUrArUrGrCrU/AltR2/ | |
| SEQ ID NO: 578 | HPRT38087_UNA16 | /AltR1/rArArUrU/iUNA- | crRNA with single UNA base |
| | | rA/rUrGrGrGrGrArUrUrArCrUrArGrGrArGrUrUrUrUrArGr | |
| | | ArGrCrUrArUrGrCrU/AltR2/ | |
| SEQ ID NO: 579 | HPRT38087_UNA15 | /AltR1/rArArUrUrA/iUNA- | crRNA with single UNA base |
| | | rU/rGrGrGrGrArUrUrArCrUrArGrGrArGrUrUrUrUrArGrAr | |
| | | GrCrUrArUrGrCrU/AltR2/ | |
| SEQ ID NO: 580 | HPRT38087_UNA14 | /AltR1/rArArUrUrArU/iUNA- | crRNA with single UNA base |
| | | rG/rGrGrGrArUrUrArCrUrArGrGrArGrUrUrUrUrArGrArGr | |
| | | CrUrArUrGrCrU/AltR2/ | |
| SEQ ID NO: 581 | HPRT38087_UNA13 | /AltR1/rArArUrUrArUrG/iUNA- | crRNA with single UNA base |
| | | rG/rGrGrArUrUrArCrUrArGrGrArGrUrUrUrUrArGrArGrCr | |
| | | UrArUrGrCrU/AltR2/ | |
| SEQ ID NO: 582 | HPRT38087_UNA12 | /AltR1/rArArUrUrArUrGrG/iUNA- | crRNA with single UNA base |
| | | rG/rGrArUrUrArCrUrArGrGrArGrUrUrUrUrArGrArGrCrUr | |
| | | ArUrGrCrU/AltR2/ | |
| SEQ ID NO: 583 | HPRT38087_UNA11 | /AltR1/rArArUrUrArUrGrGrG/iUNA- | crRNA with single UNA base |
| | | rG/rArUrUrArCrUrArGrGrArGrUrUrUrUrArGrArGrCrUrAr | |
| | | UrGrCrU/AltR2/ | |
| SEQ ID NO: 584 | HPRT38087_UNA10 | /AltR1/rArArUrUrArUrGrGrGrG/iUNA- | crRNA with single UNA base |
| | | rA/rUrUrArCrUrArGrGrArGrUrUrUrUrArGrArGrCrUrArUr | |
| | | GrCrU/AltR2/ | |
| SEQ ID NO: 585 | HPRT38087_UNA9 | /AltR1/rArArUrUrArUrGrGrGrGrA/iUNA- | crRNA with single UNA base |
| | | rU/rUrArCrUrArGrGrArGrUrUrUrUrArGrArGrCrUrArUrGr | |
| | | CrU/AltR2/ | |
| SEQ ID NO: 586 | HPRT38087_UNA8 | /AltR1/rArArUrUrArUrGrGrGrGrArU/iUNA- | crRNA with single UNA base |
| | | rU/rArCrUrArGrGrArGrUrUrUrUrArGrArGrCrUrArUrGrCr | |
| | | U/AltR2/ | |
| SEQ ID NO: 587 | HPRT38087_UNA7 | /AltR1/rArArUrUrArUrGrGrGrGrArUrU/iUNA- | crRNA with single UNA base |
| | | rA/rCrUrArGrGrArGrUrUrUrUrArGrArGrCrUrArUrGrCrU/ | |
| | | AltR2/ | |
| SEQ ID NO: 588 | HPRT38087_UNA6 | /AltR1/rArArUrUrArUrGrGrGrGrArUrUrA/iUNA- | crRNA with single UNA base |
| | | rC/rUrArGrGrArGrUrUrUrUrArGrArGrCrUrArUrGrCrU/Alt | |
| | | R2/ | |
| SEQ ID NO: 589 | HPRT38087_UNA5 | /AltR1/rArArUrUrArUrGrGrGrGrArUrUrArC/iUNA- | crRNA with single UNA base |
| | | rU/rArGrGrArGrUrUrUrUrArGrArGrCrUrArUrGrCrU/AltR2 | |
| | | / | |
| SEQ ID NO: 590 | HPRT38087_UNA4 | /AltR1/rArArUrUrArUrGrGrGrGrArUrUrArCrU/iUNA- | crRNA with single UNA base |
| | | rA/rGrGrArGrUrUrUrUrArGrArGrCrUrArUrGrCrU/AltR2/ | |
| SEQ ID NO: 591 | HPRT38087_UNA3 | /AltR1/rArArUrUrArUrGrGrGrGrArUrUrArCrUrA/iUNA- | crRNA with single UNA base |
| | | rG/rGrArGrUrUrUrUrArGrArGrCrUrArUrGrCrU/AltR2/ | |
| SEQ ID NO: 592 | HPRT38087_UNA2 | /AltR1/rArArUrUrArUrGrGrGrGrArUrUrArCrUrArG/iUNA- | crRNA with single UNA base |
| | | rG/rArGrUrUrUrUrArGrArGrCrUrArUrGrCrU/AltR2/ | |
| SEQ ID NO: 593 | HPRT38087_UNA1 | /AltR1/rArArUrUrArUrGrGrGrGrArUrUrArCrUrArGrG/iUN | crRNA with single UNA base |
| | | A-rA/rGrUrUrUrUrArGrArGrCrUrArUrGrCrU/AltR2/ | |
| SEQ ID NO: 594 | B2M_UNA20 | /AltR1//iUNA- | crRNA with single UNA base |
| | | rC/rUrUrArCrCrCrCrArCrUrUrArArCrUrArUrCrUrGrUrUrU | |
| | | rUrArGrArGrCrUrArUrGrCrU/AltR2/ | |
| SEQ ID NO: 595 | B2M_UNA19 | /AltR1/rC/iUNA- | crRNA with single UNA base |
| | | rU/rUrArCrCrCrCrArCrUrUrArArCrUrArUrCrUrGrUrUrUrU | |
| | | rArGrArGrCrUrArUrGrCrU/AltR2/ | |
| SEQ ID NO: 596 | B2M_UNA18 | /AltR1/rCrU/iUNA- | crRNA with single UNA base |
| | | rU/rArCrCrCrCrArCrUrUrArArCrUrArUrCrUrGrUrUrUrUrA | |
| | | rGrArGrCrUrArUrGrCrU/AltR2/ | |
| SEQ ID NO: 597 | B2M_UNA17 | /AltR1/rCrUrU/iUNA- | crRNA with single UNA base |
| | | rA/rCrCrCrCrArCrUrUrArArCrUrArUrCrUrGrUrUrUrUrArG | |
| | | rArGrCrUrArUrGrCrU/AltR2/ | |
| SEQ ID NO: 598 | B2M_UNA16 | /AltR1/rCrUrUrA/iUNA- | crRNA with single UNA base |
| | | rC/rCrCrCrArCrUrUrArArCrUrArUrCrUrGrUrUrUrUrArGrA | |
| | | rGrCrUrArUrGrCrU/AltR2/ | |
| SEQ ID NO: 599 | B2M_UNA15 | /AltR1/rCrUrUrArC/iUNA- | crRNA with single UNA base |
| | | rC/rCrCrArCrUrUrArArCrUrArUrCrUrGrUrUrUrUrArGrAr | |
| | | GrCrUrArUrGrCrU/AltR2/ | |
| SEQ ID NO: 600 | B2M_UNA14 | /AltR1/rCrUrUrArCrC/iUNA- | crRNA with single UNA base |
| | | rC/rCrArCrUrUrArArCrUrArUrCrUrGrUrUrUrUrArGrArGr | |
| | | CrUrArUrGrCrU/AltR2/ | |
| SEQ ID NO: 601 | B2M_UNA13 | /AltR1/rCrUrUrArCrCrC/iUNA- | crRNA with single UNA base |
| | | rC/rArCrUrUrArArCrUrArUrCrUrGrUrUrUrUrArGrArGrCr | |
| | | UrArUrGrCrU/AltR2/ | |
| SEQ ID NO: 602 | B2M_UNA12 | /AltR1/rCrUrUrArCrCrCrC/iUNA- | crRNA with single UNA base |
| | | rA/rCrUrUrArArCrUrArUrCrUrGrUrUrUrUrArGrArGrCrUr | |
| | | ArUrGrCrU/AltR2/ | |
| SEQ ID NO: 603 | B2M_UNA11 | /AltR1/rCrUrUrArCrCrCrCrA/iUNA- | crRNA with single UNA base |
| | | rC/rUrUrArArCrUrArUrCrUrGrUrUrUrUrArGrArGrCrUrAr | |
| | | UrGrCrU/AltR2/ | |
| SEQ ID NO: 604 | B2M_UNA10 | /AltR1/rCrUrUrArCrCrCrCrArC/iUNA- | crRNA with single UNA base |
| | | rU/rUrArArCrUrArUrCrUrGrUrUrUrUrArGrArGrCrUrArUr | |
| | | GrCrU/AltR2/ | |
| SEQ ID NO: 605 | B2M_UNA9 | /AltR1/rCrUrUrArCrCrCrCrArCrU/iUNA- | crRNA with single UNA base |
| | | rU/rArArCrUrArUrCrUrGrUrUrUrUrArGrArGrCrUrArUrGr | |
| | | CrU/AltR2/ | |
| SEQ ID NO: 606 | B2M_UNA8 | /AltR1/rCrUrUrArCrCrCrCrArCrUrU/iUNA- | crRNA with single UNA base |
| | | rA/rArCrUrArUrCrUrGrUrUrUrUrArGrArGrCrUrArUrGrCr | |
| | | U/AltR2/ | |
| SEQ ID NO: 607 | B2M_UNA7 | /AltR1/rCrUrUrArCrCrCrCrArCrUrUrA/iUNA- | crRNA with single UNA base |
| | | rA/rCrUrArUrCrUrGrUrUrUrUrArGrArGrCrUrArUrGrCrU/ | |
| | | AltR2/ | |
| SEQ ID NO: 608 | B2M_UNA6 | /AltR1/rCrUrUrArCrCrCrCrArCrUrUrArA/iUNA- | crRNA with single UNA base |
| | | rC/rUrArUrCrUrGrUrUrUrUrArGrArGrCrUrArUrGrCrU/Alt | |
| | | R2/ | |
| SEQ ID NO: 609 | B2M_UNA5 | /AltR1/rCrUrUrArCrCrCrCrArCrUrUrArArC/iUNA- | crRNA with single UNA base |
| | | rU/rArUrCrUrGrUrUrUrUrArGrArGrCrUrArUrGrCrU/AltR2/ | |
| SEQ ID NO: 610 | B2M_UNA4 | /AltR1/rCrUrUrArCrCrCrCrArCrUrUrArArCrU/iUNA- | crRNA with single UNA base |
| | | rA/rUrCrUrGrUrUrUrUrArGrArGrCrUrArUrGrCrU/AltR2/ | |
| SEQ ID NO: 611 | B2M_UNA3 | /AltR1/rCrUrUrArCrCrCrCrArCrUrUrArArCrUrA/iUNA- | crRNA with single UNA base |
| | | rU/rCrUrGrUrUrUrUrArGrArGrCrUrArUrGrCrU/AltR2/ | |
| SEQ ID NO: 612 | B2M_UNA2 | /AltR1/rCrUrUrArCrCrCrCrArCrUrUrArArCrUrArU/iUNA- | crRNA with single UNA base |
| | | rC/rUrGrUrUrUrUrArGrArGrCrUrArUrGrCrU/AltR2/ | |
| SEQ ID NO: 613 | B2M_UNA1 | /AltR1/rCrUrUrArCrCrCrCrArCrUrUrArArCrUrArUrC/iUN | crRNA with single UNA base |
| | | A-rU/rGrUrUrUrUrArGrArGrCrUrArUrGrCrU/AltR2/ | |
| SEQ ID NO: 614 | APOBEC3A_UNA20 | /AltR1//iUNA- | crRNA with single UNA base |
| | | rC/rGrGrUrCrArArGrArUrGrGrArCrCrArGrCrArCrGrUrUr | |
| | | UrUrArGrArGrCrUrArUrGrCrU/AltR2/ | |
| SEQ ID NO: 615 | APOBEC3A_UNA19 | /AltR1/rC/iUNA- | crRNA with single UNA base |
| | | rG/rGrUrCrArArGrArUrGrGrArCrCrArGrCrArCrGrUrUrUr | |
| | | UrArGrArGrCrUrArUrGrCrU/AltR2/ | |
| SEQ ID NO: 616 | APOBEC3A_UNA18 | /AltR1/rCrG/iUNA- | crRNA with single UNA base |
| | | rG/rUrCrArArGrArUrGrGrArCrCrArGrCrArCrGrUrUrUrUr | |
| | | ArGrArGrCrUrArUrGrCrU/AltR2/ | |
| SEQ ID NO: 617 | APOBEC3A_UNA17 | /AltR1/rCrGrG/iUNA- | crRNA with single UNA base |
| | | rU/rCrArArGrArUrGrGrArCrCrArGrCrArCrGrUrUrUrUrAr | |
| | | GrArGrCrUrArUrGrCrU/AltR2/ | |
| SEQ ID NO: 618 | APOBEC3A_UNA16 | /AltR1/rCrGrGrU/iUNA- | crRNA with single UNA base |
| | | rC/rArArGrArUrGrGrArCrCrArGrCrArCrGrUrUrUrUrArGr | |
| | | ArGrCrUrArUrGrCrU/AltR2/ | |
| SEQ ID NO: 619 | APOBEC3A_UNA15 | /AltR1/rCrGrGrUrC/iUNA- | crRNA with single UNA base |
| | | rA/rArGrArUrGrGrArCrCrArGrCrArCrGrUrUrUrUrArGrAr | |
| | | GrCrUrArUrGrCrU/AltR2/ | |
| SEQ ID NO: 620 | APOBEC3A_UNA14 | /AltR1/rCrGrGrUrCrA/iUNA- | crRNA with single UNA base |
| | | rA/rGrArUrGrGrArCrCrArGrCrArCrGrUrUrUrUrArGrArGr | |
| | | CrUrArUrGrCrU/AltR2/ | |
| SEQ ID NO: 621 | APOBEC3A_UNA13 | /AltR1/rCrGrGrUrCrArA/iUNA- | crRNA with single UNA base |
| | | rG/rArUrGrGrArCrCrArGrCrArCrGrUrUrUrUrArGrArGrCr | |
| | | UrArUrGrCrU/AltR2/ | |
| SEQ ID NO: 622 | APOBEC3A_UNA12 | /AltR1/rCrGrGrUrCrArArG/iUNA- | crRNA with single UNA base |
| | | rA/rUrGrGrArCrCrArGrCrArCrGrUrUrUrUrArGrArGrCrUr | |
| | | ArUrGrCrU/AltR2/ | |
| SEQ ID NO: 623 | APOBEC3A_UNA11 | /AltR1/rCrGrGrUrCrArArGrA/iUNA- | crRNA with single UNA base |
| | | rU/rGrGrArCrCrArGrCrArCrGrUrUrUrUrArGrArGrCrUrAr | |
| | | UrGrCrU/AltR2/ | |
| SEQ ID NO: 624 | APBB2_UNA20 | /AltR1//iUNA- | crRNA with single UNA base |
| | | rU/rUrGrGrGrArCrArArCrGrUrUrGrUrCrCrArGrCrGrUrUr | |
| | | UrUrArGrArGrCrUrArUrGrCrU/AltR2/ | |
| SEQ ID NO: 625 | APBB2_UNA19 | /AltR1/rU/iUNA- | crRNA with single UNA base |
| | | rU/rGrGrGrArCrArArCrGrUrUrGrUrCrCrArGrCrGrUrUrUr | |
| | | UrArGrArGrCrUrArUrGrCrU/AltR2/ | |
| SEQ ID NO: 626 | APBB2_UNA18 | /AltR1/rUrU/iUNA- | crRNA with single UNA base |
| | | rG/rGrGrArCrArArCrGrUrUrGrUrCrCrArGrCrGrUrUrUrUr | |
| | | ArGrArGrCrUrArUrGrCrU/AltR2/ | |
| SEQ ID NO: 627 | APBB2_UNA17 | /AltR1/rUrUrG/iUNA- | crRNA with single UNA base |
| | | rG/rGrArCrArArCrGrUrUrGrUrCrCrArGrCrGrUrUrUrUrAr | |
| | | GrArGrCrUrArUrGrCrU/AltR2/ | |
| SEQ ID NO: 628 | APBB2_UNA16 | /AltR1/rUrUrGrG/iUNA- | crRNA with single UNA base |
| | | rG/rArCrArArCrGrUrUrGrUrCrCrArGrCrGrUrUrUrUrArGr | |
| | | ArGrCrUrArUrGrCrU/AltR2/ | |
| SEQ ID NO: 629 | APBB2_UNA15 | /AltR1/rUrUrGrGrG/iUNA- | crRNA with single UNA base |
| | | rA/rCrArArCrGrUrUrGrUrCrCrArGrCrGrUrUrUrUrArGrAr | |
| | | GrCrUrArUrGrCrU/AltR2/ | |
| SEQ ID NO: 630 | APBB2_UNA14 | /AltR1/rUrUrGrGrGrA/iUNA- | crRNA with single UNA base |
| | | rC/rArArCrGrUrUrGrUrCrCrArGrCrGrUrUrUrUrArGrArGr | |
| | | CrUrArUrGrCrU/AltR2/ | |
| SEQ ID NO: 631 | APBB2_UNA13 | /AltR1/rUrUrGrGrGrArC/iUNA- | crRNA with single UNA base |
| | | rA/rArCrGrUrUrGrUrCrCrArGrCrGrUrUrUrUrArGrArGrCr | |
| | | UrArUrGrCrU/AltR2/ | |
| SEQ ID NO: 632 | APBB2_UNA12 | /AltR1/rUrUrGrGrGrArCrA/iUNA- | crRNA with single UNA base |
| | | rA/rCrGrUrUrGrUrCrCrArGrCrGrUrUrUrUrArGrArGrCrUr | |
| | | ArUrGrCrU/AltR2/ | |
| SEQ ID NO: 633 | APBB2_UNA11 | /AltR1/rUrUrGrGrGrArCrArA/iUNA- | crRNA with single UNA base |
| | | rC/rGrUrUrGrUrCrCrArGrCrGrUrUrUrUrArGrArGrCrUrAr | |
| | | UrGrCrU/AltR2/ | |
| SEQ ID NO: 634 | ADI1_UNA20 | /AltR1//iUNA- | crRNA with single UNA base |
| | | rC/rUrArCrGrArGrGrArGrCrArUrUrUrGrCrArCrUrGrUrUr | |
| | | UrUrArGrArGrCrUrArUrGrCrU/AltR2/ | |
| SEQ ID NO: 635 | ADI1_UNA19 | /AltR1/rC/iUNA- | crRNA with single UNA base |
| | | rU/rArCrGrArGrGrArGrCrArUrUrUrGrCrArCrUrGrUrUrUr | |
| | | UrArGrArGrCrUrArUrGrCrU/AltR2/ | |
| SEQ ID NO: 636 | ADI1_UNA18 | /AltR1/rCrU/iUNA- | crRNA with single UNA base |
| | | rA/rCrGrArGrGrArGrCrArUrUrUrGrCrArCrUrGrUrUrUrUr | |
| | | ArGrArGrCrUrArUrGrCrU/AltR2/ | |
| SEQ ID NO: 637 | ADI1_UNA17 | /AltR1/rCrUrA/iUNA- | crRNA with single UNA base |
| | | rC/rGrArGrGrArGrCrArUrUrUrGrCrArCrUrGrUrUrUrUrAr | |
| | | GrArGrCrUrArUrGrCrU/AltR2/ | |
| SEQ ID NO: 638 | ADI1_UNA16 | /AltR1/rCrUrArC/iUNA- | crRNA with single UNA base |
| | | rG/rArGrGrArGrCrArUrUrUrGrCrArCrUrGrUrUrUrUrArGr | |
| | | ArGrCrUrArUrGrCrU/AltR2/ | |
| SEQ ID NO: 639 | ADI1_UNA15 | /AltR1/rCrUrArCrG/iUNA- | crRNA with single UNA base |
| | | rA/rGrGrArGrCrArUrUrUrGrCrArCrUrGrUrUrUrUrArGrAr | |
| | | GrCrUrArUrGrCrU/AltR2/ | |
| SEQ ID NO: 640 | ADI1_UNA14 | /AltR1/rCrUrArCrGrA/iUNA- | crRNA with single UNA base |
| | | rG/rGrArGrCrArUrUrUrGrCrArCrUrGrUrUrUrUrArGrArGr | |
| | | CrUrArUrGrCrU/AltR2/ | |
| SEQ ID NO: 641 | ADI1_UNA13 | /AltR1/rCrUrArCrGrArG/iUNA- | crRNA with single UNA base |
| | | rG/rArGrCrArUrUrUrGrCrArCrUrGrUrUrUrUrArGrArGrCr | |
| | | UrArUrGrCrU/AltR2/ | |
| SEQ ID NO: 642 | ADI1_UNA12 | /AltR1/rCrUrArCrGrArGrG/iUNA- | crRNA with single UNA base |
| | | rA/rGrCrArUrUrUrGrCrArCrUrGrUrUrUrUrArGrArGrCrUr | |
| | | ArUrGrCrU/AltR2/ | |
| SEQ ID NO: 643 | ADI1_UNA11 | /AltR1/rCrUrArCrGrArGrGrA/iUNA- | crRNA with single UNA base |
| | | rG/rCrArUrUrUrGrCrArCrUrGrUrUrUrUrArGrArGrCrUrAr | |
| | | UrGrCrU/AltR2/ | |

### Example 8. Assessment of UNA Modifications in sgRNAs

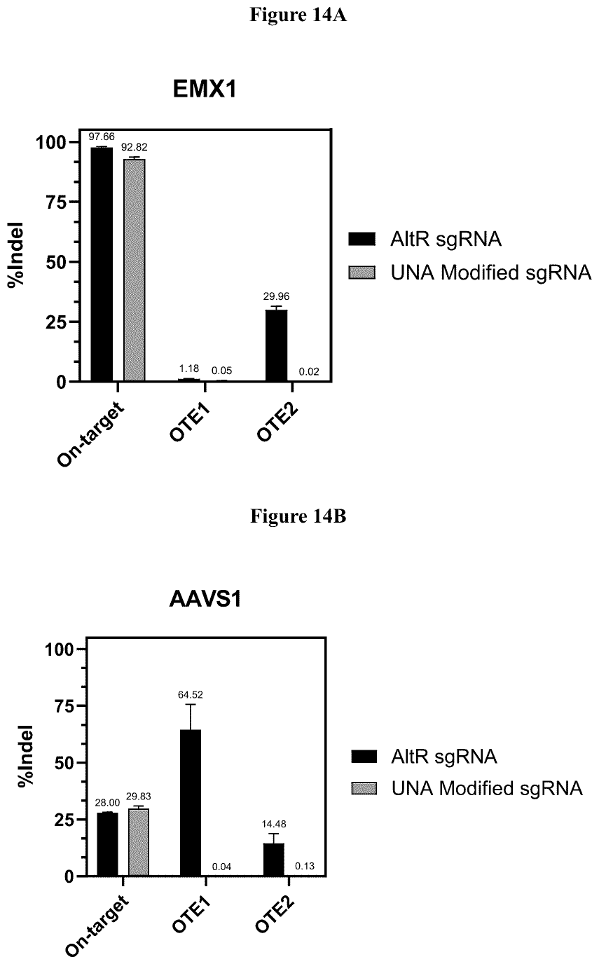

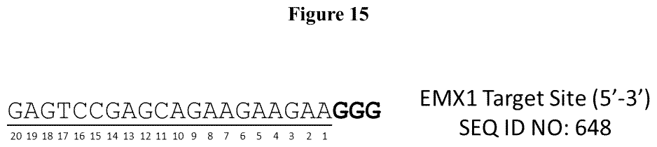

To ensure that UNA modifications showed similar attributes as was seen with 2-part gRNA systems, UNA modified sgRNAs were compared to IDT's standard ALT-R sgRNAs for on/off-target editing in cells (SEQ_ID_644, SEQ_ID_645, SEQ_ID_646, SEQ_ID_647). Cas9 editing was assessed with WT-Cas9 RNP delivery in U2OS cells. In brief, cells were nucleofected with 4 μM RNP, WT-Cas9 V3 (IDT), with 3 SM electroporation enhancer (IDT). All samples were incubated for 72 hrs., gDNA collected with QUICKEXTRACT, the on-target editing site was amplified and prepped for NGS using RHIAMPSEQ and analyzed using CRISPALTRATIONS. As was seen previously with 2-part systems, sgRNAs with UNAs placed at previously optimized positions (UNA position 18 for both EMX1 and AAVS1 target sites) showed on-target editing retention and stark decreases in off-target editing (FIG. 14A-14B). This highlights the importance of the UNA modification placed in the gRNA spacer region for modulation of editing, regardless of the gRNA format being a 2-part or single guide system.

TABLE 11

| Oligos | | | |
|---|---|---|---|
| Se- | | | |
| quence | | | |
| ID | Name | Sequence | Description |
| 644 | EMX1 AltR ™ | mG*mA*mG*rUrCrCrGrArGrCrArGrAr | AltR ™ |
| | sgRNA | ArGrArArGrArArGrUrUrUrUrArGrAr | sgRNA. |
| | | GrCrUrArGrArArArUrArGrCrArArGr | |
| | | UrUrArArArArUrArArGrGrCrUrArGr | |
| | | UrCrCrGrUrUrArUrCrArArCrUrUrGr | |
| | | ArArArArArGrUrGrGrCrArCrCrGrAr | |
| | | GrUrCrGrGrUrGrCmU*mU*mU*rU | |
| 645 | AAVS1 AltR ™ | mG*mG*mG*rGrCrCrArCrUrArGrGrGr | AltR ™ |
| | sgRNA | ArCrArGrGrArUrGrUrUrUrUrArGrAr | sgRNA. |
| | | GrCrUrArGrArArArUrArGrCrArArGr | |
| | | UrUrArArArArUrArArGrGrCrUrArGr | |
| | | UrCrCrGrUrUrArUrCrArArCrUrUrGr | |
| | | ArArArArArGrUrGrGrCrArCrCrGrAr | |
| | | GrUrCrGrGrUrGrCmU*mU*mU*rU | |
| 646 | EMX1 UNA18 | mG*mA*/iUNA-rG/*rUrCrCrGrArGrC | UNA |
| | sgRNA | rArGrArArGrArArGrArArGrUrUrUrU | Modified |
| | | rArGrArGrCrUrArGrArArArUrArGrC | sgRNA |
| | | rArArGrUrUrArArArArUrArArGrGrC | |
| | | rUrArGrUrCrCrGrUrUrArUrCrArArC | |
| | | rUrUrGrArArArArArGrUrGrGrCrArC | |
| | | rCrGrArGrUrCrGrGrUrGrCmU*mU*mU | |
| | | *rU | |
| 647 | AAVS1 UNA18 | mG*mG*/iUNA-rG/*rGrCrCrArCrUrA | UNA |
| | sgRNA | rGrGrGrArCrArGrGrArUrGrUrUrUrU | Modified |
| | | rArGrArGrCrUrArGrArArArUrArGrC | sgRNA |
| | | rArArGrUrUrArArArArUrArArGrGrC | |
| | | rUrArGrUrCrCrGrUrUrArUrCrArArC | |
| | | rUrUrGrArArArArArGrUrGrGrCrArC | |
| | | rCrGrArGrUrCrGrGrUrGrCmU*mU*mU | |
| | | *rU | |

While the invention has been described in detail and with reference to specific examples thereof, it will be apparent to one skilled in the art that various changes and modifications can be made therein without departing from the spirit and scope thereof.

### REFERENCES

1. Jinek, M., Chylinski, K., Fonfara, I., Hauer, M., Doudna, J. A., & Charpentier, E. (2012). A Programmable Dual-RNA-Guided DNA Endonuclease in Adaptive Bacterial Immunity. Science, 337(6096), 816-821.
2. Hsu, P. D., Scott, D. A., Weinstein, J. A., Ran, F. A., Konermann, S., Agarwala, V., Li, Y., Fine, E. J., Wu, X., Shalem, O., Cradick, T. J., Marraffini, L. A., Bao, G., & Zhang, F. (2013). DNA targeting specificity of RNA-guided Cas9 nucleases. Nature Biotechnology, 31(9), 827-832.
3. Fu, Y., Foden, J. A., Khayter, C., Maeder, M. L., Reyon, D., Joung, J. K., & Sander, J. D. (2013). High-frequency off-target mutagenesis induced by CRISPR-Cas nucleases in human cells. Nature Biotechnology, 31(9), 822-826.
4. Doench, J. G., Fusi, N., Sullender, M., Hegde, M., Vaimberg, E. W., Donovan, K. F., Smith, I., Tothova, Z., Wilen, C., Orchard, R., Virgin, H. W., Listgarten, J., & Root, D. E. (2016). Optimized sgRNA design to maximize activity and minimize off-target effects of CRISPR-Cas9. Nature Biotechnology, 34(2), 184-191.
5. Hendel, A., Bak, R. O., Clark, J. T., Kennedy, A. B., Ryan, D. E., Roy, S., Steinfeld, I., Lunstad, B. D., Kaiser, R. J., Wilkens, A. B., Bacchetta, R., Tsalenko, A., Dellinger, D., Bruhn, L., & Porteus, M. H. (2015). Chemically modified guide RNAs enhance CRISPR-Cas genome editing in human primary cells. Nature Biotechnology, 33(9), 985-989.
6. Fu, Y., Sander, J. D., Reyon, D., Cascio, V. M., & Joung, J. K. (2014). Improving CRISPR-Cas nuclease specificity using truncated guide RNAs. Nature Biotechnology, 32(3), 279-284.
7. Vakulskas, C. A., Dever, D. P., Rettig, G. R., Turk, R., Jacobi, A. M., Collingwood, M. A., Bode, N. M., McNeill, M. S., Yan, S., Camarena, J., Lee, C. M., Hyun Park, S., Wiebking, V., Bak, R. O., Gomez-Ospina, N., Pavel-Dinu, M., Sun, W., Bao, G., Porteus, M. H., & Behlke, M. A. (2018). A high-fidelity Cas9 mutant delivered as a ribonucleoprotein complex enables efficient gene editing in human hematopoietic stem and progenitor cells. Nature Medicine.
8. Donohoue, P. D., Pacesa, M., Lau, E., Vidal, B., Irby, M. J., Nyer, D. B., Rotstein, T., Banh, L., Toh, M. S., Gibson, J., Kohrs, B., Baek, K., Owen, A. L. G., Slorach, E. M., van Overbeek, M., Fuller, C. K., May, A. P., Jinek, M., & Cameron, P. (2021). Conformational control of Cas9 by CRISPR hybrid RNA-DNA guides mitigates off-target activity in T cells. Molecular Cell, 81(17), 3637-3649.e5.
9. Pasternak, A., Hernandez, F. J., Rasmussen, L. M., Vester, B., & Wengel, J. (2011). Improved thrombin binding aptamer by incorporation of a single unlocked nucleic acid monomer. Nucleic Acids Research, 39(3), 1155-1164. doi.org/10.1093/nar/gkg823
10. Braasch, D. A., & Corey, D. R. (2001). Locked nucleic acid (LNA): fine-tuning the recognition of DNA and RNA. Chemistry & Biology, 8(1), 1-7.
11. Manoharan, M., Akinc, A., Pandey, R. K., Qin, J., Hadwiger, P., John, M., Mills, K., Charisse, K., Maier, M. A., Nechev, L., Greene, E. M., Pallan, P. S., Rozners, E., Rajeev, K. G., & Egli, M. (2011). Unique Gene-Silencing and Structural Properties of 2′-Fluoro-Modified siRNAs. Angewandte Chemie International Edition, 50(10), 2284-2288.
12. Fontenete, S., Guimaraes, N., Leite, M., Figueiredo, C., Wengel, J., & Filipe Azevedo, N. (2013). Hybridization-Based Detection of Helicobacter pylori at Human Body Temperature Using Advanced Locked Nucleic Acid (LNA) Probes. PLOS ONE, 8(11), e81230-.
13. Jun, W. J., Park, S.-J., Cha, S. & Kim, K. Factors affecting the cleavage efficiency of the CRISPR-Cas9 system. Anim. Cells Syst. 28, 75-83 (2024).
14. Rieenberg, S., Helmbrecht, N., Kanis, P., Maricic, T. & Pääbo, S. Improved gRNA secondary structures allow editing of target sites resistant to CRISPR-Cas9 cleavage. Nat. Commun. 13, 489 (2022).
15. Kuran, G. et al. CRISPAltRations: A validated cloud-based approach for interrogation of double-strand break repair mediated by CRISPR genome editing. Mol. Ther.—Methods Clin. Dev. 21, 478-491 (2021).
16. Kinney, K. J. et al. UNCOVERseq Enables Sensitive and Controlled Gene Editing Off-Target Nomination Across CRISPR-Cas Modalities and Systems. bioRxiv 2025.05.09.653165 (2025) doi:10.1101/2025.05.09

1. A synthetic guide RNA comprising:
(i) a first nucleotide sequence comprising at least one modified nucleotide, wherein the first nucleotide is partially or completely complementary to a target nucleic acid; and
(ii) a second nucleic acid sequence which interacts with a CRISPR-associated protein (Cas) polypeptide,
wherein the synthetic guide RNA guides the Cas polypeptide to the target nucleic acid, and wherein the synthetic guide RNA exhibits a reduced off-target effect and/or enhanced on-target editing activity, relative to an unmodified gRNA.

2. The synthetic guide RNA of claim 1, wherein the at least one modified nucleotide is selected from the group consisting of unlocked nucleic acid (UNA), locked nucleic acid (LNA), 2′fluoro, C3 spacer, dSpacer, and combinations thereof.

3. The synthetic guide RNA of claim 1, wherein the first nucleotide sequence and second nucleotide sequence are a single nucleic acid strand.

4. The synthetic guide RNA of claim 1, wherein the first nucleotide sequence and second nucleotide sequence are two separate nucleic acid strands.

5. The synthetic guide RNA of claim 1, wherein the first nucleotide sequence is about 14-25 nucleotides in length.

6. The synthetic guide RNA of claim 1, wherein the at least one modified nucleotide is present at a position selected from the group consisting of nucleotide 1, nucleotide 2, nucleotide 3, nucleotide 4, nucleotide 5, nucleotide 6, nucleotide 7, nucleotide 8, nucleotide 9, nucleotide 10, nucleotide 11, nucleotide 12, nucleotide 13, nucleotide 14, nucleotide 15, nucleotide 16, nucleotide 17, nucleotide 18, nucleotide 19, nucleotide 20, and combinations thereof, wherein each position is labeled in the first nucleic acid sequence starting at nucleotide 1 from the PAM adjacent base.

7. The synthetic guide RNA of claim 1, wherein off-target editing relative to an unmodified gRNA is reduced by at least an amount selected from the group consisting of 50%, 60%, 70%, 80%, 85%, 90%, 95%, and 99%.

8. The synthetic guide RNA of claim 1, wherein the first nucleotide sequence comprises a 3′ modification.

9. The synthetic guide RNA of claim 1, wherein the first nucleotide sequence comprises a 5′ modification.

10. The synthetic guide RNA of claim 1, wherein the at least one modified nucleotide alters base-pairing thermostability.

11. The synthetic guide RNA of claim 1, wherein said at least one modified nucleotide enhances base-pairing thermostability.

12. The synthetic guide RNA of claim 1, wherein said at least one modified nucleotide decreases base-pairing thermostability.

13. The synthetic guide RNA of claim 1, wherein the at least one modified nucleotide is a specificity-altering modification.

14. The synthetic guide RNA of claim 1, wherein the specificity-altering at least one modified nucleotide is located in the guide sequence.

15. The synthetic guide RNA of claim 1, wherein at least two nucleotides in the first nucleotide sequence are modified nucleotides.

16. The synthetic guide RNA of claim 1, wherein one or more modified nucleotides are located within five nucleotides from the 5′-end of the first nucleotide sequence.

17. The synthetic guide RNA of claim 1, wherein from about 10% to about 30% of the nucleotides in the first nucleotide sequence are modified nucleotides.

18. The synthetic guide RNA of claim 1, wherein the at least one modified nucleotide is located within five nucleotides from the 3′-end of the second nucleotide sequence.

19. The synthetic guide RNA of claim 1, wherein the modified sgRNA comprises one, two, or three consecutive or non-consecutive modified nucleotides at or near the 5′-end of the first nucleotide sequence and one, two, or three consecutive or non-consecutive modified nucleotides at or near the 3′-end of the second nucleotide sequence.

20. The synthetic guide RNA of claim 1, wherein the modified sgRNA comprises three consecutive modified nucleotides at the 5′-end of the first nucleotide sequence and three consecutive modified nucleotides at the 3′-end of the second nucleotide sequence.

21-40. (canceled)
